(19) United States
(12) Patent Application Publication
(10) Pub. No.: US 2023/0210980 A1
(43) Pub. Date: Jul. 6, 2023

(54) CHIMERIC ADENOVIRAL VECTORS

(71) Applicant: Vaxart, Inc., South San Francisco, CA (US)

(72) Inventors: Sean Tucker, San Francisco, CA (US); Emery Dora, Pleasant Hill, CA (US)

(21) Appl. No.: 18/000,638

(22) PCT Filed: Jun. 4, 2021
(86) PCT No.: PCT/US21/35930

§ 371 Date: Dec. 2, 2022

Related U.S. Application Data
(60) Provisional application No. 63/035,490, filed on Jun. 5, 2020.
(60) Provisional application No. 63/045,710, filed on Jun. 29, 2020.
(60) Provisional application No. 63/074,954, filed on Sep. 4, 2020.
(60) Provisional application No. 63/144,339, filed on Feb. 1, 2021.

(51) Int. Cl. A61K 39/215; C12N 15/86; A61P 31/14

(52) U.S. Cl. CPC A61K 39/215; C12N 15/86; A61P 31/14; C12N 2710/10343; C12N 2770/20022; C12N 2770/20034; A61K 2039/53

(57) ABSTRACT

The present disclosure provides chimeric adenoviral vectors comprising a nucleic acid encoding a coronavirus disease 2019 (COVID-19) protein and an adjuvant and methods for using the vectors to elicit an immune response to the SARS-CoV-2 protein in order to treat COVID-19.

### CROSS-REFERENCE TO RELATED APPLICATIONS

This application claims priority benefit to U.S. Provisional Application No. 63/144,339, filed Feb. 1, 2021, U.S. Provisional Application No. 63/074,954, filed Sep. 4, 2020; U.S. Provisional Application No. 63/045,710, filed Jun. 29, 2020; and U.S. Provisional Application No. 63/035,490, filed Jun. 5, 2020; each of which is incorporated by reference herein for all purposes.

### BACKGROUND OF THE DISCLOSURE

Coronavirus disease 2019 (COVID-19) is an infectious disease caused by severe acute respiratory syndrome coronavirus 2 (SARS-CoV-2). Some symptoms of the disease include, for example, fever, cough, shortness of breath, muscle pain, sputum production, diarrhea, sore throat, loss of smell, and abdominal pain. While the majority of cases result in mild symptoms, some progress to viral pneumonia and multi-organ failure. The disease currently has no cure and has spread rapidly across several continents, with community outbreaks throughout the world.

### SUMMARY OF THE DISCLOSURE

In one aspect, the disclosure provides a chimeric adenoviral expression vector, comprising an expression cassette comprising the following elements: (a) a first promoter operably linked to a nucleic acid encoding a first severe acute respiratory syndrome coronavirus 2 (SARS-CoV-2) protein; and (b) a second promoter operably linked to a nucleic acid encoding a toll-like receptor-3 (TLR-3) agonist.

In some embodiments, the nucleic acid encoding the TLR-3 agonist comprises a nucleic acid encoding a dsRNA. In some embodiments, the nucleic acid encoding the TLR-3 agonist comprises a sequence selected from the group consisting of: SEQ ID NOS:13-20. In particular embodiments, the nucleic acid encoding the TLR-3 agonist comprises a sequence of SEQ ID NO:13.

Further, the chimeric adenoviral expression vector can comprise additional element (c): a third promoter operably linked to a nucleic acid encoding a second SARS-CoV-2 protein. In some embodiments, element (c) is placed between elements (a) and (b) in the expression cassette. In certain embodiments, the first SARS-CoV-2 protein in (a) and the second SARS-CoV-2 protein in (c) are different. In other embodiments, the SARS-CoV-2 protein in (a) and the SARS-CoV-2 protein in (c) are the same.

In some embodiments of this aspect, the nucleic acid encoding the first SARS-CoV-2 protein in element (a) and/or the nucleic acid encoding the second SARS-CoV-2 protein in element (c) comprises a sequence having at least 85%, 90%, 95%, 97%, 99%, or 100% identity to the sequence of SEQ ID NO:3. In some embodiments, the first and/or second SARS-CoV-2 protein comprises a SARS-CoV-2 S protein having a sequence with at least 85%, 90%, 95%, 97%, 99%, or 100% identity to the sequence of SEQ ID NO:1 or SEQ ID NO:21 or SEQ ID NO:22. In some embodiments, the nucleic acid encoding the first SARS-CoV-2 protein in element (a) and/or the nucleic acid encoding the second SARS-CoV-2 protein in element (c) comprises a sequence having at least 85%, 90%, 95%, 97%, 99%, or 100% identity to the sequence of SEQ ID NO:23.

In some embodiments, the nucleic acid encoding the first SARS-CoV-2 protein in element (a) and/or the nucleic acid encoding the second SARS-CoV-2 protein in element (c) comprises a sequence having at least 85%, 90%, 95%, 97%, 99/o, or 100% identity to the sequence of SEQ ID NO:4. In some embodiments, the first and/or the second SARS-CoV-2 protein comprises a SARS-CoV-2 N protein having a sequence with at least 85%, 90%, 95%, 97%, 99%, or 100% identity to the sequence of SEQ ID NO:2.

In some embodiments of this aspect, the nucleic acid encoding the first SARS-CoV-2 protein in element (a) and/or the nucleic acid encoding the second SARS-CoV-2 protein in element (c) comprises a sequence having at least 85%, 90%, 95%, 97%, 99%, or 100% identity to the sequence of SEQ ID NO:5. In some embodiments, the first and/or second SARS-CoV-2 protein comprises a fusion protein comprising a S1 region of a SARS-CoV-2 S protein, a furin site, and a SARS-CoV-2 N protein, and wherein the fusion protein comprises a sequence having at least 85% identity to the sequence of SEQ ID NO:12.

Moreover, the first promoter and the second promoter in the chimeric adenoviral vector can be identical or different. For example, the first promoter and the second promoter each can be a CMV promoter.

In some embodiments of the aspect, when all three elements (a)-(c) are present, the first promoter can be a CMV promoter, the second promoter can be a CMV promoter, and the third promoter can be a beta-actin promoter (e.g., a human beta-actin promoter).

In another aspect, the disclosure features a chimeric adenoviral expression vector, comprising an expression cassette comprising the following elements: (a) a first promoter operably linked to a nucleic acid encoding a SARS-CoV-2 S protein; and (b) a second promoter operably linked to a nucleic acid encoding a toll-like receptor-3 (TLR-3) agonist.

In some embodiments of this aspect, the nucleic acid encoding the SARS-CoV-2 S protein comprises the sequence of SEQ ID NO:3. In some embodiments, the SARS-CoV-2 S protein comprises the sequence of SEQ ID NO:1 or SEQ ID NO: 21 or SEQ ID NO:22.

In some embodiments, the first promoter and the second promoter are each a CMV promoter.

In some embodiments of this aspect, the elements (a) and (b) together are encoded by the sequence of SEQ ID NO:6. Further, the chimeric adenoviral expression vector of this aspect is encoded by the sequence of SEQ ID NO:9.

In another aspect, the disclosure features a chimeric adenoviral expression vector, comprising an expression cassette comprising the following elements: (a) a first promoter operably linked to a nucleic acid encoding a SARS-CoV-2 S protein; (b) a second promoter operably linked to a nucleic acid encoding a toll-like receptor-3 (TLR-3) agonist; and (c) a third promoter operably linked to a nucleic acid encoding a SARS-CoV-2 N protein, optionally in which the order of the elements in the expression cassette from the N-terminus to the C-terminus is: element (a), element (c), and element (b).

In some embodiments of this aspect, the nucleic acid encoding the SARS-CoV-2 S protein comprises the sequence of SEQ ID NO:3. In some embodiments of this aspect, the nucleic acid encoding the SARS-CoV-2 S protein comprises the sequence of SEQ ID NO:23. In some embodiments, the SARS-CoV-2 S protein comprises the sequence of SEQ ID NO:1 or SEQ ID NO:21 or SEQ ID NO:22.

In some embodiments of this aspect, the nucleic acid encoding the SARS-CoV-2 N protein comprises the sequence of SEQ ID NO:4. In some embodiments, the SARS-CoV-2 N protein comprises the sequence of SEQ ID NO:2.

Further, in some embodiments of this aspect, the first promoter in element (a) is a CMV promoter, the second promoter in element (b) is a CMV promoter, and the third promoter in element (c) is a beta-actin promoter (e.g., a human beta-actin promoter).

In some embodiments, the elements (a), (b), and (c) together are encoded by the sequence of SEQ ID NO:7. Further, the chimeric adenoviral expression vector of this aspect is encoded by the sequence of SEQ ID NO:10.

In another aspect, the disclosure features a chimeric adenoviral expression vector, comprising an expression cassette comprising the following elements: (a) a first promoter operably linked to a nucleic acid encoding a SARS-CoV-2 fusion protein, wherein the SARS-CoV-2 fusion protein comprises a S1 region of a SARS-CoV-2 S protein, a furin site, and a SARS-CoV-2 N protein; and (b) a second promoter operably linked to a nucleic acid encoding a toll-like receptor-3 (TLR-3) agonist.

In some embodiments of this aspect, the nucleic acid encoding the SARS-CoV-2 fusion protein comprises the sequence of SEQ ID NO:5. In some embodiments, the SARS-CoV-2 fusion protein comprises the sequence of SEQ ID NO:12.

In some embodiments of this aspect, the first promoter and the second promoter are each a CMV promoter.

In some embodiments of this aspect, the elements (a) and (b) together are encoded by the sequence of SEQ ID NO:8. Further, the chimeric adenoviral expression vector of this aspect is encoded by the sequence of SEQ ID NO:11.

In another aspect, the disclosure features an immunogenic composition comprising a chimeric adenoviral expression vector described herein and a pharmaceutically acceptable carrier.

In a further aspect, the disclosure additionally features a chimeric adenoviral expression vector, comprising an expression cassette comprising the following elements: (a) a first promoter operably linked to a nucleic acid encoding a first severe acute respiratory syndrome coronavirus 2 (SARS-CoV-2) protein; (b) a second promoter operably linked to a nucleic acid encoding a toll-like receptor-3 (TLR-3) agonist; and (c) a third promoter operably linked to a nucleic acid encoding a SARS-CoV-2 N protein. In some embodiments, the SARS-CoV-2 N protein comprises an amino acid sequence having at least 95%, 96%, 97%, 98%, 99%, or 100% identity to the amino acid sequence of SEQ ID NO:2. In some embodiments, element (c) is situated between elements (a) and (b) in the expression cassette. In some embodiments, the first SARS-CoV-2 protein comprises a SARS-CoV-2 S protein having a sequence with at least 95%, 96%, 97%, 98%, 99%, or 100/6 identity to the sequence of SEQ ID NO:1 or SEQ ID NO: 21 or SEQ ID NO:22. In some embodiments, the nucleic acid encoding the TLR-3 agonist comprises a nucleic acid encoding a dsRNA. In some embodiments, the nucleic acid encoding the TLR-3 agonist comprises a sequence selected from the group consisting of: SEQ ID NOS:13-20. In some embodiments, the nucleic acid encoding the first SARS-CoV-2 protein in element (a) comprises a sequence having at least 85%, 90%, 95%, 97%, 99%, or 100% identity to the sequence of SEQ ID NO:3. In some embodiments, the nucleic acid encoding the SARS-CoV-2 N protein comprises a sequence having at least 85%, 90%, 95%, 97%, 99%, or 100% identity to the sequence of SEQ ID NO:4. In some embodiments, the first promoter and the second promoter are identical. In some embodiments, the first promoter and the second promoter are each a CMV promoter. In some embodiments, the first promoter is a CMV promoter, the second promoter is a CMV promoter, and the third promoter is a beta-actin promoter. In some embodiments, element (c) is situated between elements (a) and (b), and elements (a), (c), and (b) together are encoded by a sequence having at least 95% identity to SEQ ID NO:7 or is encoded by the sequence of SEQ ID NO:7. In some embodiments, the chimeric adenoviral expression vector comprises a sequence having at least 95% identity to SEQ ID NO:10 or comprises the sequence of SEQ ID NO:10.

In another aspect, the disclosure provides a method for eliciting an immune response towards a SARS-CoV-2 protein (e.g., a SARS-CoV-2 protein having the sequence of SEQ ID NOS:1, 2, or 12, or a variant thereof as described herein (e.g., having at least 90% or at least 95% identity to SEQ ID NO:1, 2, or 12) in a subject, comprising administering to the subject an immunogenically effective amount of a chimeric adenoviral expression vector described herein or an immunogenic composition described herein. In some embodiments, the route of administration is oral, intranasal, or mucosal (e.g., oral). In certain embodiments, the route of administration is oral delivery by swallowing a tablet.

In some embodiments of the method, the immune response is elicited in an alveolar cell, an absorptive enterocyte, a ciliated cell, a goblet cell, a club cells, and/or an airway basal cell of the subject. In certain embodiments, the subject is a human.

Also provided is a chimeric polynucleotide (which can be used to induce an immune response in a subject, including but not limited to a CD8 T-cell response), comprising an expression cassette comprising the following elements: (a) a first promoter operably linked to a nucleic acid encoding a first severe acute respiratory syndrome coronavirus 2 (SARS-CoV-2) protein; and (b) a second promoter operably linked to a nucleic acid encoding a toll-like receptor-3 (TLR-3) agonist; and (c) a third promoter operably linked to a nucleic acid encoding a SARS-CoV-2 protein or a non-SARS-CoV-2 antigenic protein.

In some embodiments, the chimeric polynucleotide is a chimeric adenoviral expression vector. In some embodiments, the nucleic acid encoding the TLR-3 agonist comprises a nucleic acid encoding a dsRNA. In some embodiments, the nucleic acid encoding the TLR-3 agonist comprises a sequence selected from the group consisting of: SEQ ID NOS:13-20 In some embodiments, element (c) is placed between elements (a) and (b) in the expression cassette.

In a further aspect, the disclosure provides a chimeric polynucleotide, comprising an expression cassette comprising the following elements: (a) a first promoter operably linked to a nucleic acid encoding an antigenic protein; (b) a second promoter operably linked to a nucleic acid encoding a toll-like receptor-3 (TLR-3) agonist; and (c) a third promoter operably linked to a nucleic acid encoding a SARS-CoV-2 N-protein. In some embodiments, the SARS-CoV-2 N protein has at least 95%, 96%, 97%, 98%, 99%, or 100% identity to SEQ ID NO:2. In some embodiments, the chimeric polynucleotide is a chimeric adenoviral expression vector. In some embodiments, the nucleic acid encoding the TLR-3 agonist comprises a nucleic acid encoding a dsRNA. In some embodiments, the nucleic acid encoding the TLR-3 agonist comprises a sequence selected from the group consisting of: SEQ ID NOS:13-20. In some embodiments, element (c) is placed between elements (a) and (b) in the expression cassette. In some embodiments, the antigenic protein is from a bacteria, fungus, virus, or parasite. In some embodiments, the antigenic protein is a cancer antigen.

In a further aspect, the disclosure provides a method of inducing an immune response in a subject, the method comprising administering a chimeric polynucleotide as seat forth in the preceding paragraph to a subject.

### BRIEF DESCRIPTION OF THE DRAWINGS

FIG. 1 shows the expression of the antigens in human cells post infection.

FIG. 2 shows the IgG antibody titers to S1 following immunization of mice on days 0 and 14. Titers measured by standard ELISA.

FIGS. 3A and 3B show the IgG antibody titers to S1 and S2 following immunization of mice on days 0 and 14. MSD was used to measure the binding signal at multiple time points for both antigens. There were no significant differences in the signal at early timepoints, but more antibody responses were detected at the higher dose groups at later time points.

FIG. 4A-4D. Transgene inserts developed to test vaccine specific responses. Recombinant adenoviruses were made using these inserts a. rAd-S b. rAd-S-N c. rAd-S1-N d. rAd-S(fixed)-N.

FIGS. 5A-5D. Immunization with candidate rAd vaccines induce serum IgG and lung IgA responses. Antibody titers to S following immunization of Balb/c mice on days 0 and 14 with 1×108 IU rAd expressing full-length S (rAd-S), co-expressing full length S and N (rAd-S-N) or co-expressing a fusion protein comprising the S1 domain and N (rAd-S1-N). (FIG. 5A) IgG serum IgG endpoint titers to S1 were measured by standard ELISA (n=6 per vaccinated group, n=3 for PBS administered group). Symbols represent mean titers and bars represent the standard error) (FIG. 5B) Neutralizing antibody responses comparing rAd-S-N and rAd-S1-N using two different methods, surrogate VNT (sVNT) and cell-based VNT (cVNT). (FIG. 5C) IgA lung antibody titers to S1 and S2 in immunized mice. Endpoint titers were measured by standard ELISA (n=10 per group). Lines represent the median and inter-quartile range. ** p<0.01, *** p<0.001 defined by Mann-Whitney t-test. FIG. 5D. Neutralizing antibodies measured in the lungs post immunization.

FIGS. 6A-6B. Immunization with rAd co-expressing full length S and N vaccines induce IgG responses in a dose-dependent manner. FIGS. 6A and 6B. Balb/c mice were immunized, IN, on days 0 and 14 with 1×10⁷ IU, 1×10⁸ IU or 7.2×10⁸ IU of rAd co-expressing full length S and N (rAd-S-N). The amount of IgG specific for S1 (FIG. 6A) and S2 in serum diluted 1/4000, was evaluated using a Mesoscale binding assay. Points represent the mean and lines represent the standard deviation.

FIGS. 7A-7C. Immunization with rAd co-expressing full length S and N vaccines induce polyfunctional T cell responses in a dose-dependent manner. (FIG. 7A) Balb/c mice were immunized, IN, on days 0 and 14 with 1×10⁸ HU (Ad-S-N high), 1×10′ IU (Ad-S-N low) of rAd-S-N. The frequency of CD4+(top panel) or CD8+ T cells (bottom panel) that produced only IFN-γ, TNF-α, IL-2 or IL-4 after stimulation of spleen cells with 1 μg/ml (CD4+) or 5 μg/ml (CD8+) of the S peptide pools, as determined by ICS-FACS. (B) The frequency of polyfunctional CD4+ (top panel) or CD8+ T cells (bottom panel) that produced more than one cytokine after stimulation of spleen cells with 1 μg/ml (CD4+) or 5 μg/ml (CD8+) S peptide pools, Bars represent the mean and the lines represent the standard error of the mean. (C) IFN-γ T cell responses to S protein 4 weeks following immunization on weeks 0 and 4 with 1×10⁶ IU, 1×10⁷ IU, 1×10⁸ LU doses of rAd-S-N were measured by ELISPOT. Bars represent the mean and the lines represent the standard deviation. * p<0.05; one-way non-parametric ANOVA with multiple comparisons.

FIGS. 8A-8B: Antibodies to S were superior when the S protein expressed in the wild-type configuration compared to the fixed version. Balb/c mice were immunized on weeks 0 and 4 with 1e8 IU per mouse (n=6), and antibody titers were measured. (FIG. 8A) IgG antibody titers over time. (FIG. 8B) Neutralizing antibody responses were measured at week 6. Note that 1:1000 was the maximum dilution performed.

FIGS. 9A-9F: (FIG. 9A) (left) Frequency of CD27++CD38++ plasmablasts in peripheral blood before (day 1) and after (day 8) vaccination as measured by flow cytometry. Bars represent median values, while error bars correspond to 95% confidence intervals. Wilcoxon test was used to compare frequencies before and after vaccination; (right) Representative flow cytometry plot showing pre- and day 8 post-vaccination CD27++ CD38++ plasmablasts for one vaccine; (FIG. 9B) Fold change (day 8/day 1) in plasmablast frequencies. A total of 24/35 subjects (69%) showed a 2-fold or higher increase (with a 3.3 median fold change increase overall); (FIG. 9C) Fold change (day 8/day 1) of IgA- and B7-expressing plasmablasts in low and high dose vaccine cohorts. Mann-Whitney test was used to compare frequencies between the two different dose groups; (FIG. 9D) Fold change (day 8/day 1) in the number of IgA-positive antibody-secreting cells (ASC) reactive against the S1 domain of the Sars-CoV-2 spike antigen; (FIG. 9E) Fold change (day 29/day 1) in S-, N-, or RBD-specific IgA antibodies in the serum as measured by MSD platform. Red dotted lines represent median values. Mann-Whitney test was used to compare frequencies between the two different dose groups; (FIG. 9F) Fold change (day 29/day 1) in S-, N-, or RBD-specific IgA antibodies in nasal and saliva samples as measured by MSD platform.

FIGS. 10A-10F: (FIG. 10A) PBMCs pre- and post-immunization were restimulated with SARS-CoV-2 peptides, surface stained for CD4, CD8 and degranulation marker CD107a and intracellularly stained for cytokines. IFNγ, TNFα, and CD107a percent of CD8 T cells increase over background post immunization in response to SARS-CoV-2 Spike protein; (FIG. 10B) IFNγ, TNFα, and CD107a percent of CD4 T cells increase over background post immunization in response to SARS-CoV-2 Spike protein; (FIG. 10C) Increase in IFNγ-producing CD8 T cells post immunization on day 8 versus day 1: (FIG. 10D) Polarization toward Th1 responses versus Th2 responses in subjects immunized by VXA-CoV2-1. Fold change over baseline is displayed; (FIG. 10E) IFNγ, TNFα, and CD107a percent of CD8 T cells increase over background post immunization in response to SARS-CoV-2 Nucleoprotein; (FIG. 10F) IFNγ, TNFα, and CD107a percent of CD4 T cells increase over background post immunization in response to SARS-CoV-2 Nucleoprotein.

FIG. 11: IFNγ, TNFα, and CD107a percent of CD8 T cells increase over background post immunization in response to S&N peptides from 4 endemic coronaviruses

FIGS. 12A-12D. Oral VXA-CoV-2 elicits anti-viral CD8 T cells of higher magnitude than intramuscular mRNA vaccines. PBMCs pre- and post-immunization were restimulated with SARS-CoV-2 peptides, surface stained for CD4, CD8 and degranulation marker CD107a and intracellularly stained for cytokines. PBMCs from all 3 vaccines were evaluated at the same time. (FIG. 12A) Graph shows IFNγ, TNFα, and CD107a percent of CD8 T cells increase over background post immunization in response to SARS-CoV-2 Spike protein (FIG. 12B) IFNγ data from (FIG. 12A) is plotted alongside vaxart cohort and convalescents. Convalescent subjects are not day 1 subtracted due to no pre-infection samples obtained. (FIG. 12C) Representative facs plots comparing the three vaccines. FIG. 12 (D) Timecourse of Pfizer and Moderna vaccines

### DETAILED DESCRIPTION OF THE DISCLOSURE

### I. Introduction

Coronavirus disease 2019 (COVID-19) is an infectious disease caused by severe acute respiratory syndrome coronavirus 2 (SARS-CoV-2). SARS-CoV-2 is a mucosal viral pathogen that infects the epithelial cells of the lungs and possibly even the intestine (9). Some symptoms of the disease include, for example, fever, cough, shortness of breath, muscle pain, sputum production, diarrhea, sore throat, loss of smell, and abdominal pain. While the majority of cases result in mild symptoms, some progress to viral pneumonia and multi-organ failure.

The virus is spread mainly through close contact and via respiratory droplets produced when people cough or sneeze. People may also contract COVID-19 by touching a contaminated surface and then their face. The infection is most contagious when people are symptomatic, although spread may be possible before symptoms appear. Currently, there is no vaccine or specific antiviral treatment for COVID-19. Managing the disease involves treatment of symptoms, supportive care, isolation, and some experimental measures.

The genome of SARS-CoV-2 virus encodes four major structural proteins including spike (S), nucleocapsid (N), membrane (M), and envelope (E), which are required to make a complete virus particle. After viral entry, 16 non-structural proteins are formed from two large precursor proteins. These viruses have a relatively large positive sense RNA strand (26-32 kb), and without erroneous editing, the RNA can mutate, evolve, and undergo homologous recombination with other family members to create new viral species (6). The S protein is believed to be the major antibody target for coronavirus vaccines, as the protein is responsible for receptor binding, membrane fusion, and tissue tropism. When comparing SARS-CoV-2 Wu-1 (GenBank Accession No. QHD43416.1) to SARS-CoV (GenBank Accession No. AY525636.1), the S protein was found to have 76.2% identity, 87.2% similarity, and 2% gaps in 1273 positions (7). Both SARS-CoV and SARS-CoV-2 are believed to use the same receptor for cell entry: the angiotensin-converting enzyme 2 receptor (ACE2), which is expressed on some human cell types (8). As discussed in the article by Xu, et al., high expression levels of ACE2 are present in type II alveolar cells the lungs, absorptive enterocytes of the ileum and colon, and possibly even in oral tissues such as the tongue (9).

Provided herein are vaccines, immunogenic compositions, and methods for treating COVID-19 that involve the use of chimeric adenoviral vectors that contain one or more nucleic acids encoding one or more SARS-CoV-2 proteins and a nucleic acid encoding a TLR-3 agonist.

### II. Definitions

The term “chimeric” or “recombinant” as used herein with reference, e.g., to a nucleic acid, protein, or vector, indicates that the nucleic acid, protein or vector, has been modified by the introduction of a heterologous nucleic acid or protein or the alteration of a native nucleic acid or protein. Thus, for example, chimeric and and recombinant vectors include nucleic acid sequences that are not found within the native (non-chimeric or non-recombinant) form of the vector. A chimeric adenoviral expression vector refers to an adenoviral expression vector comprising a nucleic acid sequence encoding a heterologous polypeptide, such as a SARS-CoV-2 protein.

The term “expression vector” refers to a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The term “promoter” refers to an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as many as several thousand base pairs from the start site of transcription. Promoters include constitutive and inducible promoters. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “SARS-CoV-2” or “severe acute respiratory syndrome coronavirus 2” refers to a coronavirus within a large genus of betacoronaviruses from the viral family of Coronaviridae. Genbank Accession No. MN908947.3 is a published DNA sequence of SARS-CoV-2. The virus is spread mainly through close contact and via respiratory droplets produced when people cough or sneeze.

The term “SARS-CoV-2 protein” refers to a protein encoded by the nucleic acid of SARS-CoV-2 (e.g., Genbank Accession No. MN908947.3) or a fragment of the protein. In some embodiments, a fragment of the SARS-CoV-2 protein comprises at least 10, 20, or more contiguous amino acids from the full-length protein encoded by the sequence of Genbank Accession No. MN908947.3. For example, a SARS-CoV-2 protein can be a structural protein of the full-length protein encoded by the nucleic acid of the SARS-CoV-2 virus, such as a SARS-CoV-2 S protein (surface glycoprotein; e.g., SEQ ID NO:1 or SEQ ID NO: 21 or SEQ ID NO:22, or variants thereof, e.g., that are at least 90%, 95%, 97%, 98%, or 99% identical to SEQ ID NO:1 or or SEQ ID NO: 21 or SEQ ID NO:22) or a SARS-CoV-2 N protein (nucleocapsid phosphoprotein, SEQ ID NO:2). A SARS-CoV-2 protein can also be a fusion protein that contains different portions of the full-length protein encoded by the nucleic acid of the SARS-CoV-2 virus. For example, a SARS-CoV-2 fusion protein can contain a S1 region of a SARS-CoV-2 S protein, a furin site, and a SARS-CoV-2 N protein (e.g., SEQ ID NO:12).

The term “COVID-19” or “coronavirus disease 2019” refers to an infectious disease caused by the SARS-CoV-2 virus.

The term “TLR agonist” or “Toll-like receptor agonist” as used herein refers to a compound that binds and stimulates a Toll-like receptor including, e.g., TLR-2, TLR-3, TLR-6, TLR-7, or TLR-8. TLR agonists are reviewed in MacKichan, IAVI Report. 9:1-5 (2005) and Abreu et al., J Immmol, 174(8), 4453-4460 (2005). Agonists induce signal transduction following binding to their receptor.

The term “TLR-3 agonist” or “Toll-like receptor 3 agonist” as used herein refers to a compound that binds and stimulates the TLR-3. TLR-3 agonists have been identified including double-stranded RNA, virally derived dsRNA, several chemically synthesized analogs to double-stranded RNA including polyinosine-polycytidylic acid (poly I:C)-polyadenylic-polyuridylic acid (poly A:U) and poly I:poly C, and antibodies (or cross-linking of antibodies) to TLR-3 that lead to IFN-beta production (Matsumoto, M, et al, Biochem Biophys Res Commun 24:1364 (2002), de Bouteiller, et al, J Biol Chem 18:38133-45 (2005)). In some embodiments, a TLR-3 agonist comprises a sequence of any one of SEQ ID NOS:13-20. In some embodiments, a TLR-3 agonist is a dsRNA (e.g., dsRNA encoded by a nucleic acid comprising a sequence set forth in SEQ ID NO:13).

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The terms “nucleic acid” and “polynucleotide” are used interchangeably herein to refer to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

The term “antigen” refers to a protein or part of a polypeptide chain that can be recognized by T cell receptors and/or antibodies. Typically, antigens are derived from bacterial, viral, or fungal proteins.

The term “immunogenically effective dose or amount” of the compositions of the present disclosure is an amount that elicits or modulates an immune response specific for the SARS-CoV-2 protein. Immune responses include humoral immune responses and cell-mediated immune responses. An immunogenic composition can be used therapeutically or prophylactically to treat or prevent disease at any stage. Humoral immune responses are generally mediated by cell free components of the blood, i.e., plasma or serum; transfer of the serum or plasma from one individual to another transfers immunity. Cell mediated immune responses are generally mediated by antigen specific lymphocytes; transfer of the antigen specific lymphocytes from one individual to another transfers immunity.

The term “therapeutic dose” or “therapeutically effective amount” or “effective amount” of a chimeric adenoviral vector or a composition comprising a chimeric adenoviral vector refers to an amount of the vector or composition comprising the vector which prevents, alleviates, abates, or reduces the severity of symptoms of diseases and disorders associated with the source of the SARS-CoV-2 protein (e.g., a SARS-CoV-2 virus).

The term “adjuvant” refers to a non-specific immune response enhancer. Suitable adjuvants include, for example, cholera toxin, monophosphoryl lipid A (MPL), Freund's Complete Adjuvant, Freund's Incomplete Adjuvant, Quil A, and Al(OH). Adjuvants can also be those substances that cause antigen-presenting cell activation and enhanced presentation of T cells through secondary signaling molecules likeToll-like receptors. Examples of Toll-like receptors include the receptors that recognize double-stranded RNA, bacterial flagella, LPS, CpG DNA, and bacterial lipopeptide (Reviewed recently in Abreu et al., J Immunol, 174(8), 4453-4460 (2005)).

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the term “percent identity” or “percent identical,” used in the context of nucleic acids or polypeptides, refers to a sequence that has at least 50% sequence identity with a reference sequence. Alternatively, percent identity can be any integer from 50% to 100%. In some embodiments, a sequence is substantially identical to a reference sequence if the sequence has at least 50%, 55%, 60%, 65%, 70%, 75%, 80%, 85%, 90%, 91%, 92%, 93%, 94%, 95%, 96%, 97%, 98%, or 99% sequence identity to the reference sequence as determined using the methods described herein; preferably BLAST using standard parameters, as described below. Percent identity may also be determined by manual alignment.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A comparison window includes reference to a segment of any one of the number of contiguous positions, e.g., a segment of at least 10 residues. In some embodiments, the comparison window has from 10 to 600 residues, e.g., about 10 to about 30 residues, about 10 to about 20 residues, about 50 to about 200 residues, or about 100 to about 150 residues, in which a sequence may be compared to a reference sequence of the same number of contiguous positions after the two sequences are optimally aligned.

Algorithms that are suitable for determining percent sequence identity and sequence similarity are the BLAST and BLAST 2.0 algorithms, which are described in Altschul et al. (1990). J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff& Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, an amino acid sequence is considered similar to a reference sequence if the smallest sum probability in a comparison of the test amino acid sequence to the reference amino acid sequence is less than about 0.01, more preferably less than about 10′, and most preferably less than about 10′.

### III. Compositions and Methods of the Present Disclosure

The disclosure provides compositions comprising chimeric adenoviral vectors. The chimeric adenoviral vectors can include one or more nucleic acids encoding one or more SARS-CoV-2 proteins. The chimeric adenoviral vectors can also include a nucleic acid encoding a toll-like receptor (TLR) agonist (e.g., a TLR-3 agonist), which can serve as an effective adjuvant when administered in conjunction with viral vectors.

In some embodiments, the chimeric adenoviral vectors of the disclosure comprise an expression cassette comprising the following elements: (a) a first promoter operably linked to a nucleic acid encoding a first SARS-CoV-2 protein; and (b) a second promoter operably linked to a nucleic acid encoding a toll-like receptor-3 (TLR-3) agonist. The first SARS-CoV-2 protein can be a full-length protein (or a substantially identical protein thereof) encoded by the nucleic acid of SARS-CoV-2 (e.g., Genbank Accession No. MN908947.3) or a fragment of the protein. For example, a first SARS-CoV-2 protein can be a structural protein of the full-length protein encoded by the nucleic acid of the SARS-CoV-2 virus, such as a SARS-CoV-2 S protein (surface glycoprotein; e.g., SEQ ID NO:1 or a substantially identical protein thereof, e.g., SEQ ID NO: 21 or SEQ ID NO:22, or variants thereof, e.g., that are at least 90%, or at least 95%, 96%, 97%, 98%, or 99% identical to SEQ ID NO:1 or SEQ ID NO: 21 or SEQ ID NO:22); or a SARS-CoV-2 N protein (nucleocapsid phosphoprotein; SEQ ID NO:2 or a substantially identical protein thereof, e.g., a variant thereof, e.g., that has at least 90%, or at least 95%, 96%, 97%, 98%, or 99% identity to SEQ ID NO:2). In other embodiments, a first SARS-CoV-2 protein can be a protein encoded by other parts of the nucleic acid of the SARS-CoV-2 virus, such as a protein encoded by the ORF1ab gene, a protein encoded by the ORF3a gene, a protein encoded by the E gene (encoding an envelope protein), a protein encoded by the M gene (encoding a membrane glycoprotein), a protein encoded by the ORF6 gene, a protein encoded by the ORF7a gene, a protein encoded by the ORF8 gene, or a protein encoded by the ORF10 gene.

In further embodiments, a first SARS-CoV-2 protein can be a fusion protein that contains different portions of the full-length protein encoded by the nucleic acid of the SARS-CoV-2 virus. For example, a SARS-CoV-2 fusion protein can contain a S1 region of a SARS-CoV-2 S protein, a furin site, and a SARS-CoV-2 N protein (e.g., SEQ ID NO:12).

A nucleic acid that encodes a first SARS-CoV-2 protein in element (a) can comprise a sequence having at least 85%, 90%, 95%, 96%, 97%, 99%, or 100% (e.g., 85%, 87%, 89%, 91%, 93%, 95%, 97%, 99%, or 100%) identity to the sequence of SEQ ID NO:3, which encodes the amino acid sequence of the SARS-CoV-2 S protein (SEQ ID NO:1). In some embodiments, a first SARS-CoV-2 protein in element (a) can comprise a sequence having at least 85%, 90%, 95%, 96%, 97%, 99%, or 100% (e.g., 85%, 87%, 89%, 91%, 93%, 95%, 97%, 99%, or 100%) identity to the sequence of SEQ ID NO:3 and encode a SARS-CoV-2 S protein of SEQ ID NO: 21 or SEQ ID NO:22. In some embodiments, a first SARS-CoV-2 protein in element (a) can comprise a sequence having at least 85%, 90%, 95%, 96%, 97%, 99%, or 100% (e.g., 85%, 87%, 89%, 91%, 93%, 95%, 97%, or 99%) identity to the sequence of SEQ ID NO:3 and encodes a SARS-CoV-2 S protein variant at least 90%, 95%, 97%, 98%, or 99% identical to SEQ ID NO:1 or or SEQ ID NO: 21 or SEQ ID NO:22. In other embodiments, a nucleic acid that encodes a first SARS-CoV-2 protein in element (a) can comprises a sequence having at least 85%, 90%, 95%, 97%, 99%, or 100% (e.g., 85%, 87%, 89%, 91%, 93%, 95%, 97%, 99%, or 100%) identity to the sequence of SEQ ID NO:4, which encodes the amino acid sequence of the SARS-CoV-2 N protein (SEQ ID NO:2). In some embodiments, a first SARS-CoV-2 protein in element (a) can comprise a sequence having at least 85%, 90%, 95%, 96%, 97%, 99%, or 100% (e.g., 85%, 87%, 89%, 91%, 93%, 95%, 97%, or 99%) identity to the sequence of SEQ ID NO:4 and encodes a SARS-CoV-2 N protein variant at least 90% identical, or at least 95%, 96%, 97%, 98%, or 99% identical to SEQ ID NO:2. In further embodiments, a nucleic acid that encodes a first SARS-CoV-2 protein in element (a) can comprises a sequence having at least 85%, 90%, 95%, 97%, 99%, or 100% (e.g., 85%, 87%, 89%, 91%, 93%, 95%, 97%, 99%, or 100%) identity to the sequence of SEQ ID NO:5, which encodes the amino acid sequence of the SARS-CoV-2 fusion protein that contains a S1 region of a SARS-CoV-2 S protein, a furin site, and a SARS-CoV-2 N protein (SEQ ID NO:12).

In addition to a first SARS-CoV-2 protein, the chimeric adenoviral vectors of the disclosure can further comprise element (c) a third promoter operably linked to a nucleic acid encoding a second SARS-CoV-2 protein. In particular embodiments, the order of the elements in the expression cassette from the N-terminus to the C-terminus is: element (a), element (c), and element (b). In some embodiments, the first and second SARS-CoV-2 proteins encoded by their respective nucleic acids in elements (a) and (c) in the expression cassette are the same. In some embodiments, the first and second SARS-CoV-2 proteins encoded by their respective nucleic acids in elements (a) and (c) in the expression cassette are different.

For example, the first SARS-CoV-2 protein can be a SARS-CoV-2 S protein (e.g., SEQ ID NO:1 or SEQ ID NO: 21 or SEQ ID NO:22, or variants thereof, e.g. that are at least 90%, 95%, 96%, 97%, 98%, or 99% identical to SEQ ID NO:1 or SEQ ID NO: 21 or SEQ ID NO:22, which is encoded by a nucleic acid sequence having at least 85%, 90%, 95%, 97%, 99%, or 100% (e.g., 85%, 87%, 89%, 91%, 93%, 95%, 97%, 99/o, or 100%) identity to the sequence of SEQ ID NO:3) and the second SARS-CoV-2 protein can be a SARS-CoV-2 N protein (e.g., SEQ ID NO:2, which is encoded by a nucleic acid sequence having at least 85%, 90%, 95%, 96%, 97%, 99%, or 100% (e.g., 85%, 87%, 89%, 91%, 93%, 95%, 97%, 99%, or 100%) identity to the sequence of SEQ ID NO:4). In another example, the first SARS-CoV-2 protein can be a SARS-CoV-2 N protein (e.g., SEQ ID NO:2, which is encoded by a nucleic acid sequence having at least 85%, 90%, 95%, 97%, 99%, or 100% (e.g., 85%, 87%, 89%, 91%, 93%, 95%, 97%, 99%, or 100%) identity to the sequence of SEQ ID NO:4) and the second SARS-CoV-2 protein can be a SARS-CoV-2 S protein (e.g., SEQ ID NO:1 or SEQ ID NO: 21 or SEQ ID NO:22, or variants thereof, e.g that are at least 90%, 95%, 97%, 98%, or 99% identical to SEQ ID NO:1 or or SEQ ID NO: 21 or SEQ ID NO:22, which is encoded by a nucleic acid sequence having at least 85%, 90%, 95%, 97%, 99%, or 100% (e.g., 85%, 87%, 89%, 91%, 93%, 95%, 97%, 99%, or 100%) identity to the sequence of SEQ ID NO:3).

In another example, the first SARS-CoV-2 protein can be a SARS-CoV-2 N protein (e.g., SEQ ID NO:2; or a variant thereof, e.g., having at least 95%, 96%, 97%, 98%, or 99% identity to SEQ ID NO:2) or a SARS-CoV-2 S protein (e.g., SEQ ID NO:1 or SEQ ID NO: 21 or SEQ ID NO:22, or variants thereof, e.g that are at least 90%, 95%, 97%, 98%, or 99% identical to SEQ ID NO:1 or or SEQ ID NO: 21 or SEQ ID NO:22) and the second SARS-CoV-2 protein can be a SARS-CoV-2 fusion protein (e.g., SEQ ID NO:12, which is encoded by a nucleic acid sequence having at least 85%, 90%, 95%, 97%, 99%, or 100% (e.g., 85%, 87%, 89%, 91%, 93%, 95%, 97%, 99%, or 100%) identity to the sequence of SEQ ID NO:5).

In yet another example, the first SARS-CoV-2 protein can be a SARS-CoV-2 fusion protein (e.g., SEQ ID NO:12, which is encoded by a nucleic acid sequence having at least 85%, 90%, 95%, 97%, 99%, or 100% (e.g., 85%, 87%, 89%, 91%, 93%, 95%, 97%, 99%, or 100%) identity to the sequence of SEQ ID NO:5) and the second SARS-CoV-2 protein can be a SARS-CoV-2 N protein (e.g., SEQ ID NO:2; or a variant at least 95%, 96%, 97%, 98%, or 99% identical to SEQ ID NO:2) or a SARS-CoV-2 S protein (e.g., SEQ ID NO:1 or SEQ ID NO: 21 or SEQ ID NO:22, or variants thereof, e.g., that are at least 90%, 95%, 97%, 98%, or 99% identical to SEQ ID NO:1 or or SEQ ID NO: 21 or SEQ ID NO:22).

One of skill understands that variants of SARS-CoV-2 proteins, e.g., variants of the SARS-CoV-2 S protein, emerge rapidly. Examples of two variant S protein sequences, UK B.1.1.1.7 variant and South African B.1.351 501Y.V2 variant, are provided in SEQ ID NOS:21 and 22, respectively. Other S protein variants are known, including a Brazil variant, P.1 (L18F, T20N, P26S, D138Y, R190S, K417T, E484K, N501Y, D614G, H655Y, T1027I); and an Indian variant B.1.617 (L452R, E484Q, D614G), among others. Thus, in some embodiments, the SARS-CoV-2 S protein sequence is a variant sequence identified in a patient population.

In addition to the above-described vectors triggering an immune response to SARS-CoV-2 protein, in view of the data shown in Example 6, provided herein are embodiments in which co-introduction of a SARS-CoV-2 N protein with any second antigen, which can be from a non-SARS-CoV-2 antigen source, can be used to stimulate a CD8 T-cell immune response to the second antigen.

Accordingly the disclosure also provides for polynucleotides encoding a SARS-CoV-2 N protein (e.g., SEQ ID NO:2 or a variant thereof having at least 90% identity, or at least 95% identity, to SEQ ID NO:2, or a fragment thereof) and encoding a second antigenic protein from any source. For example the second antigenic protein can be from a non-SARS-CoV-2 virus, a bacterium, other pathogen or cancer. For example, in some embodiments, the second antigen is a protein or fragment thereof from Herpes simplex, type 1; Herpes simplex, type 2; encephalitis virus, papillomavirus, Varicella-zoster virus; Epstein-barr virus; Human cytomegalovirus; Human herpes virus, type 8; Human papillomavirus; BK virus; JC virus; Smallpox; polio virus; Hepatitis B virus; Human bocavirus; Parvovirus B19; Human astrovirus; Norwalk virus; coxsackievirus; hepatitis A virus; poliovirus; rhinovirus; Severe acute respiratory syndrome virus; Hepatitis C virus; Yellow Fever virus; Dengue virus; West Nile virus; Rubella virus; Hepatitis E virus; Human Immunodeficiency virus (HIV); Influenza virus; Guanarito virus; Junin virus; Lassa virus; Machupo virus; Sabia virus; Crimean-Congo hemorrhagic fever virus; Ebola virus; Marburg virus; Measles virus; Mumps virus; Parainfluenza virus; Respiratory syncytialvirus; Human metapneumovirus; Hendra virus; Nipah virus; Rabies virus; Hepatitis D; Rotavirus; Orbivirus; Coltivirus; Banna virus; Human Enterovirus; Hanta virus; West Nile virus; Middle East Respiratory Syndrome Corona Virus; Japanese encephalitis virus; Vesicular exanthernavirus; or Eastern equine encephalitis. See, also, U.S. Pat. No. 8,222,224 for a list of antigens that can be used.

Particular examples of second antigens that can be used as described herein in combination with a SARS-CoV-2 N protein include but are not limited to those derived from norovirus (e.g., VP1), Respiratory syncytial virus (RSV), the influenza virus (e.g., HA, NA, M1, NP), human immunodeficiency virus (HIV, e.g., gag, pol, env, etc.), human papilloma virus (HPV, e.g., capsid proteins such as L1), Venezuelan Equine Encephalomyelitis (VEE) virus, Epstein Barr virus, herpes simplex virus (HSV), human herpes virus, rhinoviruses, cocksackieviruses, enteroviruses, hepatitis A, B, C, E, and G (HAV, HBV, HCV, HEV, HGV e.g., surface antigen), mumps virus, rubella virus, measles virus, poliovirus, smallpox virus, rabies virus, and Varicella-zoster virus.

Suitable viral antigens useful as second antigens as described herein also include viral nonstructural proteins, e.g., proteins encoded by viral nucleic acid that do not encode for structural polypeptides, in contrast to those that make capsid or the protein surrounding a virus. Non-structural proteins include those proteins that promote viral nucleic acid replication, viral gene expression, or post-translational processing, such as, for example, Nonstructural proteins 1, 2, 3, and 4 (NS1, NS2, NS3, and NS4, respectively) from Venezuelan Equine encephalitis (VEE), Eastern Equine Encephalitis (EEE), or Semliki Forest.

Bacterial antigens useful as second antigens as described herein can be derived from, for example, Staphylococcus aureus, Staphylococcus epidermis, Helicobacter pylori, Streptococcus bovis, Streptococcus pyogenes, Streptococcus pneumoniae, Listeria monocytogenes, Mycobacterium tuberculosis, Mycobacterium leprae, Corynebacterium diphtheriae, Borrelia burgdorferi, Bacillus anthracis, Bacillus cereus, Clostridium botulinum, Clostridium difficile, Salmonella typhi, Vibrio chloerae, Haemophilus influenzae, Bordetella pertussis, Yersinia pestis, Neisseria gonorrhoeae, Treponema pallidum, Mycoplasm sp., Legionella pneumophila, Rickettsia typhi, Chlamydia trachomatis, and Shigella dysenteriae, Vibrio cholera (e.g., Cholera toxin subunit B, cholera toxin-coregulated pilus (TCP)); Helicobacter pylorii (e.g., VacA, CagA, NAP, Hsp, catalase, urease); E. coli (e.g., heat-labile enterotoxin, fimbrial antigens).

Parasite antigens useful as second antigens as described herein can be derived from, for example, Giardia lamblia, Leishmania sp., Trypanosoma sp., Trichomonas sp., Plasmodium sp. (e.g., P. falciparum surface protein antigens such as pfs25, pfs28, pfs45, pfs84, pfs 48/45, pfs 230, Pvs25, and Pvs28); Schistosoma sp.; Mycobacterium tuberculosis (e.g., Ag85, MPT64, ESAT-6, CFP10, R8307, MTB-32 MTB-39, CSP, LSA-1, LSA-3, EXP1, SSP-2, SALSA, STARP, GLURP, MSP-1, MSP-2, MSP-3, MSP-4, MSP-5, MSP-8, MSP-9, AMA-1, Type 1 integral membrane protein, RESA, EBA-175, and DBA).

Fungal antigens useful as second antigens as described herein can be derived from, for example, Tinea pedis, Tinea corporus, Tinea cruris, Tinea unguium, Cladosporium carionii, Coccidioides immitis, Candida sp., Aspergillus fumigatus, and Pneumocystis carinii.

Cancer antigens useful as second antigens as described herein include, for example, antigens expressed or over-expressed in colon cancer, stomach cancer, pancreatic cancer, lung cancer, ovarian cancer, prostate cancer, breast cancer, skin cancer (e.g., melanoma), leukemia, or lymphoma. Exemplary cancer antigens include, for example, HPV L1, HPV L2, HPV E1, HPV E2, placental alkaline phosphatase, AFP, BRCA1, Her2/neu, CA 15-3, CA 19-9, CA-125, CEA, Hcg, urokinase-type plasminogen activator (Upa), plasminogen activator inhibitor, CD53, CD30, CD25, C5, CD11a, CD33, CD20, ErbB2, CTLA-4. See Sliwkowski & Mellman (2013) Science 341:6151 for additional cancer targets.

While an attenuated adenovirus can be used to express a SARS-CoV-2 N protein and second antigenic protein (e.g., to generate a CD8 T-cell response), other polynucleotides or vectors can also be used. Expression vectors can include, for example, virally-derived vectors, e.g., recombinant adeno-associated virus (AA V) vectors, retroviral vectors, adenoviral vectors, modified vaccinia Ankara (MVA) vectors, and lentiviral (e.g., HSV-1-derived) vectors (see, e.g., Brouard et al. (2009) British J. Pharm. 157:153). In other embodiments, the SARS-CoV-2 N protein (e.g., SEQ ID NO:2) and second antigenic protein can be encoded by a polynucleotide, e.g., naked or encapsulated DNA or RNA, e.g., mRNA (see, e.g., U.S. Patent Publication No. 2020/0254086 for details of various aspects for RNA-based vaccines).

In some embodiments a vector that comprises a region encoding a SAR-CoV-2 N protein and a region encoding a second antigenic protein, further comprises a nucleic acid encoding a TLR agonist (e.g., a TLR-3 agonist), which can serve as an effective adjuvant when administered in conjunction with vectors, such as viral vectors.

In some embodiments, a vector, e.g., a viral vector, encodes a SARS-Co-V2 N protein (e.g., an N protein sequence of SEQ ID NO:2, or a variant thereof, e.g., at least 90% identical, or at least 95% identical to SEQ ID NO:2) and a second antigenic protein, in which expression of the N protein and second antigenic protein is driven by different promoters. In further embodiments, the vector comprises a ribosomal skipping element situated between the region of the nucleic acid that encode the N protein and the region encoding the second antigenic protein. In some embodiments, the vector comprises an IRES situated between the N protein and second antigenic protein to produce a bicistronic transcript. In some embodiments, the ribosomal skipping element is a sequence encoding a virus 2A peptide (T2A), a porcine teschovirus-1 2A peptide (P2A), a foot-and-mouth disease virus 2A peptide (F2A), equine rhinitis A virus 2A peptide (E2A), a cytoplasmic polyhedrosis virus 2A peptide (BmCPV 2A), or a flacherie virus of B. mori 2A peptide (BmIFV 2A); situated between the N protein and the second antigenic protein. In some embodiments, the construct further encodes a TLR agonist.

In some embodiments, the vector comprises a first promoter operably linked to polynucleotide sequence encoding a SARS-CoV-2 N protein and a second promoter operably linked to the second antigenic protein. In some embodiments, the vector, e.g., a viral vector, can further comprise a third promoter operably linked to a TLR agonist, e.g., a TLR-3 agonist.

In particular embodiments, the order of the elements in the expression cassette from the N-terminus to the C-terminus is: a sequence encoding an antigenic protein, a sequence encoding a SARS-Co-V2 N protein and a sequence encoding a TLR agonist, e.g., a TLR 3 agonist.

In further embodiments, an antigenic protein can be fused to the N protein sequence For example, a fusion protein can contain an antigenic protein, a furin site, and a SARS-CoV-2 N protein, or variant thereof, e.g., at least 90% identical, or at least 95% identical to SEQ ID NO:2.

In some embodiments, a SARS-CoV-2 N protein encoded by a vector has at least 90% identity to SEQ ID NO:2. In some embodiments, the N protein encoded by the vector has at least 95%, 96%, 97%, 98%, or 99% identity to SEQ ID NO:2.

In some embodiments, the vector comprises an expression cassette as described herein in which a second antigenic protein replaces a SARS-CoV-2 S protein in the constructs provided herein that encode both the N protein and SARS-CoV-2 S proteins. Thus, for example, in some embodiments, the vector comprises sequences as follows (5′-3): CMV-second antigenic protein-BGH-Actin-N protein-SPA-BGH-CM-dsRNA-SPA in which “CMV” is a CMV promoter; “second antigenic protein” is a nucleic acid sequence encoding a second antigenic protein, e.g., from an infectious disease agent or a cancer antigen as described herein, “BGH” is a bovine growth hormone polyadenylation signal sequence”; “βActin” is a beta-actin promoter, e.g., a human beta-actin promoter; “N-protein” is a nucleic acid sequence encoding a SARS-CoV2 N protein as described herein, e.g., SEQ ID NO:2, or a protein having at least 90% identity or at least 95% identity to SEQ ID NO:2, “SPA” is a synthetic polyA sequence, and “dsRNA” is a nucleic acid sequence encoding a TLR agonist, e.g., a TLR-3 agonist.

In some embodiments, an N protein from an alternative coronavirus is employed in place of the SARS-CoV-2 N protein in constructs comprising an N protein and an antigenic protein, such as an infection disease antigen or cancer antigen. Thus, for example, in some embodiments, such a construct can comprise a SARS-CoV or MERS N protein.

In some embodiments, the vector is an adenoviral vector, e.g., an adenovirus 5(Ad5) vector as described below.

Suitable Adenoviral Vectors

In some embodiments, an adenoviral vector as described herein is adenovirus 5 (Ad5), which can include, for example, Ad5 with deletions of the E1/E3 regions and Ad5 with a deletion of the E4 region. Other suitable adenoviral vectors include strains 2, orally tested strains 4 and 7, enteric adenoviruses 40 and 41, and other strains (e.g. Ad34) that are sufficient for delivering an antigen and eliciting an adaptive immune response to the transgene antigen (Lubeck el al., Proc Natl Acad Sci USA, 86(17), 6763-6767 (1989); Shen et al., J Virol, 75(9), 4297-4307 (2001); Bailey et al., Virology, 202(2), 695-706 (1994)). In some embodiments, the adenoviral vector is a live, replication incompetent adenoviral vector (such as E1 and E3 deleted rAd5), live and attenuated adenoviral vector (such as the E1B55K deletion viruses), or a live adenoviral vector with wild-type replication.

The transcriptional and translational control sequences in expression vectors to be used in transforming vertebrate cells in vivo may be provided by viral sources. For example, commonly used promoters and enhancers are derived, e.g., from beta-actin, adenovirus, simian virus (SV40), and human cytomegalovirus (CMV). For example, vectors allowing expression of proteins under the direction of the CMV promoter, beta-actin promoter, SV40 early promoter, SV40 later promoter, metallothionein promoter, murine mammary tumor virus promoter, Rous sarcoma virus promoter, transducer promoter, or other promoters shown effective for expression in mammalian cells are suitable. Further viral genomic promoter, control and/or signal sequences may be used, provided such control sequences are compatible with the host cell chosen.

Various promoters can be used in the chimeric adenoviral vectors described herein. The promoters used for elements (a), (b), and/or (c) can be identical or different. For example, in some embodiments, the first promoter used in element (a) and the second promoter used in element (b) can both be a CMV promoter. In other embodiments, when element (c) is included, the third promoter can be identical or different from the first and/or second promoter. For example, the first promoter and the second promoter can both be a CMV promoter and the third promoter can be a beta-actin promoter (e.g., a human beta-actin promoter).

TLR Agonists

The chimeric adenoviral vectors described herein can also include a nucleic acid encoding a toll-like receptor (TLR) agonist, which can serve as an effective adjuvant when administered in conjunction with viral vectors. TLR agonists can be used to enhance the immune response to the SARS-CoV-2 protein. In some embodiments, TLR-3 agonists are used. In some embodiments, the TLR agonists described herein can be delivered simultaneously with the expression vector encoding an antigen of interest (e.g., a SARS-CoV-2 protein). In other embodiments, the TLR agonists can be delivered separately (i.e., temporally or spatially) from the expression vector encoding an antigen of interest (e.g., a SARS-CoV-2 protein). For example, the expression vector can be administered via a non-parenteral route (e.g., orally, intranasally, or mucosally), while the TLR agonist can be delivered by a parenteral route (e.g., intramuscularly, intraperitoneally, or subcutaneously).

In particular embodiments, a TLR-3 agonist is can be used to stimulate immune recognition of an antigen of interest. TLR-3 agonists include, for example, short hairpin RNA, virally derived RNA, short segments of RNA that can form double-strands or short hairpin RNA, and short interfering RNA (siRNA). In one embodiment of the disclosure, the TLR-3 agonist is virally derived dsRNA, such as for example, a dsRNA derived from a Sindbis virus or dsRNA viral intermediates (Alexopoulou et al, Nature 413:732-8 (2001)). In some embodiments, the TLR-3 agonist is a short hairpin RNA. Short hairpin RNA sequences typically comprise two complementary sequences joined by a linker sequence. The particular linker sequence is not a critical aspect of the disclosure. Any appropriate linker sequence can be used so long as it does not interfere with the binding of the two complementary sequences to form a dsRNA.

In some embodiments, the TLR-3 agonist can comprise a sequence having at least 85%, 90%, 95%, 97%, 99%, or 100/a (e.g., 85%, 87%, 89%, 91%, 93%, 95%, 97%, 99%, or 100%) identity to a sequence set forth in SEQ ID NOS:13-20. In particular embodiments, the TLR-3 agonists comprises the sequence of SEQ ID NO:13. In certain embodiments, dsRNA that is a TLR-3 agonist does not encode a particular polypeptide, but produces a pro-inflammatory cytokine (e.g. IL-6, IL-8, TNF-alpha, IFN-alpha, IFN-beta) when contacted with a responder cell (e.g., a dendritic cell, a peripheral blood mononuclear cell, or a macrophage) in vitro or in-vivo.

In particular embodiments, the TLR agonist (e.g., TLR-3 agonist) described herein can be delivered simultaneously within the same the expression vector that encodes a SARS-CoV-2 protein. In other embodiments, the TLR agonist (e.g., TLR-3 agonist) can be delivered separately (i.e., temporally or spatially) from the expression vector that encodes a SARS-CoV-2 protein. In some cases when the TLR-3 agonist is delivered separately from the expression vector, the nucleic acid encoding the TLR-3 agonist (e.g., an expressed dsRNA) and the chimeric adenoviral vector comprising a nucleic acid encoding a SARS-CoV-2 protein can be administered in the same formulation. In other cases the nucleic acid encoding the TLR-3 agonist and the chimeric adenoviral vector comprising a nucleic acid encoding a SARS-CoV-2 protein can be administered in different formulations. When the nucleic acid encoding the TLR-3 agonist and the adenoviral vector comprising a nucleic acid encoding a SARS-CoV-2 protein are administered in different formulations, their administration may be simultaneous or sequential. For example, the nucleic acid encoding the TLR-3 agonist may be administered first, followed by the chimeric adenoviral vector (e.g., 1, 2, 4, 8, 12, 16, 20, or 24 hours, 2, 4, 6, 8, or 10 days later). Alternatively, the adenoviral vector may be administered first, followed by the nucleic acid encoding the TLR-3 agonist (e.g., 1, 2, 4, 8, 12, 16, 20, or 24 hours, 2, 4, 6, 8, or 10 days later). In some embodiment, the nucleic acid encoding the TLR-3 agonist and the nucleic acid encoding the SARS-CoV-2 protein are under the control of the same promoter. In other embodiments, the nucleic acid encoding the TLR-3 agonist and the nucleic acid encoding the SARS-CoV-2 protein are under the control of different promoters.

### IV. Pharmaceutical Compositions and Routes of Administration

An immunogenic pharmaceutical composition can contain a chimeric adenoviral vector described herein and a pharmaceutically acceptable carrier. Suitable carriers include, for example, water, saline, alcohol, a fat, a wax, a buffer, a solid carrier, such as mannitol, lactose, starch, magnesium stearate, sodium saccharine, talcum, cellulose, glucose, sucrose, and magnesium carbonate, or biodegradable microspheres (e.g., polylactate polyglycolate). Suitable biodegradable microspheres are disclosed, for example, in U.S. Pat. Nos. 4,897,268; 5,075,109; 5,928,647; 5,811,128; 5,820,883. The immunogenic polypeptide and/or carrier expression vector can be encapsulated within the biodegradable microsphere or associated with the surface of the microsphere.

The ingredients in an immunogenic pharmaceutical composition are closely related to factors such as, but are not limited to, the route of administration of the immunogenic pharmeutical composition, the timeline and/or duration of drug release, and the targeted delivery site. In some embodiments, a delayed release coating or an additional coating of the formulation can contain other film-forming polymers being non-sensitive to luminal conditions for technical reasons or chronographic control of the drug release. Materials to be used for such purpose includes, but are not limited to; sugar, polyethylene glycol, polyvinylpyrrolidone, polyvinyl alcohol, polyvinyl acetate, hydroxypropyl cellulose, methylcellulose, ethylcellulose, hydroxypropyl methylcellulose, carboxymethylcellulose sodium and others, used alone or in mixtures.

Additives such as dispersants, colorants, pigments, additional polymers, e.g., poly(ethylacrylat, methylmethacrylat), anti-tacking and anti-foaming agents can be included into a coating layer. Other compounds may be added to increase film thickness and to decrease diffusion of acidic gastric juices into the core material. The coating layers can also contain pharmaceutically acceptable plasticizers to obtain desired mechanical properties. Such plasticizers are for instance, but not restricted to, triacetin, citric acid esters, phthalic acid esters, dibutyl sebacate, cetyl alcohol, polyethylene glycols, glycerol monoesters, polysorbates or other plasticizers and mixtures thereof. The amount of plasticizer can be optimised for each formula, and in relation to the selected polymer(s), selected plasticizer(s) and the applied amount of said polymer(s).

Such immunogenic pharmaceutical compositions can also comprise non-immunogenic buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), suspending agents, thickening agents and/or preservatives. Alternatively, compositions of the present disclosure may be formulated as a lyophilate. Compounds may also be encapsulated within liposomes using well known technology.

Further, pharmaceutical compositions can be prepared to protect against stomach degradation such that the administered immunogenic biological agent reach the desired location. Methods for microencapsulation of DNA and drugs for oral delivery are described, e.g., in US2004043952. For the oral environment, several of these are available including the Eudragit and the TimeClock release systems as well as other methods specifically designed for adenovirus (Lubeck et al., Proc Natl Acad Sci USA, 86(17), 6763-6767 (1989); Chourasia and Jain, J Pharm Pharm Sci, 6(1), 33-66 (2003)). In some embodiments, the Eudragit system can be used to to deliver the chimeric adenoviral vector to the lower small intestine.

In particular embodiments, the immunogenic composition is in the form of a tablet or capsule, e.g., in the form of a compressed tablet covered by enteric coating. In some embodiments, the immunogenic composition is encapsulated in a polymeric capsule comprising gelatin, hydroxypropylmethylcellulose, starch, or pullulan. In some embodiments, the immunogenic composition is in the form of microparticles less than 2 mm in diameter, e.g., each microparticle covered with enteric coating as described herein. In particular embodiments, the immunogenic composition in the form of a tablet, a capsule, or a microparticle can be orally administered. In some embodiments, site-specific delivery can be achieved via tablets or capsules that release upon an externally generated signal. Early models released for a high-frequency (HF) signal, as disclosed in Digenis et al. (1998) Pharm. Sci. Tech. Today 1:160. The original HF capsule concept has since been updated and the result marketed as InteliSite®. The updated capsule is a radio-frequency activated, non-disintegrating delivery system. Radiolabeling of the capsule permits the determination of the capsule location within a specific region of the GI tract via gamma scintigraphy. When the capsule reaches the desired location in the GI tract, external activation opens a series of windows to the capsule drug reservoir.

In some embodiments, the immunogenic composition can be enclosed in a radio-controlled capsule, so that the capsule is tracked and signaled once it reaches the delivery site. In some embodiments, the capsule is signaled at a given time after administration that corresponds to when the capsule is expected to arrive at the delivery site, with or without detecting.

The compositions described herein may be administered as part of a sustained release formulation (i.e., a formulation such as a capsule or sponge that effects a slow release of compound following administration). Such formulations may generally be prepared using well known technology (see, e.g., Coombes et al. (1996) Vaccine 14:1429-1438). Sustained-release formulations may contain a polypeptide, polynucleotide or antibody dispersed in a carrier matrix and/or contained within a reservoir surrounded by a rate controlling membrane.

Carriers for use within such formulations are biocompatible and may also be biodegradable; preferably the formulation provides a relatively constant level of active component release. Such carriers include microparticles of poly(lactide-co-glycolide), as well as polyacrylate, latex, starch, cellulose and dextran. Other delayed-release carriers include supramolecular biovectors, which comprise a non-liquid hydrophilic core (e.g., a cross-linked polysaccharide or oligosaccharide) and, optionally, an external layer comprising an amphiphilic compound (see, e.g., WO 94/20078; WO 94/23701; and WO 96/06638). The amount of active compound contained within a sustained release formulation depends upon the site of implantation, the rate and expected duration of release and the nature of the condition to be treated or prevented.

In some embodiments, the immunogenic compositions are presented in unit-dose or multi-dose containers, such as sealed ampoules or vials. Such containers are preferably hermetically sealed to preserve sterility of the formulation until use. In general, formulations can be stored as suspensions, solutions, or emulsions in oily or aqueous vehicles. Alternatively, an immunogenic composition may be stored in a freeze-dried condition requiring only the addition of a sterile liquid carrier immediately prior to use.

Compositions for Targeted Delivery

In some embodiments of targeted delivery, enteric coatings are used to shield substances from the low pH environment of the stomach and delay release of the enclosed substance until it reaches a desired target later in the digestive tract. Enteric coatings are known, and commercially available. Examples include pH-sensitive polymers, bio-degradable polymers, hydrogels, time-release systems, and osmotic delivery systems (see, e.g., Chourasia & Jain (2003) J. Pharm. Pharmaceutical Sci. 6:33).

In some embodiments, the targeted delivery site is the ileum. The pH of the gastrointestinal tract (GIT) progresses from very acidic in the stomach (pH˜2), to more neutral in the ileum (pH˜5.8-7.0). pH sensitive coatings can be used that dissolve in the ileum or just before the ileum. Examples include Eudragit® L and S polymers (threshold pH's ranging from 5.5-7.0); polyvinyl acetate phthalate (pH 5.0), hydroxypropyl methylcellulose phthalate 50 and 55 (pH 5.2 and 5.4, respectively), and cellulose acetate phthalate (pH 5.0). Thakral et al. (2013) Expert Opin. Drug Deliv. 10:131 review Euragit® formulations for ileal delivery, in particular, combinations of L and S that ensure delivery at pH-7.0. Crotts el al. (2001) Eur. J Pharm. Biol. 51:71 describe Eudragit® formulations with appropriate disintegration properties. Vijay et al. (2010) J. Mater. Sci. Mater. Med. 21:2583 review acrylic acid (AA)-methyl methacrylate (MMA) based copolymers for ileal delivery at pH 6.8.

For ileal delivery, the polymer coating typically dissolves at about pH 6.8 and allows complete release within about 40 min (see, e.g., Huyghebaert et al. (2005) Int. J. Pharm. 298:26). To accomplish this, a therapeutic substance can be covered in layers of different coatings, e.g., so that the outermost layer protects the substance through low pH conditions and is dissolved when the tablet leaves the stomach, and at least one inner layer that dissolves as the tablet passes into increasing pH. Examples of layered coatings for delivery to the distal ileum are described, e.g., in WO 2015/127278, WO 2016/200951, and WO 2013/148258.

Biodegradable polymers (e.g., pectin, azo polymers) typically rely on the enzymatic activity of microflora living in the GIT. The ileum harbors larger numbers of bacteria than earlier stages, including lactobacilli and enterobacteria.

Osmotic-controlled Release Oral delivery Systems (OROS®; Alza) is an example of an osmotic system that degrades over time in aqueous conditions. Such materials can be manipulated with other coatings, or in varying thicknesses, to deliver specifically to the ileum (see, e.g., Conley el al. (2006) Curr. Med. Res. Opin. 22:1879).

Combination polymers for delivery to the ileum are reported in WO2000062820. Examples include Eudragit® L100-55 (25 mg/capsule) with triethyl citrate (2.4 mg/capsule), and Povidone K-25 (20 mg/tablet) followed by Eudragit®FS30D (30 mg/tablet). pH sensitive polymers can be applied to effect delivery to the ileum, as described above and, e.g., methacrylic acid copolymers (e.g., poly(methacylic acid-co-methyl methacrylate) 1:1), cellulose acetate phthalate, hydroxypropyl methylcellulose phthalate, hydroxypropyl methylcellulose acetate succinate, polyvinyl acetate phthalate, cellulose acetate trimellitate, carboxymethyl ethyl-cellulose, shellac or other suitable polymer(s). The coating layer can also be composed of film-forming polymers being sensitive to other luminal components than pH, such as bacterial degradation or a component that has such a sensitivity when it is mixed with another film-forming polymer Examples of such components providing delayed release to the ileum are polymers comprising azo bond(s), polysaccharides such as pectin and its salts, galactomannans, amylose and chondroitin, disulphide polymers and glycosides.

Components with varying pH, water, and enzymatic sensitivities can be used in combination to target a therapeutic composition to the ileum. The thickness of the coating can also be used to control release. The components can also be used to form a matrix, in which the therapeutic composition is embedded. See generally, Frontiers in Drug Design & Discovery (Bentham Science Pub. 2009) vol. 4.

Adjuvant

In some embodiments of the present disclosure, in addition to the TLR agonist (e.g., TLR-3 agonist) encoded in the chimeric adenoviral vector, the compositions can further comprise additional adjuvants. Suitable adjuvants include, for example, the lipids and non-lipid compounds, cholera toxin (CT), CT subunit B, CT derivative CTK63, E. coli heat labile enterotoxin (LT), LT derivative LTK63, Al(OH)₃, and polyionic organic acids as described in e.g., WO 04/020592, Anderson and Crowle, Infect. Immun. 31(1):413-418 (1981), Roterman et al., J. Physiol. Pharmacol., 44(3):213-32 (1993), Arora and Crowle, J. Reliculoendothel. 24(3):271-86 (1978), and Crowle and May, Infect. Immun. 38(3):932-7 (1982)). Suitable polyionic organic acids include for example, 6,6′-[3,3′-demithyl[1,1′-biphenyl]-4,4′-diyl]bis(azo)bis[4-amino-5-hydroxy-1,3-naphthalene-disulfonic acid] (Evans Blue) and 3,3′-[1,1′biphenyl]-4,4′-diylbis(azo)bis[4-amino-1-naphthalenesulfonic acid] (Congo Red). It will be appreciated by those of skill in the art that the polyionic organic acids may be used for any genetic vaccination method in conjunction with any type of administration.

Other suitable adjuvants include topical immunomodulators such as, members of the imidazoquinoline family such as, for example, imiquimod and resiquimod (see, e.g., Hengge et al., Lancet Infect. Dis. 1(3):189-98 (2001).

Additional suitable adjuvants are commercially available as, for example, additional alum-based adjuvants (e.g., Alhydrogel, Rehydragel, aluminum phosphate, Algammulin); oil based adjuvants (Freund's Incomplete Adjuvant and Complete Adjuvant (Difco Laboratories, Detroit, Mich.), Specol, RIBI, TiterMax, Montanide ISA50 or Seppic MONTANIDE ISA 720): nonionic block copolymer-based adjuvants, cytokines (e.g., GM-CSF or Flat3-ligand); Merck Adjuvant 65 (Merck and Company, Inc., Rahway, N.J.); AS-2 (SmithKline Beecham, Philadelphia, Pa.); salts of calcium, iron or zinc; an insoluble suspension of acylated tyrosine; acylated sugars; cationically or anionically derivatized polysaccharides; polyphosphazenes; biodegradable microspheres; monophosphoryl lipid A and Quil A. Cytokines, such as GM-CSF or interleukin-2, -7, or -12, are also suitable adjuvants. Hemocyanins (e.g., keyhole limpet hemocyanin) and hemoerythrins may also be used in the disclosure. Polysaccharide adjuvants such as, for example, chitin, chitosan, and deacetylated chitin are also suitable as adjuvants. Other suitable adjuvants include muramyl dipeptide (MDP, N acetylmuramyl L alanyl D isoglutamine) bacterial peptidoglycans and their derivatives (e.g., threonyl-MDP, and MTPPE). BCG and BCG cell wall skeleton (CWS) may also be used as adjuvants in the disclosure, with or without trehalose dimycolate. Trehalose dimycolate may be used itself (see, e.g., U.S. Pat. No. 4,579,945). Detoxified endotoxins are also useful as adjuvants alone or in combination with other adjuvants (see, e.g., U.S. Pat. Nos. 4,866,034; 4,435,386; 4,505,899; 4,436,727; 4,436,728; 4,505,900; and 4,520,019. The saponins QS21, QS17, QS7 are also useful as adjuvants (see, e.g., U.S. Pat. No. 5,057,540; EP 0362279; WO 96/33739; and WO 96/11711). Other suitable adjuvants include Montanide ISA 720 (Seppic, France), SAF (Chiron, Calif., United States), ISCOMS (CSL), MF-59 (Chiron), the SBAS series of adjuvants (e.g., SBAS-2, SBAS-4 or SBAS-6 or variants thereof, available from SmithKline Beecham, Rixensart, Belgium), Detox (Corixa, Hamilton, Mont.), and RC-529 (Corixa, Hamilton, Mont.).

Within the pharmaceutical compositions provided herein, the adjuvant composition can be designed to induce, e.g., an immune response predominantly of the Th1 or Th2 type. High levels of Th1-type cytokines (e.g., IFN-gamma, TNF-alpha, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following oral delivery of a composition comprising an immunogenic polypeptide as provided herein, an immune response that includes Th1- and Th2-type responses will typically be elicited.

Routes of Administration

A composition comprising the chimeric adenoviral vector can be administered by any non-parenteral route (e.g., orally, intranasally, or mucosally via, for example, the vagina, lungs, salivary glands, nasal cavities, small intestine, colon, rectum, tonsils, or Peyer's patches). The composition may be administered alone or with an adjuvant as described above. In particular embodiments, the immunogenic composition is administered orally in the form of a tablet or capsule. In further embodiments, the immunogenic composition is administered orally for targeted delivery in the ileum in the form of a tablet or capsule.

### V. Therapeutic Uses

One aspect of the present disclosure involves using the immunogenic compositions described herein to elicit an antigen specific immune response towards a SARS-CoV-2 protein (e.g., a SARS-CoV-2 protein having the sequence of SEQ ID NOS:1, 2, or 12) in a subject. In some embodiments, the immune response is elicited in an alveolar cell, an absorptive enterocyte, a ciliated cell, a goblet cell, a club cells, and/or an airway basal cell of the subject. As used herein, a “subject” refers to any warm-blooded animal, such as, for example, a rodent, a feline, a canine, or a primate, preferably a human. The immunogenic compositions can be used before the subject developed COVID-19 to prevent disease. The disease can be diagnosed using criteria generally accepted in the art. For example, viral infection can be diagnosed by the measurement of viral titer in a biological sample (e.g., a nostril swab or mucosal sample) from the subject.

As shown in the examples, vaccines described herein can be particularly effective in triggering a CD8⁺ T-cell immune response. In some embodiments, this significant CD8 response may be triggered by the presence of the SARS-CoV-2 N protein (e.g., SEQ ID NO:2 os substantially identical variants thereof), which acts to stimulate a CD8⁺ T-cell response to a second antigenic protein (which in the example was SARS-CoV-2 S protein, but which could be a different SARS-CoV-2 protein, or as discussed in more detail below, a non-SARS-CoV-2 protein). Accordingly, in some embodiments, a vaccine as described herein resulting in expression of a SARS-CoV-2 N protein as well as a second antigenic protein, can be used to trigger an immune response, which includes a CD8⁺ T-cell response, in a subject, e.g., a human subject. In some embodiments, the human subject is a subject with less ability to develop an antibody-based immune response or would otherwise benefit from a CD8⁺ T-cell immune response. Exemplary subjects can include, but are not limited to: elderly humans, e.g., at least 50, at least 60 or at least 70 years old, or that has an antibody deficiency disorder (see, e.g., Angel A. Justiz Vaillant; Kamleshun Ramphul, ANTIBODY DEFICIENCY DISORER (Treasure Island (FL): StatPearls Publishing: 2020) for a descrition thereof), which can include but is not limited to subjects with X-linked agammaglobulinemia (Bruton disease), transient hypogammaglobulinemia of newborn, Selective Ig immunodeficiencies, for example, IgA selective deficiency, Super IgM syndrome, and common variable immunodeficiency disord.

Immunotherapy is typically active immunotherapy, in which treatment relies on the in vivo stimulation of the endogenous host immune system to react against, e.g., virally infected cells, with the administration of immunogenic composition comprising the chimeric adenoviral vectors described herein.

Frequency of administration of the immunogenic composition described herein, as well as dosage, will vary from individual to individual, and may be readily established using standard techniques. In some embodiments, between 1 and 10 (e.g., between 2 and 10, between 3 and 10, between 4 and 10, between 5 and 10, between 6 and 10, between 7 and 10, between 8 and 10, between 9 and 10, between 1 and 9, between 1 and 8, between 1 and 7, between 1 and 6, between 1 and 5, between 1 and 4, between 1 and 3, or between 1 and 2) doses may be administered over a 52 week period. In some embodiments, 2 or 3 doses are administered at intervals of 1 month; or for example, 2-3 doses are administered every 2-3 months. It is possible that the intervals will be once a year for certain therapies. Booster vaccinations may be given periodically thereafter.

A suitable dose is an amount of a compound for example that, when administered as described above, is capable of promoting an anti-viral immune response, and is at least 10-50% above the basal (i.e., untreated) level. Such response can be monitored by measuring the anti-viral antibodies in a patient or by vaccine-dependent generation of cytolytic T cells capable of killing, e.g., the patient's virus-infected cells in vitro. Immunogenic responses can also be measured by detecting immunocomplexes formed between the immunogenic polypeptides and antibodies in body fluid which are specific for the immunogenic polypeptides. Samples of body fluid taken from an individual prior to and subsequent to initiation of therapy may be analyzed for the immunocomplexes. Briefly, the number of immunocomplexes detected in both samples can be compared. A substantial change in the number of immunocomplexes in the second sample (post-therapy initiation) relative to the first sample (pre-therapy) reflects successful therapy. Such vaccines should also be capable of causing an immune response that leads to prevention of the COVID-19 disease in vaccinated patients as compared to non-vaccinated patients.

Exemplary dosages can be measured in infectious units (I.U.). A replication-deficient recombinant Ad5 vector can be tittered and quantified using I.U. units. This is accomplished through performance of an IU assay in the adherent human embryonic kidney (HEK) 293 cell line, which is permissive for growth of replication-deficient Ad5. HEK293 cells are plated in a 24-well sterile tissue culture plate and allowed to adhere. The viral material is diluted in sequential 10-fold dilutions and infected into individual wells of plated HEK293 cells in an appropriate number of replicates, usually in duplicate or triplicate. Infection is allowed to proceed via incubation for ˜40-42 hours at 37 C, 5% C02. Cells are then fixed with methanol to allow permeability, washed, and blocked with a buffer solution containing bovine serum albumin (BSA). Cells are then incubated with a rabbit-derived primary antibody against the Ad5 hexon surface protein, washed, and probed again with an HRP-conjugated anti-rabbit secondary antibody. Infected cells are then stained via incubation with 3,3′-diaminobenzidine tetrahydrochloride (DAB) and hydrogen peroxide. Infected cells are visualized using phase-contrast microscopy and a dilution is chosen that exhibits discreet individual infection events—these are visible as darkly stained cells that are highly visible against the semi-transparent monolayer of uninfected cells. Total infected cells are counted per field-of-vision in at least ten fields-of-vision of the appropriate dilution. Viral titer can be calculated using the average number of these counts in conjunction with the total number of fields-of-vision for the objective lens/eyepiece magnification used and multiplying by the dilution factor used in the counts.

In some embodiments, the vaccines administered can have a dosage of 10⁷-10¹¹, e.g., 10⁸-10¹¹, 10⁹-10¹¹, 5×10⁹-5×10¹⁰ I.U. Suitable dose sizes will vary with the size of the patient, but will typically range from about 0.01 ml to about 10 ml for an injected vaccine, more typically from about 0.025 to about 7.5 ml, most typically from about 0.05 to about 5 ml. For a tablet or capsule final product, the size would be between 10 mg to 1000 mg, most typically between 100-400 mg. Those of skill in the art will appreciate that the dose size may be adjusted based on the particular patient or the particular disease or disorder being treated.

### EXAMPLES

The following examples are intended to illustrate, but not to limit the present disclosure.

### Example 1. Generation of Recombinant Adenoviral Constructs

Several different recombinant adenoviral (rAd) constructs to prevent SARS-CoV-2 infection were developed, using the same vector platform that was previously evaluated clinically (14, 15), with the exception that different antigens were used. Several rAd SARS-CoV-2 vaccines were generated by standard methods (e.g., as described by He, et al (17)).

Three vaccine constructs were created based on the published DNA sequence of SARS-CoV-2 publicly available as Genbank Accession No. MN908947.3. Specifically, the published amino acid sequence of the SARS-CoV-2 S protein (or surface glycoprotein; SEQ 1 below) and the SARS-CoV-2 N protein (or nucleocapsid phosphoprotein; SEQ 2 below) were used to synthesize nucleic acid sequences codon optimized for expression in Homo sapiens cells. Codon optimized nucleic acid sequences for the SARS-CoV-2 S gene and SARS-CoV-2 N gene are shown in SEQ ID NOS:3 and 4, respectively. These sequences were used to create recombinant plasmids containing transgenes cloned into the E1 region of Adenovirus Type 5 (pAd).

Two recombinant pAd plasmids were constructed using sequences from SARS-CoV-2.

1. ED81.4.1: pAd-CMV-SARS-CoV-2-S-BGH-CMV-dsRNA-SPA. Recombinant Ad5 vector containing SEQ ID NO:3 under control of the CMV promoter. Sequence of the entire transgene cassette from initial CMV promoter through the SPA following the dsRNA adjuvant is included as SEQ ID NO:6. Sequence of the entire recombinant adenoviral genome containing this transgene construct is included as SEQ ID NO:9.
2. ED84A6.4.1: pAd-CMV-SARS-CoV-2-S-BGH-bActin-SARS-CoV-2-N-SPA-BGH-CMV-dsRNA-SPA. Recombinant Ad5 vector containing SEQ ID NO:3 under control of the CMV promoter and SEQ ID NO:4 under control of the beta-actin promoter. Sequence of the entire transgene cassette from initial CMV promoter through the SPA following the dsRNA adjuvant is included as SEQ ID NO:7. Sequence of the entire recombinant adenoviral genome containing this transgene construct is included as SEQ ID NO: 10.

In addition, a third pAd plasmid was constructed using a fusion sequence (SEQ ID NO:5) combining the S1 region of SARS-CoV-2 S gene (including the native furin site between S1 and S2) with the full-length SARS-CoV-2 N gene:

3. ST05.1.3.3: pAd-CMV-SARS-CoV-2-S1-Furin-N-BGH-CMV-dsRNA-SPA. Recombinant Ad5 vector containing SEQ ID NO:5 under control of the CMV promoter. Sequence of the entire transgene cassette from initial CMV promoter through the SPA following the dsRNA adjuvant is included as SEQ ID NO:8. Sequence of the entire recombinant adenoviral genome containing this transgene construct is included as SEQ ID NO:11.

Sequences were cloned into a shuttle plasmid using the restriction sites (e.g., Sthl and Sgfl). The shuttle plasmid was used to lock the transgenes onto a plasmid (pAd) containing the full sequence of Adenovirus Type 5 deleted for the E1 gene (pAd). The pAd plasmid was transfected into human cells providing the E1 gene product in trans to allow replication and purification of recombinant adenovirus to be used as API in vaccines.

### Example 2. Expression of the Antigen Proteins

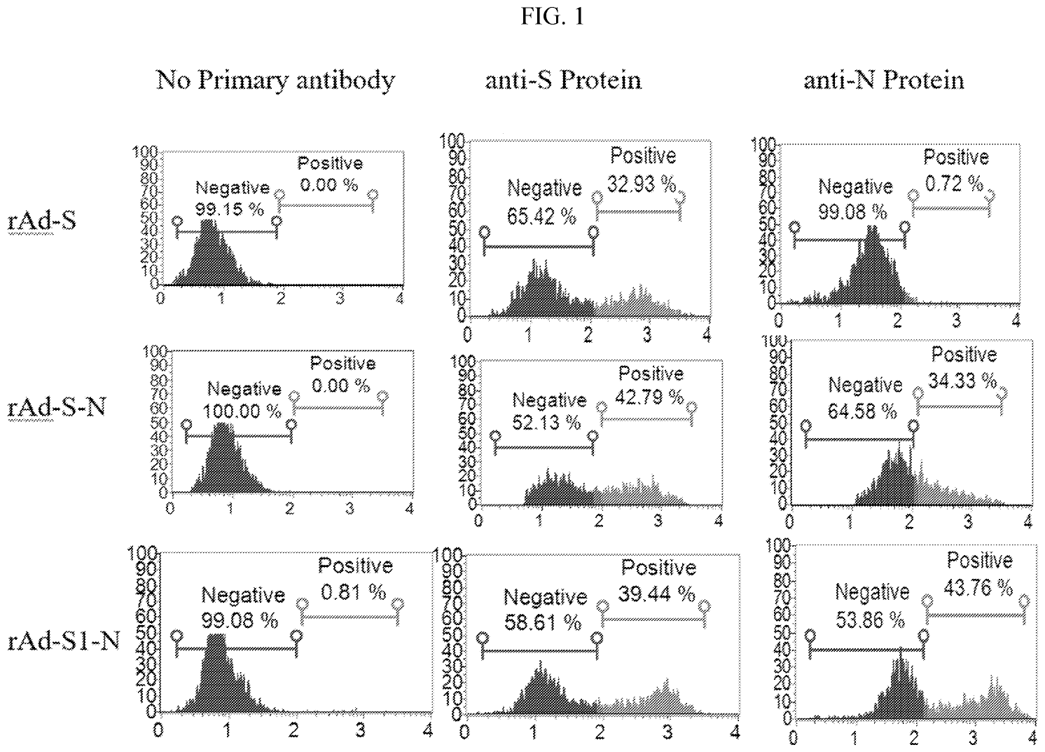

Three different candidates were evaluated for expression by intracellular staining/flow cytometry. HEK293 cells were placed in tissue culture at 3e5 cells/well in a 24-well plate. Four hrs later, the cells were infected with the various constructs at a MOI of 1. Cells were harvested 40 hours later, and human monoclonal antibodies that recognize the S1 or N proteins (Genscript) were used to stain separate wells. An anti-human IgG PE secondary antibody was used to visualize expression on the fixed cells. The candidate (rAd-S; plasmid pAd-CMV-SARS-CoV-2-S-BGH-CMV-dsRNA-SPA described above) that expressed full length SARS-CoV-2 S protein, but not the N protein clearly showed such expression patterns. The candidate (rAd-S1-N; plasmid pAd-CMV-SARS-CoV-2-S1-Furin-N-BGH-CMV-dsRNA-SPA as described above) that expressed a fusion protein of S1-N expressed both S and N proteins, as did the candidate (rAd-S-N; plasmid pAd-CMV-SARS-CoV-2-S-BGH-bActin-SARS-CoV-2-N-SPA-BGH-CMV-dsRNA-SPA as described above) that expressed S and N off separate promoters (FIG. 1).

### Example 3. Immunogenicity in Mice

The primary objective of the initial mouse immunogenicity studies was to determine which of the rAd vectors induced significant antibody responses. The results were used to determine which candidate vaccine would be selected for GMP manufacturing. Animals were immunized by i.n. (N=6) and the antibody titers were measured over time. The rAd vector expressing both S and N off separate promoters (plasmid pAd-CMV-SARS-CoV-2-S-BGH-bActin-SARS-CoV-2-N-SPA-BGH-CMV-dsRNA-SPA as described above) produced equivalent titers to the S1 component of the S protein from SARS-CoV-2. The rAd-S-N vector had slightly higher S1 antibody responses than the fusion protein expressing rAd-S1-N (FIG. 2).

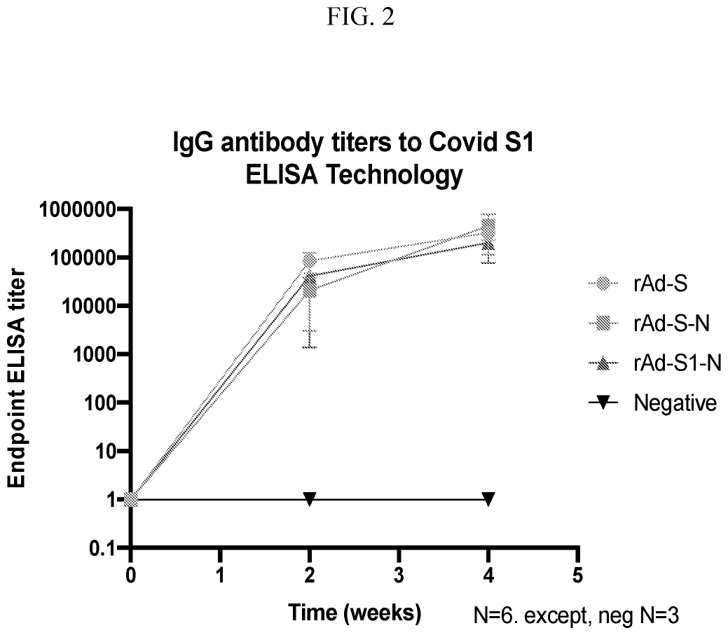

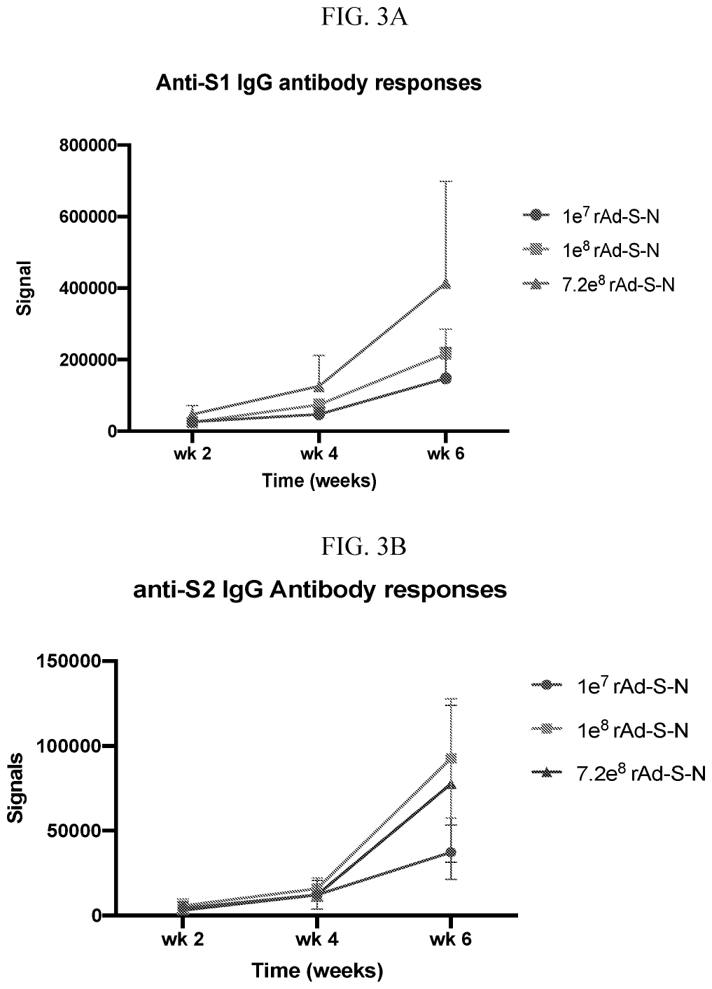

A dose response of the chosen vaccine rAd-S-N was then performed to test immunogenicity. Three different dose levels were tested, and the antibody responses to both S1 and S2 were measured using the Mesoscale device. Similar responses were seen at all three dose levels at early timepoints, but the higher dose groups had improved antibody responses at later time points (FIGS. 3A and 3B).

### Example 4. Immunogenicity in Humans

The rAd-S-N plasmid (pAd-CMV-SARS-CoV-2-S-BGH-bActin-SARS-CoV-2-N-SPA-BGH-CMV-dsRNA-SPA as described above) will be manufactured in a GMP facility, dried, and placed into tablets. A human trial will evaluate the ability of the rAd-S-N to elicit immune responses in humans at different dose levels.

### Example 5. Pre-Clinical Studies of a Recombinant Adenoviral Mucosal Vaccine to Prevent Sars-Cov-2 Infection

### Introduction

The emergence of a novel coronavirus, severe acute respiratory syndrome coronavirus 2 (SARS-CoV-2), the causative agent of COVID-19 disease, in 2019, has led to a global pandemic and significant morbidity, mortality and socio-economic disruption not seen in a century. Coronavirus disease 2019 (COVID-19) is a respiratory illness of variably severity; ranging from asymptomatic infection to mild infection, with fever and cough to severe pneumonia and acute respiratory distress1. Current reports suggest that asymptomatic spread is substantial (2), and SARS-CoV-2 infection induces a transient antibody response in most individuals (3). Therefore, development of successful interventions is an immediate requirement to protect the global population against infection and transmission of this virus and its associated clinical and societal consequences. Mass immunization with efficacious vaccines has been highly successful to prevent the spread of many other infectious diseases and can also prevent disease in the vulnerable through the induction of herd immunity. Significant effort and resources are being invested in urgently identifying efficacious SARS-CoV-2 vaccines. A number of different vaccine platforms have demonstrated pre-clinical immunogenicity and efficacy against pneumonia (4, 5). Several vaccines have demonstrated phase I or phase II safety and immunogenicity (6-8). However, at this time, no vaccine has demonstrated efficacy in the field.

The most advanced SARS-CoV-2 vaccine candidates are all given by the intramuscular (IM) route, with some requiring −80° C. storage. This is a major barrier for vaccine dissemination and deployment during a pandemic in which people are asked to practice social distancing and avoid congregation. The ultimate goal of any vaccine campaign is to protect against disease by providing enough herd immunity to inhibit viral spread, not to make a set number of doses of vaccine. An injected solution takes a long period of time to administer and distribute and requires costly logistics, which means dose availability does not immediately translate to immunity. Further, systemic immunization can induce immunity in the periphery and lower respiratory tract. However, these vaccines cannot induce mucosal immunity in the upper respiratory tract, as evidenced by the poor mucosal IgA reported from van Doremalen, et al., 4 Mucosal IgA (with the polymeric structure and addition of the secretory component), creates more potent viral neutralization (9), can block viral transmission (10, 11), and in general, is more likely to create sterilizing immunity given that this is the first line of defense for a respiratory pathogen.

Mucosal vaccines can induce mucosal immune responses, antibodies and T cells at wet surfaces. We are developing oral vaccines for multiple indications, including influenza and noroviruses, delivered in a tablet form for people. Our vaccine platform is a replication-defective adenovirus type-5 vectored vaccine that expresses antigen along with a novel toll-like receptor 3 agonist as an adjuvant. These vaccines have been well tolerated, and able to generate robust humoral and cellular immune responses to the expressed antigens (12-14). Protective efficacy in humans was demonstrated against a respiratory virus 90 days or more post vaccination, as shown in a well characterized experimental influenza infection model (15). Furthermore, the vaccine also has the advantage of room temperature stability and needle-free, ease of administration, providing several advantages over injected vaccine approaches with respect to vaccine deployment and access.

Here, we describe the pre-clinical development of a SARS-CoV-2 vaccine based on Vaxart's oral adenovirus platform. The key approach was to develop several vaccine candidates in parallel, in order to create premanufacturing seeds while initial immunogenicity experiments were in progress. Given that the vaccines were made during the pandemic, rapid decisions were required to keep the manufacturing and regulatory timelines from slipping. We assessed the relative immunogenicity of four candidate vaccines that expressed antigens based on the spike (S) and nucleocapsid (N) SARS-CoV-2 proteins. These proteins have been well characterized as antigens for related coronaviruses, such as SARS-CoV and MERS (reviewed in Yong, et al., (16)) and, increasingly, for SARS-CoV-2 spike. The aim of our vaccine is to induce immunogenicity on three levels; firstly, to induce potent serum neutralizing antibodies to S, secondly to induce mucosal immune responses, and thirdly to induce T cell responses to both vaccine antigens. This three-fold approach aims to induce robust and broad immunity capable of protecting the individual from virus infection as well as disease, promote rapid dissemination of vaccine during a pandemic, and to protect the population from virus transmission through herd immunity.

Here, we report the induction of neutralizing antibody (Nab), IgG and IgA antibody responses, and T cell responses in mice following immunization of rAd vectors expressing one or more SARS-CoV-2 antigens.

### Results

### Vector Construction

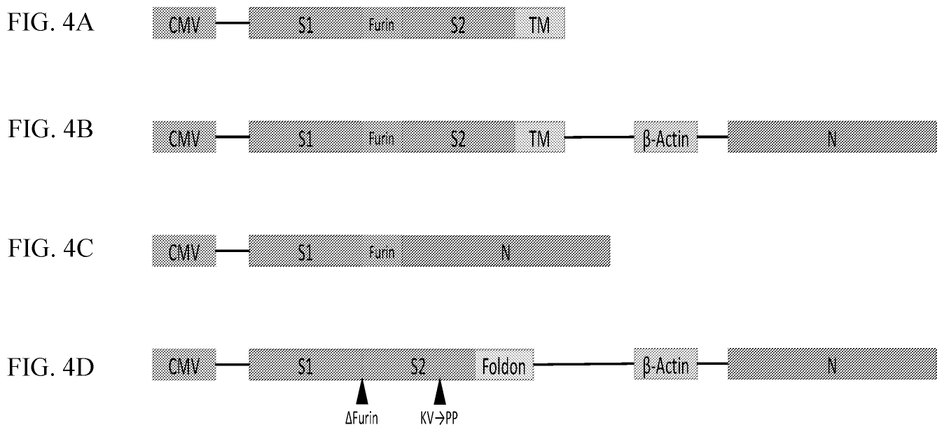

Initially, three different rAd vectors were constructed to express different SARS-CoV-2 antigens. These were a vector expressing the full-length S protein (rAd-S), a vector expressing the S protein and the N protein (rAd-S-N), and a vector expressing a fusion protein of the S1 domain with the N protein (rAd-S1-N). The N protein of rAd-S-N was expressed under control of the human beta actin promoter, which is much more potent in human cells than mouse cells. An additional construct where the expressed S protein was fixed in a prefusion conformation (rAd-S(fixed)-N) was constructed at a later date as a control for exploring neutralizing antibody responses. These are described in FIG. 4. Expression of the various transgenes was confirmed following infection of 293 cells using flow cytometry and monoclonal antibodies to the S or N protein.

### Immunogenicity of rAd Vectors Expressing S and N Antigens

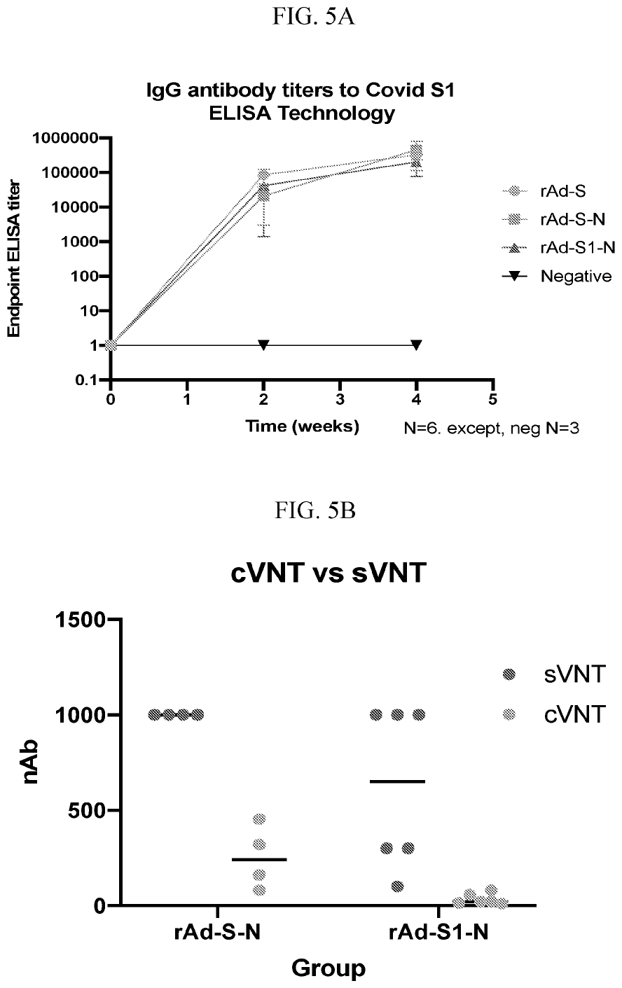

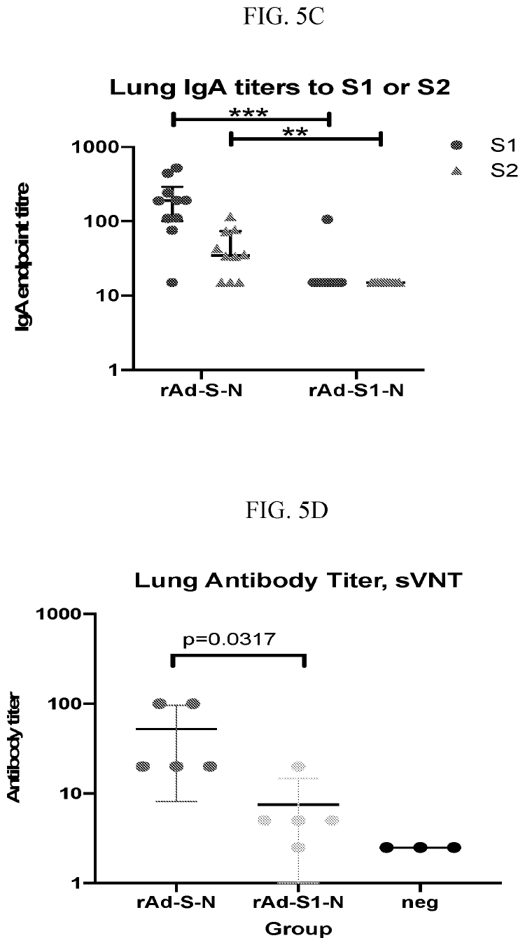

The primary objective of the initial mouse immunogenicity studies was to determine which of the rAd vectors induced significant antibody responses to S, and to obtain those results rapidly enough to provide a GMP seed in time for manufacturing. We and others (17) have observed that transgene expression by vaccine vectors orally administered to mice can be suppressed in their intestinal environment, so immunogenicity was assessed following intranasal (i.n.) immunization. Animals were immunized i.n. and the antibody titers were measured over time by IgG ELISA. All three rAd vectors induced nearly equivalent anti-S1 IgG titers, at weeks 2 and 4 and the IgG titer in all animals was significantly boosted by the second immunization (p<0.05 Mann Whitney t-tests) (FIG. 5A). However, the vector expressing full-length S (rAd-S-N) induced higher neutralizing titers compared to the vector expressing only S1 (FIG. 5B). This was measured by two different neutralizing assays, one based on SARS-CoV-2 infection of Vero cells (cVNT) and one based on a surrogate neutralizing assay (sVNT). Furthermore, rAd-S-N induced higher lung IgA responses to S1 and unsurprisingly, to S2 (FIG. 5C) compared to rAd-S1-N two weeks after the final immunization. Notably, neutralizing titers in the lung were also significantly higher when rAd-S-N was used compared to the S1-containing vaccine (rAd-S1-N) (FIG. 5D). This demonstrated that the rAd-S-N candidate induced greater functional responses (NAb and IgA) compared to the vaccine containing the just the S1 domain. Because the N protein is much more highly conserved than the S protein, and is a target of long term T cell responses induced by infection (18), the vector rAd-S-N was chosen for GMP manufacturing.

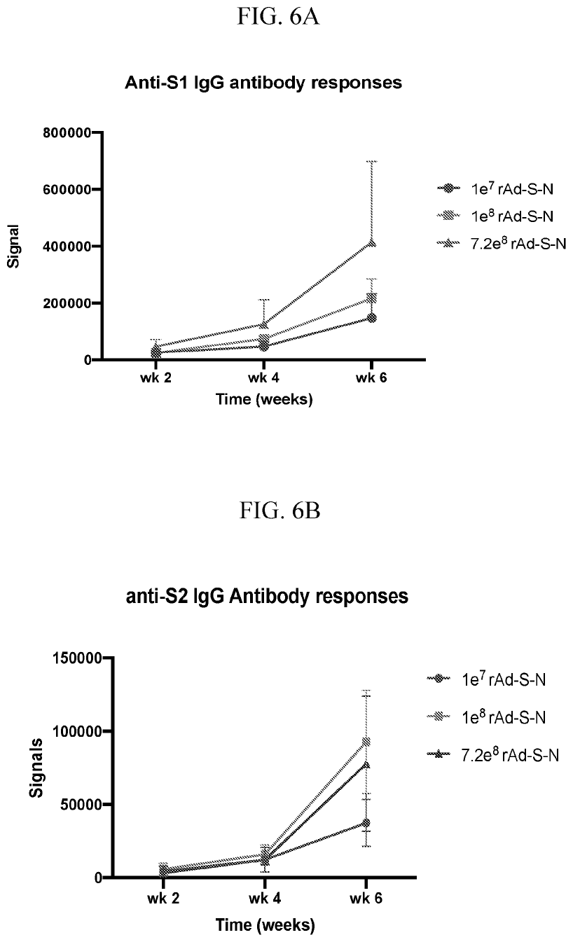

Three dose levels of rAd-S-N were then tested to understand the dose responsiveness of this vaccine. The antibody responses to both S1 (FIG. 6A) and S2 (FIG. 6B) were measured. Similar responses were seen at all three dose levels at all timepoints. Responses to S1 and S2 were significantly increased at week 6 compared to earlier times, in all groups.

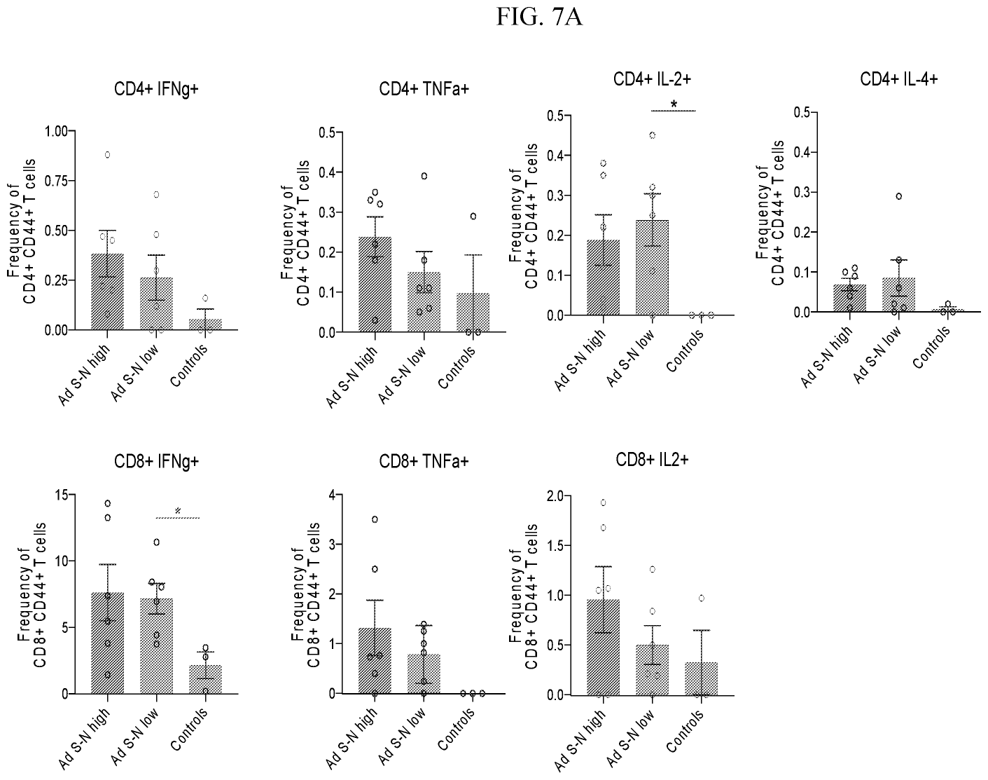

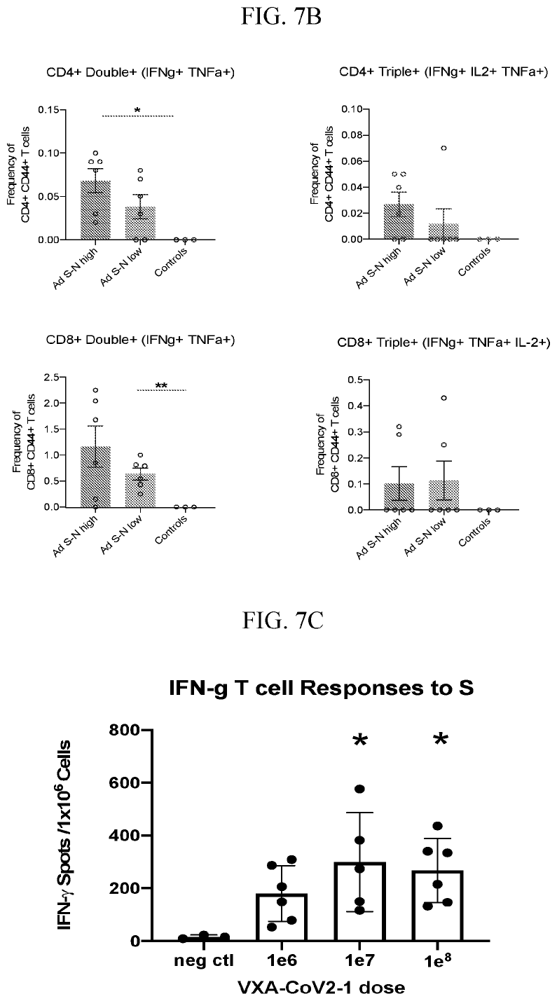

The induction of S-specific T cells by rAd-S-N at different doses was then assessed. Induction of antigen-specific CD4+ and CD8+ T cells that produced effector cytokines such as IFN-γ, TNF-α and IL-2 was observed two weeks after 2 immunizations (FIG. 7A). Notably, little IL-4 was induced by this vaccine and only in CD4+ T cells; providing a level of assurance that the risk for vaccine dependent enhancement of disease was very low. Furthermore, immunization with rAd-S-N induced double and triple positive, multi-functional IFN-γ, TNF-α and IL-2 CD4+ T cells (FIG. 7B). A second dose response experiment was performed to focus on T cell responses to the S protein, 4 weeks after the final immunization (week 8 of the study). Splenocytes were stimulated overnight with a peptide library to the S protein, divided in two separate peptide pools. T cell responses in the two pools were summed and plotted (FIG. 7C). Animals administered the 1e7 IU and the 1e8 IU dose levels had significantly higher T cell responses compared to the untreated animals but produced a similar number of IFN-γ secreting cells to each other, demonstrating a dose plateau at the 1e7 IU dose. Notably, this T cell analysis was conducted 4 weeks after the second immunization, potentially after the peak of T cell responses.

rAd-Expressed Wild-Type S Induces a Superior Neutralizing Response Compared to Stabilized Pre-Fusion S.

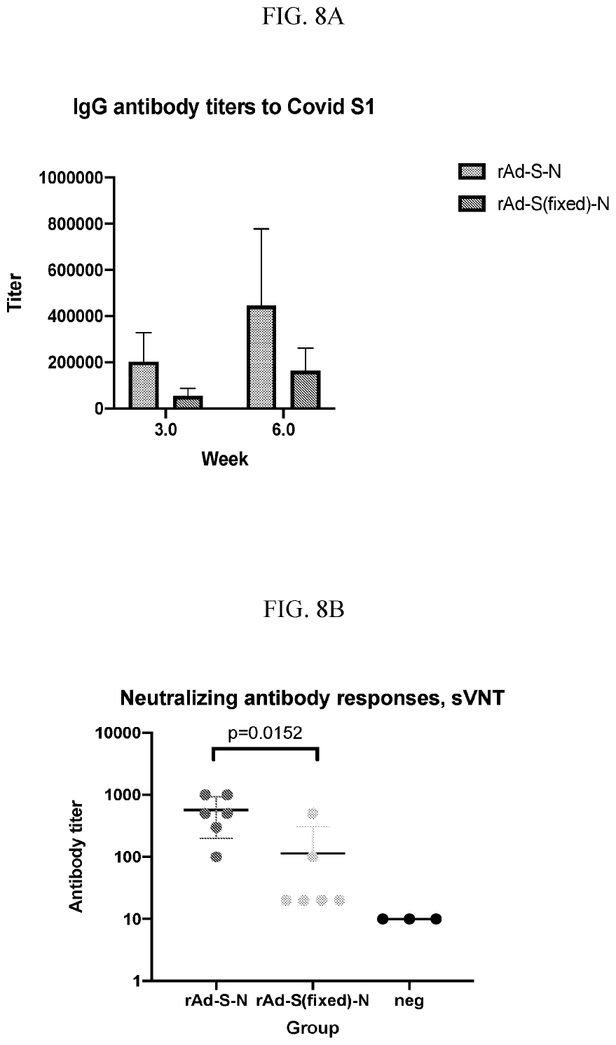

An additional study was performed to compare rAd-S-N to a vaccine candidate with the S-protein stabilized and with the transmembrane region removed (rAd-S(fixed)-N). A stabilized version of the S protein has been proposed as a way to improve neutralizing antibody responses and produce less non-neutralizing antibodies. The S protein was stabilized through modifications as described by Amanat et al., (19). rAd-S-N induced higher serum IgG titers to S1 (FIG. 8A) at both timepoints tested, although these were not statistically significant at week 6 by Mann-Whitney (p=0.067). However, rAd-S-N induced significantly higher neutralizing antibody responses (FIG. 8B) than the stabilized version (p=0.0152). These results suggest that a wild-type version of the S protein is superior for a rAd based vaccine in mice.

### Discussion

The endgame to the COVID-19 pandemic requires the identification and manufacture of a safe and effective vaccine and a subsequent global immunization campaign. A number of vaccine candidates have accelerated to phase III global efficacy testing and, if sufficiently successful in these trials, may form the first generation of an immunization campaign. However, all of these advanced candidates are S-based vaccines that are injected. Such an approach will unlikely prevent virus transmission, but should prevent pneumonia and virus growth and damage in the lower respiratory tract and periphery, as evidenced in macaque challenge studies (4, 5).

One key constraint in a global COVID-19 immunization campaign will be the cold chain distribution logistics and a bottleneck of requiring suitably trained health care workers (HCWs) to inject the vaccine. Current logistics costs, including cold chain and training, can double the cost of fully immunizing an individual in a low-middle income country (LMIC) (20). Implementing a mass immunization campaign, requiring trained HCWs for injection-based vaccines, will have a significant impact on healthcare resources in all countries. The need for cold chain, biohazardous sharps waste disposal and training will result in increased cost, inequitable vaccine access, delayed vaccine uptake and prolongation of this pandemic. These costs will be magnified if vaccines are unable to provide long-term protection (natural immunity to other beta-coronaviruses is short-lived (21)), and annual injection-based campaigns are needed. Vaxart's oral tablet vaccine platform provides a solution to these immunological as well as logistic, economic, access and acceptability problems. In this study we demonstrate, in an animal pre-clinical model, the immunogenicity of a SARS-CoV-2 vaccine using Vaxart's vaccine platform; namely the induction of serum and mucosal neutralising antibodies and poly-functional T cells.

Mouse studies were designed to test immunogenicity of candidate vaccines rapidly in the spring of 2020, before moving onto manufacturing and clinical studies critical to addressing the pandemic. Vaxart's oral tablet vaccine platform has previously proven to be able to create reliable mucosal (respiratory and intestinal), T cell, and antibody responses against several different pathogens in humans (12, 14, 22, 23). We know from our prior human influenza virus challenge study that oral immunization was able to induce protective efficacy 90 days post immunization; on par with the commercial quadrivalent inactivated vaccine (15). These features provide confidence that the adoption of the platform to COVID-19 could translate to efficacy against this pathogenic coronavirus and could provide durable protection against virus infection. Finally, a tablet vaccine campaign is much easier because qualified medical support is not needed to administer it. This ease of administration will result in increased vaccine access and potentially, acceptability, as has been evidenced by the success of easy-to-administer, oral polio vaccine, in the elimination of polio virus (24). These features could be even more important during SARS-CoV-2 immunization campaigns compared to other vaccines, as substantially more resources may be required to ensure uptake of this vaccine, given the global levels of COVID-19 denialism, mistrust and increased vaccine hesitancy (25, 26). The tablet vaccine does not need refrigerators or freezers, does not require needles or vials, and can potentially be shipped via standard mail or by a delivery drone. These qualities significantly enhance deployment and distribution logistics, even permitting access to isolated regions with fewer technical resources. Finally, from an immunological perspective, oral administration of this adenovirus is not compromised by pre-existing immunity to adenoviruses or creates substantial anti-vector immunity (12, 13), issues that have been shown to cause significantly decreased vaccine potency in an rAd5 based SARS-CoV2 vaccine (27) and can prevent durable increased immunity when the same adenovirus platform is re-administered by the IM route (28).

The choice of antigen can be difficult during a novel pandemic, a time in which key decisions are needed quickly. The S protein is believed to be the major neutralizing antibody target for coronavirus vaccines, as the protein is responsible for receptor binding, membrane fusion, and tissue tropism. When comparing SARS-CoV-2 Wu-1 to SARS-CoV, the S protein was found to have 76.2% identity (29). Both SARS-CoV and SARS-CoV-2 are believed to use the same receptor for cell entry: the angiotensin-converting enzyme 2 receptor (ACE2), which is expressed on some human cell types30. Thus, SARS-CoV-2 S protein is being used as the leading target antigen in vaccine development so far and is an ideal target given that it functions as the key mechanism for viral binding to target cells. However, the overall reliance on the S protein and an IgG serum response in the vaccines could eventually lead to viral escape. For influenza, small changes in the hemagglutinin binding protein, including a single glycosylation site, can greatly affect the ability of injected vaccines to protect (31). SARS-CoV-2 appears to be more stable than most RNA viruses, but S protein mutations have already been observed without the selective pressure of a widely distributed vaccine. Once vaccine pressure begins, escape mutations might emerge. We took two approaches to address this issue; firstly to include the more conserved N protein in the vaccine and secondly to induce broader immune responses, namely through mucosal IgA.

High expression levels of ACE2 are present in type II alveolar cells of the lungs, absorptive enterocytes of the ileum and colon, and possibly even in oral tissues such as the tongue (32). Transmission of the virus is believed to occur primarily through respiratory droplets and fomites between unprotected individuals in close contact (33), although there is some evidence of transmission via the oral-fecal route as seen with both SARS-CoV and MERS-CoV viruses where coronaviruses can be secreted in fecal samples from infected humans (34). There is also evidence that a subset of individuals exist that have gastrointestinal symptoms, rather than respiratory symptoms, are more likely to shed virus longer (35). Driving immune mucosal immune responses to S at both the respiratory and the intestinal tract may be able provide broader immunity and a greater ability to block transmission, than simply targeting one mucosal site alone. Blocking transmission, rather than just disease, will be essential to reducing infection rates and eventually eradicating SARS-CoV-2. We have previously demonstrated that an oral, tableted rAd-based vaccine can induce protection against respiratory infection and shedding following influenza virus challenge (15) as well as intestinal immunity to norovirus antigens in humans (12). Furthermore, mucosal IgA is more likely to be able address any heterogeneity of the S proteins in circulating viruses than a monomeric IgG response. mIgA has also been found to be more potent at cross reactivity than IgG for other respiratory pathogens (36). IgA may also be a more neutralizing isotype than IgG in COVID-19 infection, and in fact neutralizing IgA dominates the early immune response (37). Notably, we saw a higher ratio between neutralizing to non-neutralizing antibodies in our lung versus serum antibody results in our mouse study as well, which supports the concept that IgA may have more potency compared to IgG. Polymeric IgA, through multiple binding interactions to the antigen and to Fc receptors can turn a weak single interaction into a higher overall affinity binding and activation signal, creating more cross-protection against heterologous viruses (38).

Our second strategy to mitigate this potential vaccine-driven escape problem was to include the N protein in the vaccine construct. The N protein is highly conserved among β-coronaviruses, (greater than 90% identical) contains several immunodominant T cell epitopes, and long-term memory to N can be found in SARS-CoV recovered subjects as well as people with no known exposure to SARS-CoV or SARS-CoV-2 (18, 39). In an infection setting, T cell responses to the N protein seem to correlate to increased neutralizing antibody responses (40). All of these reasons led us to add N to our vaccine approach. The protein was expressed in 293A cells. However, as the human beta actin promoter is more active in human cells than mice, we did not explore immune responses in Balb/c mice, but will examine them more carefully in future NHP and human studies.

The optimum sequence and structure of the S protein to be included in a SARS-CoV-2 vaccine is a subject of debate. Several labs have suggested that reducing the S protein to the key neutralizing domains within the receptor binding domain (RBD) would promote higher neutralizing antibody responses, and fewer non-neutralizing antibodies (41, 42). We made a vaccine candidate composed of the S1 domain, which includes the RBD, in an attempt to promote this approach. Although the S1-based vaccine produced similar IgG binding titers to S1, neutralizing antibody responses were significantly lower compared to the full-length S antigen. Other gene-based vaccines have also shown the reductionist approach to S does not work so well, demonstrating that the DNA vaccine expressing the full-length S-protein produced higher neutralizing antibodies than shorter S segments (5). In agreement with these macaque studies, we observed that the sequence of the Ad-encoded antigen had a significant impact on antibody function, here with respect to neutralization. While reducing the potential for exposing non-neutralizing antibody epitopes seems reasonable in theory, this might reduce the T cell help that allows for greater neutralizing antibodies to develop. Indeed, of the spike protein T cell responses, which make up 54% of the responses to SARS-CoV-2, only 11% map to receptor binding domain (43). Stabilizing the S protein might be important for a protein vaccine, but not necessarily for a gene-based vaccine. The former is produced in vitro and it is produced to retain a homogenous, defined structure, ready for injection. In contrast, the latter, is expressed on the surface of a cell, in vivo, like natural infection, substantially in a prefusion form, and the additional stabilization may be unnecessary for B cells to create antibodies against the key neutralizing epitopes. We directly compared a stabilized version of S to the wild-type version in construct encoding the S and N proteins as described in this example. The wild-type version was significantly better at inducing neutralizing antibody responses. Of interest, this was also observed in a DNA vaccine study in NHPs, where the stabilized version appeared to induce lower neutralizing antibody (NAb) titers compared to the wild-type S5. A slightly different result was observed in studies of rAd26 vectors by Mercado, et al in NHPs, where expressing a stabilized version of the S protein appeared to improve NAb but lower T cell responses (44). In summary, stabilization doesn't universally improve the immune responses in gene-based or vector-based vaccines.

Multiple vaccine candidates are in, or are about to begin, clinical testing. Due to known safety and immunogenicity for epidemic pathogens such as Ebola virus, two leading candidate vaccines are based on recombinant adenovirus vectors; University of Oxford's ChAdOx1-nCov and Janssen Pharmaceutical's AdVac platforms (45-48). We saw stronger serum IgG and NAb titers in our study compared to a ChAdOx1-nCov in Balb/c mice. (4) However, this might reflect differences in assay components. An rAd36 vaccine study was performed by Hassan, et al., where doses of 1e10 VP were given by intranasal delivery (49). The results were significant from the standpoint of blocking lung infection in a mouse SARS-CoV-2 challenge model. They reported titers of serum antibody titers of 1e4 above the background titers, similar to our results, despite using doses 2- to 3-log fold higher compared to our study. Indeed, in our study, equivalently strong T cell and antibody responses were observed using 1e7 IU and 1e8 IU by the intranasal route. Using these doses, we observed high percentages of CD8+ T cell responses (up to 14%) secreting IFN-γ and TNF-α and strong CD4+ T cells after peptide restimulation. Although we did not evaluate the trafficking properties of these antigen-specific T cells, we know that oral administration of this Ad-based vaccine in humans induces high levels of mucosal homing lymphocytes (12, 15). A proportion of the antigen-specific CD4+ and CD8+ T cells were polyfunctional in this mouse study. Vaccine-induced T cells possessing multiple functions may provide more effective elimination of virus subsequent to infection and therefore could be involved in the prevention of disease, however it is uncertain at this time what is the optimum T cell phenotype required for protection against disease.

In summary, these studies in mice represent were our first step in creating a vaccine candidate, demonstrating the immunogenicity of the construct at even low vaccine doses and the elucidation of the full-length spike protein as a leading candidate antigen to induce T cell responses and superior systemic and mucosal neutralizing antibody. Future work will focus on the immune responses in humans.

### Methods

### Vaccine Constructs

For this study, four recombinant adenoviral vaccine constructs were created based on the published DNA sequence of SARS-CoV-2 publicly available as Genbank Accession No. MN908947.3. Specifically, the published amino acid sequences of the SARS-CoV-2 spike protein (S protein) and the SARS-CoV-2 Nucleocapsid protein (N protein) were used to synthesize nucleic acid sequences codon optimized for expression in Homo sapiens cells (Blue Heron Biotechnology, Bothell, Wash.). These sequences were used to create recombinant plasmids containing transgenes cloned into the E1 region of Adenovirus Type 5 (rAd5), as described by He, et al. (50), using the same vector backbone used in prior clinical trials for oral rAd tablets (12, 15). As shown in FIG. 4, the following four constructs were created:

a. rAd-S: rAd5 vector containing full-length SARS-CoV-2 S gene under control of the CMV promoter.
b. rAd-S-N: rAd5 vector containing full-length SARS-CoV-2 S gene under control of the CMV promoter and full-length SARS-CoV-2 N gene under control of the human beta-actin promoter.
c. rAd-S1-N: rAd5 vector using a fusion sequence combining the S1 region of SARS-CoV-2 S gene (including the native furin site between S1 and S2) with the full-length SARS-CoV-2 N gene.
d. rAd-S(fixed)-N: rAd5 vector containing a stabilized S gene with the transmembrane region removed under the control of the CMV promoter and full-length SARS-CoV-2 N gene under control of the human beta-actin promoter. The S gene is stabilized through the following modifications:
a) Arginine residues at aa positions 682, 683, 685 were deleted to remove the native furin cleavage site
b) Two stabilizing mutations were introduced: K986P and V987P
c) Transmembrane region was removed following P1213 and replaced with bacteriophage T4 fibritin trimerization foldon domain sequence (51) (GYIPEAPRDGQAYVRKDGEWVLLSTFL)

All vaccines were grown in the Expi293F suspension cell-line (Thermo Fisher Scientific), purified by CsCl density centrifugation and provided in a liquid form for animal experiments.

### Animal Experiments

Studies were approved for ethics by the Animal Care and Use Committees (IACUC). All of the procedures were carried out in accordance with local, state and federal guidelines and regulations. Female 6-8 week old Balb/c mice were purchased from Jackson labs (Bar Harbor, Me.). Because mice do not swallow pills, liquid formulations were instilled intranasally in 10 μl per nostril, 20 μl per mouse in order to test immunogenicity of the various constructs. Serum was acquired by cheek puncture at various timepoints.

### Antibody Assessment

### ELISAs.

Specific antibody titers to proteins were measured similarly to methods described previously (52). Briefly, microtiter plates (MaxiSorp: Nunc) were coated in 1 carbonate buffer (0.1 M at pH 9.6) with 1.0 ug/ml S1 protein (GenScript). The plates were incubated overnight at 4° C. in a humidified chamber and then blocked in PBS plus 0.05% Tween 20 (PBST) plus 1% BSA solution for 1 h before washing. Plasma samples were serially diluted in PBST. After a 2-h incubation, the plates were washed with PBST at least 5 times. Antibodies were then added as a mixture of anti-mouse IgG1-horseradish peroxidase (HRP) and anti-mouse IgG2a-HRP (Bethyl Laboratories, Montgomery, Tex.). Each secondary antibody was used at a 1:5,000 dilution. The plates were washed at least 5 times after a 1-h incubation. Antigen-specific mouse antibodies were detected with 3,3=,5,5=-tetramethyl-benzidine (TMB) substrate (Rockland, Gilbertsville, Pa.) and H2SO4 was used as a stop solution. The plates were read at 450 nm on a Spectra Max M2 Microplate Reader. Average antibody titers were reported as the reciprocal dilution giving an absorbance value greater than the average background plus 2 standard deviations, unless otherwise stated.

### Antibody Binding Antibodies

To measure responses to both S1 and S2 simultaneously, A MULTI-SPOT® 96-well, 2-Spot Plate (Mesoscale Devices; MSD) was coated with SARS CoV-2 antigens. Proteins were commercially acquired from a source (Native Antigen Company) that produced them in mammalian cells (293 cells). These were biotinylated and adhered to their respective spots by their individual U-PLEX linkers. To measure IgG antibodies, plates were blocked with MSD Blocker B for 1 hour with shaking, then washed three times prior to the addition of samples, diluted 1:4000. After incubation for 2 hours with shaking, the plates were washed three times. The plates were then incubated for 1 hour with the detection antibody at 1 μg/mL (MSD SULFO-TAG™ Anti-mouse IgG). After washing 3 times, the Read Buffer was added and the plates were read on the Meso QuickPlex SQ 120.

### SARS-CoV-2 Neutralization Assays

Neutralizing antibodies were routinely detected based on the SARS-CoV-2 Surrogate Virus Neutralization Test (sVNT) kit (GenScript). This ELISA-based kit detects antibodies that hinder the interaction between the receptor binding domain (RBD) of the SARS-CoV-2 spike glycoprotein and the ACE2 receptor on host cells, and is highly correlated to conventional virus neutralizing titers for SARS-CoV-2 infection of Vero cells (53). The advantage of this approach is that the assay can be done in a BSL-2 laboratory. Sera from mice immunized with the candidate vaccines was diluted at 1:20, 1:100, 1:300, 1:500, 1:750 and 1:1000 using the provided sample dilution buffer. Sera from non-immunized mice was diluted 1:20. Lung samples were diluted 1:5, 1:20, and 1:100. Positive and negative controls were prepared at a 1:9 volume ratio following the provided protocol. After dilution, sera or lung samples were individually incubated at a 1:1 ratio with HRP-RBD solution for 30 minutes at 37° C. Following incubation, 100 μl of the each HRP-RBD and sample or control mixture was added to the corresponding wells in the hACE2-precoated capture plate and once again incubated at 37° C. for 15 minutes. Afterwards, wells were thoroughly washed and 100 μl of the provided TMB (3,3=,5,5=-tetramethyl-benzidine) solution was added to each well and left to incubate for 15 minutes at room temperature (20-25° C.). Lastly 50 μl of Stop Solution was added to each well, and the plate was read on a Spectra Max M2 Microplate Reader at 450 nm. The absorbance of a given sample is inversely related on the titer of anti-SARS-CoV-2 RBD neutralizing antibody in a given sample. Per test kit protocol, a cut-off of 20% inhibition when comparing the OD of the sample versus the OD of the negative control was determined to be positive for the presence of neutralizing antibodies. Samples that were negative at the lowest dilution were set equal to ½ of the lowest dilution tested, either 10 for sera or 2.5 for lung samples.

Additional neutralizing antibodies responses were measured in some studies using a cVNT assay at Visimederi under BSL3 conditions. The cVNT assay has a readout of Cytopathic Effect (CPE) to detect specific neutralizing antibodies against live SARS-COV-2 in animal or human samples. The cVNT/CPE assay permits the virus to makes multiples cycles of infection and release from cells; its exponential grow in few days (usually 72 hours of incubation) causes the partial or complete cell monolayer detachment from the surface of the support, clearly identifiable as CPE. Serum samples are heat inactivated for 30 min at 56°; two-fold dilutions, starting from 1:10 are performed then mixed with an equal volume of viral solution containing 100 TCID50 of SARS-CoV-2. The serum-virus mixture is incubated for 1 hour at 37° in humidified atmosphere with 5% CO2. After incubation, 100 μL of the mixture at each dilution are added in duplicate to a cell plate containing a semiconfluent Vero E6 monolayer. After 72 hours of incubation the plates are inspected by an inverted optical microscope. The highest serum dilution that protect more than 50% of cells from CPE is taken as the neutralization titer.

### Lung IgA ELISAs.

Two weeks after the final immunization (day 28 of the study), mice were sacrificed and bled via cardiac puncture. Lungs were removed and snap frozen at −80° C. On thawing, lungs were weighed. Lungs were homogenized in 150 μl DPBS using pellet pestles (Sigma Z359947). Homogenates were centrifuged at 1300 rpm for 3 minutes and supernatants were frozen. The total protein content in lung homogenate was evaluated using a Bradford assay to ensure equivalent amounts of tissue in all samples before evaluation of IgA content. Antigen-specific IgA titers in lungs were detected using a mouse IgA ELISA kit (Mabtech) and pNPP substrate (Mabtech). Briefly, MaxiSorp plates (Nunc) were coated with S1 or S2 (The Native Antigen Company; 50 ng/well) in PBS for overnight adsorption at 4° C. and then blocked in PBS plus 0.05% Tween 20 (PBST) plus 0.1% BSA (PBS/T/B) solution for 1 h before washing. Lung homogenates were serially diluted in PBS/T/B, starting at a 1:30 dilution. After 2 hours incubation and washing, bound IgA was detected using MT39A-ALP conjugated antibody (1:1000), according to the manufacturer's protocol. Plates were read at 415 nm. Endpoint titers were taken as the x-axis intercept of the dilution curve at an absorbance value 3× standard deviations greater than the absorbance for naïve mouse serum. Non-responding animals were set a titer of 15 or ½ the value of the lowest dilution tested.

### T Cell Responses

Spleens were removed and placed in 5 ml Hanks Balanced Salt Solution (with 1M HEPES and 5% FBS) before pushing through a sterile strainer with a 5 ml syringe. After RBC lysis (Ebiosolutions), resuspension, and counting, the cells were ready for analysis. Cells were cultured at 5e5 cells/well with two peptide pools representing the full-length S protein at 1 μg/ml (Genscript) overnight in order to stimulate the cells. The culture media consisted of RPMI media (Lonza) with 0.01M HEPES, 1×l-glutamine, 1×MEM basic amino acids, 1× penstrep, 10% FBS, and 5.5e-5 mole/l beta-mercaptoethanol. Antigen specific IFN-γ ELISPOTs were measured using a Mabtech kit. Flow cytometric analysis was performed using an Attune Flow cytometer and Flow Jo version 10.7.1, after staining with the appropriate antibodies. For flow cytometry, 2e6 splenocytes per well were incubated for 18 hours at 37° C. with peptide pools representing full-length S at either 1 or 5 ug/ml, adding Brefeldin A (ThermoFisher) for the last 4 hours of incubation. The antibodies used were APC-H7 conjugated CD4, FITC conjugated CD8, BV650 conjugated CD3, PerCP-Cy5.5 conjugated IFN-γ, BV421 conjugated IL-2, PE-Cy7 conjugated TNFα, APC conjugated IL-4, Alexa Fluor conjugated CD44, and PE conjugated CD62L (BD biosciences).

### References for Example 5

1. Huang C, Wang Y, Li X, et al. Clinical features of patients infected with 2019 novel coronavirus in Wuhan, China. Lancet 2020; 395(10223): 497-506.
2. Tan J, Liu S, Zhuang L, et al. Transmission and clinical characteristics of asymptomatic patients with SARS-CoV-2 infection. Future Virol 2020: 10.2217/fvl-020-0087.
3. Long Q X, Tang X J, Shi Q L, et al. Clinical and immunological assessment of asymptomatic SARS-CoV-2 infections. Nat Med 2020; 26(8): 1200-4.
4. van Doremalen N, Lambe T, Spencer A, et al. ChAdOx1 nCoV-19 vaccine prevents SARS-CoV-2 pneumonia in rhesus macaques. Nature 2020.
5. Yu J, Tostanoski L H, Peter L, et al. DNA vaccine protection against SARS-CoV-2 in rhesus macaques. Science 2020.
6. Folegatti P M, Ewer K J, Aley P K, et al. Safety and immunogenicity of the ChAdOx1 nCoV-19 vaccine against SARS-CoV-2: a preliminary report of a phase 1/2, single-blind, randomised controlled trial. Lancet 2020.
7. Zhu F C, Guan X H, Li Y H, et al. Immunogenicity and safety of a recombinant adenovirus type-5-vectored COVID-19 vaccine in healthy adults aged 18 years or older; a randomised, double-blind, placebo-controlled, phase 2 trial. Lancet 2020; 396(10249): 479-88.
8. Jackson L A, Anderson E J, Rouphael N G, et al. An mRNA Vaccine against SARS-CoV-2—Preliminary Report. N Engl J Med 2020.
9. Renegar K B, Jackson G D, Mestecky J. In vitro comparison of the biologic activities of monoclonal monomeric IgA, polymeric IgA, and secretory IgA. Journal of immunology 1998; 160(3): 1219-23.
10. Seibert C W, Rahmat S, Krause J C, et al. Recombinant IgA is sufficient to prevent influenza virus transmission in guinea pigs. Journal of virology 2013; 87(14): 7793-804.
11. Lowen A C, Steel J, Mubareka S, Camero E, Garcia-Sastre A, Palese P. Blocking interhost transmission of influenza virus by vaccination in the guinea pig model. J Virol 2009; 83(7): 2803-18.
12. Kim L, Liebowitz D, Lin K, et al. Safety and immunogenicity of an oral tablet norovirus vaccine, a phase I randomized, placebo-controlled trial. JCI Insight 2018; 3(13): e121077.
13. Liebowitz D, Lindbloom J D, Brandl J R, Garg S J, Tucker S N. High titre neutralising antibodies to influenza after oral tablet immunisation: a phase 1, randomised, placebo-controlled trial. The Lancet infectious diseases 2015; 15(9): 1041-8.
14. Peters W, Brandl J R, Lindbloom J D, et al. Oral administration of an adenovirus vector encoding both an avian influenza A hemagglutinin and a TLR3 ligand induces antigen specific granzyme B and IFN-gamma T cell responses in humans. Vaccine 2013; 31: 1752-8.
15. Liebowitz D, Gottlieb K, Kolhatkar N S, et al. Efficacy and immune correlates of protection induced by an oral influenza vaccine evaluated in a phase 2, placebo-controlled human experimental infection study. The Lancet infectious diseases 2020; 20: 435-44.
16. Yong C Y, Ong H K, Yeap S K, Ho K L, Tan W S. Recent Advances in the Vaccine Development Against Middle East Respiratory Syndrome-Coronavirus. Front Microbiol 2019; 10: 1781.
17. Revaud J, Unterfinger Y, Rol N, et al. Firewalls Prevent Systemic Dissemination of Vectors Derived from Human Adenovirus Type 5 and Suppress Production of Transgene-Encoded Antigen in a Murine Model of Oral Vaccination. Front Cell Infect Microbiol 2018; 8: 6.
18. Peng H, Yang L T, Wang L Y, et al. Long-lived memory T lymphocyte responses against SARS coronavirus nucleocapsid protein in SARS-recovered patients. Virology 2006; 351(2): 466-75.
19. Amanat F, Stadlbauer D, Strohmeier S, et al. A serological assay to detect SARS-CoV-2 seroconversion in humans. medRxiv 2020.
20. Gandhi G, Lydon P, Cornejo S, Brenzel L, Wrobel S, Chang H. Projections of costs, financing, and additional resource requirements for low- and lower middle-income country immunization programs over the decade, 2011-2020. Vaccine 2013; 31 Suppl 2: B137-48.
21. Kissler S M, Tedijanto C, Goldstein E, Grad Y H, Lipsitch M. Projecting the transmission dynamics of SARS-CoV-2 through the postpandemic period. Science 2020; 368(6493): 860-8.
22. Kim L, Martinez C J, Hodgson K A, et al. Systemic and mucosal immune responses following oral adenoviral delivery of influenza vaccine to the human intestine by radio controlled capsule. Scientific Reports 2016; 6(1): 37295.
23. Liebowitz D, Lindbloom J D, Brandl J R, Garg S J, Tucker S N. High Titer Neutralizing Antibodies to Influenza Following Oral Tablet Immunization: A Randomized, Placebo-controlled Trial The Lancet infectious diseases 2015, 15: 1041-8.
24. Hird T R, Grassly N C. Systematic review of mucosal immunity induced by oral and inactivated poliovirus vaccines against virus shedding following oral poliovirus challenge. PLoS pathogens 2012; 8(4): e1002599.
25. Jaiswal J, LoSchiavo C, Perlman D C. Disinformation, Misinformation and Inequality-Driven Mistrust in the Time of COVID-19: Lessons Unlearned from AIDS Denialism. AIDS Behav 2020.
26. Palamenghi L, Barello S, Boccia S, Graffigna G. Mistrust in biomedical research and vaccine hesitancy: the forefront challenge in the battle against COVID-19 in Italy. Eur J Epidemiol 2020.
27. Zhu F C, Li Y H, Guan X H, et al. Safety, tolerability, and immunogenicity of a recombinant adenovirus type-5 vectored COVID-19 vaccine: a dose-escalation, open-label, non-randomised, first-in-human trial. Lancet 2020; 395(10240): 1845-54.
28. Baden L R, Walsh S R, Seaman M S, et al. First-in-Human Evaluation of the Safety and Immunogenicity of a Recombinant Adenovirus Serotype 26 HIV-1 Env Vaccine (IPCAVD 001). The Journal of infectious diseases 2012; 207(2): 240-7.
29. Kumar S, Maurya V K, Prasad A K, Bhatt M L B, Saxena S K. Structural, glycosylation and antigenic variation between 2019 novel coronavirus (2019-nCoV) and SARS coronavirus (SARS-CoV). Virusdisease 2020; 31(1): 13-21.
30. Wan Y, Shang J, Graham R, Baric R S, Li F. Receptor Recognition by the Novel Coronavirus from Wuhan: an Analysis Based on Decade-Long Structural Studies of SARS Coronavirus. Journal of virology 2020; 94(7).
31. Zost S J, Parkhouse K, Gumina M E, et al. Contemporary H3N2 influenza viruses have a glycosylation site that alters binding of antibodies elicited by egg-adapted vaccine strains. Proceedings of the National Academy of Sciences of the United States of America 2017; 114(47): 12578-83.
32. Xu H, Zhong L, Deng J, et al. High expression of ACE2 receptor of 2019-nCoV on the epithelial cells of oral mucosa. Int. J Oral Sci 2020; 12(1): 8.
33. Aylward B, Liang W. Report of the WHO-China Joint Mission on Coronavirus Disease 2019 (COVID-19). WHO Report 2020.
34. Yeo C, Kaushal S, Yeo D. Enteric involvement of coronaviruses: is faecal-oral transmission of SARS-CoV-2 possible? Lancet Gastroenterol Hepatol 2020.
35. Han C, Duan C, Zhang S, et al. Digestive Symptoms in COVID-19 Patients with Mild Disease Severity: Clinical Presentation, Stool Viral RNA Testing, and Outcomes. American Journal of Gastroenterology 2020.
36. Muramatsu M, Yoshida R, Yokoyama A, et al. Comparison of antiviral activity between IgA and IgG specific to influenza virus hemagglutinin: increased potential of IgA for heterosubtypic immunity. PLoS One 2014; 9(1): e85582.
37. Sterlin D, Mathian A, Miyara M, et al. IgA dominates the early neutralizing antibody response to SARS-CoV-2. medRxiv 2020: 2020.06.10.20126532.
38. Tamura S, Funato H, Hirabayashi Y, et al. Cross-protection against influenza A virus infection by passively transferred respiratory tract IgA antibodies to different hemagglutinin molecules. Eur J Immunol 1991; 21(6): 1337-44.
39. Le Bert N, Tan A T, Kunasegaran K, et al. SARS-CoV-2-specific T cell immunity in cases of COVID-19 and SARS, and uninfected controls. Nature 2020; 584(7821): 457-62.
40. Ni L, Ye F, Cheng M L, et al. Detection of SARS-CoV-2-Specific Humoral and Cellular Immunity in COVID-19 Convalescent Individuals. Immunity 2020; 52(6): 971-7 e3.
41. Tai W, Zhang X, Drelich A, et al. A novel receptor-binding domain (RBD)-based mRNA vaccine against SARS-CoV-2. Cell Res 2020.
42. Zha L, Zhao H, Mohsen M O, et al. Development of a COVID-19 vaccine based on the receptor binding domain displayed on virus-like particles. bioRxiv 2020: 2020.05.06.079830.
43. Mateus J, Grifoni A, Tarke A, et al. Selective and cross-reactive SARS-CoV-2 T cell epitopes in unexposed humans. Science 2020: eabd3871.
44. Mercado N B, Zahn R, Wegmann F, et al. Single-shot Ad26 vaccine protects against SARS-CoV-2 in rhesus macaques. Nature 2020.
45. Mutua G, Anzala O, Luhn K, et al. Safety and Immunogenicity of a 2-Dose Heterologous Vaccine Regimen With Ad26.ZEBOV and MVA-BN-Filo Ebola Vaccines: 12-Month Data From a Phase 1 Randomized Clinical Trial in Nairobi, Kenya. J Infect Dis 2019; 220(1): 57-67.
46. Anywaine Z, Whitworth H, Kaleebu P, et al. Safety and Immunogenicity of a 2-Dose Heterologous Vaccination Regimen With Ad26.ZEBOV and MVA-BN-Filo Ebola Vaccines: 12-Month Data From a Phase 1 Randomized Clinical Trial in Uganda and Tanzania. J Infect Dis 2019; 220(1): 46-56.
47. Antrobus R D, Coughlan L, Berthoud T K, et al. Clinical assessment of a novel recombinant simian adenovirus ChAdOx1 as a vectored vaccine expressing conserved Influenza A antigens. Mol Ther 2014; 22(3): 668-74.
48. van Doremalen N, Haddock E, Feldmann F, et al. A single dose of ChAdOx1 MERS provides broad protective immunity against a variety of MERS-CoV strains. bioRxiv 2020: 2020.04.13.036293.
49. Hassan A O, Kafai N M, Dmitriev I P, et al. A single intranasal dose of chimpanzee adenovirus-vectored vaccine confers sterilizing immunity against SARS-CoV-2 infection. bioRxiv 2020: 2020.07.16.205088.
50. He T C, Zhou S, da Costa L T, Yu J, Kinzler K W, Vogelstein B. A simplified system for generating recombinant adenoviruses. Proceedings of the National Academy of Sciences of the United States of America 1998: 95(5): 2509-14.
51. Guthe S, Kapinos L, Moglich A, Meier S, Grzesiek S, Kiefhaber T. Very fast folding and association of a trimerization domain from bacteriophage T4 fibritin. J Mol Biol 2004; 337(4): 905-15.
52. Tucker S N, Lin K, Stevens S, Scollay R, Bennett M J, Olson D C. Systemic and mucosal antibody responses following retroductal gene transfer to the salivary gland. Mol Therapy 2003: 8: 392-9.
53. Tan C W, Chia W N, Qin X, et al. A SARS-CoV-2 surrogate virus neutralization test based on antibody-mediated blockage of ACE2-spike protein-protein interaction. Nat Biotechnol 2020.

### Example 6

Study VXA-COV2-101 was a Phase 1 open-label, dose-ranging trial to evaluate the safety and immunogenicity of a SARS-CoV-2 oral tableted vaccine (rAd-S-N, SEQ ID NO:10), which is referred to in Examples 6 and 7 as VXA-CoV2-1, administered to healthy adult subjects 18-55 years of age.

The objectives of this study were to evaluate the safety and immunogenicity of VXA-CoV2-1 oral vaccine delivered by enteric tablet.

Subjects were enrolled at a single phase I unit in Southern California. Following completion of screening assessment and confirmation of eligibility 35 subjects were enrolled into this trial; Cohort 1 sentinel subjects (n=5) were vaccinated at Day 1 and have a repeat dose at Day 29. Cohort 2 and 3 subjects received a single vaccination at Day 1. The study design is shown in the table below-.

VXA-COV2-101 Study Design

| | | Dose | No. of | No of |
|---|---|---|---|---|
| Group | Vaccine | (IU ± 0.5 log)* | Doses | Subjects |
| Cohort 1 | VXA-CoV2-1 | 1 × 10¹⁰ | 2 | 5 |
| (Sentinels) | | | | |
| | SMC Review of Safety Data through Day 8 Visit | | | |
| Cohort 2 | VXA-CoV2-1 | 1 × 10¹⁰ | 1 | 15 |
| Cohort 3 | VXA-CoV2-1 | 5 × 10¹⁰ | 1 | 15 |

| | |
|---|---|
| Total | 35 |

Cohort 1 sentinel subjects received a second dose (boost) at same dose level as the first at Day 29

### B Cell Antibody Analysis

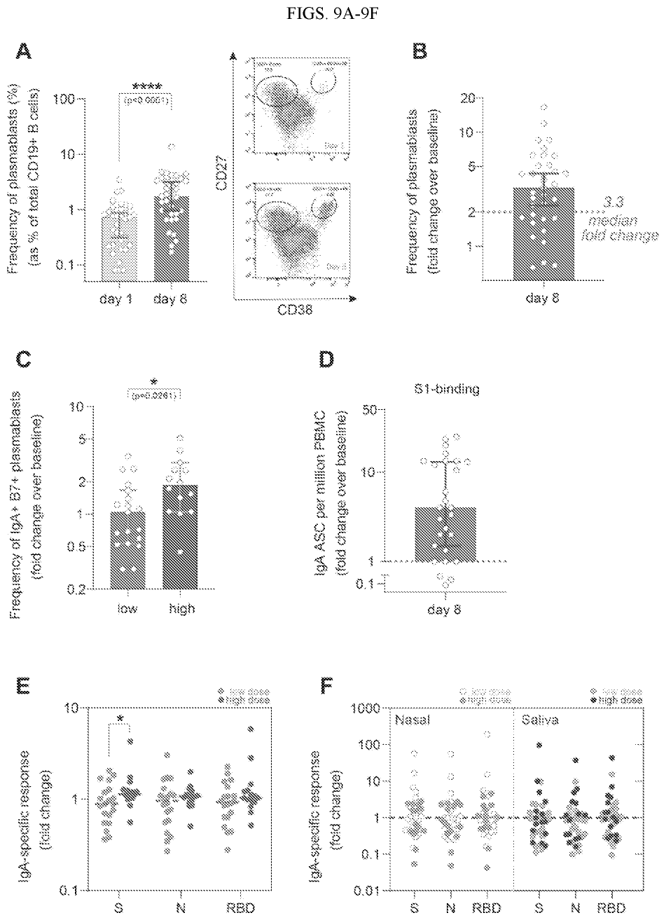

The ability of VXA-CoV2-1 in promoting B cells with high antibody-making potential was assessed using both flow cytometry-based measurements and an antibody-secreting cell (ASC) assay by ELISPOT. It has been previously well established that B cells responding to vaccination become activated at the site of administration and in local draining lymph nodes, where they differentiate into plasmablasts following germinal center reactions. Between 6 and 8 days after immunization, a significant proportion of plasmablasts leave the germinal centers and appear transiently in the peripheral circulation, where they can be found highly enriched for vaccine antigen-specific antibody-secreting cells (ASC). Accordingly, flow cytometry analysis of fixed whole blood samples collected pre- and post-vaccination in the VXA-COV2-101 study revealed a significant expansion in the overall CD27++ CD38++ plasmablast population 8 days following vaccination, with roughly 70% (24/35) of the vaccines showing a 2-fold or higher increase in plasmablast frequencies compared to baseline levels (FIG. 9A-B). Further investigation indicated upregulation of both IgA and the mucosal homing receptor α4β7 on the surface of circulating plasmablasts post vaccination, particularly in the cohort who received VXA-CoV2-1 at a higher dose level (FIG. 9C), thus suggesting vaccine-induced migration of this IgA-producing B cell population to mucosal tissues (Mora and von Andrian, 2008). Overall, these results are consistent with previous data published by the company in the context of a phase 2 Influenza A challenge study in humans, where generation of IgA plasmablasts with similar mucosal features following oral influenza vaccination was found to be a strong indicator of vaccine-induced protection (Liebowitz et al., 2020).

Additionally, an ELISpot assay was used to measure the ability of VXA-CoV2-1 to induce circulating antibody-secreting B cells that could recognize and bind the S1 domain of the SARS-CoV-2 spike (S) antigen. This analysis indicated significant vaccine-induced generation of S1-reactive, IgA-secreting ASC on day 8 post first immunization (p=0.0002 by Wilcoxon test), with an overall median 4-fold increase over baseline levels (FIG. 9D). More specifically, 8/12 (67%) subjects in the lower dose vaccine group for which both day 1 and day 8 ASC measurements were available were here classified as “responders”, as indicated by a median 2-fold or higher increase in day 8 post-vaccination IgA-secreting ASC numbers per million cells versus pre-vaccination levels (median fold increase of 2.67; 95% CI: 1.0-13.32). A slightly higher percentage of responders (11/15 subjects, 73%) was recorded in the higher vaccine dose cohort (median fold increase of 4; 95% CI: 1.3-13.32).

Levels of IgA antibodies specific to different SARS-CoV-2 antigens were measured in serum, saliva, and nasal samples pre- and post-immunization using the Meso Scale Discovery (MSD) platform. In agreement with the mucosal features of the B cell responses observed via flow cytometry and ELISPOT, IgA antibodies targeting SARS-CoV-2 spike (S), Nucleoprotein (N), and the spike receptor binding domain (RBD) could be found in both serum and mucosal compartments. Overall, 23% (8/35) of the vaccines showed a 50% or higher vaccine-specific IgA increase in the serum by day 29, with 6/8 of these subjects producing IgA targeting all three SARS-CoV-2 antigens in analysis. Consistent with the IgA+ B7+ plasmablast measurements, subjects in the higher dose cohort showed higher IgA antibody responses specific to S in the serum (FIG. 9). As we expected, given the unique characteristics of the VXA-CoV2-1 oral vaccine candidate, a higher percentage of vaccines mounted SARS-COV-2-specific IgA antibody responses in the mucosal compartments versus serum, with 54% of vaccines (19/35) reaching a 2-fold or higher increase in mucosal IgA in either saliva or nasal samples (FIG. 9F. More specifically, 10/35 (29%) of vaccines had a 2-fold or higher increase in IgA antibodies in their saliva, while 12/35 (35%) reached the same threshold in their nasal compartment by d29 post vaccination. No significant differences in vaccine-specific IgA responses in the saliva or nasal samples between the two dose groups could be observed (FIG. 9F). Measuring the ability of IgA to neutralize is difficult with the limitations of mucosal samples, but preliminary findings suggest that the subjects that had a two-fold increase in specific nasal IgA also had the ability to neutralize in a surrogate neutralization assay that measured ACE2 binding to spike protein (FIG. 9F). Secretory IgA has been shown to have higher neutralizing ability then IgG to SARS-CoV-2 as reported by Sterlin, et al, (Sterlin et al., 2021).

These findings are promising since several reports have highlighted the potential of mucosal immunity and generation of IgA antibodies in contributing to protection against COVID-19 (Ejemel et al., 2020; Russell et al., 2020; Sterlin et al., 2021). Notably, while injectable vaccines are not designed to effectively induce IgA antibodies in the respiratory mucosa, oral vaccination strategies might offer this advantage (Jeyanathan et al., 2020). Induction of SARS-CoV-2-specific IgA responses at key mucosal surfaces might also elicit sterlizing immunity and have a greater ability to block viral transmission, highly desirable features, particularly in a scenario where novel SARS-CoV-2 variants can replicate in vaccinated subjects undetected.

Analysis of IgG antibodies in the serum post-vaccination indicated an absence of an increase in SARS-CoV-2 specific antibody responses. Similarly, no significant SARS-CoV-2 antibody-mediated neutralization in the serum was observed. While the underlying reasons for the lack of vaccine-specific IgG and antibody neutralization in the serum are not defined at present, it is possible that a single oral dose of VXA-CoV2-1 at the dose levels used in this study were not sufficient to elicit robust vaccine-specific IgG neutralizing responses. Additionally, it cannot be excluded that the presence of a gene encoding for N in the VXA-CoV2-1 construct may have skewed the immunogenicity profile of this vaccine candidate away from serum neutralizing antibodies towards T cell-mediated immunity.

### T Cell Analysis

Besides B cells and antibodies, T cells also play a crucial role in the generation of protective immune responses against many microbial infections. In the context of COVID-19, T cells have been shown to target multiple SARS-CoV-2 proteins in convalescent subjects, while they also appear to be less vulnerable to SARS-CoV-2 variants compared to antibodies (Grifoni et al., 2020; Ledford, 2021; Tarke et al., 2021).

Induction of SARS-CoV-2-specific T cells and Th1/Th2 polarization following vaccination with VXA-CoV2-1 were measured using a restimulation assay using peripheral blood mononuclear cells (PBMCs) from 26 pairs of samples collected pre- and day 8 post-vaccination were cultured with SARS-CoV-2 peptides from either S or N, and Th1/Th2 cytokine responses assessed. PBMCs were thawed, rested overnight and cultured in Immunocult media (Stemcell Technologies) at a concentration of 1×10⁷ cells/ml in a 96 well round bottom plate for 5 hours at 37 degrees celsius with either the S or N peptide libraries (Miltenyi) in the presence of Brefeldin A (Invitrogen) and Monensin (Biolegend). Cells were harvested, surface stained with CD4-BV605, CD8-BV785 and zombie near-IR viability dye (Biolegend). After fixing with 4% PFA (biotium) and permeabilising with Cytoperm (BD Biosciences), antibodies to the cytokines IFNγ-BV510 (Biolegend), TNFα-e450 (Thermofisher), IL-2-APC (Thermofisher), IL-4-PerCP (biolegend), IL-5-PE (Biolegend), IL-13 (Biolegend), and CD107a-Alexa488 (Thermofisher) were used to assess the intracellular cytokine response and analysed using an Attune (Thermofisher) flow cytometer to evaluate.

No significant increase of Th2 responses, defined as intracellular production of IL5/1L4/IL13 cytokines by CD4⁺ T cells, was observed in response to S or N in any of the post vaccination samples compared to pre-vaccination levels. This is important since early reports in the field hypothesized potential adverse events related to antibody-dependent enhancement (ADE) following Th2 polarization (Lee et al., 2020).

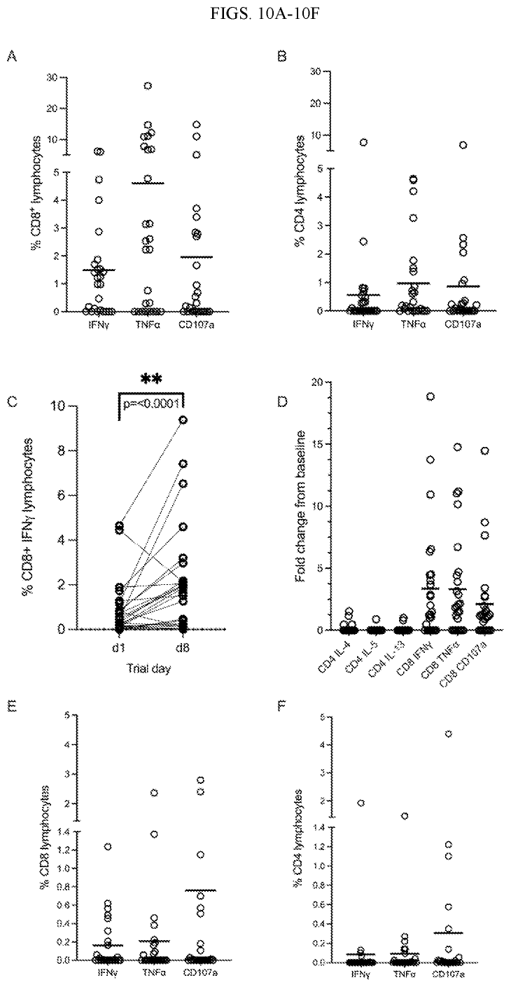

Strikingly, the majority of vaccines in this study strikingly had a strong increase in Th1 responses on day 8 post vaccination, defined as intracellular production of IFNγ/TNFα cytokines and the degranulation marker CD107a, particularly from CD8⁺ T cells (FIG. 10A, C) but also CD4⁺ T cells (FIG. 10 B) in response to restimulation with S peptides. Particularly, 13/26 (50%) subjects had a 2-fold or higher increase in Th1 cytokines in response to S, with 19/26 (73%) subjects overall showing measurable cytokine-producing CD8⁺ T cell responses above baseline levels (FIG. 10D). Increase in the production of Th1 cytokines post vaccination were also found following restimulation with SARS-COV-2 N peptides (FIG. 10E-F), particularly for CD8⁺ T cells, although of lower magnitude compared to S-specific responses. More specifically, 9/26 (35%) of subjects reached a 2-fold or higher increase in CD8⁺ T cell production of Th1 cytokines following N restimulation on day 8 compared to baseline levels. Overall, the percentage of T cells that show markers of anti-viral functionality in response to SARS-CoV-2 peptides, particularly IFNγ-producing CD8⁺ T cells, induced following oral immunization with VXA-CoV2-1 is remarkable. This percentage of responding CD8 T cells has not been reported with mRNA vaccines currently in use against COVID-19. This might represent a crucial advantage because CD8⁺ T cells with cytotoxic ability are uniquely positioned to clear virally infected cells and could also be helpful in reducing transmission by decreasing viral loads in infected patients (Ledford, 2021). Follow-up analyses will focus on a more direct comparison of vaccine-induced T cell immunity between VXA-CoV2-1 and the SARS-CoV-2 mRNA vaccines that have received EUA in the US.

In conclusion, VXA-CoV2-1 engaged both the humoral and cellular arms of the immune system by inducing SARS-CoV-2-specific IgA antibodies at key mucosal sites, as well as vaccine-specific T cells, particularly IFNγ-producing CD8⁺ T cells directed against both SARS-CoV-2 vaccine antigens S and N.

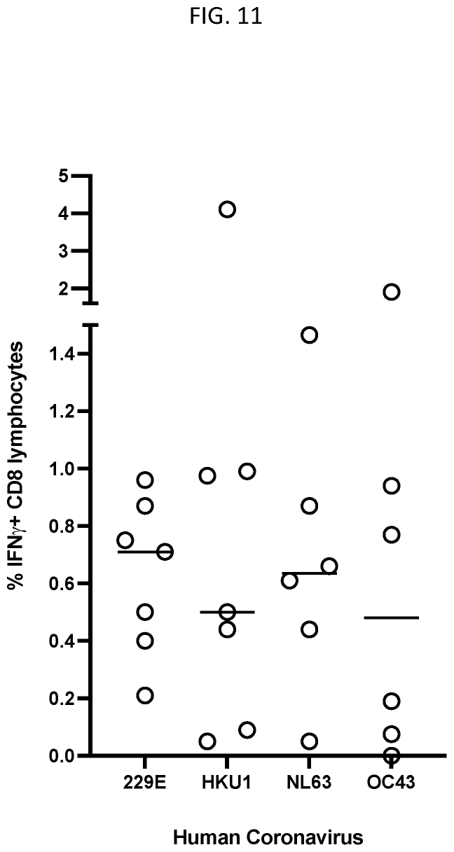

Another general goal of coronavirus vaccines is not only to protect against current strains, but to provide protection against circulating strains of other human coronaviruses, creating a pancoronavirus vaccine. To investigate this, we evaluated whether VXA-CoV2-1 induced T cells that were specific to the four endemic human coronaviruses—229E, HKU1, OC43 and NL63. We found that there was an increase over pre-vaccination levels for all four endemic human coronaviruses (FIG. 11), suggesting that the T cells induced are cross-reactive with the circulating human coronaviruses.

### Example 7

To compare the responses induced by VXA-COV2-1 with the current leading intramuscular covid vaccines, we recruited subjects that were due to be vaccinated with mRNA vaccines to donate PBMCs. PBMCs were taken at the same timepoints as our vaccines, pre and 7 days post vaccination, and T cell activity was measured in the same in vitro assay alongside PBMCs from the VXA-COV1-101 trial. Samples from all 3 vaccines were run in the same assay and subject to the same analysis to control for assay variability.

Surprisingly we found that subjects that took the VXA-COV2-1 tablets had T cell responses that were typically an order of magnitude higher than those that were vaccinated intramuscularly with either the Pfizer or Moderna vaccines approved under Emergency Use Authorization.

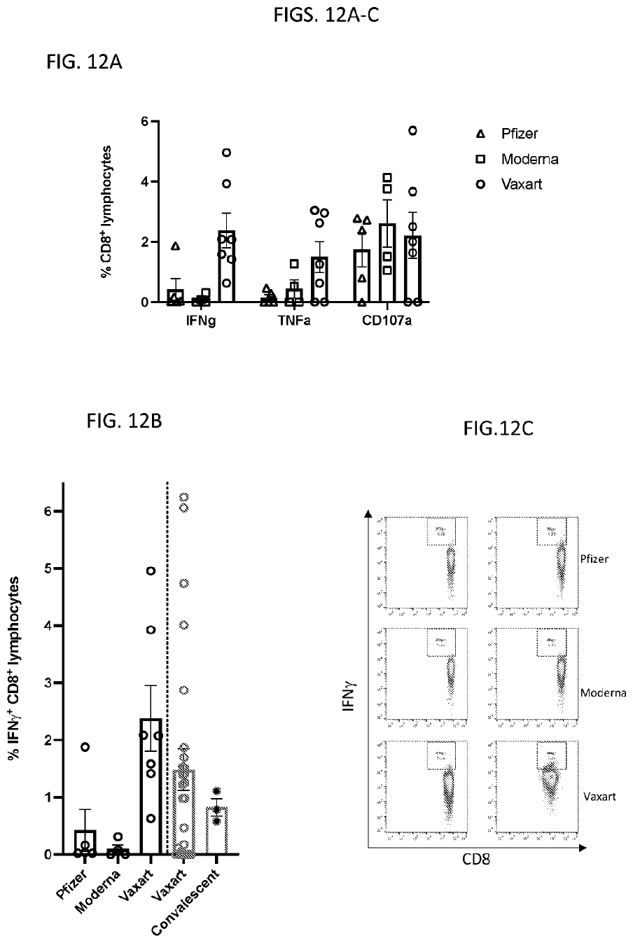

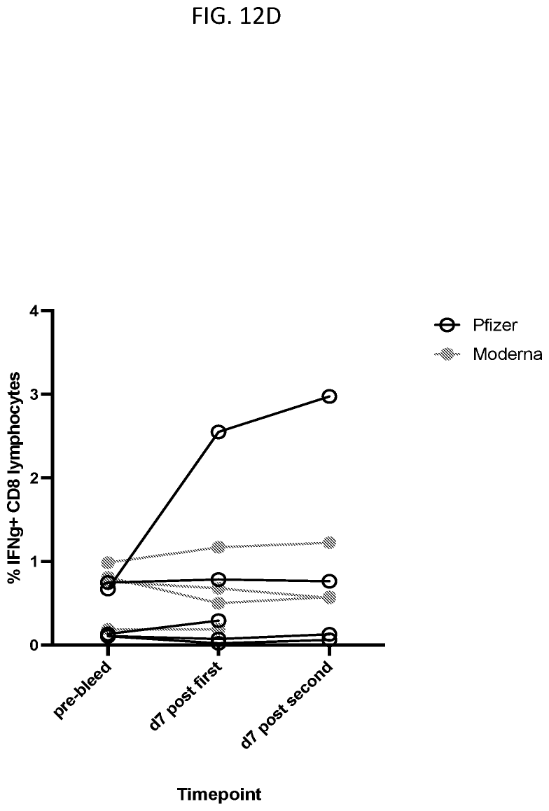

IFNγ and TNFα release from CD8 T cells were significantly increased (FIG. 12A), with CD107a degranulation showing a smaller increase over pre-vaccination baseline. The average percent increase above day 1 of IFNγ from CD8 T cells for the vaccines was 0.4/0.09/2.3 for Pfizer/Moderna/Vaxart vaccines respectively. This amounts to a >5 fold increase of those that took VXA-CoV-2 tablets versus the intramuscular vaccines.

As only a small subset of seven subjects were tested in the same assay as the other vaccines, to account for potential bias in selection of subjects, the whole cohort that was measured previously is graphed alongside for comparison, with significance still seen when comparing the whole cohort to the comparator experiment (FIG. 12B). With the average of the whole cohort including the non-responders being measured at 1.5%, a >3.5 fold increase was still seen over the intramuscular vaccines. The average IFNγ response of the four convalescent subjects was 0.8. Representative facs plots are shown in FIG. 12C, displaying the increase in VXA-CoV2-1 subjects at d7 post inoculation. The reported T cell measurements from the intramuscular vaccines were taken at a timepoint 7 days post second dose vaccine. To account for this, PBMCs were also measured at 7 days post second dose in the same assay and found to have responses of equal magnitude at both timepoints with the exception of one subject that had particularly good T cell responses that increased at both timepoints (FIG. 12D). This is similar to the data that was reported by Pfizer (Sahin et al. Nature 2021).

### Additional References Cited in Examples 5-7

Ejemel, M., Li, Q., Hou, S., Schiller, Z. A., Tree, J. A., Wallace, A., Amcheslavsky, A., Kurt Yilmaz, N., Buttigieg, K. R., Elmore, M. J., et al. (2020). A cross-reactive human IgA monoclonal antibody blocks SARS-CoV-2 spike-ACE2 interaction. Nat Commun 11, 4198.
Grifoni, A., Weiskopf, D., Ramirez, S. I., Mateus, J., Dan, J. M., Moderbacher, C. R., Rawlings, S. A., Sutherland, A., Premkumar, L., Jadi, R. S., et al. (2020). Targets of T Cell Responses to SARS-CoV-2 Coronavirus in Humans with COVID-19 Disease and Unexposed Individuals. Cell 181, 1489-1501 e1415.
He, X. S., Sasaki, S., Narvaez, C. F., Zhang, C., Liu, H., Woo, J. C., Kemble, G. W., Dekker, C. L., Davis, M. M., and Greenberg, H. B. (2011). Plasmablast-derived polyclonal antibody response after influenza vaccination. J Immunol Methods 365, 67-75.
Jeyanathan, M., Afkhami, S., Smaill, F., Miller, M. S., Lichty, B. D., and Xing, Z. (2020). Immunological considerations for COVID-19 vaccine strategies. Nat Rev Immunol 20, 615-632.
Ledford, H. (2021). How ‘killer’ T cells could boost COVID immunity in face of new variants. Nature 590, 374-375.
Lee, W. S., Wheatley, A. K., Kent, S. J., and DeKosky, B. J. (2020). Antibody-dependent enhancement and SARS-CoV-2 vaccines and therapies. Nat Microbiol 5, 1185-1191.
Liebowitz, D., Gottlieb, K., Kolhatkar, N. S., Garg, S. J., Asher, J. M., Nazareno, J., Kim, K., Mcllwain, D. R., and Tucker, S. N. (2020). Efficacy, immunogenicity, and safety of an oral influenza vaccine: a placebo-controlled and active-controlled phase 2 human challenge study. Lancet Infect Dis 20, 435-444.
Mora, J. R., and von Andrian, U. H. (2008). Differentiation and homing of IgA-secreting cells. Mucosal immunology 1, 96-109.
Russell, M. W., Moldoveanu, Z., Ogra, P. L., and Mestecky, J. (2020). Mucosal Immunity in COVID-19: A Neglected but Critical Aspect of SARS-CoV-2 Infection. Front Immunol 11, 611337.
Sterlin, D., Mathian, A., Miyara, M., Mohr, A., Anna, F., Claer, L., Quentric, P., Fadlallah, J., Devilliers, H., Ghillani, P., et al. (2021). IgA dominates the early neutralizing antibody response to SARS-CoV-2. Sci Transl Med 13.
Tarke, A., Sidney, J., Methot, N., Zhang, Y., Dan, J. M., Goodwin, B., Rubiro, P., Sutherland, A., da Silva Antunes, R., Frazier, A., et al. (2021). Negligible impact of SARS-CoV-2 variants on CD4 (+) and CD8 (+) T cell reactivity in COVID-19 exposed donors and vaccines. bioRxiv.

TABLE OF SEQUENCES

SEQ ID NO: 1: SARS-CoV-2 S Protein (surface glycoprotein) amino acid sequence
MFVFLVLLPLVSSQCVNLTTRTQLPPAYTNSFTRGVYYPDKVFRSSVLHSTQDLFLPF
FSNVTWFHAIHVSGTNGTKRFDNPVLPINDGVYFASTEKSNIIRGWIFGTTLDSKTQS
LLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRDLPQGFSALEPLVDLP
IGINITRFQTLLALHRSYLTPGDSSSGWTAGAAAYYVGYLQPRTFLLKYNENGTITDA
VDCALDPLSETKCTLKSFTVEKGIYQTSNFRVQPTESIVRFPNITNLCPFGEVFNATRF
ASVYAWNRKMSNCVADYSVLYNSASFSTFKCYGVSPTKLNDLCFTNVYADSFVIRG
DEVRQIAPGQTGKIADYNYKLPDDFTGCVIAWNSNNLDSKVGGNYNYLYRLFRKSN
LKPFERDISTEIYQAGSTPCNGVEGFNCYFPLQSYGFQPTNGVGYQPYRVVVLSFELL
HAPATVCGPKKSTNLVKNKCVNFNFNGLTGTGVLTESNKKFLPFQQFGRDIADTTD
AVRDPQTLEILDITPCSFGGVSVITPGTNTSNQVAVLYQDVNCTEVPVAIHADQLTPT
WRVYSTGSNVFQTRAGCLIGAEHVNNSYECDIPIGAGICASYQTQTNSPRRARSVAS
QSIIAYTMSLGAENSVAYSNNSIAIPTNFTISVTTEILPVSMTKTSVDCTMYICGDSTEC
SNLLLQYGSFCTQLNRALTGIAVEQDKNTQEVFAQVKQIYKTPPIKDFGGFNFSQILP
DPSKPSKRSFIEDLLFNKVTLADAGFIKQYGDCLGDIAARDLICAQKINGLTVLPPLLT
DEMIAQYTSALLAGTITSGWTFGAGAALQIPFAMQMAYRFNGIGVTQNVLYENQKLI
ANQFNSAIGKIQDSLSSTASALGKLQDVVNQNAQALNTLVKQLSSNFGAISSVLNDIL
SRLDKVEAEVQIDRLITGRLQSLQTYVTQQLIRAAEIRASANLAATKMSECVLGQSK
RVDFCGKGYHLMSFPQSAPHGVVFLHVTYVPAQEKNFTTAPAICHDGKAHFPREGV
FVSNGTHWFVTQRNFYEPQIITTDNTFVSGNCDVVIGIVNNTVYDPLQPELDSFKEEL
DKYFKNHTSPDVDLGDISGINASVVNIQKEIDRLNEVAKNLNESLIDLQELGKYEQYI
KWPWYIWLGFIAGLIAIVMVTIMLCCMTSCCSCLKGCCSCGSCCKFDEDDSEPVLKG
VKLHYT*
SEQ ID NO: 2: SARS-CoV-2 N Protein (nucleocapsid phosphoprotein) amino acid
sequence
MSDNGPQNQRNAPRITFGGPSDSTGSNQNGERSGARSKQRRPQGLPNNTASWFTAL
TQHGKEDLKFPRGQGVPINTNSSPDDQIGYYRRATRRIRGGDGKMKDLSPRWYFYY
LGTGPEAGLPYGANKDGIIWVATEGALNTPKDHIGTRNPANNAAIVLQLPQGTTLPK
GFYAEGSRGGSQASSRSSSRSRNSSRNSTPGSSRGTSPARMAGNGGDAALALLLLDR
LNQLESKMSGKGQQQQGQTVTKKSAAEASKKPRQKRTATKAYNVTQAFGRRGPEQ
TQGNFGDQELIRQGTDYKHWPQIAQFAPSASAFFGMSRIGMEVTPSGTWLTYTGAIK
LDDKDPNFKDQVILLNKHIDAYKTFPPTEPKKDKKKKADETQALPQRQKKQQTVTL
LPAADLDDFSKQLQQSMSSADSTQA*
SEQ ID NO: 3: SARS-CoV-2 S Protein (surface glycoprotein) nucleic acid sequence
ggtaccgccaccATGTTTGTTTTTCTCGTACTCCTGCCCCTGGTTTCCTCCCAATGTGTCA
ATCTGACTACCCGGACCCAACTTCCTCCCGCCTACACCAATTCCTTTACCCGAGG
TGTTTACTACCCAGACAAAGTGTTCAGGTCATCCGTCCTCCATAGTACCCAAGAC
CTCTTCCTCCCTTTTTTTTCTAACGTTACCTGGTTTCACGCTATTCACGTTAGCGGC
ACCAACGGCACCAAAAGATTCGATAACCCCGTACTGCCGTTCAACGACGGGGTA
TATTTTGCCTCTACTGAAAAATCAAACATCATACGCGGATGGATCTTTGGGACTA
CCCTGGACTCAAAAACTCAGTCCCTGCTGATTGTGAATAACGCTACCAACGTGGT
GATCAAAGTCTGTGAATTCCAGTTTTGCAACGATCCTTTTCTCGGCGTTTATTATC
ACAAAAATAACAAATCCTGGATGGAGAGCGAGTTCCGGGTGTACTCCTCCGCGA
ATAATTGCACCTTCGAATATGTGTCTCAGCCATTCCTCATGGACCTCGAGGGGAA
GCAGGGCAATTTTAAGAATCTGCGAGAATTCGTGTTCAAGAATATAGACGGTTAC
TTCAAGATTTACTCCAAACACACCCCGATTAACCTGGTTAGGGACTTGCCTCAGG
GCTTTTCTGCATTGGAGCCCCTCGTGGACCTCCCAATCGGCATAAACATTACAAG
ATTTCAGACTTTGCTTGCATTGCACAGGAGCTATTTGACACCCGGCGATTCTTCTT
CCGGATGGACCGCTGGAGCAGCTGCTTATTACGTGGGCTATCTGCAGCCTCGAAC
CTTTCTTTTGAAGTACAACGAAAATGGAACTATCACCGATGCAGTTGACTGCGCC
CTGGACCCCCTGTCCGAAACTAAGTGCACGCTCAAAAGTTTCACAGTAGAGAAG
GGGATATACCAGACTAGCAATTTCCGCGTTCAGCCAACCGAAAGTATAGTGCGC
TTTCCTAATATAACTAACCTGTGTCCTTTCGGGGAAGTGTTTAACGCCACTAGATT
CGCTTCCGTCTACGCCTGGAATAGAAAGAGGATCTCAAATTGCGTTGCTGACTAT
AGTGTTTTGTACAATTCCGCCTCTTTCTCAACCTTCAAATGTTACGGGGTGAGCCC
TACCAAACTGAACGACCTGTGCTTTACAAACGTATACGCCGACAGCTTTGTTATC
AGAGGAGACGAGGTTCGCCAGATTGCTCCGGGTCAGACAGGCAAGATTGCTGAT
TATAATTACAAACTGCCCGACGACTTTACAGGATGTGTGATCGCGTGGAACAGTA
ACAATCTTGACTCAAAGGTTGGGGGTAATTATAATTATCTTTACCGGCTGTTCAG
AAAAAGCAATTTGAAACCCTTCGAAAGGGACATATCCACCGAGATCTATCAGGC
CGGGTCCACTCCATGCAATGGTGTGGAAGGTTTTAATTGCTACTTCCCATTGCAG
TCTTATGGATTCCAACCAACCAATGGCGTAGGCTACCAGCCGTATCGCGTTGTCG
TGCTCAGCTTCGAGCTGCTCCACGCCCCCGCGACCGTATGCGGTCCTAAGAAGTC
CACCAATCTTGTTAAGAACAAGTGTGTAAACTTTAACTTTAACGGGCTGACCGGG
ACCGGCGTTCTGACTGAATCTAACAAAAAATTCCTGCCTTTCCAGCAGTTCGGCC
GCGATATTGCTGACACCACTGACGCTGTAAGAGACCCTCAGACCCTTGAAATTCT
CGATATCACACCTTGCAGCTTTGGGGGCGTGTCCGTCATCACTCCAGGAACTAAC
ACAAGCAACCAGGTGGCAGTGTTGTACCAGGATGTTAATTGTACCGAGGTGCCA
GTGGCCATCCACGCCGATCAATTGACACCTACCTGGAGGGTTTACAGCACAGGG
TCCAATGTTTTTCAGACAAGAGCCGGATGTCTGATCGGTGCCGAGCATGTCAACA
ATTCCTACGAGTGTGATATCCCCATTGGTGCGGGAATTTGTGCATCATATCAGAC
CCAGACTAATAGCCCAAGAAGAGCTAGATCCGTCGCTAGTCAATCCATCATTGC
ATATACAATGTCCCTGGGAGCTGAGAATTCAGTCGCGTATTCAAACAATTCCATT
GCTATTCCTACTAATTTCACTATCTCCGTCACGACCGAGATCCTGCCAGTTTCCAT
GACTAAGACTTCTGTTGACTGCACCATGTATATCTGTGGCGATAGCACCGAGTGC
AGTAATCTGCTTCTGCAGTACGGCTCCTTCTGCACACAACTCAATCGAGCACTGA
CCGGTATTGCAGTTGAGCAGGACAAGAACACACAGGAGGTCTTTGCACAGGTCA
AACAAATTTACAAAACCCCCCCCATAAAAGACTTTGGTGGGTTCAACTTCAGCCA
AATCCTCCCAGATCCCAGCAAGCCCTCCAAAAGATCCTTCATCGAAGACCTTTTG
TTCAATAAGGTAACCCTGGCCGACGCAGGCTTCATCAAACAATATGGCGATTGCC
TTGGAGACATTGCTGCGCGCGATTTGATCTGTGCTCAGAAATTTAACGGTTTGAC
CGTGCTGCCCCCACTTCTGACTGATGAGATGATAGCACAGTATACTTCTGCTCTT
CTGGCAGGAACAATCACTTCCGGGTGGACCTTTGGCGCTGGTGCAGCACTGCAA
ATCCCCTTCGCAATGCAAATGGCCTACCGATTCAATGGTATTGGTGTTACCCAGA
ACGTGCTCTATGAGAATCAGAAACTCATCGCCAATCAGTTCAATAGCGCTATTGG
CAAGATTCAGGATTCCCTCAGCTCTACCGCCAGCGCTCTGGGGAAGCTCCAGGAC
GTGGTGAACCAAAATGCTCAAGCGCTCAATACCCTTGTGAAACAGCTCAGCTCC
AATTTTGGCGCAATTAGCAGCGTTCTGAATGATATTCTGTCCCGGCTGGACAAGG
TAGAAGCAGAAGTCCAGATCGACAGGCTGATCACCGGGCGGTTGCAGAGTCTCC
AGACCTATGTCACACAACAGCTGATCCGCGCCGCCGAGATCAGGGCTTCCGCTA
ACCTGGCCGCCACTAAGATGTCCGAATGCGTGTTGGGGCAGAGTAAGCGGGTCG
ACTTTTGCGGGAAGGGATACCATCTGATGAGCTTCCCTCAGTCTGCACCCCACGG
AGTAGTGTTCCTCCACGTCACATATGTGCCCGCTCAGGAAAAGAATTTCACAACC
GCACCTGCTATCTGTCACGACGGCAAGGCCCACTTTCCTAGAGAAGGAGTTTTCG
TATCTAACGGCACCCACTGGTTCGTGACACAGCGGAACTTTTACGAGCCTCAGAT
TATAACTACGGACAACACTTTCGTGTCAGGCAACTGTGACGTGGTGATTGGGATC
GTGAACAACACAGTCTACGACCCATTGCAGCCCGAGTTGGACTCCTTCAAAGAG
GAGCTTGATAAGTATTTCAAGAACCATACCTCTCCCGACGTGGACCTGGGGGAC
ATTAGCGGCATCAATGCATCCGTTGTGAATATCCAGAAAGAAATCGATAGGCTG
AATGAGGTCGCAAAAAATCTTAATGAGTCACTGATTGATCTGCAGGAACTCGGC
AAATATGAGCAGTATATTAAGTGGCCGTGGTACATATGGCTCGGCTTTATCGCCG
GTCTGATTGCCATCGTGATGGTGACCATTATGCTGTGTTGTATGACAAGCTGCTG
TTCATGTCTCAAAGGATGCTGCTCCTGCGGTAGCTGCTGTAAGTTCGATGAAGAC
GACAGTGAGCCCGTGCTCAAAGGAGTGAAACTCCACTACACATAAcgatcg
SEQ ID NO: 4: SARS-CoV-2 N Protein (nacleocapsid phosphoprotein) nucleic arid
sequence
ggtaccgccaccATGTCCGATAACGGCCCCCAGAATCAGAGAAACGCTCCCCGCATCA
CGTTCGGCGGACCAAGTGACAGCACAGGCAGTAACCAGAACGGAGAACGCTCCG
GTGCTCGCTCCAAGCAGCGACGGCCGCAAGGGCTTCCCAACAATACCGCCAGCT
GGTTTACGGCTCTGACCCAACACGGGAAAGAAGATCTTAAATTCCCCAGGGGCC
AGGGCGTCCCTATCAATACTAACTCCAGCCCGGATGATCAGATAGGCTACTATAG
ACGCGCTACCCGACGGATACGAGGGGGGGACGGCAAAATGAAGGACCTTTCCCC
CCGGTGGTATTTCTATTACTTGGGCACCGGACCAGAAGCCGGACTGCCTTACGGC
GCTAACAAAGACGGAATAATCTGGGTTGCGACGGAGGGCGCCCTGAATACACCT
AAAGACCATATCGGCACAAGAAATCCTGCTAACAATGCCGCGATTGTGCTCCAG
CTGCCTCAGGGAACCACGCTGCCTAAAGGGTTTTACGCTGAGGGGTCAAGGGGG
GGGAGTCAAGCGTCTAGTAGGTCATCCTCTCGCTCTCGCAATAGTTCCCGGAACT
CAACCCCAGGCAGCAGCAGAGGAACCTCTCCCGCACGGATGGCTGGCAATGGGG
GAGATGCTGCCCTTGCTCTCCTTCTGCTGGATCGCCTTAACCAGCTCGAATCAAA
GATGTCTGGAAAAGGTCAGCAGCAGCAAGGCCAGACCGTGACAAAGAAGAGTG
CAGCTGAAGCTAGTAAAAAGCCACGCCAAAAACGGACCGCAACTAAGGCATATA
ACGTAACACAGGCCTTCGGCAGAAGAGGTCCAGAACAAACACAGGGAAACTTTG
GCGATCAAGAGCTGATTAGACAGGGCACAGATTACAAACACTGGCCACAGATCG
CGCAGTTTGCACCAAGCGCCTCTGCATTCTTCGGGATGAGTCGGATTGGGATGGA
AGTCACTCCATCCGGGACCTGGCTTACCTACACAGGGGCAATAAAACTCGACGA
CAAAGACCCAAACTTTAAAGATCAGGTCATCCTGCTGAATAAACACATCGATGC
CTACAAAACTTTCCCCCCAACCGAACCAAAGAAAGACAAGAAAAAAAAGGCAG
ACGAAACGCAAGCGCTCCCTCAGCGCCAGAAGAAGCAGCAGACCGTTACACTGT
TGCCAGCAGCAGATCTGGATGATTTTTCCAAGCAGCTTCAACAGAGTATGTCAAG
CGCTGACAGCACTCAGGCTTGAcgatcg
SEQ ID NO: 5: SARS-CoV-2 Fusion: S1-Furin-N nucleic acid sequence
ggtaccgccaccATGTTTGTTTTTCTCGTACTCCTGCCCCTGGTTTCCTCCCAATGTGTCA
ATCTGACTACCCGGACCCAACTTCCTCCCGCCTACACCAATTCCTTTACCCGAGG
TGTTTACTACCCAGACAAAGTGTTCAGGTCATCCGTCCTCCATAGTACCCAAGAC
CTCTTCCTCCCTTTTTTTTCTAACGTTACCTGGTTTCACGCTATTCACGTTAGCGGC
ACCAACGGCACCAAAAGATTCGATAACCCCGTACTGCCGTTCAACGACGGGGTA
TATTTTGCCTCTACTGAAAAATCAAACATCATACGCGGATGGATCTTTGGGACTA
CCCTGGACTCAAAAACTCAGTCCCTGCTGATTGTGAATAACGCTACCAACGTGGT
GATCAAAGTCTGTGAATTCCAGTTTTGCAACGATCCTTTTCTCGGCGTTTATTATC
ACAAAAATAACAAATCCTGGATGGAGAGCGAGTTCCGGGTGTACTCCTCCGCGA
ATAATTGCACCTTCGAATATGTGTCTCAGCCATTCCTCATGGACCTCGAGGGGAA
GCAGGGCAATTTTAAGAATCTGCGAGAATTCGTGTTCAAGAATATAGACGGTTAC
TTCAAGATTTACTCCAAACACACCCCGATTAACCTGGTTAGGGACTTGCCTCAGG
GCTTTTCTGCATTGGAGCCCCTCGTGGACCTCCCAATCGGCATAAACATTACAAG
ATTTCAGACTTTGCTTGCATTGCACAGGAGCTATTTGACACCCGGCGATTCTTCTT
CCGGATGGACCGCTGGAGCAGCTGCTTATTACGTGGGCTATCTGCAGCCTCGAAC
CTTTCTTTTGAAGTACAACGAAAATGGAACTATCACCGATGCAGTTGACTGCGCC
CTGGACCCCCTGTCCGAAACTAAGTGCACGCTCAAAAGTTTCACAGTAGAGAAG
GGGATATACCAGACTAGCAATTTCCGCGTTCAGCCAACCGAAAGTATAGTGCGC
TTTCCTAATATAACTAACCTGTGTCCTTTCGGGGAAGTGTTTAACGCCACTAGATT
CGCTTCCGTCTACGCCTGGAATAGAAAGAGGATCTCAAATTGCGTTGCTGACTAT
AGTGTTTTGTACAATTCCGCCTCTTTCTCAACCTTCAAATGTTACGGGGTGAGCCC
TACCAAACTGAACGACCTGTGCTTTACAAACGTATACGCCGACAGCTTTGTTATC
AGAGGAGACGAGGTTCGCCAGATTGCTCCGGGTCAGACAGGCAAGATTGCTGAT
TATAATTACAAACTGCCCGACGACTTTACAGGATGTGTGATCGCGTGGAACAGTA
ACAATCTTGACTCAAAGGTTGGGGGTAATTATAATTATCTTTACCGGCTGTTCAG
AAAAAGCAATTTGAAACCCTTCGAAAGGGACATATCCACCGAGATCTATCAGGC
CGGGTCCACTCCATGCAATGGTGTGGAAGGTTTTAATTGCTACTTCCCATTGCAG
TCTTATGGATTCCAACCAACCAATGGCGTAGGCTACCAGCCGTATCGCGTTGTCG
TGCTCAGCTTCGAGCTGCTCCACGCCCCCGCGACCGTATGCGGTCCTAAGAAGTC
CACCAATCTTGTTAAGAACAAGTGTGTAAACTTTAACTTTAACGGGCTGACCGGG
ACCGGCGTTCTGACTGAATCTAACAAAAAATTCCTGCCTTTCCAGCAGTTCGGCC
GCGATATTGCTGACACCACTGACGCTGTAAGAGACCCTCAGACCCTTGAAATTCT
CGATATCACACCTTGCAGCTTTGGGGGCGTGTCCGTCATCACTCCAGGAACTAAC
ACAAGCAACCAGGTGGCAGTGTTGTACCAGGATGTTAATTGTACCGAGGTGCCA
GTGGCCATCCACGCCGATCAATTGACACCTACCTGGAGGGTTTACAGCACAGGG
TCCAATGTTTTTCAGACAAGAGCCGGATGTCTGATCGGTGCCGAGCATGTCAACA
ATTCCTACGAGTGTGATATCCCCATTGGTGCGGGAATTTGTGCATCATATCAGAC
CCAGACTAATAGCCCAAGAAGAGCTAGATCCGTCGCTAGTCAATCCATCATTGC
ATATACAATGATGTCCGATAACGGCCCCCAGAATCAGAGAAACGCTCCCCGCAT
CACGTTCGGCGGACCAAGTGACAGCACAGGCAGTAACCAGAACGGAGAACGCTC
CGGTGCTCGCTCCAAGCAGCGACGGCCGCAAGGGCTTCCCAACAATACCGCCAG
CTGGTTTACGGCTCTGACCCAACACGGGAAAGAAGATCTTAAATTCCCCAGGGG
CCAGGGCGTCCCTATCAATACTAACTCCAGCCCGGATGATCAGATAGGCTACTAT
AGACGCGCTACCCGACGGATACGAGGGGGGGACGGCAAAATGAAGGACCTTTCC
CCCCGGTGGTATTTCTATTACTTGGGCACCGGACCAGAAGCCGGACTGCCTTACG
GCGCTAACAAAGACGGAATAATCTGGGTTGCGACGGAGGGCGCCCTGAATACAC
CTAAAGACCATATCGGCACAAGAAATCCTGCTAACAATGCCGCGATTGTGCTCC
AGCTGCCTCAGGGAACCACGCTGCCTAAAGGGTTTTACGCTGAGGGGTCAAGGG
GGGGGAGTCAAGCGTCTAGTAGGTCATCCTCTCGCTCTCGCAATAGTTCCCGGAA
CTCAACCCCAGGCAGCAGCAGAGGAACCTCTCCCGCACGGATGGCTGGCAATGG
GGGAGATGCTGCCCTTGCTCTCCTTCTGCTGGATCGCCTTAACCAGCTCGAATCA
AAGATGTCTGGAAAAGGTCAGCAGCAGCAAGGCCAGACCGTGACAAAGAAGAG
TGCAGCTGAAGCTAGTAAAAAGCCACGCCAAAAACGGACCGCAACTAAGGCATA
TAACGTAACACAGGCCTTCGGCAGAAGAGGTCCAGAACAAACACAGGGAAACTT
TGGCGATCAAGAGCTGATTAGACAGGGCACAGATTACAAACACTGGCCACAGAT
CGCGCAGTTTGCACCAAGCGCCTCTGCATTCTTCGGGATGAGTCGGATTGGGATG
GAAGTCACTCCATCCGGGACCTGGCTTACCTACACAGGGGCAATAAAACTCGAC
GACAAAGACCCAAACTTTAAAGATCAGGTCATCCTGCTGAATAAACACATCGAT
GCCTACAAAACTTTCCCCCCAACCGAACCAAAGAAAGACAAGAAAAAAAAGGC
AGACGAAACGCAAGCGCTCCCTCAGCGCCAGAAGAAGCAGCAGACCGTTACACT
GTTGCCAGCAGCAGATCTGGATGATTTTTCCAAGCAGCTTCAACAGAGTATGTCA
AGCGCTGACAGCACTCAGGCTTGAcgatcg
SEQ ID NO: 6: CMV-SARS-CoV-2-S-BGH-CMV-dSRNA-SPA
TAGTAATCAATTACGGGGTCATTAGTTCATAGCCCATATATGGAGTTCCGCGTTA
CATAACTTACGGTAAATGGCCCGCCTGGCTGACCGCCCAACGACCCCCGCCCATT
GACGTCAATAATGACGTATGTTCCCATAGTAACGCCAATAGGGACTTTCCATTGA
CGTCAATGGGTGGAGTATTTACGGTAAACTGCCCACTTGGCAGTACATCAAGTGT
ATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTAC
GTATTAGTCATCGCTATTACCATGGTGATGCGGTTTTGGCAGTACATCAATGGGC
GTGGATAGCGGTTTGACTCACGGGGATTTCCAAGTCTCCACCCCATTGACGTCAA
TGGGAGTTTGTTTTGGCACCAAAATCAACGGGACTTTCCAAAATGTCGTAACAAC
TCCGCCCCATTGACGCAAATGGGCGGTAGGCGTGTACGGTGGGAGGTCTATATA
AGCAGAGCTCGTTTAGTGAACCGTCAGatcgcctggagacgccatccacgctgttttgacctccatagaaga
caccgggaccgatccagcctgactctagCctAGCTCtgaagttggtggtgaggccctgggcaggttggtatcaaggttacaaga
caggtttaaggagaccaatagaaactgggcatgtggagacagagaagactcttgggtttctgataggcactgactctctctgcctattgg
tctattttcccacccttaggctgctggtctgagcctagGAGATCTCTCGAGGTCGACGGTATCGATGggtacc
gccaccATGTTTGTTTTTCTCGTACTCCTGCCCCTGGTTTCCTCCCAATGTGTCAATCT
GACTACCCGGACCCAACTTCCTCCCGCCTACACCAATTCCTTTACCCGAGGTGTT
TACTACCCAGACAAAGTGTTCAGGTCATCCGTCCTCCATAGTACCCAAGACCTCT
TCCTCCCTTTTTTTTCTAACGTTACCTGGTTTCACGCTATTCACGTTAGCGGCACC
AACGGCACCAAAAGATTCGATAACCCCGTACTGCCGTTCAACGACGGGGTATAT
TTTGCCTCTACTGAAAAATCAAACATCATACGCGGATGGATCTTTGGGACTACCC
TGGACTCAAAAACTCAGTCCCTGCTGATTGTGAATAACGCTACCAACGTGGTGAT
CAAAGTCTGTGAATTCCAGTTTTGCAACGATCCTTTTCTCGGCGTTTATTATCACA
AAAATAACAAATCCTGGATGGAGAGCGAGTTCCGGGTGTACTCCTCCGCGAATA
ATTGCACCTTCGAATATGTGTCTCAGCCATTCCTCATGGACCTCGAGGGGAAGCA
GGGCAATTTTAAGAATCTGCGAGAATTCGTGTTCAAGAATATAGACGGTTACTTC
AAGATTTACTCCAAACACACCCCGATTAACCTGGTTAGGGACTTGCCTCAGGGCT
TTTCTGCATTGGAGCCCCTCGTGGACCTCCCAATCGGCATAAACATTACAAGATT
TCAGACTTTGCTTGCATTGCACAGGAGCTATTTGACACCCGGCGATTCTTCTTCCG
GATGGACCGCTGGAGCAGCTGCTTATTACGTGGGCTATCTGCAGCCTCGAACCTT
TCTTTTGAAGTACAACGAAAATGGAACTATCACCGATGCAGTTGACTGCGCCCTG
GACCCCCTGTCCGAAACTAAGTGCACGCTCAAAAGTTTCACAGTAGAGAAGGGG
ATATACCAGACTAGCAATTTCCGCGTTCAGCCAACCGAAAGTATAGTGCGCTTTC
CTAATATAACTAACCTGTGTCCTTTCGGGGAAGTGTTTAACGCCACTAGATTCGC
TTCCGTCTACGCCTGGAATAGAAAGAGGATCTCAAATTGCGTTGCTGACTATAGT
GTTTTGTACAATTCCGCCTCTTTCTCAACCTTCAAATGTTACGGGGTGAGCCCTAC
CAAACTGAACGACCTGTGCTTTACAAACGTATACGCCGACAGCTTTGTTATCAGA
GGAGACGAGGTTCGCCAGATTGCTCCGGGTCAGACAGGCAAGATTGCTGATTAT
AATTACAAACTGCCCGACGACTTTACAGGATGTGTGATCGCGTGGAACAGTAAC
AATCTTGACTCAAAGGTTGGGGGTAATTATAATTATCTTTACCGGCTGTTCAGAA
AAAGCAATTTGAAACCCTTCGAAAGGGACATATCCACCGAGATCTATCAGGCCG
GGTCCACTCCATGCAATGGTGTGGAAGGTTTTAATTGCTACTTCCCATTGCAGTC
TTATGGATTCCAACCAACCAATGGCGTAGGCTACCAGCCGTATCGCGTTGTCGTG
CTCAGCTTCGAGCTGCTCCACGCCCCCGCGACCGTATGCGGTCCTAAGAAGTCCA
CCAATCTTGTTAAGAACAAGTGTGTAAACTTTAACTTTAACGGGCTGACCGGGAC
CGGCGTTCTGACTGAATCTAACAAAAAATTCCTGCCTTTCCAGCAGTTCGGCCGC
GATATTGCTGACACCACTGACGCTGTAAGAGACCCTCAGACCCTTGAAATTCTCG
ATATCACACCTTGCAGCTTTGGGGGCGTGTCCGTCATCACTCCAGGAACTAACAC
AAGCAACCAGGTGGCAGTGTTGTACCAGGATGTTAATTGTACCGAGGTGCCAGT
GGCCATCCACGCCGATCAATTGACACCTACCTGGAGGGTTTACAGCACAGGGTC
CAATGTTTTTCAGACAAGAGCCGGATGTCTGATCGGTGCCGAGCATGTCAACAAT
TCCTACGAGTGTGATATCCCCATTGGTGCGGGAATTTGTGCATCATATCAGACCC
AGACTAATAGCCCAAGAAGAGCTAGATCCGTCGCTAGTCAATCCATCATTGCAT
ATACAATGTCCCTGGGAGCTGAGAATTCAGTCGCGTATTCAAACAATTCCATTGC
TATTCCTACTAATTTCACTATCTCCGTCACGACCGAGATCCTGCCAGTTTCCATGA
CTAAGACTTCTGTTGACTGCACCATGTATATCTGTGGCGATAGCACCGAGTGCAG
TAATCTGCTTCTGCAGTACGGCTCCTTCTGCACACAACTCAATCGAGCACTGACC
GGTATTGCAGTTGAGCAGGACAAGAACACACAGGAGGTCTTTGCACAGGTCAAA
CAAATTTACAAAACCCCCCCCATAAAAGACTTTGGTGGGTTCAACTTCAGCCAAA
TCCTCCCAGATCCCAGCAAGCCCTCCAAAAGATCCTTCATCGAAGACCTTTTGTT
CAATAAGGTAACCCTGGCCGACGCAGGCTTCATCAAACAATATGGCGATTGCCTT
GGAGACATTGCTGCGCGCGATTTGATCTGTGCTCAGAAATTTAACGGTTTGACCG
TGCTGCCCCCACTTCTGACTGATGAGATGATAGCACAGTATACTTCTGCTCTTCTG
GCAGGAACAATCACTTCCGGGTGGACCTTTGGCGCTGGTGCAGCACTGCAAATC
CCCTTCGCAATGCAAATGGCCTACCGATTCAATGGTATTGGTGTTACCCAGAACG
TGCTCTATGAGAATCAGAAACTCATCGCCAATCAGTTCAATAGCGCTATTGGCAA
GATTCAGGATTCCCTCAGCTCTACCGCCAGCGCTCTGGGGAAGCTCCAGGACGTG
GTGAACCAAAATGCTCAAGCGCTCAATACCCTTGTGAAACAGCTCAGCTCCAATT
TTGGCGCAATTAGCAGCGTTCTGAATGATATTCTGTCCCGGCTGGACAAGGTAGA
AGCAGAAGTCCAGATCGACAGGCTGATCACCGGGCGGTTGCAGAGTCTCCAGAC
CTATGTCACACAACAGCTGATCCGCGCCGCCGAGATCAGGGCTTCCGCTAACCTG
GCCGCCACTAAGATGTCCGAATGCGTGTTGGGGCAGAGTAAGCGGGTCGACTTT
TGCGGGAAGGGATACCATCTGATGAGCTTCCCTCAGTCTGCACCCCACGGAGTA
GTGTTCCTCCACGTCACATATGTGCCCGCTCAGGAAAAGAATTTCACAACCGCAC
CTGCTATCTGTCACGACGGCAAGGCCCACTTTCCTAGAGAAGGAGTTTTCGTATC
TAACGGCACCCACTGGTTCGTGACACAGCGGAACTTTTACGAGCCTCAGATTATA
ACTACGGACAACACTTTCGTGTCAGGCAACTGTGACGTGGTGATTGGGATCGTGA
ACAACACAGTCTACGACCCATTGCAGCCCGAGTTGGACTCCTTCAAAGAGGAGC
TTGATAAGTATTTCAAGAACCATACCTCTCCCGACGTGGACCTGGGGGACATTAG
CGGCATCAATGCATCCGTTGTGAATATCCAGAAAGAAATCGATAGGCTGAATGA
GGTCGCAAAAAATCTTAATGAGTCACTGATTGATCTGCAGGAACTCGGCAAATA
TGAGCAGTATATTAAGTGGCCGTGGTACATATGGCTCGGCTTTATCGCCGGTCTG
ATTGCCATCGTGATGGTGACCATTATGCTGTGTTGTATGACAAGCTGCTGTTCAT
GTCTCAAAGGATGCTGCTCCTGCGGTAGCTGCTGTAAGTTCGATGAAGACGACA
GTGAGCCCGTGCTCAAAGGAGTGAAACTCCACTACACATAAcgatcgGATATCGCT
AGCGTACCGGCGGCCGCCCTATTCTATAGTGTCACCTAAATGCTAGAGCTCGCTG
ATCAGCCTCGACTGTGCCTTCTAGTTGCCAGCCATCTGTTGTTTGCCCCTCCCCCG
TGCCTTCCTTGACCCTGGAAGGTGCCACTCCCACTGTCCTTTCCTAATAAAATGA
GGAAATTGCATCGCATTGTCTGAGTAGGTGTCATTCTATTCTGGGGGGTGGGGTG
GGGCAGGACAGCAAGGGGGAGGATTGGGAAGACAATAGCAGGCATGCTGGGGA
TGCGGTGGGCTCTATGGCTTCTGAGGCGGAAAGAACCAAAGCTTAcgcgttagttattaata
GTAATCAATTACGGGGTCATTAGTTCATAGCCCATATATGGAGTTCCGCGTTACA
TAACTTACGGTAAATGGCCCGCCTGGCTGACCGCCCAACGACCCCCGCCCATTGA
CGTCAATAATGACGTATGTTCCCATAGTAACGCCAATAGGGACTTTCCATTGACG
TCAATGGGTGGAGTATTTACGGTAAACTGCCCACTTGGCAGTACATCAAGTGTAT
CATATGCCAAGTACGCCCCCTATTGACGTCAATGACGGTAAATGGCCCGCCTGGC
ATTATGCCCAGTACATGACCTTATGGGACTTTCCTACTTGGCAGTACATCTACGT
ATTAGTCATCGCTATTACCATGGTGATGCGGTTTTGGCAGTACATCAATGGGCGT
GGATAGCGGTTTGACTCACGGGGATTTCCAAGTCTCCACCCCATTGACGTCAATG
GGAGTTTGTTTTGGCACCAAAATCAACGGGACTTTCCAAAATGTCGTAACAACTC
CGCCCCATTGACGCAAATGGGCGGTAGGCGTGTACGGTGGGAGGTCTATATAAG
CAGAGCTGGTTTAGTGAACCGTCAGATCCGCTAGAGATATCGGGCCACTGCAGG
AAACGATATGGGCTGAATACGGATCCGTATTCAGCCCATATCGTTTCTCTAGAAA
TAAAATATCTTTATTTTCATTACATCTGTGTGTTGGTTTTTTGTGTG
SEQ ID NO: 7: CMV-SARS-CoV-2-S-BGH-bActin-SARS-CoV-2-N-SPA-BGH-CMV-
dsRNA-SPA
TAGTAATCAATTACGGGGTCATTAGTTCATAGCCCATATATGGAGTTCCGCGTTA
CATAACTTACGGTAAATGGCCCGCCTGGCTGACCGCCCAACGACCCCCGCCCATT
GACGTCAATAATGACGTATGTTCCCATAGTAACGCCAATAGGGACTTTCCATTGA
CGTCAATGGGTGGAGTATTTACGGTAAACTGCCCACTTGGCAGTACATCAAGTGT
ATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTAC
GTATTAGTCATCGCTATTACCATGGTGATGCGGTTTTGGCAGTACATCAATGGGC
GTGGATAGCGGTTTGACTCACGGGGATTTCCAAGTCTCCACCCCATTGACGTCAA
TGGGAGTTTGTTTTGGCACCAAAATCAACGGGACTTTCCAAAATGTCGTAACAAC
TCCGCCCCATTGACGCAAATGGGCGGTAGGCGTGTACGGTGGGAGGTCTATATA
AGCAGAGCTCGTTTAGTGAACCGTCAGATCGCCTGGAGACGCCATCCACGCTGTT
TTGACCTCCATAGAAGACACCGGGACCGATCCAGCCTGACTCTAGCCTAGCTCTG
AAGTTGGTGGTGAGGCCCTGGGCAGGTTGGTATCAAGGTTacaagacaggtttaaggagacca
atagaaactgggcatgtggagacagagaagactcttgggtttctgataggcactgactctctctgcctattggtctattttcccaccctt
aggctgctggtctgagcctagGAGATCTCTCGAGGTCGACGGTATCGATGggtaccgccaccATGTTT
GTTTTTCTCGTACTCCTGCCCCTGGTTTCCTCCCAATGTGTCATCTGACTACCCG
GACCCAACTTCCTCCCGCCTACACCAATTCCTTTACCCGAGGTGTTTACTACCCA
GACAAAGTGTTCAGGTCATCCGTCCTCCATAGTACCCAAGACCTCTTCCTCCCTTT
TTTTTCTAACGTTACCTGGTTTCACGCTATTCACGTTAGCGGCACCAACGGCACC
AAAAGATTCGATAACCCCGTACTGCCGTTCAACGACGGGGTATATTTTGCCTCTA
CTGAAAAATCAAACATCATACGCGGATGGATCTTTGGGACTACCCTGGACTCAA
AAACTCAGTCCCTGCTGATTGTGAATAACGCTACCAACGTGGTGATCAAAGTCTG
TGAATTCCAGTTTTGCAACGATCCTTTTCTCGGCGTTTATTATCACAAAAATAACA
AATCCTGGATGGAGAGCGAGTTCCGGGTGTACTCCTCCGCGAATAATTGCACCTT
CGAATATGTGTCTCAGCCATTCCTCATGGACCTCGAGGGGAAGCAGGGCAATTTT
AAGAATCTGCGAGAATTCGTGTTCAAGAATATAGACGGTTACTTCAAGATTTACT
CCAAACACACCCCGATTAACCTGGTTAGGGACTTGCCTCAGGGCTTTTCTGCATT
GGAGCCCCTCGTGGACCTCCCAATCGGCATAAACATTACAAGATTTCAGACTTTG
CTTGCATTGCACAGGAGCTATTTGACACCCGGCGATTCTTCTTCCGGATGGACCG
CTGGAGCAGCTGCTTATTACGTGGGCTATCTGCAGCCTCGAACCTTTCTTTTGAA
GTACAACGAAAATGGAACTATCACCGATGCAGTTGACTGCGCCCTGGACCCCCT
GTCCGAAACTAAGTGCACGCTCAAAAGTTTCACAGTAGAGAAGGGGATATACCA
GACTAGCAATTTCCGCGTTCAGCCAACCGAAAGTATAGTGCGCTTTCCTAATATA
ACTAACCTGTGTCCTTTCGGGGAAGTGTTTAACGCCACTAGATTCGCTTCCGTCT
ACGCCTGGAATAGAAAGAGGATCTCAAATTGCGTTGCTGACTATAGTGTTTTGTA
CAATTCCGCCTCTTTCTCAACCTTCAAATGTTACGGGGTGAGCCCTACCAAACTG
AACGACCTGTGCTTTACAAACGTATACGCCGACAGCTTTGTTATCAGAGGAGACG
AGGTTCGCCAGATTGCTCCGGGTCAGACAGGCAAGATTGCTGATTATAATTACAA
ACTGCCCGACGACTTTACAGGATGTGTGATCGCGTGGAACAGTAACAATCTTGAC
TCAAAGGTTGGGGGTAATTATAATTATCTTTACCGGCTGTTCAGAAAAAGCAATT
TGAAACCCTTCGAAAGGGACATATCCACCGAGATCTATCAGGCCGGGTCCACTC
CATGCAATGGTGTGGAAGGTTTTAATTGCTACTTCCCATTGCAGTCTTATGGATTC
CAACCAACCAATGGCGTAGGCTACCAGCCGTATCGCGTTGTCGTGCTCAGCTTCG
AGCTGCTCCACGCCCCCGCGACCGTATGCGGTCCTAAGAAGTCCACCAATCTTGT
TAAGAACAAGTGTGTAAACTTTAACTTTAACGGGCTGACCGGGACCGGCGTTCTG
ACTGAATCTAACAAAAAATTCCTGCCTTTCCAGCAGTTCGGCCGCGATATTGCTG
ACACCACTGACGCTGTAAGAGACCCTCAGACCCTTGAAATTCTCGATATCACACC
TTGCAGCTTTGGGGGCGTGTCCGTCATCACTCCAGGAACTAACACAAGCAACCA
GGTGGCAGTGTTGTACCAGGATGTTAATTGTACCGAGGTGCCAGTGGCCATCCAC
GCCGATCAATTGACACCTACCTGGAGGGTTTACAGCACAGGGTCCAATGTTTTTC
AGACAAGAGCCGGATGTCTGATCGGTGCCGAGCATGTCAACAATTCCTACGAGT
GTGATATCCCCATTGGTGCGGGAATTTGTGCATCATATCAGACCCAGACTAATAG
CCCAAGAAGAGCTAGATCCGTCGCTAGTCAATCCATCATTGCATATACAATGTCC
CTGGGAGCTGAGAATTCAGTCGCGTATTCAAACAATTCCATTGCTATTCCTACTA
ATTTCACTATCTCCGTCACGACCGAGATCCTGCCAGTTTCCATGACTAAGACTTCT
GTTGACTGCACCATGTATATCTGTGGCGATAGCACCGAGTGCAGTAATCTGCTTC
TGCAGTACGGCTCCTTCTGCACACAACTCAATCGAGCACTGACCGGTATTGCAGT
TGAGCAGGACAAGAACACACAGGAGGTCTTTGCACAGGTCAAACAAATTTACAA
AACCCCCCCCATAAAAGACTTTGGTGGGTTCAACTTCAGCCAAATCCTCCCAGAT
CCCAGCAAGCCCTCCAAAAGATCCTTCATCGAAGACCTTTTGTTCAATAAGGTAA
CCCTGGCCGACGCAGGCTTCATCAAACAATATGGCGATTGCCTTGGAGACATTGC
TGCGCGCGATTTGATCTGTGCTCAGAAATTTAACGGTTTGACCGTGCTGCCCCCA
CTTCTGACTGATGAGATGATAGCACAGTATACTTCTGCTCTTCTGGCAGGAACAA
TCACTTCCGGGTGGACCTTTGGCGCTGGTGCAGCACTGCAAATCCCCTTCGCAAT
GCAAATGGCCTACCGATTCAATGGTATTGGTGTTACCCAGAACGTGCTCTATGAG
AATCAGAAACTCATCGCCAATCAGTTCAATAGCGCTATTGGCAAGATTCAGGATT
CCCTCAGCTCTACCGCCAGCGCTCTGGGGAAGCTCCAGGACGTGGTGAACCAAA
ATGCTCAAGCGCTCAATACCCTTGTGAAACAGCTCAGCTCCAATTTTGGCGCAAT
TAGCAGCGTTCTGAATGATATTCTGTCCCGGCTGGACAAGGTAGAAGCAGAAGT
CCAGATCGACAGGCTGATCACCGGGCGGTTGCAGAGTCTCCAGACCTATGTCAC
ACAACAGCTGATCCGCGCCGCCGAGATCAGGGCTTCCGCTAACCTGGCCGCCAC
TAAGATGTCCGAATGCGTGTTGGGGCAGAGTAAGCGGGTCGACTTTTGCGGGAA
GGGATACCATCTGATGAGCTTCCCTCAGTCTGCACCCCACGGAGTAGTGTTCCTC
CACGTCACATATGTGCCCGCTCAGGAAAAGAATTTCACAACCGCACCTGCTATCT
GTCACGACGGCAAGGCCCACTTTCCTAGAGAAGGAGTTTTCGTATCTAACGGCAC
CCACTGGTTCGTGACACAGCGGAACTTTTACGAGCCTCAGATTATAACTACGGAC
AACACTTTCGTGTCAGGCAACTGTGACGTGGTGATTGGGATCGTGAACAACACA
GTCTACGACCCATTGCAGCCCGAGTTGGACTCCTTCAAAGAGGAGCTTGATAAGT
ATTTCAAGAACCATACCTCTCCCGACGTGGACCTGGGGGACATTAGCGGCATCA
ATGCATCCGTTGTGAATATCCAGAAAGAAATCGATAGGCTGAATGAGGTCGCAA
AAAATCTTAATGAGTCACTGATTGATCTGCAGGAACTCGGCAAATATGAGCAGT
ATATTAAGTGGCCGTGGTACATATGGCTCGGCTTTATCGCCGGTCTGATTGCCAT
CGTGATGGTGACCATTATGCTGTGTTGTATGACAAGCTGCTGTTCATGTCTCAAA
GGATGCTGCTCCTGCGGTAGCTGCTGTAAGTTCGATGAAGACGACAGTGAGCCC
GTGCTCAAAGGAGTGAAACTCCACTACACATAAcgatcgacgcgtAGAGCTCGCTGATC
AGCCTCGACTGTGCCTTCTAGTTGCCAGCCATCTGTTGTTTGCCCCTCCCCCGTGC
CTTCCTTGACCCTGGAAGGTGCCACTCCCACTGTCCTTTCCTAATAAAATGAGGA
AATTGCATCGCATTGTCTGAGTAGGTGTCATTCTATTCTGGGGGGTGGGGTGGGG
CAGGACAGCAAGGGGGAGGATTGGGAAGACAATAGCAGGCATGCTGGGGATGC
GGTGGGCTCTATGGCTTCTGAGGCGGAAAGAACCAaagcttgcggccgcGCCCAGCACC
CCAAGGCGGCCAACGCCAAAACTCTCCCTCCTCCTCTTCCTCAATCTCGCTCTCG
CTCTTTTTTTTTTTCGCAAAAGGAGGGGAGAGGGGGTAAAAAAATGCTGCACTGT
GCGGCGAAGCCGGTGAGTGAGCGGCGCGGGGCCAATCAGCGTGCGCCGTTCCGA
AAGTTGCCTTTTATGGCTCGAGCGGCCGCGGCGGCGCCCTATAAAACCCAGCGG
CGCGACGCGCCACCACCGCCGAGACcctgcaggccgccaccATGTCCGATAACGGCCCCC
AGAATCAGAGAAACGCTCCCCGCATCACGTTCGGCGGACCAAGTGACAGCACAG
GCAGTAACCAGAACGGAGAACGCTCCGGTGCTCGCTCCAAGCAGCGACGGCCGC
AAGGGCTTCCCAACAATACCGCCAGCTGGTTTACGGCTCTGACCCAACACGGGA
AAGAAGATCTTAAATTCCCCAGGGGCCAGGGCGTCCCTATCAATACTAACTCCA
GCCCGGATGATCAGATAGGCTACTATAGACGCGCTACCCGACGGATACGAGGGG
GGGACGGCAAAATGAAGGACCTTTCCCCCCGGTGGTATTTCTATTACTTGGGCAC
CGGACCAGAAGCCGGACTGCCTTACGGCGCTAACAAAGACGGAATAATCTGGGT
TGCGACGGAGGGCGCCCTGAATACACCTAAAGACCATATCGGCACAAGAAATCC
TGCTAACAATGCCGCGATTGTGCTCCAGCTGCCTCAGGGAACCACGCTGCCTAAA
GGGTTTTACGCTGAGGGGTCAAGGGGGGGGAGTCAAGCGTCTAGTAGGTCATCC
TCTCGCTCTCGCAATAGTTCCCGGAACTCAACCCCAGGCAGCAGCAGAGGAACC
TCTCCCGCACGGATGGCTGGCAATGGGGGAGATGCTGCCCTTGCTCTCCTTCTGC
TGGATCGCCTTAACCAGCTCGAATCAAAGATGTCTGGAAAAGGTCAGCAGCAGC
AAGGCCAGACCGTGACAAAGAAGAGTGCAGCTGAAGCTAGTAAAAAGCCACGC
CAAAAACGGACCGCAACTAAGGCATATAACGTAACACAGGCCTTCGGCAGAAGA
GGTCCAGAACAAACACAGGGAAACTTTGGCGATCAAGAGCTGATTAGACAGGGC
ACAGATTACAAACACTGGCCACAGATCGCGCAGTTTGCACCAAGCGCCTCTGCA
TTCTTCGGGATGAGTCGGATTGGGATGGAAGTCACTCCATCCGGGACCTGGCTTA
CCTACACAGGGGCAATAAAACTCGACGACAAAGACCCAAACTTTAAAGATCAGG
TCATCCTGCTGAATAAACACATCGATGCCTACAAAACTTTCCCCCCAACCGAACC
AAAGAAAGACAAGAAAAAAAAGGCAGACGAAACGCAAGCGCTCCCTCAGCGCC
AGAAGAAGCAGCAGACCGTTACACTGTTGCCAGCAGCAGATCTGGATGATTTTT
CCAAGCAGCTTCAACAGAGTATGTCAAGCGCTGACAGCACTCAGGCTTGAggcgcg
ccgctgaccgatAAATAAAATATCTTTATTTTCATTACATCTGTGTGTTGGTTTTTTGTGT
GacgcgttagttattaataGTAATCAATTACGGGGTCATTAGTTCATAGCCCATATATGGAGT
TCCGCGTTACATAACTTACGGTAAATGGCCCGCCTGGCTGACCGCCCAACGACCC
CCGCCCATTGACGTCAATAATGACGTATGTTCCCATAGTAACGCCAATAGGGACT
TTCCATTGACGTCAATGGGTGGAGTATTTACGGTAAACTGCCCACTTGGCAGTAC
ATCAAGTGTATCATATGCCAAGTACGCCCCCTATTGACGTCAATGACGGTAAATG
GCCCGCCTGGCATTATGCCCAGTACATGACCTTATGGGACTTTCCTACTTGGCAG
TACATCTACGTATTAGTCATCGCTATTACCATGGTGATGCGGTTTTGGCAGTACAT
CAATGGGCGTGGATAGCGGTTTGACTCACGGGGATTTCCAAGTCTCCACCCCATT
GACGTCAATGGGAGTTTGTTTTGGCACCAAAATCAACGGGACTTTCCAAAATGTC
GTAACAACTCCGCCCCATTGACGCAAATGGGCGGTAGGCGTGTACGGTGGGAGG
TCTATATAAGCAGAGCTGGTTTAGTGAACCGTCAGATCCGCTAGAGATATCGGGC
CACTGCAGGAAACGATATGGGCTGAATACGGATCCGTATTCAGCCCATATCGTTT
CTCTAGAAATAAAATATCTTTATTTTCATTACATCTGTGTGTGGTTTTTTGTGTG
SEQ ID NO: 8: CMV-SARS-CoV-2-S1-Furin-N-BGH-CMV-dsRNA-SPA
TAGTAATCAATTACGGGGTCATTAGTTCATAGCCCATATATGGAGTTCCGCGTTA
CATAACTTACGGTAAATGGCCCGCCTGGCTGACCGCCCAACGACCCCCGCCCATT
GACGTCAATAATGACGTATGTTCCCATAGTAACGCCAATAGGGACTTTCCATTGA
CGTCAATGGGTGGAGTATTTACGGTAAACTGCCCACTTGGCAGTACATCAAGTGT
ATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTAC
GTATTAGTCATCGCTATTACCATGGTGATGCGGTTTTGGCAGTACATCAATGGGC
GTGGATAGCGGTTTGACTCACGGGGATTTCCAAGTCTCCACCCCATTGACGTCAA
TGGGAGTTTGTTTTGGCACCAAAATCAACGGGACTTTCCAAAATGTCGTAACAAC
TCCGCCCCATTGACGCAAATGGGCGGTAGGCGTGTACGGTGGGAGGTCTATATA
AGCAGAGCTCGTTTAGTGAACCGTCAGatcgcctggagacgccatccacgctgttttgacctccatagaaga
caccgggaccgatccagcctgactctagCctAGCTCtgaagttggtggtgaggccctgggcaggttggtatcaaggttacaaga
caggtttaaggagaccaatagaaactgggcatgtggagacagagaagactcttgggtttctgataggcactgactctctctgcctattgg
tctattttcccacccttaggctgctggtctgagcctagGAGATCTCTCGAGGTCGACGGTATCGATGggtacc
gccaccATGTTTGTTTTTCTCGTACTCCTGCCCCTGGTTTCCTCCCAATGTGTCAATCT
GACTACCCGGACCCAACTTCCTCCCGCCTACACCAATTCCTTTACCCGAGGTGTT
TACTACCCAGACAAAGTGTTCAGGTCATCCGTCCTCCATAGTACCCAAGACCTCT
TCCTCCCTTTTTTTTCTAACGTTACCTGGTTTCACGCTATTCACGTTAGCGGCACC
AACGGCACCAAAAGATTCGATAACCCCGTACTGCCGTTCAACGACGGGGTATAT
TTTGCCTCTACTGAAAAATCAAACATCATACGCGGATGGATCTTTGGGACTACCC
TGGACTCAAAAACTCAGTCCCTGCTGATTGTGAATAACGCTACCAACGTGGTGAT
CAAAGTCTGTGAATTCCAGTTTTGCAACGATCCTTTTCTCGGCGTTTATTATCACA
AAAATAACAAATCCTGGATGGAGAGCGAGTTCCGGGTGTACTCCTCCGCGAATA
ATTGCACCTTCGAATATGTGTCTCAGCCATTCCTCATGGACCTCGAGGGGAAGCA
GGGCAATTTTAAGAATCTGCGAGAATTCGTGTTCAAGAATATAGACGGTTACTTC
AAGATTTACTCCAAACACACCCCGATTAACCTGGTTAGGGACTTGCCTCAGGGCT
TTTCTGCATTGGAGCCCCTCGTGGACCTCCCAATCGGCATAAACATTACAAGATT
TCAGACTTTGCTTGCATTGCACAGGAGCTATTTGACACCCGGCGATTCTTCTTCCG
GATGGACCGCTGGAGCAGCTGCTTATTACGTGGGCTATCTGCAGCCTCGAACCTT
TCTTTTGAAGTACAACGAAAATGGAACTATCACCGATGCAGTTGACTGCGCCCTG
GACCCCCTGTCCGAAACTAAGTGCACGCTCAAAAGTTTCACAGTAGAGAAGGGG
ATATACCAGACTAGCAATTTCCGCGTTCAGCCAACCGAAAGTATAGTGCGCTTTC
CTAATATAACTAACCTGTGTCCTTTCGGGGAAGTGTTTAACGCCACTAGATTCGC
TTCCGTCTACGCCTGGAATAGAAAGAGGATCTCAAATTGCGTTGCTGACTATAGT
GTTTTGTACAATTCCGCCTCTTTCTCAACCTTCAAATGTTACGGGGTGAGCCCTAC
CAAACTGAACGACCTGTGCTTTACAAACGTATACGCCGACAGCTTTGTTATCAGA
GGAGACGAGGTTCGCCAGATTGCTCCGGGTCAGACAGGCAAGATTGCTGATTAT
AATTACAAACTGCCCGACGACTTTACAGGATGTGTGATCGCGTGGAACAGTAAC
AATCTTGACTCAAAGGTTGGGGGTAATTATAATTATCTTTACCGGCTGTTCAGAA
AAAGCAATTTGAAACCCTTCGAAAGGGACATATCCACCGAGATCTATCAGGCCG
GGTCCACTCCATGCAATGGTGTGGAAGGTTTTAATTGCTACTTCCCATTGCAGTC
TTATGGATTCCAACCAACCAATGGCGTAGGCTACCAGCCGTATCGCGTTGTCGTG
CTCAGCTTCGAGCTGCTCCACGCCCCCGCGACCGTATGCGGTCCTAAGAAGTCCA
CCAATCTTGTTAAGAACAAGTGTGTAAACTTTAACTTTAACGGGCTGACCGGGAC
CGGCGTTCTGACTGAATCTAACAAAAAATTCCTGCCTTTCCAGCAGTTCGGCCGC
GATATTGCTGACACCACTGACGCTGTAAGAGACCCTCAGACCCTTGAAATTCTCG
ATATCACACCTTGCAGCTTTGGGGGCGTGTCCGTCATCACTCCAGGAACTAACAC
AAGCAACCAGGTGGCAGTGTTGTACCAGGATGTTAATTGTACCGAGGTGCCAGT
GGCCATCCACGCCGATCAATTGACACCTACCTGGAGGGTTTACAGCACAGGGTC
CAATGTTTTTCAGACAAGAGCCGGATGTCTGATCGGTGCCGAGCATGTCAACAAT
TCCTACGAGTGTGATATCCCCATTGGTGCGGGAATTTGTGCATCATATCAGACCC
AGACTAATAGCCCAAGAAGAGCTAGATCCGTCGCTAGTCAATCCATCATTGCAT
ATACAATGATGTCCGATAACGGCCCCCAGAATCAGAGAAACGCTCCCCGCATCA
CGTTCGGCGGACCAAGTGACAGCACAGGCAGTAACCAGAACGGAGAACGCTCCG
GTGCTCGCTCCAAGCAGCGACGGCCGCAAGGGCTTCCCAACAATACCGCCAGCT
GGTTTACGGCTCTGACCCAACACGGGAAAGAAGATCTTAAATTCCCCAGGGGCC
AGGGCGTCCCTATCAATACTAACTCCAGCCCGGATGATCAGATAGGCTACTATAG
ACGCGCTACCCGACGGATACGAGGGGGGGACGGCAAAATGAAGGACCTTTCCCC
CCGGTGGTATTTCTATTACTTGGGCACCGGACCAGAAGCCGGACTGCCTTACGGC
GCTAACAAAGACGGAATAATCTGGGTTGCGACGGAGGGCGCCCTGAATACACCT
AAAGACCATATCGGCACAAGAAATCCTGCTAACAATGCCGCGATTGTGCTCCAG
CTGCCTCAGGGAACCACGCTGCCTAAAGGGTTTTACGCTGAGGGGTCAAGGGGG
GGGAGTCAAGCGTCTAGTAGGTCATCCTCTCGCTCTCGCAATAGTTCCCGGAACT
CAACCCCAGGCAGCAGCAGAGGAACCTCTCCCGCACGGATGGCTGGCAATGGGG
GAGATGCTGCCCTTGCTCTCCTTCTGCTGGATCGCCTTAACCAGCTCGAATCAAA
GATGTCTGGAAAAGGTCAGCAGCAGCAAGGCCAGACCGTGACAAAGAAGAGTG
CAGCTGAAGCTAGTAAAAAGCCACGCCAAAAACGGACCGCAACTAAGGCATATA
ACGTAACACAGGCCTTCGGCAGAAGAGGTCCAGAACAAACACAGGGAAACTTTG
GCGATCAAGAGCTGATTAGACAGGGCACAGATTACAAACACTGGCCACAGATCG
CGCAGTTTGCACCAAGCGCCTCTGCATTCTTCGGGATGAGTCGGATTGGGATGGA
AGTCACTCCATCCGGGACCTGGCTTACCTACACAGGGGCAATAAAACTCGACGA
CAAAGACCCAAACTTTAAAGATCAGGTCATCCTGCTGAATAAACACATCGATGC
CTACAAAACTTTCCCCCCAACCGAACCAAAGAAAGACAAGAAAAAAAAGGCAG
ACGAAACGCAAGCGCTCCCTCAGCGCCAGAAGAAGCAGCAGACCGTTACACTGT
TGCCAGCAGCAGATCTGGATGATTTTTCCAAGCAGCTTCAACAGAGTATGTCAAG
CGCTGACAGCACTCAGGCTTGAcgatcgGATATCGCTAGCGTACCGGCGGCCGCCCT
ATTCTATAGTGTCACCTAAATGCTAGAGCTCGCTGATCAGCCTCGACTGTGCCTT
CTAGTTGCCAGCCATCTGTTGTTTGCCCCTCCCCCGTGCCTTCCTTGACCCTGGAA
GGTGCCACTCCCACTGTCCTTTCCTAATAAAATGAGGAAATTGCATCGCATTGTC
TGAGTAGGTGTCATTCTATTCTGGGGGGTGGGGTGGGGCAGGACAGCAAGGGGG
AGGATTGGGAAGACAATAGCAGGCATGCTGGGGATGCGGTGGGCTCTATGGCTT
CTGAGGCGGAAAGAACCAAAGCTTAcgcgttagttattaataGTAATCAATTACGGGGTCAT
TAGTTCATAGCCCATATATGGAGTTCCGCGTTACATAACTTACGGTAAATGGCCC
GCCTGGCTGACCGCCCAACGACCCCCGCCCATTGACGTCAATAATGACGTATGTT
CCCATAGTAACGCCAATAGGGACTTTCCATTGACGTCAATGGGTGGAGTATTTAC
GGTAAACTGCCCACTTGGCAGTACATCAAGTGTATCATATGCCAAGTACGCCCCC
TATTGACGTCAATGACGGTAAATGGCCCGCCTGGCATTATGCCCAGTACATGACC
TTATGGGACTTTCCTACTTGGCAGTACATCTACGTATTAGTCATCGCTATTACCAT
GGTGATGCGGTTTTGGCAGTACATCAATGGGCGTGGATAGCGGTTTGACTCACGG
GGATTTCCAAGTCTCCACCCCATTGACGTCAATGGGAGTTTGTTTTGGCACCAAA
ATCAACGGGACTTTCCAAAATGTCGTAACAACTCCGCCCCATTGACGCAAATGG
GCGGTAGGCGTGTACGGTGGGAGGTCTATATAAGCAGAGCTGGTTTAGTGAACC
GTCAGATCCGCTAGAGATATCGGGCCACTGCAGGAAACGATATGGGCTGAATAC
GGATCCGTATTCAGCCCATATCGTTTCTCTAGAAATAAAATATCTTTATTTTCATT
ACATCTGTGTGTTGGTTTTTTGTGTG
SEQ ID NO: 9: rAd-CMV-SARS-CoV-2-S-BGH-CMV-dsRNA-SPA
TAAGGATCCCATCATCAATAATATACCTTATTTTGGATTGAAGCCAATATGATAA
TGAGGGGGTGGAGTTTGTGACGTGGCGCGGGGCGTGGGAACGGGGCGGGTGAC
GTAGTAGTGTGGCGGAAGTGTGATGTTGCAAGTGTGGCGGAACACATGTAAGCG
ACGGATGTGGCAAAAGTGACGTTTTTGGTGTGCGCCGGTGTACACAGGAAGTGA
CAATTTTCGCGCGGTTTTAGGCGGATGTTGTAGTAAATTTGGGCGTAACCGAGTA
AGATTTGGCCATTTTCGCGGGAAAACTGAATAAGAGGAAGTGAAATCTGAATAA
TTTTGTGTTACTCATAGCGCGTAATACTGCTAGAGATTGGCGAAAGGGGGATGT
GCTGCAAGGCGATTAAGTTGGGTAACGCCAGGGTTTTCCCAGTCACGACGTTGTA
AAACGACGGCCAGTGAATTGTAATACGACTCACTATAGGGCGAATTGGGTACTG
GCCACAGGAGCTTGGCCCATTGCATACGTTGTATCCATATCATAATATGTACATT
TATATTGGCTCATGTCCAACATTACCGCCATGTTGACATTGATTATTGACTAGTTA
TTAATAGTAATCAATTACGGGGTCATTAGTTCATAGCCCATATATGGAGTTCCGC
GTTACATAACTTACGGTAAATGGCCCGCCTGGCTGACCGCCCAACGACCCCCGCC
CATTGACGTCAATAATGACGTATGTTCCCATAGTAACGCCAATAGGGACTTTCCA
TTGACGTCAATGGGTGGAGTATTTACGGTAAACTGCCCACTTGGCAGTACATCAA
GTGTATCATATGCCAAGTACGCCCCCTATTGACGTCAATGACGGTAAATGGCCCG
CCTGGCATTATGCCCAGTACATGACCTTATGGGACTTTCCTACTTGGCAGTACAT
CTACGTATTAGTCATCGCTATTACCATGGTGATGCGGTTTTGGCAGTACATCAAT
GGGCGTGGATAGCGGTTTGACTCACGGGGATTTCCAAGTCTCCACCCCATTGACG
TCAATGGGAGTTTGTTTTGGCACCAAAATCAACGGGACTTTCCAAAATGTCGTAA
CAACTCCGCCCCATTGACGCAAATGGGCGGTAGGCGTGTACGGTGGGAGGTCTA
TATAAGCAGAGCTCGTTTAGTGAACCGTCAGatcgcctggagacgccatccacgctgttttgacctccat
agaagacaccgggaccgatccagcctgactctagCctAGCTCtgaagttggtggtgaggccctgggcaggttggtatcaaggtt
acaagacaggtttaaggagaccaatagaaactgggcatgtggagacagagaagactcttgggtttctgataggcactgactctctctgc
ctattggtctattttcccacccttaggctgctggtctgagcctagGAGATCTCTCGAGGTCGACGGTATCGAT
GggtaccgccaccATGTTTGTTTTTCTCGTACTCCTGCCCCTGGTTTCCTCCCAATGTGTC
AATCTGACTACCCGGACCCAACTTCCTCCCGCCTACACCAATTCCTTTACCCGAG
GTGTTTACTACCCAGACAAAGTGTTCAGGTCATCCGTCCTCCATAGTACCCAAGA
CCTCTTCCTCCCTTTTTTTTCTAACGTTACCTGGTTTCACGCTATTCACGTTAGCGG
CACCAACGGCACCAAAAGATTCGATAACCCCGTACTGCCGTTCAACGACGGGGT
ATATTTTGCCTCTACTGAAAAATCAAACATCATACGCGGATGGATCTTTGGGACT
ACCCTGGACTCAAAAACTCAGTCCCTGCTGATTGTGAATAACGCTACCAACGTGG
TGATCAAAGTCTGTGAATTCCAGTTTTGCAACGATCCTTTTCTCGGCGTTTATTAT
CACAAAAATAACAAATCCTGGATGGAGAGCGAGTTCCGGGTGTACTCCTCCGCG
AATAATTGCACCTTCGAATATGTGTCTCAGCCATTCCTCATGGACCTCGAGGGGA
AGCAGGGCAATTTTAAGAATCTGCGAGAATTCGTGTTCAAGAATATAGACGGTT
ACTTCAAGATTTACTCCAAACACACCCCGATTAACCTGGTTAGGGACTTGCCTCA
GGGCTTTTCTGCATTGGAGCCCCTCGTGGACCTCCCAATCGGCATAAACATTACA
AGATTTCAGACTTTGCTTGCATTGCACAGGAGCTATTTGACACCCGGCGATTCTT
CTTCCGGATGGACCGCTGGAGCAGCTGCTTATTACGTGGGCTATCTGCAGCCTCG
AACCTTTCTTTTGAAGTACAACGAAAATGGAACTATCACCGATGCAGTTGACTGC
GCCCTGGACCCCCTGTCCGAAACTAAGTGCACGCTCAAAAGTTTCACAGTAGAG
AAGGGGATATACCAGACTAGCAATTTCCGCGTTCAGCCAACCGAAAGTATAGTG
CGCTTTCCTAATATAACTAACCTGTGTCCTTTCGGGGAAGTGTTTAACGCCACTA
GATTCGCTTCCGTCTACGCCTGGAATAGAAAGAGGATCTCAAATTGCGTTGCTGA
CTATAGTGTTTTGTACAATTCCGCCTCTTTCTCAACCTTCAAATGTTACGGGGTGA
GCCCTACCAAACTGAACGACCTGTGCTTTACAAACGTATACGCCGACAGCTTTGT
TATCAGAGGAGACGAGGTTCGCCAGATTGCTCCGGGTCAGACAGGCAAGATTGC
TGATTATAATTACAAACTGCCCGACGACTTTACAGGATGTGTGATCGCGTGGAAC
AGTAACAATCTTGACTCAAAGGTTGGGGGTAATTATAATTATCTTTACCGGCTGT
TCAGAAAAAGCAATTTGAAACCCTTCGAAAGGGACATATCCACCGAGATCTATC
AGGCCGGGTCCACTCCATGCAATGGTGTGGAAGGTTTTAATTGCTACTTCCCATT
GCAGTCTTATGGATTCCAACCAACCAATGGCGTAGGCTACCAGCCGTATCGCGTT
GTCGTGCTCAGCTTCGAGCTGCTCCACGCCCCCGCGACCGTATGCGGTCCTAAGA
AGTCCACCAATCTTGTTAAGAACAAGTGTGTAAACTTTAACTTTAACGGGCTGAC
CGGGACCGGCGTTCTGACTGAATCTAACAAAAAATTCCTGCCTTTCCAGCAGTTC
GGCCGCGATATTGCTGACACCACTGACGCTGTAAGAGACCCTCAGACCCTTGAA
ATTCTCGATATCACACCTTGCAGCTTTGGGGGCGTGTCCGTCATCACTCCAGGAA
CTAACACAAGCAACCAGGTGGCAGTGTTGTACCAGGATGTTAATTGTACCGAGG
TGCCAGTGGCCATCCACGCCGATCAATTGACACCTACCTGGAGGGTTTACAGCAC
AGGGTCCAATGTTTTTCAGACAAGAGCCGGATGTCTGATCGGTGCCGAGCATGTC
AACAATTCCTACGAGTGTGATATCCCCATTGGTGCGGGAATTTGTGCATCATATC
AGACCCAGACTAATAGCCCAAGAAGAGCTAGATCCGTCGCTAGTCAATCCATCA
TTGCATATACAATGTCCCTGGGAGCTGAGAATTCAGTCGCGTATTCAAACAATTC
CATTGCTATTCCTACTAATTTCACTATCTCCGTCACGACCGAGATCCTGCCAGTTT
CCATGACTAAGACTTCTGTTGACTGCACCATGTATATCTGTGGCGATAGCACCGA
GTGCAGTAATCTGCTTCTGCAGTACGGCTCCTTCTGCACACAACTCAATCGAGCA
CTGACCGGTATTGCAGTTGAGCAGGACAAGAACACACAGGAGGTCTTTGCACAG
GTCAAACAAATTTACAAAACCCCCCCCATAAAAGACTTTGGTGGGTTCAACTTCA
GCCAAATCCTCCCAGATCCCAGCAAGCCCTCCAAAAGATCCTTCATCGAAGACCT
TTTGTTCAATAAGGTAACCCTGGCCGACGCAGGCTTCATCAAACAATATGGCGAT
TGCCTTGGAGACATTGCTGCGCGCGATTTGATCTGTGCTCAGAAATTTAACGGTT
TGACCGTGCTGCCCCCACTTCTGACTGATGAGATGATAGCACAGTATACTTCTGC
TCTTCTGGCAGGAACAATCACTTCCGGGTGGACCTTTGGCGCTGGTGCAGCACTG
CAAATCCCCTTCGCAATGCAAATGGCCTACCGATTCAATGGTATTGGTGTTACCC
AGAACGTGCTCTATGAGAATCAGAAACTCATCGCCAATCAGTTCAATAGCGCTAT
TGGCAAGATTCAGGATTCCCTCAGCTCTACCGCCAGCGCTCTGGGGAAGCTCCAG
GACGTGGTGAACCAAAATGCTCAAGCGCTCAATACCCTTGTGAAACAGCTCAGC
TCCAATTTTGGCGCAATTAGCAGCGTTCTGAATGATATTCTGTCCCGGCTGGACA
AGGTAGAAGCAGAAGTCCAGATCGACAGGCTGATCACCGGGCGGTTGCAGAGTC
TCCAGACCTATGTCACACAACAGCTGATCCGCGCCGCCGAGATCAGGGCTTCCGC
TAACCTGGCCGCCACTAAGATGTCCGAATGCGTGTTGGGGCAGAGTAAGCGGGT
CGACTTTTGCGGGAAGGGATACCATCTGATGAGCTTCCCTCAGTCTGCACCCCAC
GGAGTAGTGTTCCTCCACGTCACATATGTGCCCGCTCAGGAAAAGAATTTCACAA
CCGCACCTGCTATCTGTCACGACGGCAAGGCCCACTTTCCTAGAGAAGGAGTTTT
CGTATCTAACGGCACCCACTGGTTCGTGACACAGCGGAACTTTTACGAGCCTCAG
ATTATAACTACGGACAACACTTTCGTGTCAGGCAACTGTGACGTGGTGATTGGGA
TCGTGAACAACACAGTCTACGACCCATTGCAGCCCGAGTTGGACTCCTTCAAAGA
GGAGCTTGATAAGTATTTCAAGAACCATACCTCTCCCGACGTGGACCTGGGGGA
CATTAGCGGCATCAATGCATCCGTTGTGAATATCCAGAAAGAAATCGATAGGCT
GAATGAGGTCGCAAAAAATCTTAATGAGTCACTGATTGATCTGCAGGAACTCGG
CAAATATGAGCAGTATATTAAGTGGCCGTGGTACATATGGCTCGGCTTTATCGCC
GGTCTGATTGCCATCGTGATGGTGACCATTATGCTGTGTTGTATGACAAGCTGCT
GTTCATGTCTCAAAGGATGCTGCTCCTGCGGTAGCTGCTGTAAGTTCGATGAAGA
CGACAGTGAGCCCGTGCTCAAAGGAGTGAAACTCCACTACACATAAcgatcgGATA
TCGCTAGCGTACCGGCGGCCGCCCTATTCTATAGTGTCACCTAAATGCTAGAGCT
CGCTGATCAGCCTCGACTGTGCCTTCTAGTTGCCAGCCATCTGTTGTTTGCCCCTC
CCCCGTGCCTTCCTTGACCCTGGAAGGTGCCACTCCCACTGTCCTTTCCTAATAAA
ATGAGGAAATTGCATCGCATTGTCTGAGTAGGTGTCATTCTATTCTGGGGGGTGG
GGTGGGGCAGGACAGCAAGGGGGAGGATTGGGAAGACAATAGCAGGCATGCTG
GGGATGCGGTGGGCTCTATGGCTTCTGAGGCGGAAAGAACCAAAGCTTAcgcgttagt
tattaataGTAATCAATTACGGGGTCATTAGTTCATAGCCCATATATGGAGTTCCGCGT
TACATAACTTACGGTAAATGGCCCGCCTGGCTGACCGCCCAACGACCCCCGCCCA
TTGACGTCAATAATGACGTATGTTCCCATAGTAACGCCAATAGGGACTTTCCATT
GACGTCAATGGGTGGAGTATTTACGGTAAACTGCCCACTTGGCAGTACATCAAGT
GTATCATATGCCAAGTACGCCCCCTATTGACGTCAATGACGGTAAATGGCCCGCC
TGGCATTATGCCCAGTACATGACCTTATGGGACTTTCCTACTTGGCAGTACATCT
ACGTATTAGTCATCGCTATTACCATGGTGATGCGGTTTTGGCAGTACATCAATGG
GCGTGGATAGCGGTTTGACTCACGGGGATTTCCAAGTCTCCACCCCATTGACGTC
AATGGGAGTTTGTTTTGGCACCAAAATCAACGGGACTTTCCAAAATGTCGTAACA
ACTCCGCCCCATTGACGCAAATGGGCGGTAGGCGTGTACGGTGGGAGGTCTATA
TAAGCAGAGCTGGTTTAGTGAACCGTCAGATCCGCTAGAGATATCGGGCCACTG
CAGGAAACGATATGGGCTGAATACGGATCCGTATTCAGCCCATATCGTTTCTCTA
GAAATAAAATATCTTTATTTTCATTACATCTGTGTGTTGGTTTTTTGTGTGAATCG
ATAGTACTAACATACGCTCTCCATCTCGAGCCTAAGCTTGTCGACTCGAAGATCT
GGGCGTGGTTAAGGGTGGGAAAGAATATATAAGGTGGGGGTCTTATGTAGTTTT
GTATCTGTTTTGCAGCAGCCGCCGCCGCCATGAGCACCAACTCGTTTGATGGAAG
CATTGTGAGCTCATATTTGACAACGCGCATGCCCCCATGGGCCGGGGTGCGTCAG
AATGTGATGGGCTCCAGCATTGATGGTCGCCCCGTCCTGCCCGCAAACTCTACTA
CCTTGACCTACGAGACCGTGTCTGGAACGCCGTTGGAGACTGCAGCCTCCGCCGC
CGCTTCAGCCGCTGCAGCCACCGCCCGCGGGATTGTGACTGACTTTGCTTTCCTG
AGCCCGCTTGCAAGCAGTGCAGCTTCCCGTTCATCCGCCCGCGATGACAAGTTGA
CGGCTCTTTTGGCACAATTGGATTCTTTGACCCGGGAACTTAATGTCGTTTCTCAG
CAGCTGTTGGATCTGCGCCAGCAGGTTTCTGCCCTGAAGGCTTCCTCCCCTCCCA
ATGCGGTTTAAAACATAAATAAAAAACCAGACTCTGTTTGGATTTGGATCAAGCA
AGTGTCTTGCTGTCTTTATTTAGGGGTTTTGCGCGCGCGGTAGGCCCGGGACCAG
CGGTCTCGGTCGTTGAGGGTCCTGTGTATTTTTTCCAGGACGTGGTAAAGGTGAC
TCTGGATGTTCAGATACATGGGCATAAGCCCGTCTCTGGGGTGGAGGTAGCACC
ACTGCAGAGCTTCATGCTGCGGGGTGGTGTTGTAGATGATCCAGTCGTAGCAGG
AGCGCTGGGCGTGGTGCCTAAAAATGTCTTTCAGTAGCAAGCTGATTGCCAGGG
GCAGGCCCTTGGTGTAAGTGTTTACAAAGCGGTTAAGCTGGGATGGGTGCATAC
GTGGGGATATGAGATGCATCTTGGACTGTATTTTTAGGTTGGCTATGTTCCCAGC
CATATCCCTCCGGGGATTCATGTTGTGCAGAACCACCAGCACAGTGTATCCGGTG
CACTTGGGAAATTTGTCATGTAGCTTAGAAGGAAATGCGTGGAAGAACTTGGAG
ACGCCCTTGTGACCTCCAAGATTTTCCATGCATTCGTCCATAATGATGGCAATGG
GCCCACGGGCGGCGGCCTGGGCGAAGATATTTCTGGGATCACTAACGTCATAGT
TGTGTTCCAGGATGAGATCGTCATAGGCCATTTTTACAAAGCGCGGGCGGAGGG
TGCCAGACTGCGGTATAATGGTTCCATCCGGCCCAGGGGCGTAGTTACCCTCACA
GATTTGCATTTCCCACGCTTTGAGTTCAGATGGGGGGATCATGTCTACCTGCGGG
GCGATGAAGAAAACGGTTTCCGGGGTAGGGGAGATCAGCTGGGAAGAAAGCAG
GTTCCTGAGCAGCTGCGACTTACCGCAGCCGGTGGGCCCGTAAATCACACCTATT
ACCGGCTGCAACTGGTAGTTAAGAGAGCTGCAGCTGCCGTCATCCCTGAGCAGG
GGGGCCACTTCGTTAAGCATGTCCCTGACTCGCATGTTTTCCCTGACCAAATCCG
CCAGAAGGCGCTCGCCGCCCAGCGATAGCAGTTCTTGCAAGGAAGCAAAGTTTT
TCAACGGTTTGAGACCGTCCGCCGTAGGCATGCTTTTGAGCGTTTGACCAAGCAG
TTCCAGGCGGTCCCACAGCTCGGTCACCTGCTCTACGGCATCTCGATCCAGCATA
TCTCCTCGTTTCGCGGGTTGGGGCGGCTTTCGCTGTACGGCAGTAGTCGGTGCTC
GTCCAGACGGGCCAGGGTCATGTCTTTCCACGGGCGCAGGGTCCTCGTCAGCGTA
GTCTGGGTCACGGTGAAGGGGTGCGCTCCGGGCTGCGCGCTGGCCAGGGTGCGC
TTGAGGCTGGTCCTGCTGGTGCTGAAGCGCTGCCGGTCTTCGCCCTGCGCGTCGG
CCAGGTAGCATTTGACCATGGTGTCATAGTCCAGCCCCTCCGCGGCGTGGCCCTT
GGCGCGCAGCTTGCCCTTGGAGGAGGCGCCGCACGAGGGGCAGTGCAGACTTTT
GAGGGCGTAGAGCTTGGGCGCGAGAAATACCGATTCCGGGGAGTAGGCATCCGC
GCCGCAGGCCCCGCAGACGGTCTCGCATTCCACGAGCCAGGTGAGCTCTGGCCG
TTCGGGGTCAAAAACCAGGTTTCCCCCATGCTTTTTGATGCGTTTCTTACCTCTGG
TTTCCATGAGCCGGTGTCCACGCTCGGTGACGAAAAGGCTGTCCGTGTCCCCGTA
TACAGACTTGAGAGGCCTGTCCTCGAGCGGTGTTCCGCGGTCCTCCTCGTATAGA
AACTCGGACCACTCTGAGACAAAGGCTCGCGTCCAGGCCAGCACGAAGGAGGCT
AAGTGGGAGGGGTAGCGGTCGTTGTCCACTAGGGGGTCCACTCGCTCCAGGGTG
TGAAGACACATGTCGCCCTCTTCGGCATCAAGGAAGGTGATTGGTTTGTAGGTGT
AGGCCACGTGACCGGGTGTTCCTGAAGGGGGGCTATAAAAGGGGGTGGGGGCGC
GTTCGTCCTCACTCTCTTCCGCATCGCTGTCTGCGAGGGCCAGCTGTTGGGGTGA
GTACTCCCTCTGAAAAGCGGGCATGACTTCTGCGCTAAGATTGTCAGTTTCCAAA
AACGAGGAGGATTTGATATTCACCTGGCCCGCGGTGATGCCTTTGAGGGTGGCC
GCATCCATCTGGTCAGAAAAGACAATCTTTTTGTTGTCAAGCTTGGTGGCAAACG
ACCCGTAGAGGGCGTTGGACAGCAACTTGGCGATGGAGCGCAGGGTTTGGTTTT
TGTCGCGATCGGCGCGCTCCTTGGCCGCGATGTTTAGCTGCACGTATTCGCGCGC
AACGCACCGCCATTCGGGAAAGACGGTGGTGCGCTCGTCGGGCACCAGGTGCAC
GCGCCAACCGCGGTTGTGCAGGGTGACAAGGTCAACGCTGGTGGCTACCTCTCC
GCGTAGGCGCTCGTTGGTCCAGCAGAGGCGGCCGCCCTTGCGCGAGCAGAATGG
CGGTAGGGGGTCTAGCTGCGTCTCGTCCGGGGGGTCTGCGTCCACGGTAAAGAC
CCCGGGCAGCAGGCGCGCGTCGAAGTAGTCTATCTTGCATCCTTGCAAGTCTAGC
GCCTGCTGCCATGCGCGGGCGGCAAGCGCGCGCTCGTATGGGTTGAGTGGGGGA
CCCCATGGCATGGGGTGGGTGAGCGCGGAGGCGTACATGCCGCAAATGTCGTAA
ACGTAGAGGGGCTCTCTGAGTATTCCAAGATATGTAGGGTAGCATCTTCCACCGC
GGATGCTGGCGCGCACGTAATCGTATAGTTCGTGCGAGGGAGCGAGGAGGTCGG
GACCGAGGTTGCTACGGGCGGGCTGCTCTGCTCGGAAGACTATCTGCCTGAAGA
TGGCATGTGAGTTGGATGATATGGTTGGACGCTGGAAGACGTTGAAGCTGGCGT
CTGTGAGACCTACCGCGTCACGCACGAAGGAGGCGTAGGAGTCGCGCAGCTTGT
TGACCAGCTCGGCGGTGACCTGCACGTCTAGGGCGCAGTAGTCCAGGGTTTCCTT
GATGATGTCATACTTATCCTGTCCCTTTTTTTTCCACAGCTCGCGGTTGAGGACAA
ACTCTTCGCGGTCTTTCCAGTACTCTTGGATCGGAAACCCGTCGGCCTCCGAACG
GTAAGAGCCTAGCATGTAGAACTGGTTGACGGCCTGGTAGGCGCAGCATCCCTTT
TCTACGGGTAGCGCGTATGCCTGCGCGGCCTTCCGGAGCGAGGTGTGGGTGAGC
GCAAAGGTGTCCCTGACCATGACTTTGAGGTACTGGTATTTGAAGTCAGTGTCGT
CGCATCCGCCCTGCTCCCAGAGCAAAAAGTCCGTGCGCTTTTTGGAACGCGGATT
TGGCAGGGCGAAGGTGACATCGTTGAAGAGTATCTTTCCCGCGCGAGGCATAAA
GTTGCGTGTGATGCGGAAGGGTCCCGGCACCTCGGAACGGTTGTTAATTACCTGG
GCGGCGAGCACGATCTCGTCAAAGCCGTTGATGTTGTGGCCCACAATGTAAAGTT
CCAAGAAGCGCGGGATGCCCTTGATGGAAGGCAATTTTTTAAGTTCCTCGTAGGT
GAGCTCTTCAGGGGAGCTGAGCCCGTGCTCTGAAAGGGCCCAGTCTGCAAGATG
AGGGTTGGAAGCGACGAATGAGCTCCACAGGTCACGGGCCATTAGCATTTGCAG
GTGGTCGCGAAAGGTCCTAAACTGGCGACCTATGGCCATTTTTTCTGGGGTGATG
CAGTAGAAGGTAAGCGGGTCTTGTTCCCAGCGGTCCCATCCAAGGTTCGCGGCTA
GGTCTCGCGCGGCAGTCACTAGAGGCTCATCTCCGCCGAACTTCATGACCAGCAT
GAAGGGCACGAGCTGCTTCCCAAAGGCCCCCATCCAAGTATAGGTCTCTACATC
GTAGGTGACAAAGAGACGCTCGGTGCGAGGATGCGAGCCGATCGGGAAGAACT
GGATCTCCCGCCACCAATTGGAGGAGTGGCTATTGATGTGGTGAAAGTAGAAGT
CCCTGCGACGGGCCGAACACTCGTGCTGGCTTTTGTAAAAACGTGCGCAGTACTG
GCAGCGGTGCACGGGCTGTACATCCTGCACGAGGTTGACCTGACGACCGCGCAC
AAGGAAGCAGAGTGGGAATTTGAGCCCCTCGCCTGGCGGGTTTGGCTGGTGGTC
TTCTACTTCGGCTGCTTGTCCTTGACCGTCTGGCTGCTCGAGGGGAGTTACGGTG
GATCGGACCACCACGCCGCGCGAGCCCAAAGTCCAGATGTCCGCGCGCGGCGGT
CGGAGCTTGATGACAACATCGCGCAGATGGGAGCTGTCCATGGTCTGGAGCTCC
CGCGGCGTCAGGTCAGGCGGGAGCTCCTGCAGGTTTACCTCGCATAGACGGGTC
AGGGCGCGGGCTAGATCCAGGTGATACCTAATTTCCAGGGGCTGGTTGGTGGCG
GCGTCGATGGCTTGCAAGAGGCCGCATCCCCGCGGCGCGACTACGGTACCGCGC
GGCGGGCGGTGGGCCGCGGGGGTGTCCTTGGATGATGCATCTAAAAGCGGTGAC
GCGGGCGAGCCCCCGGAGGTAGGGGGGGCTCCGGACCCGCCGGGAGAGGGGGC
AGGGGCACGTCGGCGCCGCGCGCGGGCAGGAGCTGGTGCTGCGCGCGTAGGTTG
CTGGCGAACGCGACGACGCGGCGGTTGATCTCCTGAATCTGGCGCCTCTGCGTGA
AGACGACGGGCCCGGTGAGCTTGAACCTGAAAGAGAGTTCGACAGAATCAATTT
CGGTGTCGTTGACGGCGGCCTGGCGCAAAATCTCCTGCACGTCTCCTGAGTTGTC
TTGATAGGCGATCTCGGCCATGAACTGCTCGATCTCTTCCTCCTGGAGATCTCCG
CGTCCGGCTCGCTCCACGGTGGCGGCGAGGTCGTTGGAAATGCGGGCCATGAGC
TGCGAGAAGGCGTTGAGGCCTCCCTCGTTCCAGACGCGGCTGTAGACCACGCCC
CCTTCGGCATCGCGGGCGCGCATGACCACCTGCGCGAGATTGAGCTCCACGTGCC
GGGCGAAGACGGCGTAGTTTCGCAGGCGCTGAAAGAGGTAGTTGAGGGTGGTGG
CGGTGTGTTCTGCCACGAAGAAGTACATAACCCAGCGTCGCAACGTGGATTCGTT
GATATCCCCCAAGGCCTCAAGGCGCTCCATGGCCTCGTAGAAGTCCACGGCGAA
GTTGAAAAACTGGGAGTTGCGCGCCGACACGGTTAACTCCTCCTCCAGAAGACG
GATGAGCTCGGCGACAGTGTCGCGCACCTCGCGCTCAAAGGCTACAGGGGCCTC
TTCTTCTTCTTCAATCTCCTCTTCCATAAGGGCCTCCCCTTCTTCTTCTTCTGGCGG
CGGTGGGGGAGGGGGGACACGGCGGCGACGACGGCGCACCGGGAGGCGGTCGA
CAAAGCGCTCGATCATCTCCCCGCGGCGACGGCGCATGGTCTCGGTGACGGCGC
GGCCGTTCTCGCGGGGGCGCAGTTGGAAGACGCCGCCCGTCATGTCCCGGTTATG
GGTTGGCGGGGGGCTGCCATGCGGCAGGGATACGGCGCTAACGATGCATCTCAA
CAATTGTTGTGTAGGTACTCCGCCGCCGAGGGACCTGAGCGAGTCCGCATCGACC
GGATCGGAAAACCTCTCGAGAAAGGCGTCTAACCAGTCACAGTCGCAAGGTAGG
CTGAGCACCGTGGCGGGCGGCAGCGGGCGGCGGTCGGGGTTGTTTCTGGCGGAG
GTGCTGCTGATGATGTAATTAAAGTAGGCGGTCTTGAGACGGCGGATGGTCGAC
AGAAGCACCATGTCCTTGGGTCCGGCCTGCTGAATGCGCAGGCGGTCGGCCATG
CCCCAGGCTTCGTTTTGACATCGGCGCAGGTCTTTGTAGTAGTCTTGCATGAGCC
TTTCTACCGGCACTTCTTCTTCTCCTTCCTCTTGTCCTGCATCTCTTGCATCTATCG
CTGCGGCGGCGGCGGAGTTTGGCCGTAGGTGGCGCCCTCTTCCTCCCATGCGTGT
GACCCCGAAGCCCCTCATCGGCTGAAGCAGGGCTAGGTCGGCGACAACGCGCTC
GGCTAATATGGCCTGCTGCACCTGCGTGAGGGTAGACTGGAAGTCATCCATGTCC
ACAAAGCGGTGGTATGCGCCCGTGTTGATGGTGTAAGTGCAGTTGGCCATAACG
GACCAGTTAACGGTCTGGTGACCCGGCTGCGAGAGCTCGGTGTACCTGAGACGC
GAGTAAGCCCTCGAGTCAAATACGTAGTCGTTGCAAGTCCGCACCAGGTACTGG
TATCCCACCAAAAAGTGCGGCGGCGGCTGGCGGTAGAGGGGCCAGCGTAGGGTG
GCCGGGGCTCCGGGGGCGAGATCTTCCAACATAAGGCGATGATATCCGTAGATG
TACCTGGACATCCAGGTGATGCCGGCGGCGGTGGTGGAGGCGCGCGGAAAGTCG
CGGACGCGGTTCCAGATGTTGCGCAGCGGCAAAAAGTGCTCCATGGTCGGGACG
CTCTGGCCGGTCAGGCGCGCGCAATCGTTGACGCTCTAGCGTGCAAAAGGAGAG
CCTGTAAGCGGGCACTCTTCCGTGGTCTGGTGGATAAATTCGCAAGGGTATCATG
GCGGACGACCGGGGTTCGAGCCCCGTATCCGGCCGTCCGCCGTGATCCATGCGG
TTACCGCCCGCGTGTCGAACCCAGGTGTGCGACGTCAGACAACGGGGGAGTGCT
CCTTTTGGCTTCCTTCCAGGCGCGGCGGCTGCTGCGCTAGCTTTTTTGGCCACTGG
CCGCGCGCAGCGTAAGCGGTTAGGCTGGAAAGCGAAAGCATTAAGTGGCTCGCT
CCCTGTAGCCGGAGGGTTATTTTCCAAGGGTTGAGTCGCGGGACCCCCGGTTCGA
GTCTCGGACCGGCCGGACTGCGGCGAACGGGGGTTTGCCTCCCCGTCATGCAAG
ACCCCGCTTGCAAATTCCTCCGGAAACAGGGACGAGCCCCTTTTTTGCTTTTCCC
AGATGCATCCGGTGCTGCGGCAGATGCGCCCCCCTCCTCAGCAGCGGCAAGAGC
AAGAGCAGCGGCAGACATGCAGGGCACCCTCCCCTCCTCCTACCGCGTCAGGAG
GGGCGACATCCGCGGTTGACGCGGCAGCAGATGGTGATTACGAACCCCCGCGGC
GCCGGGCCCGGCACTACCTGGACTTGGAGGAGGGCGAGGGCCTGGCGCGGCTAG
GAGCGCCCTCTCCTGAGCGGCACCCAAGGGTGCAGCTGAAGCGTGATACGCGTG
AGGCGTACGTGCCGCGGCAGAACCTGTTTCGCGACCGCGAGGGAGAGGAGCCCG
AGGAGATGCGGGATCGAAAGTTCCACGCAGGGCGCGAGCTGCGGCATGGCCTGA
ATCGCGAGCGGTTGCTGCGCGAGGAGGACTTTGAGCCCGACGCGCGAACCGGGA
TTAGTCCCGCGCGCGCACACGTGGCGGCCGCCGACCTGGTAACCGCATACGAGC
AGACGGTGAACCAGGAGATTAACTTTCAAAAAAGCTTTAACAACCACGTGCGTA
CGCTTGTGGCGCGCGAGGAGGTGGCTATAGGACTGATGCATCTGTGGGACTTTGT
AAGCGCGCTGGAGCAAAACCCAAATAGCAAGCCGCTCATGGCGCAGCTGTTCCT
TATAGTGCAGCACAGCAGGGACAACGAGGCATTCAGGGATGCGCTGCTAAACAT
AGTAGAGCCCGAGGGCCGCTGGCTGCTCGATTTGATAAACATCCTGCAGAGCAT
AGTGGTGCAGGAGCGCAGCTTGAGCCTGGCTGACAAGGTGGCCGCCATCAACTA
TTCCATGCTTAGCCTGGGCAAGTTTTACGCCCGCAAGATATACCATACCCCTTAC
GTTCCCATAGACAAGGAGGTAAAGATCGAGGGGTTCTACATGCGCATGGCGCTG
AAGGTGCTTACCTTGAGCGACGACCTGGGCGTTTATCGCAACGAGCGCATCCAC
AAGGCCGTGAGCGTGAGCCGGCGGCGCGAGCTCAGCGACCGCGAGCTGATGCAC
AGCCTGCAAAGGGCCCTGGCTGGCACGGGCAGCGGCGATAGAGAGGCCGAGTCC
TACTTTGACGCGGGCGCTGACCTGCGCTGGGCCCCAAGCCGACGCGCCCTGGAG
GCAGCTGGGGCCGGACCTGGGCTGGCGGTGGCACCCGCGCGCGCTGGCAACGTC
GGCGGCGTGGAGGAATATGACGAGGACGATGAGTACGAGCCAGAGGACGGCGA
GTACTAAGCGGTGATGTTTCTGATCAGATGATGCAAGACGCAACGGACCCGGCG
GTGCGGGCGGCGCTGCAGAGCCAGCCGTCCGGCCTTAACTCCACGGACGACTGG
CGCCAGGTCATGGACCGCATCATGTCGCTGACTGCGCGCAATCCTGACGCGTTCC
GGCAGCAGCCGCAGGCCAACCGGCTCTCCGCAATTCTGGAAGCGGTGGTCCCGG
CGCGCGCAAACCCCACGCACGAGAAGGTGCTGGCGATCGTAAACGCGCTGGCCG
AAAACAGGGCCATCCGGCCCGACGAGGCCGGCCTGGTCTACGACGCGCTGCTTC
AGCGCGTGGCTCGTTACAACAGCGGCAACGTGCAGACCAACCTGGACCGGCTGG
TGGGGGATGTGCGCGAGGCCGTGGCGCAGCGTGAGCGCGCGCAGCAGCAGGGC
AACCTGGGCTCCATGGTTGCACTAAACGCCTTCCTGAGTACACAGCCCGCCAACG
TGCCGCGGGGACAGGAGGACTACACCAACTTTGTGAGCGCACTGCGGCTAATGG
TGACTGAGACACCGCAAAGTGAGGTGTACCAGTCTGGGCCAGACTATTTTTTCCA
GACCAGTAGACAAGGCCTGCAGACCGTAAACCTGAGCCAGGCTTTCAAAAACTT
GCAGGGGCTGTGGGGGGTGCGGGCTCCCACAGGCGACCGCGCGACCGTGTCTAG
CTTGCTGACGCCCAACTCGCGCCTGTTGCTGCTGCTAATAGCGCCCTTCACGGAC
AGTGGCAGCGTGTCCCGGGACACATACCTAGGTCACTTGCTGACACTGTACCGCG
AGGCCATAGGTCAGGCGCATGTGGACGAGCATACTTTCCAGGAGATTACAAGTG
TCAGCCGCGCGCTGGGGCAGGAGGACACGGGCAGCCTGGAGGCAACCCTAAACT
ACCTGCTGACCAACCGGCGGCAGAAGATCCCCTCGTTGCACAGTTTAAACAGCG
AGGAGGAGCGCATTTTGCGCTACGTGCAGCAGAGCGTGAGCCTTAACCTGATGC
GCGACGGGGTAACGCCCAGCGTGGCGCTGGACATGACCGCGCGCAACATGGAAC
CGGGCATGTATGCCTCAAACCGGCCGTTTATCAACCGCCTAATGGACTACTTGCA
TCGCGCGGCCGCCGTGAACCCCGAGTATTTCACCAATGCCATCTTGAACCCGCAC
TGGCTACCGCCCCCTGGTTTCTACACCGGGGGATTCGAGGTGCCCGAGGGTAACG
ATGGATTCCTCTGGGACGACATAGACGACAGCGTGTTTTCCCCGCAACCGCAGAC
CCTGCTAGAGTTGCAACAGCGCGAGCAGGCAGAGGCGGCGCTGCGAAAGGAAA
GCTTCCGCAGGCCAAGCAGCTTGTCCGATCTAGGCGCTGCGGCCCCGCGGTCAG
ATGCTAGTAGCCCATTTCCAAGCTTGATAGGGTCTCTTACCAGCACTCGCACCAC
CCGCCCGCGCCTGCTGGGCGAGGAGGAGTACCTAAACAACTCGCTGCTGCAGCC
GCAGCGCGAAAAAAACCTGCCTCCGGCATTTCCCAACAACGGGATAGAGAGCCT
AGTGGACAAGATGAGTAGATGGAAGACGTACGCGCAGGAGCACAGGGACGTGC
CAGGCCCGCGCCCGCCCACCCGTCGTCAAAGGCACGACCGTCAGCGGGGTCTGG
TGTGGGAGGACGATGACTCGGCAGACGACAGCAGCGTCCTGGATTTGGGAGGGA
GTGGCAACCCGTTTGCGCACCTTCGCCCCAGGCTGGGGAGAATGTTTTAAAAAA
AAAAAAGCATGATGCAAAATAAAAAACTCACCAAGGCCATGGCACCGAGCGTTG
GTTTTCTTGTATTCCCCTTAGTATGCGGCGCGCGGCGATGTATGAGGAAGGTCCT
CCTCCCTCCTACGAGAGTGTGGTGAGCGCGGCGCCAGTGGCGGCGGCGCTGGGT
TCTCCCTTCGATGCTCCCCTGGACCCGCCGTTTGTGCCTCCGCGGTACCTGGGGCC
TACCGGGGGGAGAAACAGCATCCGTTACTCTGAGTTGGCACCCCTATTCGACACC
ACCCGTGTGTACCTGGTGGACAACAAGTCAACGGATGTGGCATCCCTGAACTAC
CAGAACGACCACAGCAACTTTCTGACCACGGTCATTCAAAACAATGACTACAGC
CCGGGGGAGGCAAGCACACAGACCATCAATCTTGACGACCGGTCGCACTGGGGC
GGCGACCTGAAAACCATCCTGCATACCAACATGCCAAATGTGAACGAGTTCATG
TTTACCAATAAGTTTAAGGCGCGGGTGATGGTGTCGCGCTTGCCTACTAAGGACA
ATCAGGTGGAGCTGAAATACGAGTGGGTGGAGTTCACGCTGCCCGAGGGCAACT
ACTCCGAGACCATGACCATAGACCTTATGAACAACGCGATCGTGGAGCACTACT
TGAAAGTGGGCAGACAGAACGGGGTTCTGGAAAGCGACATCGGGGTAAAGTTTG
ACACCCGCAACTTCAGACTGGGGTTTGACCCCGTCACTGGTCTTGTCATGCCTGG
GGTATATACAAACGAAGCCTTCCATCCAGACATCATTTTGCTGCCAGGATGCGGG
GTGGACTTCACCCACAGCCGCCTGAGCAACTTGTTGGGCATCCGCAAGCGGCAA
CCCTTCCAGGAGGGCTTTAGGATCACCTACGATGATCTGGAGGGTGGTAACATTC
CCGCACTGTTGGATGTGGACGCCTACCAGGCGAGCTTGAAAGATGACACCGAAC
AGGGCGGGGGTGGCGCAGGCGGCAGCAACAGCAGTGGCAGCGGCGCGGAAGAG
AACTCCAACGCGGCAGCCGCGGCAATGCAGCCGGTGGAGGACATGAACGATCAT
GCCATTCGCGGCGACACCTTTGCCACACGGGCTGAGGAGAAGCGCGCTGAGGCC
GAAGCAGCGGCCGAAGCTGCCGCCCCCGCTGCGCAACCCGAGGTCGAGAAGCCT
CAGAAGAAACCGGTGATCAAACCCCTGACAGAGGACAGCAAGAAACGCAGTTA
CAACCTAATAAGCAATGACAGCACCTTCACCCAGTACCGCAGCTGGTACCTTGCA
TACAACTACGGCGACCCTCAGACCGGAATCCGCTCATGGACCCTGCTTTGCACTC
CTGACGTAACCTGCGGCTCGGAGCAGGTCTACTGGTCGTTGCCAGACATGATGCA
AGACCCCGTGACCTTCCGCTCCACGCGCCAGATCAGCAACTTTCCGGTGGTGGGC
GCCGAGCTGTTGCCCGTGCACTCCAAGAGCTTCTACAACGACCAGGCCGTCTACT
CCCAACTCATCCGCCAGTTTACCTCTCTGACCCACGTGTTCAATCGCTTTCCCGAG
AACCAGATTTTGGCGCGCCCGCCAGCCCCCACCATCACCACCGTCAGTGAAAAC
GTTCCTGCTCTCACAGATCACGGGACGCTACCGCTGCGCAACAGCATCGGAGGA
GTCCAGCGAGTGACCATTACTGACGCCAGACGCCGCACCTGCCCCTACGTTTACA
AGGCCCTGGGCATAGTCTCGCCGCGCGTCCTATCGAGCCGCACTTTTTGAGCAAG
CATGTCCATCCTTATATCGCCCAGCAATAACACAGGCTGGGGCCTGCGCTTCCCA
AGCAAGATGTTTGGCGGGGCCAAGAAGCGCTCCGACCAACACCCAGTGCGCGTG
CGCGGGCACTACCGCGCGCCCTGGGGCGCGCACAAACGCGGCCGCACTGGGCGC
ACCACCGTCGATGACGCCATCGACGCGGTGGTGGAGGAGGCGCGCAACTACACG
CCCACGCCGCCACCAGTGTCCACAGTGGACGCGGCCATTCAGACCGTGGTGCGC
GGAGCCCGGCGCTATGCTAAAATGAAGAGACGGCGGAGGCGCGTAGCACGTCGC
CACCGCCGCCGACCCGGCACTGCCGCCCAACGCGCGGCGGCGGCCCTGCTTAAC
CGCGCACGTCGCACCGGCCGACGGGCGGCCATGCGGGCCGCTCGAAGGCTGGCC
GCGGGTATTGTCACTGTGCCCCCCAGGTCCAGGCGACGAGCGGCCGCCGCAGCA
GCCGCGGCCATTAGTGCTATGACTCAGGGTCGCAGGGGCAACGTGTATTGGGTG
CGCGACTCGGTTAGCGGCCTGCGCGTGCCCGTGCGCACCCGCCCCCCGCGCAACT
AGATTGCAAGAAAAAACTACTTAGACTCGTACTGTTGTATGTATCCAGCGGCGGC
GGCGCGCAACGAAGCTATGTCCAAGCGCAAAATCAAAGAAGAGATGCTCCAGGT
CATCGCGCCGGAGATCTATGGCCCCCCGAAGAAGGAAGAGCAGGATTACAAGCC
CCGAAAGCTAAAGCGGGTCAAAAAGAAAAAGAAAGATGATGATGATGAACTTG
ACGACGAGGTGGAACTGCTGCACGCTACCGCGCCCAGGCGACGGGTACAGTGGA
AAGGTCGACGCGTAAAACGTGTTTTGCGACCCGGCACCACCGTAGTCTTTACGCC
CGGTGAGCGCTCCACCCGCACCTACAAGCGCGTGTATGATGAGGTGTACGGCGA
CGAGGACCTGCTTGAGCAGGCCAACGAGCGCCTCGGGGAGTTTGCCTACGGAAA
GCGGCATAAGGACATGCTGGCGTTGCCGCTGGACGAGGGCAACCCAACACCTAG
CCTAAAGCCCGTAACACTGCAGCAGGTGCTGCCCGCGCTTGCACCGTCCGAAGA
AAAGCGCGGCCTAAAGCGCGAGTCTGGTGACTTGGCACCCACCGTGCAGCTGAT
GGTACCCAAGCGCCAGCGACTGGAAGATGTCTTGGAAAAAATGACCGTGGAACC
TGGGCTGGAGCCCGAGGTCCGCGTGCGGCCAATCAAGCAGGTGGCGCCGGGACT
GGGCGTGCAGACCGTGGACGTTCAGATACCCACTACCAGTAGCACCAGTATTGC
CACCGCCACAGAGGGCATGGAGACACAAACGTCCCCGGTTGCCTCAGCGGTGGC
GGATGCCGCGGTGCAGGCGGTCGCTGCGGCCGCGTCCAAGACCTCTACGGAGGT
GCAAACGGACCCGTGGATGTTTCGCGTTTCAGCCCCCCGGCGCCCGCGCCGTTCG
AGGAAGTACGGCGCCGCCAGCGCGCTACTGCCCGAATATGCCCTACATCCTTCCA
TTGCGCCTACCCCCGGCTATCGTGGCTACACCTACCGCCCCAGAAGACGAGCAAC
TACCCGACGCCGAACCACCACTGGAACCCGCCGCCGCCGTCGCCGTCGCCAGCC
CGTGCTGGCCCCGATTTCCGTGCGCAGGGTGGCTCGCGAAGGAGGCAGGACCCT
GGTGCTGCCAACAGCGCGCTACCACCCCAGCATCGTTTAAAAGCCGGTCTTTGTG
GTTCTTGCAGATATGGCCCTCACCTGCCGCCTCCGTTTCCCGGTGCCGGGATTCC
GAGGAAGAATGCACCGTAGGAGGGGCATGGCCGGCCACGGCCTGACGGGCGGC
ATGCGTCGTGCGCACCACCGGCGGCGGCGCGCGTCGCACCGTCGCATGCGCGGC
GGTATCCTGCCCCTCCTTATTCCACTGATCGCCGCGGCGATTGGCGCCGTGCCCG
GAATTGCATCCGTGGCCTTGCAGGCGCAGAGACACTGATTAAAAACAAGTTGCA
TGTGGAAAAATCAAAATAAAAAGTCTGGACTCTCACGCTCGCTTGGTCCTGTAAC
TATTTTGTAGAATGGAAGACATCAACTTTGCGTCTCTGGCCCCGCGACACGGCTC
GCGCCCGTTCATGGGAAACTGGCAAGATATCGGCACCAGCAATATGAGCGGTGG
CGCCTTCAGCTGGGGCTCGCTGTGGAGCGGCATTAAAAATTTCGGTTCCACCGTT
AAGAACTATGGCAGCAAGGCCTGGAACAGCAGCACAGGCCAGATGCTGAGGGA
TAAGTTGAAAGAGCAAAATTTCCAACAAAAGGTGGTAGATGGCCTGGCCTCTGG
CATTAGCGGGGTGGTGGACCTGGCCAACCAGGCAGTGCAAAATAAGATTAACAG
TAAGCTTGATCCCCGCCCTCCCGTAGAGGAGCCTCCACCGGCCGTGGAGACAGT
GTCTCCAGAGGGGCGTGGCGAAAAGCGTCCGCGCCCCGACAGGGAAGAAACTCT
GGTGACGCAAATAGACGAGCCTCCCTCGTACGAGGAGGCACTAAAGCAAGGCCT
GCCCACCACCCGTCCCATCGCGCCCATGGCTACCGGAGTGCTGGGCCAGCACAC
ACCCGTAACGCTGGACCTGCCTCCCCCCGCCGACACCCAGCAGAAACCTGTGCTG
CCAGGCCCGACCGCCGTTGTTGTAACCCGTCCTAGCCGCGCGTCCCTGCGCCGCG
CCGCCAGCGGTCCGCGATCGTTGCGGCCCGTAGCCAGTGGCAACTGGCAAAGCA
CACTGAACAGCATCGTGGGTCTGGGGGTGCAATCCCTGAAGCGCCGACGATGCT
TCTGATAGCTAACGTGTCGTATGTGTGTCATGTATGCGTCCATGTCGCCGCCAGA
GGAGCTGCTGAGCCGCCGCGCGCCCGCTTTCCAAGATGGCTACCCCTTCGATGAT
GCCGCAGTGGTCTTACATGCACATCTCGGGCCAGGACGCCTCGGAGTACCTGAG
CCCCGGGCTGGTGCAGTTTGCCCGCGCCACCGAGACGTACTTCAGCCTGAATAAC
AAGTTTAGAAACCCCACGGTGGCGCCTACGCACGACGTGACCACAGACCGGTCC
CAGCGTTTGACGCTGCGGTTCATCCCTGTGGACCGTGAGGATACTGCGTACTCGT
ACAAGGCGCGGTTCACCCTAGCTGTGGGTGATAACCGTGTGCTGGACATGGCTTC
CACGTACTTTGACATCCGCGGCGTGCTGGACAGGGGCCCTACTTTTAAGCCCTAC
TCTGGCACTGCCTACAACGCCCTGGCTCCCAAGGGTGCCCCAAATCCTTGCGAAT
GGGATGAAGCTGCTACTGCTCTTGAAATAAACCTAGAAGAAGAGGACGATGACA
ACGAAGACGAAGTAGACGAGCAAGCTGAGCAGCAAAAAACTCACGTATTTGGG
CAGGCGCCTTATTCTGGTATAAATATTACAAAGGAGGGTATTCAAATAGGTGTCG
AAGGTCAAACACCTAAATATGCCGATAAAACATTTCAACCTGAACCTCAAATAG
GAGAATCTCAGTGGTACGAAACAGAAATTAATCATGCAGCTGGGAGAGTCCTAA
AAAAGACTACCCCAATGAAACCATGTTACGGTTCATATGCAAAACCCACAAATG
AAAATGGAGGGCAAGGCATTCTTGTAAAGCAACAAAATGGAAAGCTAGAAAGT
CAAGTGGAAATGCAATTTTTCTCAACTACTGAGGCAGCCGCAGGCAATGGTGAT
AACTTGACTCCTAAAGTGGTATTGTACAGTGAAGATGTAGATATAGAAACCCCA
GACACTCATATTTCTTACATGCCCACTATTAAGGAAGGTAACTCACGAGAACTAA
TGGGCCAACAATCTATGCCCAACAGGCCTAATTACATTGCTTTTAGGGACAATTT
TATTGGTCTAATGTATTACAACAGCACGGGTAATATGGGTGTTCTGGCGGGCCAA
GCATCGCAGTTGAATGCTGTTGTAGATTTGCAAGACAGAAACACAGAGCTTTCAT
ACCAGCTTTTGCTTGATTCCATTGGTGATAGAACCAGGTACTTTTCTATGTGGAAT
CAGGCTGTTGACAGCTATGATCCAGATGTTAGAATTATTGAAAATCATGGAACTG
AAGATGAACTTCCAAATTACTGCTTTCCACTGGGAGGTGTGATTAATACAGAGAC
TCTTACCAAGGTAAAACCTAAAACAGGTCAGGAAAATGGATGGGAAAAAGATGC
TACAGAATTTTCAGATAAAAATGAAATAAGAGTTGGAAATAATTTTGCCATGGA
AATCAATCTAAATGCCAACCTGTGGAGAAATTTCCTGTACTCCAACATAGCGCTG
TATTTGCCCGACAAGCTAAAGTACAGTCCTTCCAACGTAAAAATTTCTGATAACC
CAAACACCTACGACTACATGAACAAGCGAGTGGTGGCTCCCGGGCTAGTGGACT
GCTACATTAACCTTGGAGCACGCTGGTCCCTTGACTATATGGACAACGTCAACCC
ATTTAACCACCACCGCAATGCTGGCCTGCGCTACCGCTCAATGTTGCTGGGCAAT
GGTCGCTATGTGCCCTTCCACATCCAGGTGCCTCAGAAGTTCTTTGCCATTAAAA
ACCTCCTTCTCCTGCCGGGCTCATACACCTACGAGTGGAACTTCAGGAAGGATGT
TAACATGGTTCTGCAGAGCTCCCTAGGAAATGACCTAAGGGTTGACGGAGCCAG
CATTAAGTTTGATAGCATTTGCCTTTACGCCACCTTCTTCCCCATGGCCCACAACA
CCGCCTCCACGCTTGAGGCCATGCTTAGAAACGACACCAACGACCAGTCCTTTAA
CGACTATCTCTCCGCCGCCAACATGCTCTACCCTATACCCGCCAACGCTACCAAC
GTGCCCATATCCATCCCCTCCCGCAACTGGGCGGCTTTCCGCGGCTGGGCCTTCA
CGCGCCTTAAGACTAAGGAAACCCCATCACTGGGCTCGGGCTACGACCCTTATTA
CACCTACTCTGGCTCTATACCCTACCTAGATGGAACCTTTTACCTCAACCACACCT
TTAAGAAGGTGGCCATTACCTTTGACTCTTCTGTCAGCTGGCCTGGCAATGACCG
CCTGCTTACCCCCAACGAGTTTGAAATTAAGCGCTCAGTTGACGGGGAGGGTTAC
AACGTTGCCCAGTGTAACATGACCAAAGACTGGTTCCTGGTACAAATGCTAGCTA
ACTATAACATTGGCTACCAGGGCTTCTATATCCCAGAGAGCTACAAGGACCGCAT
GTACTCCTTCTTTAGAAACTTCCAGCCCATGAGCCGTCAGGTGGTGGATGATACT
AAATACAAGGACTACCAACAGGTGGGCATCCTACACCAACACAACAACTCTGGA
TTTGTTGGCTACCTTGCCCCCACCATGCGCGAAGGACAGGCCTACCCTGCTAACT
TCCCCTATCCGCTTATAGGCAAGACCGCAGTTGACAGCATTACCCAGAAAAAGTT
TCTTTGCGATCGCACCCTTTGGCGCATCCCATTCTCCAGTAACTTTATGTCCATGG
GCGCACTCACAGACCTGGGCCAAAACCTTCTCTACGCCAACTCCGCCCACGCGCT
AGACATGACTTTTGAGGTGGATCCCATGGACGAGCCCACCCTTCTTTATGTTTTG
TTTGAAGTCTTTGACGTGGTCCGTGTGCACCAGCCGCACCGCGGCGTCATCGAAA
CCGTGTACCTGCGCACGCCCTTCTCGGCCGGCAACGCCACAACATAAAGAAGCA
AGCAACATCAACAACAGCTGCCGCCATGGGCTCCAGTGAGCAGGAACTGAAAGC
CATTGTCAAAGATCTTGGTTGTGGGCCATATTTTTTGGGCACCTATGACAAGCGC
TTTCCAGGCTTTGTTTCTCCACACAAGCTCGCCTGCGCCATAGTCAATACGGCCG
GTCGCGAGACTGGGGGCGTACACTGGATGGCCTTTGCCTGGAACCCGCACTCAA
AAACATGCTACCTCTTTGAGCCCTTTGGCTTTTCTGACCAGCGACTCAAGCAGGT
TTACCAGTTTGAGTACGAGTCACTCCTGCGCCGTAGCGCCATTGCTTCTTCCCCCG
ACCGCTGTATAACGCTGGAAAAGTCCACCCAAAGCGTACAGGGGCCCAACTCGG
CCGCCTGTGGACTATTCTGCTGCATGTTTCTCCACGCCTTTGCCAACTGGCCCCAA
ACTCCCATGGATCACAACCCCACCATGAACCTTATTACCGGGGTACCCAACTCCA
TGCTCAACAGTCCCCAGGTACAGCCCACCCTGCGTCGCAACCAGGAACAGCTCT
ACAGCTTCCTGGAGCGCCACTCGCCCTACTTCCGCAGCCACAGTGCGCAGATTAG
GAGCGCCACTTCTTTTTGTCACTTGAAAAACATGTAAAAATAATGTACTAGAGAC
ACTTTCAATAAAGGCAAATGCTTTTATTTGTACACTCTCGGGTGATTATTTACCCC
CACCCTTGCCGTCTGCGCCGTTTAAAAATCAAAGGGGTTCTGCCGCGCATCGCTA
TGCGCCACTGGCAGGGACACGTTGCGATACTGGTGTTTAGTGCTCCACTTAAACT
CAGGCACAACCATCCGCGGCAGCTCGGTGAAGTTTTCACTCCACAGGCTGCGCA
CCATCACCAACGCGTTTAGCAGGTCGGGCGCCGATATCTTGAAGTCGCAGTTGGG
GCCTCCGCCCTGCGCGCGCGAGTTGCGATACACAGGGTTGCAGCACTGGAACAC
TATCAGCGCCGGGTGGTGCACGCTGGCCAGCACGCTCTTGTCGGAGATCAGATCC
GCGTCCAGGTCCTCCGCGTTGCTCAGGGCGAACGGAGTCAACTTTGGTAGCTGCC
TTCCCAAAAAGGGCGCGTGCCCAGGCTTTGAGTTGCACTCGCACCGTAGTGGCAT
CAAAAGGTGACCGTGCCCGGTCTGGGCGTTAGGATACAGCGCCTGCATAAAAGC
CTTGATCTGCTTAAAAGCCACCTGAGCCTTTGCGCCTTCAGAGAAGAACATGCCG
CAAGACTTGCCGGAAAACTGATTGGCCGGACAGGCCGCGTCGTGCACGCAGCAC
CTTGCGTCGGTGTTGGAGATCTGCACCACATTTCGGCCCCACCGGTTCTTCACGA
TCTTGGCCTTGCTAGACTGCTCCTTCAGCGCGCGCTGCCCGTTTTCGCTCGTCACA
TCCATTTCAATCACGTGCTCCTTATTTATCATAATGCTTCCGTGTAGACACTTAAG
CTCGCCTTCGATCTCAGCGCAGCGGTGCAGCCACAACGCGCAGCCCGTGGGCTC
GTGATGCTTGTAGGTCACCTCTGCAAACGACTGCAGGTACGCCTGCAGGAATCGC
CCCATCATCGTCACAAAGGTCTTGTTGCTGGTGAAGGTCAGCTGCAACCCGCGGT
GCTCCTCGTTCAGCCAGGTCTTGCATACGGCCGCCAGAGCTTCCACTTGGTCAGG
CAGTAGTTTGAAGTTCGCCTTTAGATCGTTATCCACGTGGTACTTGTCCATCAGC
GCGCGCGCAGCCTCCATGCCCTTCTCCCACGCAGACACGATCGGCACACTCAGCG
GGTTCATCACCGTAATTTCACTTTCCGCTTCGCTGGGCTCTTCCTCTTCCTCTTGC
GTCCGCATACCACGCGCCACTGGGTCGTCTTCATTCAGCCGCCGCACTGTGCGCT
TACCTCCTTTGCCATGCTTGATTAGCACCGGTGGGTTGCTGAAACCCACCATTTGT
AGCGCCACATCTTCTCTTTCTTCCTCGCTGTCCACGATTACCTCTGGTGATGGCGG
GCGCTCGGGCTTGGGAGAAGGGCGCTTCTTTTTCTTCTTGGGCGCAATGGCCAAA
TCCGCCGCCGAGGTCGATGGCCGCGGGCTGGGTGTGCGCGGCACCAGCGCGTCT
TGTGATGAGTCTTCCTCGTCCTCGGACTCGATACGCCGCCTCATCCGCTTTTTTGG
GGGCGCCCGGGGAGGCGGCGGCGACGGGGACGGGGACGACACGTCCTCCATGG
TTGGGGGACGTCGCGCCGCACCGCGTCCGCGCTCGGGGGTGGTTTCGCGCTGCTC
CTCTTCCCGACTGGCCATTTCCTTCTCCTATAGGCAGAAAAAGATCATGGAGTCA
GTCGAGAAGAAGGACAGCCTAACCGCCCCCTCTGAGTTCGCCACCACCGCCTCC
ACCGATGCCGCCAACGCGCCTACCACCTTCCCCGTCGAGGCACCCCCGCTTGAGG
AGGAGGAAGTGATTATCGAGCAGGACCCAGGTTTTGTAAGCGAAGACGACGAGG
ACCGCTCAGTACCAACAGAGGATAAAAAGCAAGACCAGGACAACGCAGAGGCA
AACGAGGAACAAGTCGGGCGGGGGGACGAAAGGCATGGCGACTACCTAGATGT
GGGAGACGACGTGCTGTTGAAGCATCTGCAGCGCCAGTGCGCCATTATCTGCGA
CGCGTTGCAAGAGCGCAGCGATGTGCCCCTCGCCATAGCGGATGTCAGCCTTGCC
TACGAACGCCACCTATTCTCACCGCGCGTACCCCCCAAACGCCAAGAAAACGGC
ACATGCGAGCCCAACCCGCGCCTCAACTTCTACCCCGTATTTGCCGTGCCAGAGG
TGCTTGCCACCTATCACATCTTTTTCCAAAACTGCAAGATACCCCTATCCTGCCGT
GCCAACCGCAGCCGAGCGGACAAGCAGCTGGCCTTGCGGCAGGGCGCTGTCATA
CCTGATATCGCCTCGCTCAACGAAGTGCCAAAAATCTTTGAGGGTCTTGGACGCG
ACGAGAAGCGCGCGGCAAACGCTCTGCAACAGGAAAACAGCGAAAATGAAAGT
CACTCTGGAGTGTTGGTGGAACTCGAGGGTGACAACGCGCGCCTAGCCGTACTA
AAACGCAGCATCGAGGTCACCCACTTTGCCTACCCGGCACTTAACCTACCCCCCA
AGGTCATGAGCACAGTCATGAGTGAGCTGATCGTGCGCCGTGCGCAGCCCCTGG
AGAGGGATGCAAATTTGCAAGAACAAACAGAGGAGGGCCTACCCGCAGTTGGC
GACGAGCAGCTAGCGCGCTGGCTTCAAACGCGCGAGCCTGCCGACTTGGAGGAG
CGACGCAAACTAATGATGGCCGCAGTGCTCGTTACCGTGGAGCTTGAGTGCATG
CAGCGGTTCTTTGCTGACCCGGAGATGCAGCGCAAGCTAGAGGAAACATTGCAC
TACACCTTTCGACAGGGCTACGTACGCCAGGCCTGCAAGATCTCCAACGTGGAG
CTCTGCAACCTGGTCTCCTACCTTGGAATTTTGCACGAAAACCGCCTTGGGCAAA
ACGTGCTTCATTCCACGCTCAAGGGCGAGGCGCGCCGCGACTACGTCCGCGACT
GCGTTTACTTATTTCTATGCTACACCTGGCAGACGGCCATGGGCGTTTGGCAGCA
GTGCTTGGAGGAGTGCAACCTCAAGGAGCTGCAGAAACTGCTAAAGCAAAACTT
GAAGGACCTATGGACGGCCTTCAACGAGCGCTCCGTGGCCGCGCACCTGGCGGA
CATCATTTTCCCCGAACGCCTGCTTAAAACCCTGCAACAGGGTCTGCCAGACTTC
ACCAGTCAAAGCATGTTGCAGAACTTTAGGAACTTTATCCTAGAGCGCTCAGGA
ATCTTGCCCGCCACCTGCTGTGCACTTCCTAGCGACTTTGTGCCCATTAAGTACCG
CGAATGCCCTCCGCCGCTTTGGGGCCACTGCTACCTTCTGCAGCTAGCCAACTAC
CTTGCCTACCACTCTGACATAATGGAAGACGTGAGCGGTGACGGTCTACTGGAGT
GTCACTGTCGCTGCAACCTATGCACCCCGCACCGCTCCCTGGTTTGCAATTCGCA
GCTGCTTAACGAAAGTCAAATTATCGGTACCTTTGAGCTGCAGGGTCCCTCGCCT
GACGAAAAGTCCGCGGCTCCGGGGTTGAAACTCACTCCGGGGCTGTGGACGTCG
GCTTACCTTCGCAAATTTGTACCTGAGGACTACCACGCCCACGAGATTAGGTTCT
ACGAAGACCAATCCCGCCCGCCTAATGCGGAGCTTACCGCCTGCGTCATTACCCA
GGGCCACATTCTTGGCCAATTGCAAGCCATCAACAAAGCCCGCCAAGAGTTTCTG
CTACGAAAGGGACGGGGGGTTTACTTGGACCCCCAGTCCGGCGAGGAGCTCAAC
CCAATCCCCCCGCCGCCGCAGCCCTATCAGCAGCAGCCGCGGGCCCTTGCTTCCC
AGGATGGCACCCAAAAAGAAGCTGCAGCTGCCGCCGCCACCCACGGACGAGGA
GGAATACTGGGACAGTCAGGCAGAGGAGGTTTTGGACGAGGAGGAGGAGGACA
TGATGGAAGACTGGGAGAGCCTAGACGAGGAAGCTTCCGAGGTCGAAGAGGTGT
CAGACGAAACACCGTCACCCTCGGTCGCATTCCCCTCGCCGGCGCCCCAGAAATC
GGCAACCGGTTCCAGCATGGCTACAACCTCCGCTCCTCAGGCGCCGCCGGCACTG
CCCGTTCGCCGACCCAACCGTAGATGGGACACCACTGGAACCAGGGCCGGTAAG
TCCAAGCAGCCGCCGCCGTTAGCCCAAGAGCAACAACAGCGCCAAGGCTACCGC
TCATGGCGCGGGCACAAGAACGCCATAGTTGCTTGCTTGCAAGACTGTGGGGGC
AACATCTCCTTCGCCCGCCGCTTTCTTCTCTACCATCACGGCGTGGCCTTCCCCCG
TAACATCCTGCATTACTACCGTCATCTCTACAGCCCATACTGCACCGGCGGCAGC
GGCAGCAACAGCAGCGGCCACACAGAAGCAAAGGCGACCGGATAGCAAGACTC
TGACAAAGCCCAAGAAATCCACAGCGGCGGCAGCAGCAGGAGGAGGAGCGCTG
CGTCTGGCGCCCAACGAACCCGTATCGACCCGCGAGCTTAGAAACAGGATTTTTC
CCACTCTGTATGCTATATTTCAACAGAGCAGGGGCCAAGAACAAGAGCTGAAAA
TAAAAAACAGGTCTCTGCGATCCCTCACCCGCAGCTGCCTGTATCACAAAAGCG
AAGATCAGCTTCGGCGCACGCTGGAAGACGCGGAGGCTCTCTTCAGTAAATACT
GCGCGCTGACTCTTAAGGACTAGTTTCGCGCCCTTTCTCAAATTTAAGCGCGAAA
ACTACGTCATCTCCAGCGGCCACACCCGGCGCCAGCACCTGTTGTCAGCGCCATT
ATGAGCAAGGAAATTCCCACGCCCTACATGTGGAGTTACCAGCCACAAATGGGA
CTTGCGGCTGGAGCTGCCCAAGACTACTCAACCCGAATAAACTACATGAGCGCG
GGACCCCACATGATATCCCGGGTCAACGGAATACGCGCCCACCGAAACCGAATT
CTCCTGGAACAGGCGGCTATTACCACCACACCTCGTAATAACCTTAATCCCCGTA
GTTGGCCCGCTGCCCTGGTGTACCAGGAAAGTCCCGCTCCCACCACTGTGGTACT
TCCCAGAGACGCCCAGGCCGAAGTTCAGATGACTAACTCAGGGGCGCAGCTTGC
GGGCGGCTTTCGTCACAGGGTGCGGTCGCCCGGGCAGGGTATAACTCACCTGAC
AATCAGAGGGCGAGGTATTCAGCTCAACGACGAGTCGGTGAGCTCCTCGCTTGG
TCTCCGTCCGGACGGGACATTTCAGATCGGCGGCGCCGGCCGCTCTTCATTCACG
CCTCGTCAGGCAATCCTAACTCTGCAGACCTCGTCCTCTGAGCCGCGCTCTGGAG
GCATTGGAACTCTGCAATTTATTGAGGAGTTTGTGCCATCGGTCTACTTTAACCC
CTTCTCGGGACCTCCCGGCCACTATCCGGATCAATTTATTCCTAACTTTGACGCG
GTAAAGGACTCGGCGGACGGCTACGACTGAATGTTAAGTGGAGAGGCAGAGCA
ACTGCGCCTGAAACACCTGGTCCACTGTCGCCGCCACAAGTGCTTTGCCCGCGAC
TCCGGTGAGTTTTGCTACTTTGAATTGCCCGAGGATCATATCGAGGGCCcGGCGC
ACGGCGTCCGGCTTACCGCCCAGGGAGAGCTTGCCCGTAGCCTGATTCGGGAGTT
TACCCAGCGCCCCCTGCTAGTTGAGCGGGACAGGGGACCCTGTGTTCTCACTGTG
ATTTGCAACTGTCCTAACCCTGGATTACATCAAGATCCTCTAGTTAATGTCAGGT
CGCCTAAGTCGATTAACTAGAGTACCCGGGGATCTTATTCCCTTTAACTAATAAA
AAAAAATAATAAAGCATCACTTACTTAAAATCAGTTAGCAAATTTCTGTCCAGTT
TATTCAGCAGCACCTCCTTGCCCTCCTCCCAGCTCTGGTATTGCAGCTTCCTCCTG
GCTGCAAACTTTCTCCACAATCTAAATGGAATGTCAGTTTCCTCCTGTTCCTGTCC
ATCCGCACCCACTATCTTCATGTTGTTGCAGATGAAGCGCGCAAGACCGTCTGAA
GATACCTTCAACCCCGTGTATCCATATGACACGGAAACCGGTCCTCCAACTGTGC
CTTTTCTTACTCCTCCCTTTGTATCCCCCAATGGGTTTCAAGAGAGTCCCCCTGGG
GTACTCTCTTTGCGCCTATCCGAACCTCTAGTTACCTCCAATGGCATGCTTGCGCT
CAAAATGGGCAACGGCCTCTCTCTGGACGAGGCCGGCAACCTTACCTCCCAAAA
TGTAACCACTGTGAGCCCACCTCTCAAAAAAACCAAGTCAAACATAAACCTGGA
AATATCTGCACCCCTCACAGTTACCTCAGAAGCCCTAACTGTGGCTGCCGCCGCA
CCTCTAATGGTCGCGGGCAACACACTCACCATGCAATCACAGGCCCCGCTAACC
GTGCACGACTCCAAACTTAGCATTGCCACCCAAGGACCCCTCACAGTGTCAGAA
GGAAAGCTAGCCCTGCAAACATCAGGCCCCCTCACCACCACCGATAGCAGTACC
CTTACTATCACTGCCTCACCCCCTCTAACTACTGCCACTGGTAGCTTGGGCATTGA
CTTGAAAGAGCCCATTTATACACAAAATGGAAAACTAGGACTAAAGTACGGGGC
TCCTTTGCATGTAACAGACGACCTAAACACTTTGACCGTAGCAACTGGTCCAGGT
GTGACTATTAATAATACTTCCTTGCAAACTAAAGTTACTGGAGCCTTGGGTTTTG
ATTCACAAGGCAATATGCAACTTAATGTAGCAGGAGGACTAAGGATTGATTCTC
AAAACAGACGCCTTATACTTGATGTTAGTTATCCGTTTGATGCTCAAAACCAACT
AAATCTAAGACTAGGACAGGGCCCTCTTTTTATAAACTCAGCCCACAACTTGGAT
ATTAACTACAACAAAGGCCTTTACTTGTTTACAGCTTCAAACAATTCCAAAAAGC
TTGAGGTTAACCTAAGCACTGCCAAGGGGTTGATGTTTGACGCTACAGCCATAGC
CATTAATGCAGGAGATGGGCTTGAATTTGGTTCACCTAATGCACCAAACACAAAT
CCCCTCAAAACAAAAATTGGCCATGGCCTAGAATTTGATTCAAACAAGGCTATG
GTTCCTAAACTAGGAACTGGCCTTAGTTTTGACAGCACAGGTGCCATTACAGTAG
GAAACAAAAATAATGATAAGCTAACTTTGTGGACCACACCAGCTCCATCTCCTA
ACTGTAGACTAAATGCAGAGAAAGATGCTAAACTCACTTTGGTCTTAACAAAAT
GTGGCAGTCAAATACTTGCTACAGTTTCAGTTTTGGCTGTTAAAGGCAGTTTGGC
TCCAATATCTGGAACAGTTCAAAGTGCTCATCTTATTATAAGATTTGACGAAAAT
GGAGTGCTACTAAACAATTCCTTCCTGGACCCAGAATATTGGAACTTTAGAAATG
GAGATCTTACTGAAGGCACAGCCTATACAAACGCTGTTGGATTTATGCCTAACCT
ATCAGCTTATCCAAAATCTCACGGTAAAACTGCCAAAAGTAACATTGTCAGTCAA
GTTTACTTAAACGGAGACAAAACTAAACCTGTAACACTAACCATTACACTAAAC
GGTACACAGGAAACAGGAGACACAACTCCAAGTGCATACTCTATGTCATTTTCAT
GGGACTGGTCTGGCCACAACTACATTAATGAAATATTTGCCACATCCTCTTACAC
TTTTTCATACATTGCCCAAGAATAAAGAATCGTTTGTGTTATGTTTCAACGTGTTT
ATTTTTCAATTGCAGAAAATTTCAAGTCATTTTTCATTCAGTAGTATAGCCCCACC
ACCACATAGCTTATACAGATCACCGTACCTTAATCAAACTCACAGAACCCTAGTA
TTCAACCTGCCACCTCCCTCCCAACACACAGAGTACACAGTCCTTTCTCCCCGGC
TGGCCTTAAAAAGCATCATATCATGGGTAACAGACATATTCTTAGGTGTTATATT
CCACACGGTTTCCTGTCGAGCCAAACGCTCATCAGTGATATTAATAAACTCCCCG
GGCAGCTCACTTAAGTTCATGTCGCTGTCCAGCTGCTGAGCCACAGGCTGCTGTC
CAACTTGCGGTTGCTTAACGGGCGGCGAAGGAGAAGTCCACGCCTACATGGGGG
TAGAGTCATAATCGTGCATCAGGATAGGGCGGTGGTGCTGCAGCAGCGCGCGAA
TAAACTGCTGCCGCCGCCGCTCCGTCCTGCAGGAATACAACATGGCAGTGGTCTC
CTCAGCGATGATTCGCACCGCCCGCAGCATAAGGCGCCTTGTCCTCCGGGCACAG
CAGCGCACCCTGATCTCACTTAAATCAGCACAGTAACTGCAGCACAGCACCACA
ATATTGTTCAAAATCCCACAGTGCAAGGCGCTGTATCCAAAGCTCATGGCGGGG
ACCACAGAACCCACGTGGCCATCATACCACAAGCGCAGGTAGATTAAGTGGCGA
CCCCTCATAAACACGCTGGACATAAACATTACCTCTTTTGGCATGTTGTAATTCA
CCACCTCCCGGTACCATATAAACCTCTGATTAAACATGGCGCCATCCACCACCAT
CCTAAACCAGCTGGCCAAAACCTGCCCGCCGGCTATACACTGCAGGGAACCGGG
ACTGGAACAATGACAGTGGAGAGCCCAGGACTCGTAACCATGGATCATCATGCT
CGTCATGATATCAATGTTGGCACAACACAGGCACACGTGCATACACTTCCTCAGG
ATTACAAGCTCCTCCCGCGTTAGAACCATATCCCAGGGAACAACCCATTCCTGAA
TCAGCGTAAATCCCACACTGCAGGGAAGACCTCGCACGTAACTCACGTTGTGCAT
TGTCAAAGTGTTACATTCGGGCAGCAGCGGATGATCCTCCAGTATGGTAGCGCG
GGTTTCTGTCTCAAAAGGAGGTAGACGATCCCTACTGTACGGAGTGCGCCGAGA
CAACCGAGATCGTGTTGGTCGTAGTGTCATGCCAAATGGAACGCCGGACGTAGT
CATATTTCCTGAAGCAAAACCAGGTGCGGGCGTGACAAACAGATCTGCGTCTCC
GGTCTCGCCGCTTAGATCGCTCTGTGTAGTAGTTGTAGTATATCCACTCTCTCAAA
GCATCCAGGCGCCCCCTGGCTTCGGGTTCTATGTAAACTCCTTCATGCGCCGCTG
CCCTGATAACATCCACCACCGCAGAATAAGCCACACCCAGCCAACCTACACATT
CGTTCTGCGAGTCACACACGGGAGGAGCGGGAAGAGCTGGAAGAACCATGTTTT
TTTTTTTATTCCAAAAGATTATCCAAAACCTCAAAATGAAGATCTATTAAGTGAA
CGCGCTCCCCTCCGGTGGCGTGGTCAAACTCTACAGCCAAAGAACAGATAATGG
CATTTGTAAGATGTTGCACAATGGCTTCCAAAAGGCAAACGGCCCTCACGTCCAA
GTGGACGTAAAGGCTAAACCCTTCAGGGTGAATCTCCTCTATAAACATTCCAGCA
CCTTCAACCATGCCCAAATAATTCTCATCTCGCCACCTTCTCAATATATCTCTAAG
CAAATCCCGAATATTAAGTCCGGCCATTGTAAAAATCTGCTCCAGAGCGCCCTCC
ACCTTCAGCCTCAAGCAGCGAATCATGATTGCAAAAATTCAGGTTCCTCACAGAC
CTGTATAAGATTCAAAAGCGGAACATTAACAAAAATACCGCGATCCCGTAGGTC
CCTTCGCAGGGCCAGCTGAACATAATCGTGCAGGTCTGCACGGACCAGCGCGGC
CACTTCCCCGCCAGGAACCATGACAAAAGAACCCACACTGATTATGACACGCAT
ACTCGGAGCTATGCTAACCAGCGTAGCCCCGATGTAAGCTTGTTGCATGGGCGGC
GATATAAAATGCAAGGTGCTGCTCAAAAAATCAGGCAAAGCCTCGCGCAAAAAA
GAAAGCACATCGTAGTCATGCTCATGCAGATAAAGGCAGGTAAGCTCCGGAACC
ACCACAGAAAAAGACACCATTTTTCTCTCAAACATGTCTGCGGGTTTCTGCATAA
ACACAAAATAAAATAACAAAAAAACATTTAAACATTAGAAGCCTGTCTTACAAC
AGGAAAAACAACCCTTATAAGCATAAGACGGACTACGGCCATGCCGGCGTGACC
GTAAAAAAACTGGTCACCGTGATTAAAAAGCACCACCGACAGCTCCTCGGTCAT
GTCCGGAGTCATAATGTAAGACTCGGTAAACACATCAGGTTGATTCACATCGGTC
AGTGCTAAAAAGCGACCGAAATAGCCCGGGGGAATACATACCCGCAGGCGTAG
AGACAACATTACAGCCCCCATAGGAGGTATAACAAAATTAATAGGAGAGAAAA
ACACATAAACACCTGAAAAACCCTCCTGCCTAGGCAAAATAGCACCCTCCCGCT
CCAGAACAACATACAGCGCTTCCACAGCGGCAGCCATAACAGTCAGCCTTACCA
GTAAAAAAGAAAACCTATTAAAAAAACACCACTCGACACGGCACCAGCTCAATC
AGTCACAGTGTAAAAAAGGGCCAAGTGCAGAGCGAGTATATATAGGACTAAAA
AATGACGTAACGGTrAAAGTCCACAAAAAACACCCAGAAAACCGCACGCGAACC
TACGCCCAGAAACGAAAGCCAAAAAACCCACAACTTCCTCAAATCGTCACTTCC
GTTTTCCCACGTTACGTCACTTCCCATTTTAAGAAAACTACAATTCCCAACACAT
ACAAGTTACTCCGCCCTAAAACCTACGTCACCCGCCCCGTTCCCACGCCCCGCGC
CACGTCACAAACTCCACCCCCTCATTATCATATTGGCTTCAATCCAAAATAAGGT
ATATT
SEQ ID NO: 10: rAd-CMV-SARS-CoV-2-S-BGH-bActin-SARS-CoV-2-N-SPA-BGH-
CMV-dsRNA-SPA
TAAGGATCCCATCATCAATAATATACCTTATTTTGGATTGAAGCCAATATGATAA
TGAGGGGGTGGAGTTTGTGACGTGGCGCGGGGCGTGGGAACGGGGCGGGTGAC
GTAGTAGTGTGGCGGAAGTGTGATGTTGCAAGTGTGGCGGAACACATGTAAGCG
ACGGATGTGGCAAAAGTGACGTTTTTGGTGTGCGCCGGTGTACACAGGAAGTGA
CAATTTTCGCGCGGTTTTAGGCGGATGTTGTAGTAAATTTGGGCGTAACCGAGTA
AGATTTGGCCATTTTCGCGGGAAAACTGAATAAGAGGAAGTGAAATCTGAATAA
TTTTGTGTTACTCATAGCGCGTAATACTGCTAGAGATCTGGCGAAAGGGGGATGT
GCTGCAAGGCGATTAAGTTGGGTAACGCCAGGGTTTTCCCAGTCACGACGTTGTA
AAACGACGGCCAGTGAATTGTAATACGACTCACTATAGGGCGAATTGGGTACTG
GCCACAGGAGCTTGGCCCATTGCATACGTTGTATCCATATCATAATATGTACATT
TATATTGGCTCATGTCCAACATTACCGCCATGTTGACATTGATTATTGACTAGTTA
TTAATAGTAATCAATTACGGGGTCATTAGTTCATAGCCCATATATGGAGTTCCGC
GTTACATAACTTACGGTAAATGGCCCGCCTGGCTGACCGCCCAACGACCCCCGCC
CATTGACGTCAATAATGACGTATGTTCCCATAGTAACGCCAATAGGGACTTTCCA
TTGACGTCAATGGGTGGAGTATTTACGGTAAACTGCCCACTTGGCAGTACATCAA
GTGTATCATATGCCAAGTACGCCCCCTATTGACGTCAATGACGGTAAATGGCCCG
CCTGGCATTATGCCCAGTACATGACCTTATGGGACTTTCCTACTTGGCAGTACAT
CTACGTATTAGTCATCGCTATTACCATGGTGATGCGGTTTTGGCAGTACATCAAT
GGGCGTGGATAGCGGTTTGACTCACGGGGATTTCCAAGTCTCCACCCCATTGACG
TCAATGGGAGTTTGTTTTGGCACCAAAATCAACGGGACTTTCCAAAATGTCGTAA
CAACTCCGCCCCATTGACGCAAATGGGCGGTAGGCGTGTACGGTGGGAGGTCTA
TATAAGCAGAGCTCGTTTAGTGAACCGTCAGATCGCCTGGAGACGCCATCCACG
CTGTTTTGACCTCCATAGAAGACACCGGGACCGATCCAGCCTGACTCTAGCCTAG
CTCTGAAGTTGGTGGTGAGGCCCTGGGCAGGTTGGTATCAAGGTTacaagacaggtttaag
gagaccaatagaaactgggcatgtggagacagagaagactcttgggtttctgataggcactgactctctctgcctattggtctattttccc
acccttaggctgctggtctgagcctagGAGATCTCTCGAGGTCGACGGTATCGATGggtaccgccaccA
TGTTTGTTTTTCTCGTACTCCTGCCCCTGGTTTCCTCCCAATGTGTCAATCTGACTA
CCCGGACCCAACTTCCTCCCGCCTACACCAATTCCTTTACCCGAGGTGTTTACTAC
CCAGACAAAGTGTTCAGGTCATCCGTCCTCCATAGTACCCAAGACCTCTTCCTCC
CTTTTTTTTCTAACGTTACCTGGTTTCACGCTATTCACGTTAGCGGCACCAACGGC
ACCAAAAGATTCGATAACCCCGTACTGCCGTTCAACGACGGGGTATATTTTGCCT
CTACTGAAAAATCAAACATCATACGCGGATGGATCTTTGGGACTACCCTGGACTC
AAAAACTCAGTCCCTGCTGATTGTGAATAACGCTACCAACGTGGTGATCAAAGTC
TGTGAATTCCAGTTTTGCAACGATCCTTTTCTCGGCGTTTATTATCACAAAAATAA
CAAATCCTGGATGGAGAGCGAGTTCCGGGTGTACTCCTCCGCGAATAATTGCACC
TTCGAATATGTGTCTCAGCCATTCCTCATGGACCTCGAGGGGAAGCAGGGCAATT
TTAAGAATCTGCGAGAATTCGTGTTCAAGAATATAGACGGTTACTTCAAGATTTA
CTCCAAACACACCCCGATTAACCTGGTTAGGGACTTGCCTCAGGGCTTTTCTGCA
TTGGAGCCCCTCGTGGACCTCCCAATCGGCATAAACATTACAAGATTTCAGACTT
TGCTTGCATTGCACAGGAGCTATTTGACACCCGGCGATTCTTCTTCCGGATGGAC
CGCTGGAGCAGCTGCTTATTACGTGGGCTATCTGCAGCCTCGAACCTTTCTTTTG
AAGTACAACGAAAATGGAACTATCACCGATGCAGTTGACTGCGCCCTGGACCCC
CTGrCCGAAACTAAGTGCACGCTCAAAAGTTTCACAGTAGAGAAGGGGATATAC
CAGACTAGCAATTTCCGCGTTCAGCCAACCGAAAGTATAGTGCGCTTTCCTAATA
TAACTAACCTGTGTCCTTTCGGGGAAGTGTTTAACGCCACTAGATTCGCTTCCGT
CTACGCCTGGAATAGAAAGAGGATCTCAAATTGCGTTGCTGACTATAGTGTTTTG
TACAATTCCGCCTCTTTCTCAACCTTCAAATGTTACGGGGTGAGCCCTACCAAAC
TGAACGACCTGTGCTTTACAAACGTATACGCCGACAGCTTTGTTATCAGAGGAGA
CGAGGTTCGCCAGATTGCTCCGGGTCAGACAGGCAAGATTGCTGATTATAATTAC
AAACTGCCCGACGACTTTACAGGATGTGTGATCGCGTGGAACAGTAACAATCTT
GACTCAAAGGTTGGGGGTAATTATAATTATCTTTACCGGCTGTTCAGAAAAAGCA
ATTTGAAACCCTTCGAAAGGGACATATCCACCGAGATCTATCAGGCCGGGTCCA
CTCCATGCAATGGTGTGGAAGGTTTTAATTGCTACTTCCCATTGCAGTCTTATGGA
TTCCAACCAACCAATGGCGTAGGCTACCAGCCGTATCGCGTTGTCGTGCTCAGCT
TCGAGCTGCTCCACGCCCCCGCGACCGTATGCGGTCCTAAGAAGTCCACCAATCT
TGTTAAGAACAAGTGTGTAAACTTTAACTTTAACGGGCTGACCGGGACCGGCGTT
CTGACTGAATCTAACAAAAAATTCCTGCCTTTCCAGCAGTTCGGCCGCGATATTG
CTGACACCACTGACGCTGTAAGAGACCCTCAGACCCTTGAAATTCTCGATATCAC
ACCTTGCAGCTTTGGGGGCGTGTCCGTCATCACTCCAGGAACTAACACAAGCAAC
CAGGTGGCAGTGTTGTACCAGGATGTTAATTGTACCGAGGTGCCAGTGGCCATCC
ACGCCGATCAATTGACACCTACCTGGAGGGTTTACAGCACAGGGTCCAATGTTTT
TCAGACAAGAGCCGGATGTCTGATCGGTGCCGAGCATGTCAACAATTCCTACGA
GTGTGATATCCCCATTGGTGCGGGAATTTGTGCATCATATCAGACCCAGACTAAT
AGCCCAAGAAGAGCTAGATCCGTCGCTAGTCAATCCATCATTGCATATACAATGT
CCCTGGGAGCTGAGAATTCAGTCGCGTATTCAAACAATTCCATTGCTATTCCTAC
TAATTTCACTATCTCCGTCACGACCGAGATCCTGCCAGTTTCCATGACTAAGACT
TCTGTTGACTGCACCATGTATATCTGTGGCGATAGCACCGAGTGCAGTAATCTGC
TTCTGCAGTACGGCTCCTTCTGCACACAACTCAATCGAGCACTGACCGGTATTGC
AGTTGAGCAGGACAAGAACACACAGGAGGTCTTTGCACAGGTCAAACAAATTTA
CAAAACCCCCCCCATAAAAGACTTTGGTGGGTTCAACTTCAGCCAAATCCTCCCA
GATCCCAGCAAGCCCTCCAAAAGATCCTTCATCGAAGACCTTTTGTTCAATAAGG
TAACCCTGGCCGACGCAGGCTTCATCAAACAATATGGCGATTGCCTTGGAGACAT
TGCTGCGCGCGATTTGATCTGTGCTCAGAAATTTAACGGTTTGACCGTGCTGCCC
CCACTTCTGACTGATGAGATGATAGCACAGTATACTTCTGCTCTTCTGGCAGGAA
CAATCACTTCCGGGTGGACCTTTGGCGCTGGTGCAGCACTGCAAATCCCCTTCGC
AATGCAAATGGCCTACCGATTCAATGGTATTGGTGTTACCCAGAACGTGCTCTAT
GAGAATCAGAAACTCATCGCCAATCAGTTCAATAGCGCTATTGGCAAGATTCAG
GATTCCCTCAGCTCTACCGCCAGCGCTCTGGGGAAGCTCCAGGACGTGGTGAACC
AAAATGCTCAAGCGCTCAATACCCTTGTGAAACAGCTCAGCTCCAATTTTGGCGC
AATTAGCAGCGTTCTGAATGATATTCTGTCCCGGCTGGACAAGGTAGAAGCAGA
AGTCCAGATCGACAGGCTGATCACCGGGCGGTTGCAGAGTCTCCAGACCTATGT
CACACAACAGCTGATCCGCGCCGCCGAGATCAGGGCTTCCGCTAACCTGGCCGC
CACTAAGATGTCCGAATGCGTGTTGGGGCAGAGTAAGCGGGTCGACTTTTGCGG
GAAGGGATACCATCTGATGAGCTTCCCTCAGTCTGCACCCCACGGAGTAGTGTTC
CTCCACGTCACATATGTGCCCGCTCAGGAAAAGAATTTCACAACCGCACCTGCTA
TCTGTCACGACGGCAAGGCCCACTTTCCTAGAGAAGGAGTTTTCGTATCTAACGG
CACCCACTGGTTCGTGACACAGCGGAACTTTTACGAGCCTCAGATTATAACTACG
GACAACACTTTCGTGTCAGGCAACTGTGACGTGGTGATTGGGATCGTGAACAAC
ACAGTCTACGACCCATTGCAGCCCGAGTTGGACTCCTTCAAAGAGGAGCTTGATA
AGTATTTCAAGAACCATACCTCTCCCGACGTGGACCTGGGGGACATTAGCGGCAT
CAATGCATCCGTTGTGAATATCCAGAAAGAAATCGATAGGCTGAATGAGGTCGC
AAAAAATCTTAATGAGTCACTGATTGATCTGCAGGAACTCGGCAAATATGAGCA
GTATATTAAGTGGCCGTGGTACATATGGCTCGGCTTTATCGCCGGTCTGATTGCC
ATCGTGATGGTGACCATTATGCTGTGTTGTATGACAAGCTGCTGTTCATGTCTCA
AAGGATGCTGCTCCTGCGGTAGCTGCTGTAAGTTCGATGAAGACGACAGTGAGC
CCGTGCTCAAAGGAGTGAAACTCCACTACACATAAcgatcgacgcgtAGAGCTCGCTGA
TCAGCCTCGACTGTGCCTTCTAGTTGCCAGCCATCTGTTGTTTGCCCCTCCCCCGT
GCCTTCCTTGACCCTGGAAGGTGCCACTCCCACTGTCCTTTCCTAATAAAATGAG
GAAATTGCATCGCATTGTCTGAGTAGGTGTCATTCTATTCTGGGGGGTGGGGTGG
GGCAGGACAGCAAGGGGGAGGATTGGGAAGACAATAGCAGGCATGCTGGGGAT
GCGGTGGGCTCTATGGCTTCTGAGGCGGAAAGAACCAaagcttgcggccgcGCCCAGCA
CCCCAAGGCGGCCAACGCCAAAACTCTCCCTCCTCCTCTTCCTCAATCTCGCTCTC
GCTCTTTTTTTTTTTCGCAAAAGGAGGGGAGAGGGGGTAAAAAAATGCTGCACTG
TGCGGCGAAGCCGGTGAGTGAGCGGCGCGGGGCCAATCAGCGTGCGCCGTTCCG
AAAGTTGCCTTTTATGGCTCGAGCGGCCGCGGCGGCGCCCTATAAAACCCAGCG
GCGCGACGCGCCACCACCGCCGAGACcctgcaggccgccaccATGTCCGATAACGGCCCC
CAGAATCAGAGAAACGCTCCCCGCATCACGTTCGGCGGACCAAGTGACAGCACA
GGCAGTAACCAGAACGGAGAACGCTCCGGTGCTCGCTCCAAGCAGCGACGGCCG
CAAGGGCTTCCCAACAATACCGCCAGCTGGTTTACGGCTCTGACCCAACACGGG
AAAGAAGATCTTAAATTCCCCAGGGGCCAGGGCGTCCCTATCAATACTAACTCC
AGCCCGGATGATCAGATAGGCTACTATAGACGCGCTACCCGACGGATACGAGGG
GGGGACGGCAAAATGAAGGACCTTTCCCCCCGGTGGTATTTCTATTACTTGGGCA
CCGGACCAGAAGCCGGACTGCCTTACGGCGCTAACAAAGACGGAATAATCTGGG
TTGCGACGGAGGGCGCCCTGAATACACCTAAAGACCATATCGGCACAAGAAATC
CTGCTAACAATGCCGCGATTGTGCTCCAGCTGCCTCAGGGAACCACGCTGCCTAA
AGGGTTTTACGCTGAGGGGTCAAGGGGGGGGAGTCAAGCGTCTAGTAGGTCATC
CTCTCGCTCTCGCAATAGTTCCCGGAACTCAACCCCAGGCAGCAGCAGAGGAAC
CTCTCCCGCACGGATGGCTGGCAATGGGGGAGATGCTGCCCTTGCTCTCCTTCTG
CTGGATCGCCTTAACCAGCTCGAATCAAAGATGTCTGGAAAAGGTCAGCAGCAG
CAAGGCCAGACCGTGACAAAGAAGAGTGCAGCTGAAGCTAGTAAAAAGCCACG
CCAAAAACGGACCGCAACTAAGGCATATAACGTAACACAGGCCTTCGGCAGAAG
AGGTCCAGAACAAACACAGGGAAACTTTGGCGATCAAGAGCTGATTAGACAGGG
CACAGATTACAAACACTGGCCACAGATCGCGCAGTTTGCACCAAGCGCCTCTGC
ATTCTTCGGGATGAGTCGGATTGGGATGGAAGTCACTCCATCCGGGACCTGGCTT
ACCTACACAGGGGCAATAAAACTCGACGACAAAGACCCAAACTTTAAAGATCAG
GTCATCCTGCTGAATAAACACATCGATGCCTACAAAACTTTCCCCCCAACCGAAC
CAAAGAAAGACAAGAAAAAAAAGGCAGACGAAACGCAAGCGCTCCCTCAGCGC
CAGAAGAAGCAGCAGACCGTTACACTGTTGCCAGCAGCAGATCTGGATGATTTT
TCCAAGCAGCTTCAACAGAGTATGTCAAGCGCTGACAGCACTCAGGCTTGAggcgC
gccgctgaccgatAAATAAAATATCTTTATTTTCATTACATCTGTGTGTTGGTTTTTTGTG
TGacgcgttagttattaataGTAATCAATTACGGGGTCATTAGTTCATAGCCCATATATGGAG
TTCCGCGTTACATAACTTACGGTAAATGGCCCGCCTGGCTGACCGCCCAACGACC
CCCGCCCATTGACGTCAATAATGACGTATGTTCCCATAGTAACGCCAATAGGGAC
TTTCCATTGACGTCAATGGGTGGAGTATTTACGGTAAACTGCCCACTTGGCAGTA
CATCAAGTGTATCATATGCCAAGTACGCCCCCTATTGACGTCAATGACGGTAAAT
GGCCCGCCTGGCATTATGCCCAGTACATGACCTTATGGGACTTTCCTACTTGGCA
GTACATCTACGTATTAGTCATCGCTATTACCATGGTGATGCGGTTTTGGCAGTAC
ATCAATGGGCGTGGATAGCGGTTTGACTCACGGGGATTTCCAAGTCTCCACCCCA
TTGACGTCAATGGGAGTTTGTTTTGGCACCAAAATCAACGGGACTTTCCAAAATG
TCGTAACAACTCCGCCCCATTGACGCAAATGGGCGGTAGGCGTGTACGGTGGGA
GGTCTATATAAGCAGAGCTGGTTTAGTGAACCGTCAGATCCGCTAGAGATATCG
GGCCACTGCAGGAAACGATATGGGCTGAATACGGATCCGTATTCAGCCCATATC
GTTTCTCTAGAAATAAAATATCTTTATTTTCATTACATCTGTGTGTTGGTTTTTTGT
GTGAATCGATAGTACTAACATACGCTCTCCATCTCGAGCCTAAGCTTGTCGACTC
GAAGATCTGGGCGTGGTTAAGGGTGGGAAAGAATATATAAGGTGGGGGTCTTAT
GTAGTTTTGTATCTGTTTTGCAGCAGCCGCCGCCGCCATGAGCACCAACTCGTTT
GATGGAAGCATTGTGAGCTCATATTTGACAACGCGCATGCCCCCATGGGCcGGG
GTGCGTCAGAATGTGATGGGCTCCAGCATTGATGGTCGCCCCGTCCTGCCCGCAA
ACTCTACTACCTTGACCTACGAGACCGTGTCTGGAACGCCGTTGGAGACTGCAGC
CTCCGCCGCCGCTTCAGCCGCTGCAGCCACCGCCCGCGGGATTGTGACTGACTTT
GCTTTCCTGAGCCCGCTTGCAAGCAGTGCAGCTTCCCGTTCATCCGCCCGCGATG
ACAAGTTGACGGCTCTTTTGGCACAATTGGATTCTTTGACCCGGGAACTTAATGT
CGTTTCTCAGCAGCTGTTGGATCTGCGCCAGCAGGTTTCTGCCCTGAAGGCTTCC
TCCCCTCCCAATGCGGTTTAAAACATAAATAAAAAACCAGACTCTGTTTGGATTT
GGATCAAGCAAGTGTCTTGCTGTCTTTATTTAGGGGTTTTGCGCGCGCGGTAGGC
CCGGGACCAGCGGTCTCGGTCGTTGAGGGTCCTGTGTATTTTTTCCAGGACGTGG
TAAAGGTGACTCTGGATGTTCAGATACATGGGCATAAGCCCGTCTCTGGGGTGG
AGGTAGCACCACTGCAGAGCTTCATGCTGCGGGGTGGTGTTGTAGATGATCCAGT
CGTAGCAGGAGCGCTGGGCGTGGTGCCTAAAAATGTCTTTCAGTAGCAAGCTGA
TTGCCAGGGGCAGGCCCTTGGTGTAAGTGTTTACAAAGCGGTTAAGCTGGGATG
GGTGCATACGTGGGGATATGAGATGCATCTTGGACTGTATTTTTAGGTTGGCTAT
GTTCCCAGCCATATCCCTCCGGGGATTCATGTTGTGCAGAACCACCAGCACAGTG
TATCCGGTGCACTTGGGAAATTTGTCATGTAGCTTAGAAGGAAATGCGTGGAAG
AACTTGGAGACGCCCTTGTGACCTCCAAGATTTTCCATGCATTCGTCCATAATGA
TGGCAATGGGCCCACGGGCGGCGGCCTGGGCGAAGATATTTCTGGGATCACTAA
CGTCATAGTTGTGTTCCAGGATGAGATCGTCATAGGCCATTTTTACAAAGCGCGG
GCGGAGGGTGCCAGACTGCGGTATAATGGTTCCATCCGGCCCAGGGGCGTAGTT
ACCCTCACAGATTTGCATTTCCCACGCTTTGAGTTCAGATGGGGGGATCATGTCT
ACCTGCGGGGCGATGAAGAAAACGGTTTCCGGGGTAGGGGAGATCAGCTGGGA
AGAAAGCAGGTTCCTGAGCAGCTGCGACTTACCGCAGCCGGTGGGCCCGTAAAT
CACACCTATTACCGGCTGCAACTGGTAGTTAAGAGAGCTGCAGCTGCCGTCATCC
CTGAGCAGGGGGGCCACTTCGTTAAGCATGTCCCTGACTCGCATGTTTTCCCTGA
CCAAATCCGCCAGAAGGCGCTCGCCGCCCAGCGATAGCAGTTCTTGCAAGGAAG
CAAAGTTTTTCAACGGTTTGAGACCGTCCGCCGTAGGCATGCTTTTGAGCGTTTG
ACCAAGCAGTTCCAGGCGGTCCCACAGCTCGGTCACCTGCTCTACGGCATCTCGA
TCCAGCATATCTCCTCGTTTCGCGGGTTGGGGCGGCTTTCGCTGTACGGCAGTAG
TCGGTGCTCGTCCAGACGGGCCAGGGTCATGTCTTTCCACGGGCGCAGGGTCCTC
GTCAGCGTAGTCTGGGTCACGGTGAAGGGGTGCGCTCCGGGCTGCGCGCTGGCC
AGGGTGCGCTTGAGGCTGGTCCTGCTGGTGCTGAAGCGCTGCCGGTCTTCGCCCT
GCGCGTCGGCCAGGTAGCATTTGACCATGGTGTCATAGTCCAGCCCCTCCGCGGC
GTGGCCCTTGGCGCGCAGCTTGCCCTTGGAGGAGGCGCCGCACGAGGGGCAGTG
CAGACTTTTGAGGGCGTAGAGCTTGGGCGCGAGAAATACCGATTCCGGGGAGTA
GGCATCCGCGCCGCAGGCCCCGCAGACGGTCTCGCATTCCACGAGCCAGGTGAG
CTCTGGCCGTTCGGGGTCAAAAACCAGGTTTCCCCCATGCTTTTTGATGCGTTTCT
TACCTCTGGTTTCCATGAGCCGGTGTCCACGCTCGGTGACGAAAAGGCTGTCCGT
GTCCCCGTATACAGACTTGAGAGGCCTGTCCTCGAGCGGTGTTCCGCGGTCCTCC
TCGTATAGAAACTCGGACCACTCTGAGACAAAGGCTCGCGTCCAGGCCAGCACG
AAGGAGGCTAAGTGGGAGGGGTAGCGGTCGTTGTCCACTAGGGGGTCCACTCGC
TCCAGGGTGTGAAGACACATGTCGCCCTCTTCGGCATCAAGGAAGGTGATTGGTT
TGTAGGTGTAGGCCACGTGACCGGGTGTTCCTGAAGGGGGGCTATAAAAGGGGG
TGGGGGCGCGTTCGTCCTCACTCTCTTCCGCATCGCTGTCTGCGAGGGCCAGCTG
TTGGGGTGAGTACTCCCTCTGAAAAGCGGGCATGACTTCTGCGCTAAGATTGTCA
GTTTCCAAAAACGAGGAGGATTTGATATTCACCTGGCCCGCGGTGATGCCTTTGA
GGGTGGCCGCATCCATCTGGTCAGAAAAGACAATCTTTTTGTTGTCAAGCTTGGT
GGCAAACGACCCGTAGAGGGCGTTGGACAGCAACTTGGCGATGGAGCGCAGGGT
TTGGTTTTTGTCGCGATCGGCGCGCTCCTTGGCCGCGATGTTTAGCTGCACGTATT
CGCGCGCAACGCACCGCCATTCGGGAAAGACGGTGGTGCGCTCGTCGGGCACCA
GGTGCACGCGCCAACCGCGGTTGTGCAGGGTGACAAGGTCAACGCTGGTGGCTA
CCTCTCCGCGTAGGCGCTCGTTGGTCCAGCAGAGGCGGCCGCCCTTGCGCGAGCA
GAATGGCGGTAGGGGGTCTAGCTGCGTCTCGTCCGGGGGGTCTGCGTCCACGGT
AAAGACCCCGGGCAGCAGGCGCGCGTCGAAGTAGTCTATCTTGCATCCTTGCAA
GTCTAGCGCCTGCTGCCATGCGCGGGCGGCAAGCGCGCGCTCGTATGGGTTGAG
TGGGGGACCCCATGGCATGGGGTGGGTGAGCGCGGAGGCGTACATGCCGCAAAT
GTCGTAAACGTAGAGGGGCTCTCTGAGTATTCCAAGATATGTAGGGTAGCATCTT
CCACCGCGGATGCTGGCGCGCACGTAATCGTATAGTTCGTGCGAGGGAGCGAGG
AGGTCGGGACCGAGGTTGCTACGGGCGGGCTGCTCTGCTCGGAAGACTATCTGC
CTGAAGATGGCATGTGAGTTGGATGATATGGTTGGACGCTGGAAGACGTTGAAG
CTGGCGTCTGTGAGACCTACCGCGTCACGCACGAAGGAGGCGTAGGAGTCGCGC
AGCTTGTTGACCAGCTCGGCGGTGACCTGCACGTCTAGGGCGCAGTAGTCCAGG
GTTTCCTTGATGATGTCATACTTATCCTGTCCCTTTTTTTTCCACAGCTCGCGGTTG
AGGACAAACTCTTCGCGGTCTTTCCAGTACTCTTGGATCGGAAACCCGTCGGCCT
CCGAACGGTAAGAGCCTAGCATGTAGAACTGGTTGACGGCCTGGTAGGCGCAGC
ATCCCTTTTCTACGGGTAGCGCGTATGCCTGCGCGGCCTTCCGGAGCGAGGTGTG
GGTGAGCGCAAAGGTGTCCCTGACCATGACTTTGAGGTACTGGTATTTGAAGTCA
GTGTCGTCGCATCCGCCCTGCTCCCAGAGCAAAAAGTCCGTGCGCTTTTTGGAAC
GCGGATTTGGCAGGGCGAAGGTGACATCGTTGAAGAGTATCTTTCCCGCGCGAG
GCATAAAGTTGCGTGTGATGCGGAAGGGTCCCGGCACCTCGGAACGGTTGTTAA
TTACCTGGGCGGCGAGCACGATCTCGTCAAAGCCGTTGATGTTGTGGCCCACAAT
GTAAAGTTCCAAGAAGCGCGGGATGCCCTTGATGGAAGGCAATTTTTTAAGTTCC
TCGTAGGTGAGCTCTTCAGGGGAGCTGAGCCCGTGCTCTGAAAGGGCCCAGTCT
GCAAGATGAGGGTTGGAAGCGACGAATGAGCTCCACAGGTCACGGGCCATTAGC
ATTTGCAGGTGGTCGCGAAAGGTCCTAAACTGGCGACCTATGGCCATTTTTTCTG
GGGTGATGCAGTAGAAGGTAAGCGGGTCTTGTTCCCAGCGGTCCCATCCAAGGT
TCGCGGCTAGGTCTCGCGCGGCAGTCACTAGAGGCTCATCTCCGCCGAACTTCAT
GACCAGCATGAAGGGCACGAGCTGCTTCCCAAAGGCCCCCATCCAAGTATAGGT
CTCTACATCGTAGGTGACAAAGAGACGCTCGGTGCGAGGATGCGAGCCGATCGG
GAAGAACTGGATCTCCCGCCACCAATTGGAGGAGTGGCTATTGATGTGGTGAAA
GTAGAAGTCCCTGCGACGGGCCGAACACTCGTGCTGGCTTTTGTAAAAACGTGC
GCAGTACTGGCAGCGGTGCACGGGCTGTACATCCTGCACGAGGTTGACCTGACG
ACCGCGCACAAGGAAGCAGAGTGGGAATTTGAGCCCCTCGCCTGGCGGGTTTGG
CTGGTGGTCTTCTACTTCGGCTGCTTGTCCTTGACCGTCTGGCTGCTCGAGGGGA
GTTACGGTGGATCGGACCACCACGCCGCGCGAGCCCAAAGTCCAGATGTCCGCG
CGCGGCGGTCGGAGCTTGATGACAACATCGCGCAGATGGGAGCTGTCCATGGTC
TGGAGCTCCCGCGGCGTCAGGTCAGGCGGGAGCTCCTGCAGGTTTACCTCGCATA
GACGGGTCAGGGCGCGGGCTAGATCCAGGTGATACCTAATTTCCAGGGGCTGGT
TGGTGGCGGCGTCGATGGCTTGCAAGAGGCCGCATCCCCGCGGCGCGACTACGG
TACCGCGCGGCGGGCGGTGGGCCGCGGGGGTGTCCTTGGATGATGCATCTAAAA
GCGGTGACGCGGGCGAGCCCCCGGAGGTAGGGGGGGCTCCGGACCCGCCGGGA
GAGGGGGCAGGGGCACGTCGGCGCCGCGCGCGGGCAGGAGCTGGTGCTGCGCG
CGTAGGTTGCTGGCGAACGCGACGACGCGGCGGTTGATCTCCTGAATCTGGCGC
CTCTGCGTGAAGACGACGGGCCCGGTGAGCTTTGAACCTGAAAGAGAGTTCGAGA
GAATCAATTTCGGTGTCGTTGACGGCGGCCTGGCGCAAAATCTCCTGCACGTCTC
CTGAGTTGTCTTGATAGGCGATCTCGGCCATGAACTGCTCGATCTCTTCCTCCTGG
AGATCTCCGCGTCCGGCTCGCTCCACGGTGGCGGCGAGGTCGTTGGAAATGCGG
GCCATGAGCTGCGAGAAGGCGTTGAGGCCTCCCTCGTTCCAGACGCGGCTGTAG
ACCACGCCCCCTTCGGCATCGCGGGCGCGCATGACCACCTGCGCGAGATTGAGC
TCCACGTGCCGGGCGAAGACGGCGTAGTTTCGCAGGCGCTGAAAGAGGTAGTTG
AGGGTGGTGGCGGTGTGTTCTGCCACGAAGAAGTACATAACCCAGCGTCGCAAC
GTGGATTCGTTGATATCCCCCAAGGCCTCAAGGCGCTCCATGGCCTCGTAGAAGT
CCACGGCGAAGTTGAAAAACTGGGAGTTGCGCGCCGACACGGTTAACTCCTCCT
CCAGAAGACGGATGAGCTCGGCGACAGTGTCGCGCACCTCGCGCTCAAAGGCTA
CAGGGGCCTCTTCTTCTTCTTCAATCTCCTCTTCCATAAGGGCCTCCCCTTCTTCTT
CTTCTGGCGGCGGTGGGGGAGGGGGGACACGGCGGCGACGACGGCGCACCGGG
AGGCGGTCGACAAAGCGCTCGATCATCTCCCCGCGGCGACGGCGCATGGTCTCG
GTGACGGCGCGGCCGTTCTCGCGGGGGCGCAGTTGGAAGACGCCGCCCGTCATG
TCCCGGTTATGGGTTGGCGGGGGGCTGCCATGCGGCAGGGATACGGCGCTAACG
ATGCATCTCAACAATTGTTGTGTAGGTACTCCGCCGCCGAGGGACCTGAGCGAGT
CCGCATCGACCGGATCGGAAAACCTCTCGAGAAAGGCGTCTAACCAGTCACAGT
CGCAAGGTAGGCTGAGCACCGTGGCGGGCGGCAGCGGGCGGCGGTCGGGGTTGT
TTCTGGCGGAGGTGCTGCTGATGATGTAATTAAAGTAGGCGGTCTTGAGACGGCG
GATGGTCGACAGAAGCACCATGTCCTTGGGTCCGGCCTGCTGAATGCGCAGGCG
GTCGGCCATGCCCCAGGCTTCGTTTTGACATCGGCGCAGGTCTTTGTAGTAGTCT
TGCATGAGCCTTTCTACCGGCACTTCTTCTTCTCCTTCCTCTTGTCCTGCATCTCTT
GCATCTATCGCTGCGGCGGCGGCGGAGTTTGGCCGTAGGTGGCGCCCTCTTCCTC
CCATGCGTGTGACCCCGAAGCCCCTCATCGGCTGAAGCAGGGCTAGGTCGGCGA
CAACGCGCTCGGCTAATATGGCCTGCTGCACCTGCGTGAGGGTAGACTGGAAGT
CATCCATGTCCACAAAGCGGTGGTATGCGCCCGTGTTGATGGTGTAAGTGCAGTT
GGCCATAACGGACCAGTTAACGGTCTGGTGACCCGGCTGCGAGAGCTCGGTGTA
CCTGAGACGCGAGTAAGCCCTCGAGTCAAATACGTAGTCGTTGCAAGTCCGCAC
CAGGTACTGGTATCCCACCAAAAAGTGCGGCGGCGGCTGGCGGTAGAGGGGCCA
GCGTAGGGTGGCCGGGGCTCCGGGGGCGAGATCTTCCAACATAAGGCGATGATA
TCCGTAGATGTACCTGGACATCCAGGTGATGCCGGCGGCGGTGGTGGAGGCGCG
CGGAAAGTCGCGGACGCGGTTCCAGATGTTGCGCAGCGGCAAAAAGTGCTCCAT
GGTCGGGACGCTCTGGCCGGTCAGGCGCGCGCAATCGTTGACGCTCTAGCGTGC
AAAAGGAGAGCCTGTAAGCGGGCACTCTTCCGTGGTCTGGTGGATAAATTCGCA
AGGGTATCATGGCGGACGACCGGGGTTCGAGCCCCGTATCCGGCCGTCCGCCGT
GATCCATGCGGTTACCGCCCGCGTGTCGAACCCAGGTGTGCGACGTCAGACAAC
GGGGGAGTGCTCCTTTTGGCTTCCTTCCAGGCGCGGCGGCTGCTGCGCTAGCTTT
TTTGGCCACTGGCCGCGCGCAGCGTAAGCGGTTAGGCTGGAAAGCGAAAGCATT
AAGTGGCTCGCTCCCTGTAGCCGGAGGGTTATTTTCCAAGGGTTGAGTCGCGGGA
CCCCCGGTTCGAGTCTCGGACCGGCCGGACTGCGGCGAACGGGGGTTTGCCTCCC
CGTCATGCAAGACCCCGCTTGCAAATTCCTCCGGAAACAGGGACGAGCCCCTTTT
TTGCTTTTCCCAGATGCATCCGGTGCTGCGGCAGATGCGCCCCCCTCCTCAGCAG
CGGCAAGAGCAAGAGCAGCGGCAGACATGCAGGGCACCCTCCCCTCCTCCTACC
GCGTCAGGAGGGGCGACATCCGCGGTTGACGCGGCAGCAGATGGTGATTACGAA
CCCCCGCGGCGCCGGGCCCGGCACTACCTGGACTTGGAGGAGGGCGAGGGCCTG
GCGCGGCTAGGAGCGCCCTCTCCTGAGCGGCACCCAAGGGTGCAGCTGAAGCGT
GATACGCGTGAGGCGTACGTGCCGCGGCAGAACCTGTTTCGCGACCGCGAGGGA
GAGGAGCCCGAGGAGATGCGGGATCGAAAGTTCCACGCAGGGCGCGAGCTGCG
GCATGGCCTGAATCGCGAGCGGTTGCTGCGCGAGGAGGACTTTGAGCCCGACGC
GCGAACCGGGATTAGTCCCGCGCGCGCACACGTGGCGGCCGCCGACCTGGTAAC
CGCATACGAGCAGACGGTGAACCAGGAGATTAACTTTCAAAAAAGCTTTAACAA
CCACGTGCGTACGCTTGTGGCGCGCGAGGAGGTGGCTATAGGACTGATGCATCT
GTGGGACTTTGTAAGCGCGCTGGAGCAAAACCCAAATAGCAAGCCGCTCATGGC
GCAGCTGTTCCTTATAGTGCAGCACAGCAGGGACAACGAGGCATTCAGGGATGC
GCTGCTAAACATAGTAGAGCCCGAGGGCCGCTGGCTGCTCGATTTGATAAACAT
CCTGCAGAGCATAGTGGTGCAGGAGCGCAGCTTGAGCCTGGCTGACAAGGTGGC
CGCCATCAACTATTCCATGCTTAGCCTGGGCAAGTTTTACGCCCGCAAGATATAC
CATACCCCTTACGTTCCCATAGACAAGGAGGTAAAGATCGAGGGGTTCTACATG
CGCATGGCGCTGAAGGTGCTTACCTTGAGCGACGACCTGGGCGTTTATCGCAACG
AGCGCATCCACAAGGCCGTGAGCGTGAGCCGGCGGCGCGAGCTCAGCGACCGCG
AGCTGATGCACAGCCTGCAAAGGGCCCTGGCTGGCACGGGCAGCGGCGATAGAG
AGGCCGAGTCCTACTTTGACGCGGGCGCTGACCTGCGCTGGGCCCCAAGCCGAC
GCGCCCTGGAGGCAGCTGGGGCCGGACCTGGGCTGGCGGTGGCACCCGCGCGCG
CTGGCAACGTCGGCGGCGTGGAGGAATATGACGAGGACGATGAGTACGAGCCA
GAGGACGGCGAGTACTAAGCGGTGATGTTTCTGATCAGATGATGCAAGACGCAA
CGGACCCGGCGGTGCGGGCGGCGCTGCAGAGCCAGCCGTCCGGCCTTAACTCCA
CGGACGACTGGCGCCAGGTCATGGACCGCATCATGTCGCTGACTGCGCGCAATC
CTGACGCGTTCCGGCAGCAGCCGCAGGCCAACCGGCTCTCCGCAATTCTGGAAG
CGGTGGTCCCGGCGCGCGCAAACCCCACGCACGAGAAGGTGCTGGCGATCGTAA
ACGCGCTGGCCGAAAACAGGGCCATCCGGCCCGACGAGGCCGGCCTGGTCTACG
ACGCGCTGCTTCAGCGCGTGGCTCGTTACAACAGCGGCAACGTGCAGACCAACC
TGGACCGGCTGGTGGGGGATGTGCGCGAGGCCGTGGCGCAGCGTGAGCGCGCGC
AGCAGCAGGGCAACCTGGGCTCCATGGTTGCACTAAACGCCTTCCTGAGTACAC
AGCCCGCCAACGTGCCGCGGGGACAGGAGGACTACACCAACTTTGTGAGCGCAC
TGCGGCTAATGGTGACTGAGACACCGCAAAGTGAGGTGTACCAGTCTGGGCCAG
ACTATTTTTTCCAGACCAGTAGACAAGGCCTGCAGACCGTAAACCTGAGCCAGG
CTTTCAAAAACTTGCAGGGGCTGTGGGGGGTGCGGGCTCCCACAGGCGACCGCG
CGACCGTGTCTAGCTTGCTGACGCCCAACTCGCGCCTGTTGCTGCTGCTAATAGC
GCCCTTCACGGACAGTGGCAGCGTGTCCCGGGACACATACCTAGGTCACTTGCTG
ACACTGTACCGCGAGGCCATAGGTCAGGCGCATGTGGACGAGCATACTTTCCAG
GAGATTACAAGTGTCAGCCGCGCGCTGGGGCAGGAGGACACGGGCAGCCTGGA
GGCAACCCTAAACTACCTGCTGACCAACCGGCGGCAGAAGATCCCCTCGTTGCA
CAGTTTAAACAGCGAGGAGGAGCGCATTTTGCGCTACGTGCAGCAGAGCGTGAG
CCTTAACCTGATGCGCGACGGGGTAACGCCCAGCGTGGCGCTGGACATGACCGC
GCGCAACATGGAACCGGGCATGTATGCCTCAAACCGGCCGTTTATCAACCGCCT
AATGGACTACTTGCATCGCGCGGCCGCCGTGAACCCCGAGTATTTCACCAATGCC
ATCTTGAACCCGCACTGGCTACCGCCCCCTGGTTTCTACACCGGGGGATTCGAGG
TGCCCGAGGGTAACGATGGATTCCTCTGGGACGACATAGACGACAGCGTGTTTTC
CCCGCAACCGCAGACCCTGCTAGAGTTGCAACAGCGCGAGCAGGCAGAGGCGGC
GCTGCGAAAGGAAAGCTTCCGCAGGCCAAGCAGCTTGTCCGATCTAGGCGCTGC
GGCCCCGCGGTCAGATGCTAGTAGCCCATTTCCAAGCTTGATAGGGTCTCTTACC
AGCACTCGCACCACCCGCCCGCGCCTGCTGGGCGAGGAGGAGTACCTAAACAAC
TCGCTGCTGCAGCCGCAGCGCGAAAAAAACCTGCCTCCGGCATTTCCCAACAAC
GGGATAGAGAGCCTAGTGGACAAGATGAGTAGATGGAAGACGTACGCGCAGGA
GCACAGGGACGTGCCAGGCCCGCGCCCGCCCACCCGTCGTCAAAGGCACGACCG
TCAGCGGGGTCTGGTGTGGGAGGACGATGACTCGGCAGACGACAGCAGCGTCCT
GGATTTGGGAGGGAGTGGCAACCCGTTTGCGCACCTTCGCCCCAGGCTGGGGAG
AATGTTTTAAAAAAAAAAAAGCATGATGCAAAATAAAAAACTCACCAAGGCCAT
GGCACCGAGCGTTGGTTTTCTTGTATTCCCCTTAGTATGCGGCGCGCGGCGATGT
ATGAGGAAGGTCCTCCTCCCTCCTACGAGAGTGTGGTGAGCGCGGCGCCAGTGG
CGGCGGCGCTGGGTTCTCCCTTCGATGCTCCCCTGGACCCGCCGTTTGTGCCTCC
GCGGTACCTGCGGCCTACCGGGGGGAGAAACAGCATCCGTTACTCTGAGTTGGC
ACCCCTATTCGACACCACCCGTGTGTACCTGGTGGACAACAAGTCAACGGATGTG
GCATCCCTGAACTACCAGAACGACCACAGCAACTTTCTGACCACGGTCATTCAAA
ACAATGACTACAGCCCGGGGGAGGCAAGCACACAGACCATCAATCTTGACGACC
GGTCGCACTGGGGCGGCGACCTGAAAACCATCCTGCATACCAACATGCCAAATG
TGAACGAGTTCATGTTTACCAATAAGTTTAAGGCGCGGGTGATGGTGTCGCGCTT
GCCTACTAAGGACAATCAGGTGGAGCTGAAATACGAGTGGGTGGAGTTCACGCT
GCCCGAGGGCAACTACTCCGAGACCATGACCATAGACCTTATGAACAACGCGAT
CGTGGAGCACTACTTGAAAGTGGGCAGACAGAACGGGGTTCTGGAAAGCGACAT
CGGGGTAAAGTTTGACACCCGCAACTTCAGACTGGGGTTTGACCCCGTCACTGGT
CTTGTCATGCCTGGGGTATATACAAACGAAGCCTTCCATCCAGACATCATTTTGC
TGCCAGGATGCGGGGTGGACTTCACCCACAGCCGCCTGAGCAACTTGTTGGGCA
TCCGCAAGCGGCAACCCTTCCAGGAGGGCTTTAGGATCACCTACGATGATCTGG
AGGGTGGTAACATTCCCGCACTGTTGGATGTGGACGCCTACCAGGCGAGCTTGA
AAGATGACACCGAACAGGGCGGGGGTGGCGCAGGCGGCAGCAACAGCAGTGGC
AGCGGCGCGGAAGAGAACTCCAACGCGGCAGCCGCGGCAATGCAGCCGGTGGA
GGACATGAACGATCATGCCATTCGCGGCGACACCTTTGCCACACGGGCTGAGGA
GAAGCGCGCTGAGGCCGAAGCAGCGGCCGAAGCTGCCGCCCCCGCTGCGCAACC
CGAGGTCGAGAAGCCTCAGAAGAAACCGGTGATCAAACCCCTGACAGAGGACA
GCAAGAAACGCAGTTACAACCTAATAAGCAATGACAGCACCTTCACCCAGTACC
GCAGCTGGTACCTTGCATACAACTACGGCGACCCTCAGACCGGAATCCGCTCATG
GACCCTGCTTTGCACTCCTGACGTAACCTGCGGCTCGGAGCAGGTCTACTGGTCG
TTGCCAGACATGATGCAAGACCCCGTGACCTTCCGCTCCACGCGCCAGATCAGCA
ACTTTCCGGTGGTGGGCGCCGAGCTGTTGCCCGTGCACTCCAAGAGCTTCTACAA
CGACCAGGCCGTCTACTCCCAACTCATCCGCCAGTTTACCTCTCTGACCCACGTG
TTCAATCGCTTTCCCGAGAACCAGATTTTGGCGCGCCCGCCAGCCCCCACCATCA
CCACCGTCAGTGAAAACGTTCCTGCTCTCACAGATCACGGGACGCTACCGCTGCG
CAACAGCATCGGAGGAGTCCAGCGAGTGACCATTACTGACGCCAGACGCCGCAC
CTGCCCCTACGTTTACAAGGCCCTGGGCATAGTCTCGCCGCGCGTCCTATCGAGC
CGCACTTTTTGAGCAAGCATGTCCATCCTTATATCGCCCAGCAATAACACAGGCT
GGGGCCTGCGCTTCCCAAGCAAGATGTTTGGCGGGGCCAAGAAGCGCTCCGACC
AACACCCAGTGCGCGTGCGCGGGCACTACCGCGCGCCCTGGGGCGCGCACAAAC
GCGGCCGCACTGGGCGCACCACCGTCGATGACGCCATCGACGCGGTGGTGGAGG
AGGCGCGCAACTACACGCCCACGCCGCCACCAGTGTCCACAGTGGACGCGGCCA
TTCAGACCGTGGTGCGCGGAGCCCGGCGCTATGCTAAAATGAAGAGACGGCGGA
GGCGCGTAGCACGTCGCCACCGCCGCCGACCCGGCACTGCCGCCCAACGCGCGG
CGGCGGCCCTGCTTAACCGCGCACGTCGCACCGGCCGACGGGCGGCCATGCGGG
CCGCTCGAAGGCTGGCCGCGGGTATTGTCACTGTGCCCCCCAGGTCCAGGCGAC
GAGCGGCCGCCGCAGCAGCCGCGGCCATTAGTGCTATGACTCAGGGTCGCAGGG
GCAACGTGTATTGGGTGCGCGACTCGGTTAGCGGCCTGCGCGTGCCCGTGCGCAC
CCGCCCCCCGCGCAACTAGATTGCAAGAAAAAACTACTTAGACTCGTACTGTTGT
ATGTATCCAGCGGCGGCGGCGCGCAACGAAGCTATGTCCAAGCGCAAAATCAAA
GAAGAGATGCTCCAGGTCATCGCGCCGGAGATCTATGGCCCCCCGAAGAAGGAA
GAGCAGGATTACAAGCCCCGAAAGCTAAAGCGGGTCAAAAAGAAAAAGAAAGA
TGATGATGATGAACTTGACGACGAGGTGGAACTGCTGCACGCTACCGCGCCCAG
GCGACGGGTACAGTGGAAAGGTCGACGCGTAAAACGTGTTTTGCGACCCGGCAC
CACCGTAGTCTTTACGCCCGGTGAGCGCTCCACCCGCACCTACAAGCGCGTGTAT
GATGAGGTGTACGGCGACGAGGACCTGCTTGAGCAGGCCAACGAGCGCCTCGGG
GAGTTTGCCTACGGAAAGCGGCATAAGGACATGCTGGCGTTGCCGCTGGACGAG
GGCAACCCAACACCTAGCCTAAAGCCCGTAACACTGCAGCAGGTGCTGCCCGCG
CTTGCACCGTCCGAAGAAAAGCGCGGCCTAAAGCGCGAGTCTGGTGACTTGGCA
CCCACCGTGCAGCTGATGGTACCCAAGCGCCAGCGACTGGAAGATGTCTTGGAA
AAAATGACCGTGGAACCTGGGCTGGAGCCCGAGGTCCGCGTGCGGCCAATCAAG
CAGGTGGCGCCGGGACTGGGCGTGCAGACCGTGGACGTTCAGATACCCACTACC
AGTAGCACCAGTATTGCCACCGCCACAGAGGGCATGGAGACACAAACGTCCCCG
GTTGCCTCAGCGGTGGCGGATGCCGCGGTGCAGGCGGTCGCTGCGGCCGCGTCC
AAGACCTCTACGGAGGTGCAAACGGACCCGTGGATGTTTCGCGTTTCAGCCCCCC
GGCGCCCGCGCCGTTCGAGGAAGTACGGCGCCGCCAGCGCGCTACTGCCCGAAT
ATGCCCTACATCCTTCCATTGCGCCTACCCCCGGCTATCGTGGCTACACCTACCG
CCCCAGAAGACGAGCAACTACCCGACGCCGAACCACCACTGGAACCCGCCGCCG
CCGTCGCCGTCGCCAGCCCGTGCTGGCCCCGATTTCCGTGCGCAGGGTGGCTCGC
GAAGGAGGCAGGACCCTGGTGCTGCCAACAGCGCGCTACCACCCCAGCATCGTT
TAAAAGCCGGTCTTTGTGGTTCTTGCAGATATGGCCCTCACCTGCCGCCTCCGTTT
CCCGGTGCCGGGATTCCGAGGAAGAATGCACCGTAGGAGGGGCATGGCCGGCCA
CGGCCTGACGGGCGGCATGCGTCGTGCGCACCACCGGCGGCGGCGCGCGTCGCA
CCGTCGCATGCGCGGCGGTATCCTGCCCCTCCTTATTCCACTGATCGCCGCGGCG
ATTGGCGCCGTGCCCGGAATTGCATCCGTGGCCTTGCAGGCGCAGAGACACTGA
TTAAAAACAAGTTGCATGTGGAAAAATCAAAATAAAAAGTCTGGACTCTCACGC
TCGCTTGGTCCTGTAACTATTTTGTAGAATGGAAGACATCAACTTTGCGTCTCTGG
CCCCGCGACACGGCTCGCGCCCGTTCATGGGAAACTGGCAAGATATCGGCACCA
GCAATATGAGCGGTGGCGCCTTCAGCTGGGGCTCGCTGTGGAGCGGCATTAAAA
ATTTCGGTTCCACCGTTAAGAACTATGGCAGCAAGGCCTGGAACAGCAGCACAG
GCCAGATGCTGAGGGATAAGTTGAAAGAGCAAAATTTCCAACAAAAGGTGGTAG
ATGGCCTGGCCTCTGGCATTAGCGGGGTGGTGGACCTGGCCAACCAGGCAGTGC
AAAATAAGATTAACAGTAAGCTTGATCCCCGCCCTCCCGTAGAGGAGCCTCCAC
CGGCCGTGGAGACAGTGTCTCCAGAGGGGCGTGGCGAAAAGCGTCCGCGCCCCG
ACAGGGAAGAAACTCTGGTGACGCAAATAGACGAGCCTCCCTCGTACGAGGAGG
CACTAAAGCAAGGCCTGCCCACCACCCGTCCCATCGCGCCCATGGCTACCGGAG
TGCTGGGCCAGCACACACCCGTAACGCTGGACCTGCCTCCCCCCGCCGACACCCA
GCAGAAACCTGTGCTGCCAGGCCCGACCGCCGTTGTTGTAACCCGTCCTAGCCGC
GCGTCCCTGCGCCGCGCCGCCAGCGGTCCGCGATCGTTGCGGCCCGTAGCCAGTG
GCAACTGGCAAAGCACACTGAACAGCATCGTGGGTCTGGGGGTGCAATCCCTGA
AGCGCCGACGATGCTTCTGATAGCTAACGTGTCGTATGTGTGTCATGTATGCGTC
CATGTCGCCGCCAGAGGAGCTGCTGAGCCGCCGCGCGCCCGCTTTCCAAGATGG
CTACCCCTTCGATGATGCCGCAGTGGTCTTACATGCACATCTCGGGCCAGGACGC
CTCGGAGTACCTGAGCCCCGGGCTGGTGCAGTTTGCCCGCGCCACCGAGACGTA
CTTCAGCCTGAATAACAAGTTTAGAAACCCCACGGTGGCGCCTACGCACGACGT
GACCACAGACCGGTCCCAGCGTTTGACGCTGCGGTTCATCCCTGTGGACCGTGAG
GATACTGCGTACTCGTACAAGGCGCGGTTCACCCTAGCTGTGGGTGATAACCGTG
TGCTGGACATGGCTTCCACGTACTTTGACATCCGCGGCGTGCTGGACAGGGGCCC
TACTTTTAAGCCCTACTCTGGCACTGCCTACAACGCCCTGGCTCCCAAGGGTGCC
CCAAATCCTTGCGAATGGGATGAAGCTGCTACTGCTCTTGAAATAAACCTAGAA
GAAGAGGACGATGACAACGAAGACGAAGTAGACGAGCAAGCTGAGCAGCAAAA
AACTCACGTATTTGGGCAGGCGCCTTATTCTGGTATAAATATTACAAAGGAGGGT
ATTCAAATAGGTGTCGAAGGTCAAACACCTAAATATGCCGATAAAACATTTCAA
CCTGAACCTCAAATAGGAGAATCTCAGTGGTACGAAACAGAAATTAATCATGCA
GCTGGGAGAGTCCTAAAAAAGACTACCCCAATGAAACCATGTTACGGTTCATAT
GCAAAACCCACAAATGAAAATGGAGGGCAAGGCATTCTTGTAAAGCAACAAAAT
GGAAAGCTAGAAAGTCAAGTGGAAATGCAATTTTTCTCAACTACTGAGGCAGCC
GCAGGCAATGGTGATAACTTGACTCCTAAAGTGGTATTGTACAGTGAAGATGTA
GATATAGAAACCCCAGACACTCATATTTCTTACATGCCCACTATTAAGGAAGGTA
ACTCACGAGAACTAATGGGCCAACAATCTATGCCCAACAGGCCTAATTACATTG
CTTTTAGGGACAATTTTATTGGTCTAATGTATTACAACAGCACGGGTAATATGGG
TGTTCTGGCGGGCCAAGCATCGCAGTTGAATGCTGTTGTAGATTTGCAAGACAGA
AACACAGAGCTTTCATACCAGCTTTTGCTTGATTCCATTGGTGATAGAACCAGGT
ACTTTTCTATGTGGAATCAGGCTGTTGACAGCTATGATCCAGATGTTAGAATTAT
TGAAAATCATGGAACTGAAGATGAACTTCCAAATTACTGCTTTCCACTGGGAGGT
GTGATTAATACAGAGACTCTTACCAAGGTAAAACCTAAAACAGGTCAGGAAAAT
GGATGGGAAAAAGATGCTACAGAATTTTCAGATAAAAATGAAATAAGAGTTGGA
AATAATTTTGCCATGGAAATCAATCTAAATGCCAACCTGTGGAGAAATTTCCTGT
ACTCCAACATAGCGCTGTATTTGCCCGACAAGCTAAAGTACAGTCCTTCCAACGT
AAAAATTTCTGATAACCCAAACACCTACGACTACATGAACAAGCGAGTGGTGGC
TCCCGGGCTAGTGGACTGCTACATTAACCTTGGAGCACGCTGGTCCCTTGACTAT
ATGGACAACGTCAACCCATTTAACCACCACCGCAATGCTGGCCTGCGCTACCGCT
CAATGTTGCTGGGCAATGGTCGCTATGTGCCCTTCCACATCCAGGTGCCTCAGAA
GTTCTTTGCCATTAAAAACCTCCTTCTCCTGCCGGGCTCATACACCTACGAGTGG
AACTTCAGGAAGGATGTTAACATGGTTCTGCAGAGCTCCCTAGGAAATGACCTA
AGGGTTGACGGAGCCAGCATTAAGTTTGATAGCATTTGCCTTTACGCCACCTTCT
TCCCCATGGCCCACAACACCGCCTCCACGCTTGAGGCCATGCTTAGAAACGACAC
CAACGACCAGTCCTTTAACGACTATCTCTCCGCCGCCAACATGCTCTACCCTATA
CCCGCCAACGCTACCAACGTGCCCATATCCATCCCCTCCCGCAACTGGGCGGCTT
TCCGCGGCTGGGCCTTCACGCGCCTTAAGACTAAGGAAACCCCATCACTGGGCTC
GGGCTACGACCCTTATTACACCTACTCTGGCTCTATACCCTACCTAGATGGAACC
TTTTACCTCAACCACACCTTTAAGAAGGTGGCCATTACCTTTGACTCTTCTGTCAG
CTGGCCTGGCAATGACCGCCTGCTTACCCCCAACGAGTTTGAAATTAAGCGCTCA
GTTGACGGGGAGGGTTACAACGTTGCCCAGTGTAACATGACCAAAGACTGGTTC
CTGGTACAAATGCTAGCTAACTATAACATTGGCTACCAGGGCTTCTATATCCCAG
AGAGCTACAAGGACCGCATGTACTCCTTCTTTAGAAACTTCCAGCCCATGAGCCG
TCAGGTGGTGGATGATACTAAATACAAGGACTACCAACAGGTGGGCATCCTACA
CCAACACAACAACTCTGGATTTGTTGGCTACCTTGCCCCCACCATGCGCGAAGGA
CAGGCCTACCCTGCTAACTTCCCCTATCCGCTTATAGGCAAGACCGCAGTTGACA
GCATTACCCAGAAAAAGTTTCTTTGCGATCGCACCCTTTGGCGCATCCCATTCTC
CAGTAACTTTATGTCCATGGGCGCACTCACAGACCTGGGCCAAAACCTTCTCTAC
GCCAACTCCGCCCACGCGCTAGACATGACTTTTGAGGTGGATCCCATGGACGAG
CCCACCCTTCTTTATGTTTTGTTTGAAGTCTTTGACGTGGTCCGTGTGCACCAGCC
GCACCGCGGCGTCATCGAAACCGTGTACCTGCGCACGCCCTTCTCGGCCGGCAAC
GCCACAACATAAAGAAGCAAGCAACATCAACAACAGCTGCCGCCATGGGCTCCA
GTGAGCAGGAACTGAAAGCCATTGTCAAAGATCTTGGTTGTGGGCCATATTTTTT
GGGCACCTATGACAAGCGCTTTCCAGGCTTTGTTTCTCCACACAAGCTCGCCTGC
GCCATAGTCAATACGGCCGGTCGCGAGACTGGGGGCGTACACTGGATGGCCTTT
GCCTGGAACCCGCACTCAAAAACATGCTACCTCTTTGAGCCCTTTGGCTTTTCTG
ACCAGCGACTCAAGCAGGTTTACCAGTTTGAGTACGAGTCACTCCTGCGCCGTAG
CGCCATTGCTTCTTCCCCCGACCGCTGTATAACGCTGGAAAAGTCCACCCAAAGC
GTACAGGGGCCCAACTCGGCCGCCTGTGGACTATTCTGCTGCATGTTTCTCCACG
CCTTTGCCAACTGGCCCCAAACTCCCATGGATCACAACCCCACCATGAACCTTAT
TACCGGGGTACCCAACTCCATGCTCAACAGTCCCCAGGTACAGCCCACCCTGCGT
CGCAACCAGGAACAGCTCTACAGCTTCCTGGAGCGCCACTCGCCCTACTTCCGCA
GCCACAGTGCGCAGATTAGGAGCGCCACTTCTTTTTGTCACTTGAAAAACATGTA
AAAATAATGTACTAGAGACACTTTCAATAAAGGCAAATGCTTTTATTTGTACACT
CTCGGGTGATTATTTACCCCCACCCTTGCCGTCTGCGCCGTTTAAAAATCAAAGG
GGTTCTGCCGCGCATCGCTATGCGCCACTGGCAGGGACACGTTGCGATACTGGTG
TTTAGTGCTCCACTTAAACTCAGGCACAACCATCCGCGGCAGCTCGGTGAAGTTT
TCACTCCACAGGCTGCGCACCATCACCAACGCGTTTAGCAGGTCGGGCGCCGAT
ATCTTGAACTCGCAGTTGGGGCCTCCGCCCTCCGCGCGCGAGTTCGATACACAG
GGTTGCAGCACTGGAACACTATCAGCGCCGGGTGGTGCACGCTGGCCAGCACGC
TCTTGTCGGAGATCAGATCCGCGTCCAGGTCCTCCGCGTTGCTCAGGGCGAACGG
AGTCAACTTTGGTAGCTGCCTTCCCAAAAAGGGCGCGTGCCCAGGCTTTGAGTTG
CACTCGCACCGTAGTGGCATCAAAAGGTGACCGTGCCCGGTCTGGGCGTTAGGA
TACAGCGCCTGCATAAAAGCCTTGATCTGCTTAAAAGCCACCTGAGCCTTTGCGC
CTTCAGAGAAGAACATGCCGCAAGACTTGCCGGAAAACTGATTGGCCGGACAGG
CCGCGTCGTGCACGCAGCACCTTGCGTCGGTGTTGGAGATCTGCACCACATTTCG
GCCCCACCGGTTCTTCACGATCTTGGCCTTGCTAGACTGCTCCTTCAGCGCGCGCT
GCCCGTTTTCGCTCGTCACATCCATTTCAATCACGTGCTCCTTATTTATCATAATG
CTTCCGTGTAGACACTTAAGCTCGCCTTCGATCTCAGCGCAGCGGTGCAGCCACA
ACGCGCAGCCCGTGGGCTCGTGATGCTTGTAGGTCACCTCTGCAAACGACTGCAG
GTACGCCTGCAGGAATCGCCCCATCATCGTCACAAAGGTCTTGTTGCTGGTGAAG
GTCAGCTGCAACCCGCGGTGCTCCTCGTTCAGCCAGGTCTTGCATACGGCCGCCA
GAGCTTCCACTTGGTCAGGCAGTAGTTTGAAGTTCGCCTTTAGATCGTTATCCAC
GTGGTACTTGTCCATCAGCGCGCGCGCAGCCTCCATGCCCTTCTCCCACGCAGAC
ACGATCGGCACACTCAGCGGGTTCATCACCGTAATTTCACTTTCCGCTTCGCTGG
GCTCTTCCTCTTCCTCTTGCGTCCGCATACCACGCGCCACTGGGTCGTCTTCATTC
AGCCGCCGCACTGTGCGCTTACCTCCTTTGCCATGCTTGATTAGCACCGGTGGGT
TGCTGAAACCCACCATTTGTAGCGCCACATCTTCTCTTTCTTCCTCGCTGTCCACG
ATTACCTCTGGTGATGGCGGGCGCTCGGGCTTGGGAGAAGGGCGCTTCTTTTTCT
TCTTGGGCGCAATGGCCAAATCCGCCGCCGAGGTCGATGGCCGCGGGCTGGGTG
TGCGCGGCACCAGCGCGTCTTGTGATGAGTCTTCCTCGTCCTCGGACTCGATACG
CCGCCTCATCCGCTTTTTTGGGGGCGCCCGGGGAGGCGGCGGCGACGGGGACGG
GGACGACACGTCCTCCATGGTTGGGGGACGTCGCGCCGCACCGCGTCCGCGCTC
GGGGGTGGTTTCGCGCTGCTCCTCTTCCCGACTGGCCATTTCCTTCTCCTATAGGC
AGAAAAAGATCATGGAGTCAGTCGAGAAGAAGGACAGCCTAACCGCCCCCTCTG
AGTTCGCCACCACCGCCTCCACCGATGCCGCCAACGCGCCTACCACCTTCCCCGT
CGAGGCACCCCCGCTTGAGGAGGAGGAAGTGATTATCGAGCAGGACCCAGGTTT
TGTAAGCGAAGACGACGAGGACCGCTCAGTACCAACAGAGGATAAAAAGCAAG
ACCAGGACAACGCAGAGGCAAACGAGGAACAAGTCGGGCGGGGGGACGAAAGG
CATGGCGACTACCTAGATGTGGGAGACGACGTGCTGTTGAAGCATCTGCAGCGC
CAGTGCGCCATTATCTGCGACGCGTTGCAAGAGCGCAGCGATGTGCCCCTCGCCA
TAGCGGATGTCAGCCTTGCCTACGAACGCCACCTATTCTCACCGCGCGTACCCCC
CAAACGCCAAGAAAACGGCACATGCGAGCCCAACCCGCGCCTCAACTTCTACCC
CGTATTTGCCGTGCCAGAGGTGCTTGCCACCTATCACATCTTTTTCCAAAACTGC
AAGATACCCCTATCCTGCCGTGCCAACCGCAGCCGAGCGGACAAGCAGCTGGCC
TTGCGGCAGGGCGCTGTCATACCTGATATCGCCTCGCTCAACGAAGTGCCAAAA
ATCTTTGAGGGTCTTGGACGCGACGAGAAGCGCGCGGCAAACGCTCTGCAACAG
GAAAACAGCGAAAATGAAAGTCACTCTGGAGTGTTGGTGGAACTCGAGGGTGAC
AACGCGCGCCTAGCCGTACTAAAACGCAGCATCGAGGTCACCCACTTTGCCTACC
CGGCACTTAACCTACCCCCCAAGGTCATGAGCACAGTCATGAGTGAGCTGATCGT
GCGCCGTGCGCAGCCCCTGGAGAGGGATGCAAATTTGCAAGAACAAACAGAGG
AGGGCCTACCCGCAGTTGGCGACGAGCAGCTAGCGCGCTGGCTTCAAACGCGCG
AGCCTGCCGACTTGGAGGAGCGACGCAAACTAATGATGGCCGCAGTGCTCGTTA
CCGTGGAGCTTGAGTGCATGCAGCGGTTCTTTGCTGACCCGGAGATGCAGCGCA
AGCTAGAGGAAACATTGCACTACACCTTTCGACAGGGCTACGTACGCCAGGCCT
GCAAGATCTCCAACGTGGAGCTCTGCAACCTGGTCTCCTACCTTGGAATTTTGCA
CGAAAACCGCCTTGGGCAAAACGTGCTTCATTCCACGCTCAAGGGCGAGGCGCG
CCGCGACTACGTCCGCGACTGCGTTTACTTATTTCTATGCTACACCTGGCAGACG
GCCATGGGCGTTTGGCAGCAGTGCTTGGAGGAGTGCAACCTCAAGGAGCTGCAG
AAACTGCTAAAGCAAAACTTGAAGGACCTATGGACGGCCTTCAACGAGCGCTCC
GTGGCCGCGCACCTGGCGGACATCATTTTCCCCGAACGCCTGCTTAAAACCCTGC
AACAGGGTCTGCCAGACTTCACCAGTCAAAGCATGTTGCAGAACTTTAGGAACTT
TATCCTAGAGCGCTCAGGAATCTTGCCCGCCACCTGCTGTGCACTTCCTAGCGAC
TTTGTGCCCATTAAGTACCGCGAATGCCCTCCGCCGCTTTGGGGCCACTGCTACC
TTCTGCAGCTAGCCAACTACCTTGCCTACCACTCTGACATAATGGAAGACGTGAG
CGGTGACGGTCTACTGGAGTGTCACTGTCGCTGCAACCTATGCACCCCGCACCGC
TCCCTGGTTTGCAATTCGCAGCTGCTTAACGAAAGTCAAATTATCGGTACCTTTG
AGCTGCAGGGTCCCTCGCCTGACGAAAAGTCCGCGGCTCCGGGGTTGAAACTCA
CTCCGGGGCTGTGGACGTCGGCTTACCTTCGCAAATTTGTACCTGAGGACTACCA
CGCCCACGAGATTAGGTTCTACGAAGACCAATCCCGCCCGCCTAATGCGGAGCTT
ACCGCCTGCGTCATTACCCAGGGCCACATTCTTGGCCAATTGCAAGCCATCAACA
AAGCCCGCCAAGAGTTTCTGCTACGAAAGGGACGGGGGGTTTACTTGGACCCCC
AGTCCGGCGAGGAGCTCAACCCAATCCCCCCGCCGCCGCAGCCCTATCAGCAGC
AGCCGCGGGCCCTTGCTTCCCAGGATGGCACCCAAAAAGAAGCTGCAGCTGCCG
CCGCCACCCACGGACGAGGAGGAATACTGGGACAGTCAGGCAGAGGAGGTTTTG
GACGAGGAGGAGGAGGACATGATGGAAGACTGGGAGAGCCTAGACGAGGAAGC
TTCCGAGGTCGAAGAGGTGTCAGACGAAACACCGTCACCCTCGGTCGCATTCCCC
TCGCCGGCGCCCCAGAAATCGGCAACCGGTTCCAGCATGGCTACAACCTCCGCTC
CTCAGGCGCCGCCGGCACTGCCCGTTCGCCGACCCAACCGTAGATGGGACACCA
CTGGAACCAGGGCCGGTAAGTCCAAGCAGCCGCCGCCGTTAGCCCAAGAGCAAC
AACAGCGCCAAGGCTACCGCTCATGGCGCGGGCACAAGAACGCCATAGTTGCTT
GCTTGCAAGACTGTGGGGGCAACATCTCCTTCGCCCGCCGCTTTCTTCTCTACCAT
CACGGCGTGGCCTTCCCCCGTAACATCCTGCATTACTACCGTCATCTCTACAGCC
CATACTGCACCGGCGGCAGCGGCAGCAACAGCAGCGGCCACACAGAAGCAAAG
GCGACCGGATAGCAAGACTCTGACAAAGCCCAAGAAATCCACAGCGGCGGCAG
CAGCAGGAGGAGGAGCGCTGCGTCTGGCGCCCAACGAACCCGTATCGACCCGCG
AGCTTAGAAACAGGATTTTTCCCACTCTGTATGCTATATTTCAACAGAGCAGGGG
CCAAGAACAAGAGCTGAAAATAAAAAACAGGTCTCTGCGATCCCTCACCCGCAG
CTGCCTGTATCACAAAAGCGAAGATCAGCTTCGGCGCACGCTGGAAGACGCGGA
GGCTCTCTTCAGTAAATACTGCGCGCTGACTCTTAAGGACTAGTTTCGCGCCCTTT
CTCAAATTTAAGCGCGAAAACTACGTCATCTCCAGCGGCCACACCCGGCGCCAG
CACCTGTTGTCAGCGCCATTATGAGCAAGGAAATTCCCACGCCCTACATGTGGAG
TTACCAGCCACAAATGGGACTTGCGGCTGGAGCTGCCCAAGACTACTCAACCCG
AATAAACTACATGAGCGCGGGACCCCACATGATATCCCGGGTCAACGGAATACG
CGCCCACCGAAACCGAATTCTCCTGGAACAGGCGGCTATTACCACCACACCTCGT
AATAACCTTAATCCCCGTAGTTGGCCCGCTGCCCTGGTGTACCAGGAAAGTCCCG
CTCCCACCACTGTGGTACTTCCCAGAGACGCCCAGGCCGAAGTTCAGATGACTAA
CTCAGGGGCGCAGCTTGCGGGCGGCTTTCGTCACAGGGTGCGGTCGCCCGGGCA
GGGTATAACTCACCTGACAATCAGAGGGCGAGGTATTCAGCTCAACGACGAGTC
GGTGAGCTCCTCGCTTGGTCTCCGTCCGGACGGGACATTTCAGATCGGCGGCGCC
GGCCGCTCTTCATTCACGCCTCGTCAGGCAATCCTAACTCTGCAGACCTCGTCCT
CTGAGCCGCGCTCTGGAGGCATTGGAACTCTGCAATTTATTGAGGAGTTTGTGCC
ATCGGTCTACTTTAACCCCTTCTCGGGACCTCCCGGCCACTATCCGGATCAATTTA
TTCCTAACTTTGACGCGGTAAAGGACTCGGCGGACGGCTACGACTGAATGTTAA
GTGGAGAGGCAGAGCAACTGCGCCTGAAACACCTGGTCCACTGTCGCCGCCACA
AGTGCTTTGCCCGCGACTCCGGTGAGTTTTGCTACTTTGAATTGCCCGAGGATCA
TATCGAGGGCCCGGCGCACGGCGTCCGGCTTACCGCCCAGGGAGAGCTTGCCCG
TAGCCTGATTCGGGAGTTTACCCAGCGCCCCCTGCTAGTTGAGCGGGACAGGGG
ACCCTGTGTTCTCACTGTGATTTGCAACTGTCCTAACCCTGGATTACATCAAGATC
CTCTAGTTAATGTCAGGTCGCCTAAGTCGATTAACTAGAGTACCCGGGGATCTTA
TTCCCTTTAACTAATAAAAAAAAATAATAAAGCATCACTTACTTAAAATCAGTTA
GCAAATTTCTGTCCAGTTTATTCAGCAGCACCTCCTTGCCCTCCTCCCAGCTCTGG
TATTGCAGCTTCCTCCTGGCTGCAAACTTTCTCCACAATCTAAATGGAATGTCAG
TTTCCTCCTGTTCCTGTCCATCCGCACCCACTATCTTCATGTTGTTGCAGATGAAG
CGCGCAAGACCGTCTGAAGATACCTTCAACCCCGTGTATCCATATGACACGGAA
ACCGGTCCTCCAACTGTGCCTTTTCTTACTCCTCCCTTTGTATCCCCCAATGGGTT
TCAAGAGAGTCCCCCTGGGGTACTCTCTTTGCGCCTATCCGAACCTCTAGTTACC
TCCAATGGCATGCTTGCGCTCAAAATGGGCAACGGCCTCTCTCTGGACGAGGCCG
GCAACCTTACCTCCCAAAATGTAACCACTGTGAGCCCACCTCTCAAAAAAACCA
AGTCAAACATAAACCTGGAAATATCTGCACCCCTCACAGTTACCTCAGAAGCCCT
AACTGTGGCTGCCGCCGCACCTCTAATGGTCGCGGGCAACACACTCACCATGCA
ATCACAGGCCCCGCTAACCGTGCACGACTCCAAACTTAGCATTGCCACCCAAGG
ACCCCTCACAGTGTCAGAAGGAAAGCTAGCCCTGCAAACATCAGGCCCCCTCAC
CACCACCGATAGCAGTACCCTTACTATCACTGCCTCACCCCCTCTAACTACTGCC
ACTGGTAGCTTGGGCATTGACTTGAAAGAGCCCATTTATACACAAAATGGAAAA
CTAGGACTAAAGTACGGGGCTCCTTTGCATGTAACAGACGACCTAAACACTTTGA
CCGTAGCAACTGGTCCAGGTGTGACTATTAATAATACTTCCTTGCAAACTAAAGT
TACTGGAGCCTTGGGTTTTGATTCACAAGGCAATATGCAACTTAATGTAGCAGGA
GGACTAAGGATTGATTCTCAAAACAGACGCCTTATACTTGATGTTAGTTATCCGT
TTGATGCTCAAAACCAACTAAATCTAAGACTAGGACAGGGCCCTCTTTTTATAAA
CTCAGCCCACAACTTGGATATTAACTACAACAAAGGCCTTTACTTGTTTACAGCT
TCAAACAATTCCAAAAAGCTTGAGGTTAACCTAAGCACTGCCAAGGGGTTGATG
TTTGACGCTACAGCCATAGCCATTAATGCAGGAGATGGGCTTGAATTTGGTTCAC
CTAATGCACCAAACACAAATCCCCTCAAAACAAAAATTGGCCATGGCCTAGAAT
TTGATTCAAACAAGGCTATGGTTCCTAAACTAGGAACTGGCCTTAGTTTTGACAG
CACAGGTGCCATTACAGTAGGAAACAAAAATAATGATAAGCTAACTTTGTGGAC
CACACCAGCTCCATCTCCTAACTGTAGACTAAATGCAGAGAAAGATGCTAAACT
CACTTTGGTCTTAACAAAATGTGGCAGTCAAATACTTGCTACAGTTTCAGTTTTG
GCTGTTAAAGGCAGTTTGGCTCCAATATCTGGAACAGTTCAAAGTGCTCATCTTA
TTATAAGATTTGACGAAAATGGAGTGCTACTAAACAATTCCTTCCTGGACCCAGA
ATATTGGAACTTTAGAAATGGAGATCTTACTGAAGGCACAGCCTATACAAACGC
TGTTGGATTTATGCCTAACCTATCAGCTTATCCAAAATCTCACGGTAAAACTGCC
AAAAGTAACATTGTCAGTCAAGTTTACTTAAACGGAGACAAAACTAAACCTGTA
ACACTAACCATTACACTAAACGGTACACAGGAAACAGGAGACACAACTCCAAGT
GCATACTCTATGTCATTTTCATGGGACTGGTCTGGCCACAACTACATTAATGAAA
TATTTGCCACATCCTCTTACACTTTTTCATACATTGCCCAAGAATAAAGAATCGTT
TGTGTTATGTTTCAACGTGTTTATTTTTCAATTGCAGAAAATTTCAAGTCATTTTT
CATTCAGTAGTATAGCCCCACCACCACATAGCTTATACAGATCACCGTACCTTAA
TCAAACTCACAGAACCCTAGTATTCAACCTGCCACCTCCCTCCCAACACACAGAG
TACACAGTCCTTTCTCCCCGGCTGGCCTTAAAAAGCATCATATCATGGGTAACAG
ACATATTCTTAGGTGTTATATTCCACACGGTTTCCTGTCGAGCCAAACGCTCATC
AGTGATATTAATAAACTCCCCGGGCAGCTCACTTAAGTTCATGTCGCTGTCCAGC
TGCTGAGCCACAGGCTGCTGTCCAACTTGCGGTTGCTTAACGGGCGGCGAAGGA
GAAGTCCACGCCTACATGGGGGTAGAGTCATAATCGTGCATCAGGATAGGGCGG
TGGTGCTGCAGCAGCGCGCGAATAAACTGCTGCCGCCGCCGCTCCGTCCTGCAG
GAATACAACATGGCAGTGGTCTCCTCAGCGATGATTCGCACCGCCCGCAGCATA
AGGCGCCTTGTCCTCCGGGCACAGCAGCGCACCCTGATCTCACTTAAATCAGCAC
AGTAACTGCAGCACAGCACCACAATATTGTTCAAAATCCCACAGTGCAAGGCGC
TGTATCCAAAGCTCATGGCGGGGACCACAGAACCCACGTGGCCATCATACCACA
AGCGCAGGTAGATTAAGTGGCGACCCCTCATAAACACGCTGGACATAAACATTA
CCTCTTTTGGCATGTTGTAATTCACCACCTCCCGGTACCATATAAACCTCTGATTA
AACATGGCGCCATCCACCACCATCCTAAACCAGCTGGCCAAAACCTGCCCGCCG
GCTATACACTGCAGGGAACCGGGACTGGAACAATGACAGTGGAGAGCCCAGGA
CTCGTAACCATGGATCATCATGCTCGTCATGATATCAATGTTGGCACAACACAGG
CACACGTGCATACACTTCCTCAGGATTACAAGCTCCTCCCGCGTTAGAACCATAT
CCCAGGGAACAACCCATTCCTGAATCAGCGTAAATCCCACACTGCAGGGAAGAC
CTCGCACGTAACTCACGTTGTGCATTGTCAAAGTGTTACATTCGGGCAGCAGCGG
ATGATCCTCCAGTATGGTAGCGCGGGTTTCTGTCTCAAAAGGAGGTAGACGATCC
CTACTGTACGGAGTGCGCCGAGACAACCGAGATCGTGTTGGTCGTAGTGTCATGC
CAAATGGAACGCCGGACGTAGTCATATTTCCTGAAGCAAAACCAGGTGCGGGCG
TGACAAACAGATCTGCGTCTCCGGTCTCGCCGCTTAGATCGCTCTGTGTAGTAGT
TGTAGTATATCCACTCTCTCAAAGCATCCAGGCGCCCCCTGGCTTCGGGTTCTAT
GTAAACTCCTTCATGCGCCGCTGCCCTGATAACATCCACCACCGCAGAATAAGCC
ACACCCAGCCAACCTACACATTCGTTCTGCGAGTCACACACGGGAGGAGCGGGA
AGAGCTGGAAGAACCATGTTTTTTTTTTTATTCCAAAAGATTATCCAAAACCTCA
AAATGAAGATCTATTAAGTGAACGCGCTCCCCTCCGGTGGCGTGGTCAAACTCTA
CAGCCAAAGAACAGATAATGGCATTTGTAAGATGTTGCACAATGGCTTCCAAAA
GGCAAACGGCCCTCACGTCCAAGTGGACGTAAAGGCTAAACCCTTCAGGGTGAA
TCTCCTCTATAAACATTCCAGCACCTTCAACCATGCCCAAATAATTCTCATCTCGC
CACCTTCTCAATATATCTCTAAGCAAATCCCGAATATTAAGTCCGGCCATTGTAA
AAATCTGCTCCAGAGCGCCCTCCACCTTCAGCCTCAAGCAGCGAATCATGATTGC
AAAAATTCAGGTTCCTCACAGACCTGTATAAGATTCAAAAGCGGAACATTAACA
AAAATACCGCGATCCCGTAGGTCCCTTCGCAGGGCCAGCTGAACATAATCGTGC
AGGTCTGCACGGACCAGCGCGGCCACTTCCCCGCCAGGAACCATGACAAAAGAA
CCCACACTGATTATGACACGCATACTCGGAGCTATGCTAACCAGCGTAGCCCCGA
TGTAAGCTTGTTGCATGGGCGGCGATATAAAATGCAAGGTGCTGCTCAAAAAAT
CAGGCAAAGCCTCGCGCAAAAAAGAAAGCACATCGTAGTCATGCTCATGCAGAT
AAAGGCAGGTAAGCTCCGGAACCACCACAGAAAAAGACACCATTTTTCTCTCAA
ACATGTCTGCGGGTTTCTGCATAAACACAAAATAAAATAACAAAAAAACATTTA
AACATTAGAAGCCTGTCTTACAACAGGAAAAACAACCCTTATAAGCATAAGACG
GACTACGGCCATGCCGGCGTGACCGTAAAAAAACTGGTCACCGTGATTAAAAAG
CACCACCGACAGCTCCTCGGTCATGTCCGGAGTCATAATGTAAGACTCGGTAAAC
ACATCAGGTTGATTCACATCGGTCAGTGCTAAAAAGCGACCGAAATAGCCCGGG
GGAATACATACCCGCAGGCGTAGAGACAACATTACAGCCCCCATAGGAGGTATA
ACAAAATTAATAGGAGAGAAAAACACATAAACACCTGAAAAACCCTCCTGCCTA
GGCAAAATAGCACCCTCCCGCTCCAGAACAACATACAGCGCTTCCACAGCGGCA
GCCATAACAGTCAGCCTTACCAGTAAAAAAGAAAACCTATTAAAAAAACACCAC
TCGACACGGCACCAGCTCAATCAGTCACAGTGTAAAAAAGGGCCAAGTGCAGAG
CGAGTATATATAGGACTAAAAAATGACGTAACGGTTAAAGTCCACAAAAAACAC
CCAGAAAACCGCACGCGAACCTACGCCCAGAAACGAAAGCCAAAAAACCCACA
ACTTCCTCAAATCGTCACTTCCGTTTTCCCACGTTACGTCACTTCCCATTTTAAGA
AAACTACAATTCCCAACACATACAAGTTACTCCGCCCTAAAACCTACGTCACCCG
CCCCGTTCCCACGCCCCGCGCCACGTCACAAACTCCACCCCCTCATTATCATATT
GGCTTCAATCCAAAATAAGGTATATT
SEQ ID NO: 11: rAd-CMV-SARS-CoV-2-S1-Furin-N-BGH-CMV-dsRNA-SPA
TAAGGATCCCATCATCAATAATATACCTTATTTTGGATTGAAGCCAATATGATAA
TGAGGGGGTGGAGTTTGTGACGTGGCGCGGGGCGTGGGAACGGGGCGGGTGAC
GTAGTAGTGTGGCGGAAGTGTGATGTTGCAAGTGTGGCGGAACACATGTAAGCG
ACGGATGTGGCAAAAGTGACGTTTTTGGTGTGCGCCGGTGTACACAGGAAGTGA
CAATTTTCGCGCGGTTTTAGGCGGATGTTGTAGTAAATTTGGGCGTAACCGAGTA
AGATTTGGCCATTTTCGCGGGAAAACTGAATAAGAGGAAGTGAAATCTGAATAA
TTTTGTGTTACTCATAGCGCGTAATACTGCTAGAGATCTGGCGAAAGGGGGATGT
GCTGCAAGGCGATTAAGTTGGGTAACGCCAGGGTTTTCCCAGTCACGACGTTGTA
AAACGACGGCCAGTGAATTGTAATACGACTCACTATAGGGCGAATTGGGTACTG
GCCACAGGAGCTTGGCCCATTGCATACGTTGTATCCATATCATAATATGTACATT
TATATTGGCTCATGTCCAACATTACCGCCATGTTGACATTGATTATTGACTAGTTA
TTAATAGTAATCAATTACGGGGTCATTAGTTCATAGCCCATATATGGAGTTCCGC
GTTACATAACTTACGGTAAATGGCCCGCCTGGCTGACCGCCCAACGACCCCCGCC
CATTGACGTCAATAATGACGTATGTTCCCATAGTAACGCCAATAGGGACTTTCCA
TTGACGTCAATGGGTGGAGTATTTACGGTAAACTGCCCACTTGGCAGTACATCAA
GTGTATCATATGCCAAGTACGCCCCCTATTGACGTCAATGACGGTAAATGGCCCG
CCTGGCATTATGCCCAGTACATGACCTTATGGGACTTTCCTACTTGGCAGTACAT
CTACGTATTAGTCATCGCTATTACCATGGTGATGCGGTTTTGGCAGTACATCAAT
GGGCGTGGATAGCGGTTTGACTCACGGGGATTTCCAAGTCTCCACCCCATTGACG
TCAATGGGAGTTTGTTTTGGCACCAAAATCAACGGGACTTTCCAAAATGTCGTAA
CAACTCCGCCCCATTGACGCAAATGGGCGGTAGGCGTGTACGGTGGGAGGTCTA
TATAAGCAGAGCTCGTTTAGTGAACCGTCAGatcgcctggagacgccatccacgctgttttgacctccat
agaagacaccgggaccgatccagcctgactctagCctAGCTCtgaagttggtggtgaggccctgggcaggttggtatcaaggtt
acaagacaggtttaaggagaccaatagaaactgggcatgtggagacagagaagactcttgggtttctgataggcactgactctctctgc
ctattggtctattttcccacccttaggctgctggtctgagcctagGAGATCTCTCGAGGTCGACGGTATCGAT
GggtaccgccaccATGTTTGTTTTTCTCGTACTCCTGCCCCTGGTTTCCTCCCAATGTGTC
AATCTGACTACCCGGACCCAACTTCCTCCCGCCTACACCAATTCCTTTACCCGAG
GTGTTTACTACCCAGACAAAGTGTTCAGGTCATCCGTCCTCCATAGTACCCAAGA
CCTCTTCCTCCCTTTTTTTTCTAACGTTACCTGGTTTCACGCTATTCACGTTAGCGG
CACCAACGGCACCAAAAGATTCGATAACCCCGTACTGCCGTTCAACGACGGGGT
ATATTTTGCCTCTACTGAAAAATCAAACATCATACGCGGATGGATCTTTGGGACT
ACCCTGGACTCAAAAACTCAGTCCCTGCTGATTGTGAATAACGCTACCAACGTGG
TGATCAAAGTCTGTGAATTCCAGTTTTGCAACGATCCTTTTCTCGGCGTTTATTAT
CACAAAAATAACAAATCCTGGATGGAGAGCGAGTTCCGGGTGTACTCCTCCGCG
AATAATTGCACCTTCGAATATGTGTCTCAGCCATTCCTCATGGACCTCGAGGGGA
AGCAGGGCAATTTTAAGAATCTGCGAGAATTCGTGTTCAAGAATATAGACGGTT
ACTTCAAGATTTACTCCAAACACACCCCGATTAACCTGGTTAGGGACTTGCCTCA
GGGCTTTTCTGCATTGGAGCCCCTCGTGGACCTCCCAATCGGCATAAACATTACA
AGATTTCAGACTTTGCTTGCATTGCACAGGAGCTATTTGACACCCGGCGATTCTT
CTTCCGGATGGACCGCTGGAGCAGCTGCTTATTACGTGGGCTATCTGCAGCCTCG
AACCTTTCTTTTGAAGTACAACGAAAATGGAACTATCACCGATGCAGTTGACTGC
GCCCTGGACCCCCTGTCCGAAACTAAGTGCACGCTCAAAAGTTTCACAGTAGAG
AAGGGGATATACCAGACTAGCAATTTCCGCGTTCAGCCAACCGAAAGTATAGTG
CGCTTTCCTAATATAACTAACCTGTGTCCTTTCGGGGAAGTGTTTAACGCCACTA
GATTCGCTTCCGTCTACGCCTGGAATAGAAAGAGGATCTCAAATTGCGTTGCTGA
CTATAGTGTTTTGTACAATTCCGCCTCTTTCTCAACCTTCAAATGTTACGGGGTGA
GCCCTACCAAACTGAACGACCTGTGCTTTACAAACGTATACGCCGACAGCTTTGT
TATCAGAGGAGACGAGGTTCGCCAGATTGCTCCGGGTCAGACAGGCAAGATTGC
TGATrATAATrACAAACTGCCCGACGACTTrACAGGATGTGTGATCGCGTGGAAC
AGTAACAATCTTGACTCAAAGGTTGGGGGTAATTATAATTATCTTTACCGGCTGT
TCAGAAAAAGCAATTTGAAACCCTTCGAAAGGGACATATCCACCGAGATCTATC
AGGCCGGGTCCACTCCATGCAATGGTGTGGAAGGTTTTAATTGCTACTTCCCATT
GCAGTCTTATGGATTCCAACCAACCAATGGCGTAGGCTACCAGCCGTATCGCGTT
GTCGTGCTCAGCTTCGAGCTGCTCCACGCCCCCGCGACCGTATGCGGTCCTAAGA
AGTCCACCAATCTTGTTAAGAACAAGTGTGTAAACTTTAACTTTAACGGGCTGAC
CGGGACCGGCGTTCTGACTGAATCTAACAAAAAATTCCTGCCTTTCCAGCAGTTC
GGCCGCGATATTGCTGACACCACTGACGCTGTAAGAGACCCTCAGACCCTTGAA
ATTCTCGATATCACACCTTGCAGCTTTGGGGGCGTGTCCGTCATCACTCCAGGAA
CTAACACAAGCAACCAGGTGGCAGTGTTGTACCAGGATGTTAATTGTACCGAGG
TGCCAGTGGCCATCCACGCCGATCAATTGACACCTACCTGGAGGGTTTACAGCAC
AGGGTCCAATGTTTTTCAGACAAGAGCCGGATGTCTGATCGGTGCCGAGCATGTC
AACAATTCCTACGAGTGTGATATCCCCATTGGTGCGGGAATTTGTGCATCATATC
AGACCCAGACTAATAGCCCAAGAAGAGCTAGATCCGTCGCTAGTCAATCCATCA
TTGCATATACAATGATGTCCGATAACGGCCCCCAGAATCAGAGAAACGCTCCCC
GCATCACGTTCGGCGGACCAAGTGACAGCACAGGCAGTAACCAGAACGGAGAA
CGCTCCGGTGCTCGCTCCAAGCAGCGACGGCCGCAAGGGCTTCCCAACAATACC
GCCAGCTGGTTTACGGCTCTGACCCAACACGGGAAAGAAGATCTTAAATTCCCC
AGGGGCCAGGGCGTCCCTATCAATACTAACTCCAGCCCGGATGATCAGATAGGC
TACTATAGACGCGCTACCCGACGGATACGAGGGGGGGACGGCAAAATGAAGGA
CCTTTCCCCCCGGTGGTATTTCTATTACTTGGGCACCGGACCAGAAGCCGGACTG
CCTTACGGCGCTAACAAAGACGGAATAATCTGGGTTGCGACGGAGGGCGCCCTG
AATACACCTAAAGACCATATCGGCACAAGAAATCCTGCTAACAATGCCGCGATT
GTGCTCCAGCTGCCTCAGGGAACCACGCTGCCTAAAGGGTTTTACGCTGAGGGGT
CAAGGGGGGGGAGTCAAGCGTCTAGTAGGTCATCCTCTCGCTCTCGCAATAGTTC
CCGGAACTCAACCCCAGGCAGCAGCAGAGGAACCTCTCCCGCACGGATGGCTGG
CAATGGGGGAGATGCTGCCCTTGCTCTCCTTCTGCTGGATCGCCTTAACCAGCTC
GAATCAAAGATGTCTGGAAAAGGTCAGCAGCAGCAAGGCCAGACCGTGACAAA
GAAGAGTGCAGCTGAAGCTAGTAAAAAGCCACGCCAAAAACGGACCGCAACTA
AGGCATATAACGTAACACAGGCCTTCGGCAGAAGAGGTCCAGAACAAACACAG
GGAAACTTTGGCGATCAAGAGCTGATTAGACAGGGCACAGATTACAAACACTGG
CCACAGATCGCGCAGTTTGCACCAAGCGCCTCTGCATTCTTCGGGATGAGTCGGA
TTGGGATGGAAGTCACTCCATCCGGGACCTGGCTTACCTACACAGGGGCAATAA
AACTCGACGACAAAGACCCAAACTTTAAAGATCAGGTCATCCTGCTGAATAAAC
ACATCGATGCCTACAAAACTTTCCCCCCAACCGAACCAAAGAAAGACAAGAAAA
AAAAGGCAGACGAAACGCAAGCGCTCCCTCAGCGCCAGAAGAAGCAGCAGACC
GTTACACTGTTGCCAGCAGCAGATCTGGATGATTTTTCCAAGCAGCTTCAACAGA
GTATGTCAAGCGCTGACAGCACTCAGGCTTGAcgatcgGATATCGCTAGCGTACCGG
CGGCCGCCCTATTCTATAGTGTCACCTAAATGCTAGAGCTCGCTGATCAGCCTCG
ACTGTGCCTTCTAGTTGCCAGCCATCTGTTGTTTGCCCCTCCCCCGTGCCTTCCTT
GACCCTGGAAGGTGCCACTCCCACTGTCCTTTCCTAATAAAATGAGGAAATTGCA
TCGCATTGTCTGAGTAGGTGTCATTCTATTCTGGGGGGTGGGGTGGGGCAGGACA
GCAAGGGGGAGGATTGGGAAGACAATAGCAGGCATGCTGGGGATGCGGTGGGC
TCTATGGCTTCTGAGGCGGAAAGAACCAAAGCTTAcgcgttagttattaataGTAATCAATT
ACGGGGTCATTAGTTCATAGCCCATATATGGAGTTCCGCGTTACATAACTTACGG
TAAATGGCCCGCCTGGCTGACCGCCCAACGACCCCCGCCCATTGACGTCAATAAT
GACGTATGTTCCCATAGTAACGCCAATAGGGACTTTCCATTGACGTCAATGGGTG
GAGTATTTACGGTAAACTGCCCACTTGGCAGTACATCAAGTGTATCATATGCCAA
GTACGCCCCCTATTGACGTCAATGACGGTAAATGGCCCGCCTGGCATTATGCCCA
GTACATGACCTTATGGGACTTTCCTACTTGGCAGTACATCTACGTATTAGTCATC
GCTATTACCATGGTGATGCGGTTTTGGCAGTACATCAATGGGCGTGGATAGCGGT
TTGACTCACGGGGATTTCCAAGTCTCCACCCCATTGACGTCAATGGGAGTTTGTT
TTGGCACCAAAATCAACGGGACTTTCCAAAATGTCGTAACAACTCCGCCCCATTG
ACGCAAATGGGCGGTAGGCGTGTACGGTGGGAGGTCTATATAAGCAGAGCTGGT
TTAGTGAACCGTCAGATCCGCTAGAGATATCGGGCCACTGCAGGAAACGATATG
GGCTGAATACGGATCCGTATTCAGCCCATATCGTTTCTCTAGAAATAAAATATCT
TTATTTTCATTACATCTGTGTGTTGGTTTTTTGTGTGAATCGATAGTACTAACATA
CGCTCTCCATCTCGAGCCTAAGCTTGTCGACTCGAAGATCTGGGCGTGGTTAAGG
GTGGGAAAGAATATATAAGGTGGGGGTCTTATGTAGTTTTGTATCTGTTTTGCAG
CAGCCGCCGCCGCCATGAGCACCAACTCGTTTGATGGAAGCATTGTGAGCTCATA
TTTGACAACGCGCATGCCCCCATGGGCCGGGGTGCGTCAGAATGTGATGGGCTC
CAGCATTGATGGTCGCCCCGTCCTGCCCGCAAACTCTACTACCTTGACCTACGAG
ACCGTGTCTGGAACGCCGTTGGAGACTGCAGCCTCCGCCGCCGCTTCAGCCGCTG
CAGCCACCGCCCGCGGGATTGTGACTGACTTTGCTTTCCTGAGCCCGCTTGCAAG
CAGTGCAGCTTCCCGTTCATCCGCCCGCGATGACAAGTTGACGGCTCTTTTGGCA
CAATTGGATTCTTTGACCCGGGAACTTAATGTCGTTTCTCAGCAGCTGTTGGATCT
GCGCCAGCAGGTTTCTGCCCTGAAGGCTTCCTCCCCTCCCAATGCGGTTTAAAAC
ATAAATAAAAAACCAGACTCTGTTTGGATTTGGATCAAGCAAGTGTCTTGCTGTC
TTTATTTAGGGGTTTTGCGCGCGCGGTAGGCCCGGGACCAGCGGTCTCGGTCGTT
GAGGGTCCTGTGTATTTTTTCCAGGACGTGGTAAAGGTGACTCTGGATGTTCAGA
TACATGGGCATAAGCCCGTCTCTGGGGTGGAGGTAGCACCACTGCAGAGCTTCA
TGCTGCGGGGTGGTGTTGTAGATGATCCAGTCGTAGCAGGAGCGCTGGGCGTGG
TGCCTAAAAATGTCTTTCAGTAGCAAGCTGATTGCCAGGGGCAGGCCCTTGGTGT
AAGTGTTTACAAAGCGGTTAAGCTGGGATGGGTGCATACGTGGGGATATGAGAT
GCATCTTGGACTGTATTTTTAGGTTGGCTATGTTCCCAGCCATATCCCTCCGGGGA
TTCATGTTGTGCAGAACCACCAGCACAGTGTATCCGGTGCACTTGGGAAATTTGT
CATGTAGCTTAGAAGGAAATGCGTGGAAGAACTTGGAGACGCCCTTGTGACCTC
CAAGATTTTCCATGCATTCGTCCATAATGATGGCAATGGGCCCACGGGCGGCGGC
CTGGGCGAAGATATTTCTGGGATCACTAACGTCATAGTTGTGTTCCAGGATGAGA
TCGTCATAGGCCATTTTTACAAAGCGCGGGCGGAGGGTGCCAGACTGCGGTATA
ATGGTTCCATCCGGCCCAGGGGCGTAGTTACCCTCACAGATTTGCATTTCCCACG
CTTTGAGTTCAGATGGGGGGATCATGTCTACCTGCGGGGCGATGAAGAAAACGG
TTTCCGGGGTAGGGGAGATCAGCTGGGAAGAAAGCAGGTTCCTGAGCAGCTGCG
ACTTACCGCAGCCGGTGGGCCCGTAAATCACACCTATTACCGGCTGCAACTGGTA
GTTAAGAGAGCTGCAGCTGCCGTCATCCCTGAGCAGGGGGGCCACTTCGTTAAG
CATGTCCCTGACTCGCATGTTTTCCCTGACCAAATCCGCCAGAAGGCGCTCGCCG
CCCAGCGATAGCAGTTCTTGCAAGGAAGCAAAGTTTTTCAACGGTTTGAGACCGT
CCGCCGTAGGCATGCTTTTGAGCGTTTGACCAAGCAGTTCCAGGCGGTCCCACAG
CTCGGTCACCTGCTCTACGGCATCTCGATCCAGCATATCTCCTCGTTTCGCGGGTT
GGGGCGGCTTTCGCTGTACGGCAGTAGTCGGTGCTCGTCCAGACGGGCCAGGGT
CATGTCTTTCCACGGGCGCAGGGTCCTCGTCAGCGTAGTCTGGGTCACGGTGAAG
GGGTGCGCTCCGGGCTGCGCGCTGGCCAGGGTGCGCTTGAGGCTGGTCCTGCTG
GTGCTGAAGCGCTGCCGGTCTTCGCCCTGCGCGTCGGCCAGGTAGCATTTGACCA
TGGTGTCATAGTCCAGCCCCTCCGCGGCGTGGCCCTTGGCGCGCAGCTTGCCCTT
GGAGGAGGCGCCGCACGAGGGGCAGTGCAGACTTTTGAGGGCGTAGAGCTTGGG
CGCGAGAAATACCGATTCCGGGGAGTAGGCATCCGCGCCGCAGGCCCCGCAGAC
GGTCTCGCATTCCACGAGCCAGGTGAGCTCTGGCCGTTCGGGGTCAAAAACCAG
GTTTCCCCCATGCTTTTTGATGCGTTTCTTACCTCTGGTTTCCATGAGCCGGTGTC
CACGCTCGGTGACGAAAAGGCTGTCCGTGTCCCCGTATACAGACTTGAGAGGCC
TGTCCTCGAGCGGTGTTCCGCGGTCCTCCTCGTATAGAAACTCGGACCACTCTGA
GACAAAGGCTCGCGTCCAGGCCAGCACGAAGGAGGCTAAGTGGGAGGGGTAGC
GGTCGTTGTCCACTAGGGGGTCCACTCGCTCCAGGGTGTGAAGACACATGTCGCC
CTCTTCGGCATCAAGGAAGGTGATTGGTTTGTAGGTGTAGGCCACGTGACCGGGT
GTTCCTGAAGGGGGGCTATAAAAGGGGGTGGGGGCGCGTTCGTCCTCACTCTCTT
CCGCATCGCTGTCTGCGAGGGCCAGCTGTTGGGGTGAGTACTCCCTCTGAAAAGC
GGGCATGACTTCTGCGCTAAGATTGTCAGTTTCCAAAAACGAGGAGGATTTGATA
TTCACCTGGCCCGCGGTGATGCCTTTGAGGGTGGCCGCATCCATCTGGTCAGAAA
AGACAATCTTTTTGTTGTCAAGCTTGGTGGCAAACGACCCGTAGAGGGCGTTGGA
CAGCAACTTGGCGATGGAGCGCAGGGTTTGGTTTTTGTCGCGATCGGCGCGCTCC
TTGGCCGCGATGTTTAGCTGCACGTATTCGCGCGCAACGCACCGCCATTCGGGAA
AGACGGTGGTGCGCTCGTCGGGCACCAGGTGCACGCGCCAACCGCGGTTGTGCA
GGGTGACAAGGTCAACGCTGGTGGCTACCTCTCCGCGTAGGCGCTCGTTGGTCCA
GCAGAGGCGGCCGCCCTTGCGCGAGCAGAATGGCGGTAGGGGGTCTAGCTGCGT
CTCGTCCGGGGGGTCTGCGTCCACGGTAAAGACCCCGGGCAGCAGGCGCGCGTC
GAAGTAGTCTATCTTGCATCCTTGCAAGTCTAGCGCCTGCTGCCATGCGCGGGCG
GCAAGCGCGCGCTCGTATGGGTTGAGTGGGGGACCCCATGGCATGGGGTGGGTG
AGCGCGGAGGCGTACATGCCGCAAATGTCGTAAACGTAGAGGGGCTCTCTGAGT
ATTCCAAGATATGTAGGGTAGCATCTTCCACCGCGGATGCTGGCGCGCACGTAAT
CGTATAGTTCGTGCGAGGGAGCGAGGAGGTCGGGACCGAGGTTGCTACGGGCGG
GCTGCTCTGCTCGGAAGACTATCTGCCTGAAGATGGCATGTGAGTTGGATGATAT
GGTTGGACGCTGGAAGACGTTGAAGCTGGCGTCTGTGAGACCTACCGCGTCACG
CACGAAGGAGGCGTAGGAGTCGCGCAGCTTGTTGACCAGCTCGGCGGTGACCTG
CACGTCTAGGGCGCAGTAGTCCAGGGTTTCCTTGATGATGTCATACTTATCCTGT
CCCTTTTTTTTCCACAGCTCGCGGTTGAGGACAAACTCTTCGCGGTCTTTCCAGTA
CTCTTGGATCGGAAACCCGTCGGCCTCCGAACGGTAAGAGCCTAGCATGTAGAA
CTGGTTGACGGCCTGGTAGGCGCAGCATCCCTTTTCTACGGGTAGCGCGTATGCC
TGCGCGGCCTTCCGGAGCGAGGTGTGGGTGAGCGCAAAGGTGTCCCTGACCATG
ACTTTGAGGTACTGGTATTTGAAGTCAGTGTCGTCGCATCCGCCCTGCTCCCAGA
GCAAAAAGTCCGTGCGCTTTTTGGAACGCGGATTTGGCAGGGCGAAGGTGACAT
CGTTGAAGAGTATCTTTCCCGCGCGAGGCATAAAGTTGCGTGTGATGCGGAAGG
GTCCCGGCACCTCGGAACGGTTGTTAATTACCTGGGCGGCGAGCACGATCTCGTC
AAAGCCGTTGATGTTGTGGCCCACAATGTAAAGTTCCAAGAAGCGCGGGATGCC
CTTGATGGAAGGCAATTTTTTAAGTTCCTCGTAGGTGAGCTCTTCAGGGGAGCTG
AGCCCGTGCTCTGAAAGGGCCCAGTCTGCAAGATGAGGGTTGGAAGCGACGAAT
GAGCTCCACAGGTCACGGGCCATTAGCATTTGCAGGTGGTCGCGAAAGGTCCTA
AACTGGCGACCTATGGCCATTTTTTCTGGGGTGATGCAGTAGAAGGTAAGCGGGT
CTTGTTCCCAGCGGTCCCATCCAAGGTTCGCGGCTAGGTCTCGCGCGGCAGTCAC
TAGAGGCTCATCTCCGCCGAACTTCATGACCAGCATGAAGGGCACGAGCTGCTTC
CCAAAGGCCCCCATCCAAGTATAGGTCTCTACATCGTAGGTGACAAAGAGACGC
TCGGTGCGAGGATGCGAGCCGATCGGGAAGAACTGGATCTCCCGCCACCAATTG
GAGGAGTGGCTATTGATGTGGTGAAAGTAGAAGTCCCTGCGACGGGCCGAACAC
TCGTGCTGGCTTTTGTAAAAACGTGCGCAGTACTGGCAGCGGTGCACGGGCTGTA
CATCCTGCACGAGGTTGACCTGACGACCGCGCACAAGGAAGCAGAGTGGGAATT
TGAGCCCCTCGCCTGGCGGGTTTGGCTGGTGGTCTTCTACTTCGGCTGCTTGTCCT
TGACCGTCTGGCTGCTCGAGGGGAGTTACGGTGGATCGGACCACCACGCCGCGC
GAGCCCAAAGTCCAGATGTCCGCGCGCGGCGGTCGGAGCTTGATGACAACATCG
CGCAGATGGGAGCTGTCCATGGTCTGGAGCTCCCGCGGCGTCAGGTCAGGCGGG
AGCTCCTGCAGGTTTACCTCGCATAGACGGGTCAGGGCGCGGGCTAGATCCAGG
TGATACCTAATTTCCAGGGGCTGGTTGGTGGCGGCGTCGATGGCTTGCAAGAGGC
CGCATCCCCGCGGCGCGACTACGGTACCGCGCGGCGGGCGGTGGGCCGCGGGGG
TGTCCTTGGATGATGCATCTAAAAGCGGTGACGCGGGCGAGCCCCCGGAGGTAG
GGGGGGCTCCGGACCCGCCGGGAGAGGGGGCAGGGGCACGTCGGCGCCGCGCG
CGGGCAGGAGCTGGTGCTGCGCGCGTAGGTTGCTGGCGAACGCGACGACGCGGC
GGTTGATCTCCTGAATCTGGCGCCTCTGCGTGAAGACGACGGGCCCGGTGAGCTT
GAACCTGAAAGAGAGTTCGACAGAATCAATTTCGGTGTCGTTGACGGCGGCCTG
GCGCAAAATCTCCTGCACGTCTCCTGAGTTGTCTTGATAGGCGATCTCGGCCATG
AACTGCTCGATCTCTTCCTCCTGGAGATCTCCGCGTCCGGCTCGCTCCACGGTGG
CGGCGAGGTCGTTGGAAATGCGGGCCATGAGCTGCGAGAAGGCGTTGAGGCCTC
CCTCGTTCCAGACGCGGCTGTAGACCACGCCCCCTTCGGCATCGCGGGCGCGCAT
GACCACCTGCGCGAGATTGAGCTCCACGTGCCGGGCGAAGACGGCGTAGTTTCG
CAGGCGCTGAAAGAGGTAGTTGAGGGTGGTGGCGGTGTGTTCTGCCACGAAGAA
GTACATAACCCAGCGTCGCAACGTGGATTCGTTGATATCCCCCAAGGCCTCAAGG
CGCTCCATGGCCTCGTAGAAGTCCACGGCGAAGTTGAAAAACTGGGAGTTGCGC
GCCGACACGGTTAACTCCTCCTCCAGAAGACGGATGAGCTCGGCGACAGTGTCG
CGCACCTCGCGCTCAAAGGCTACAGGGGCCTCTTCTTCTTCTTCAATCTCCTCTTC
CATAAGGGCCTCCCCTTCTTCTTCTTCTGGCGGCGGTGGGGGAGGGGGGACACGG
CGGCGACGACGGCGCACCGGGAGGCGGTCGACAAAGCGCTCGATCATCTCCCCG
CGGCGACGGCGCATGGTCTCGGTGACGGCGCGGCCGTTCTCGCGGGGGCGCAGT
TGGAAGACGCCGCCCGTCATGTCCCGGTTATGGGTTGGCGGGGGGCTGCCATGC
GGCAGGGATACGGCGCTAACGATGCATCTCAACAATTGTTGTGTAGGTACTCCGC
CGCCGAGGGACCTGAGCGAGTCCGCATCGACCGGATCGGAAAACCTCTCGAGAA
AGGCGTCTAACCAGTCACAGTCGCAAGGTAGGCTGAGCACCGTGGCGGGCGGCA
GCGGGCGGCGGTCGGGGTTGTTTCTGGCGGAGGTGCTGCTGATGATGTAATTAAA
GTAGGCGGTCTTGAGACGGCGGATGGTCGACAGAAGCACCATGTCCTTGGGTCC
GGCCTGCTGAATGCGCAGGCGGTCGGCCATGCCCCAGGCTTCGTTTTGACATCGG
CGCAGGTCTTTGTAGTAGTCTTGCATGAGCCTTTCTACCGGCACTTCTTCTTCTCC
TTCCTCTTGTCCTGCATCTCTTGCATCTATCGCTGCGGCGGCGGCGGAGTTTGGCC
GTAGGTGGCGCCCTCTTCCTCCCATGCGTGTGACCCCGAAGCCCCTCATCGGCTG
AAGCAGGGCTAGGTCGGCGACAACGCGCTCGGCTAATATGGCCTGCTGCACCTG
CGTGAGGGTAGACTGGAAGTCATCCATGTCCACAAAGCGGTGGTATGCGCCCGT
GTTGATGGTGTAAGTGCAGTTGGCCATAACGGACCAGTTAACGGTCTGGTGACCC
GGCTGCGAGAGCTCGGTGTACCTGAGACGCGAGTAAGCCCTCGAGTCAAATACG
TAGTCGTTGCAAGTCCGCACCAGGTACTGGTATCCCACCAAAAAGTGCGGCGGC
GGCTGGCGGTAGAGGGGCCAGCGTAGGGTGGCCGGGGCTCCGGGGGCGAGATCT
TCCAACATAAGGCGATGATATCCGTAGATGTACCTGGACATCCAGGTGATGCCG
GCGGCGGTGGTGGAGGCGCGCGGAAAGTCGCGGACGCGGTTCCAGATGTTGCGC
AGCGGCAAAAAGTGCTCCATGGTCGGGACGCTCTGGCCGGTCAGGCGCGCGCAA
TCGTTGACGCTCTAGCGTGCAAAAGGAGAGCCTGTAAGCGGGCACTCTTCCGTG
GTCTGGTGGATAAATTCGCAAGGGTATCATGGCGGACGACCGGGGTTCGAGCCC
CGTATCCGGCCGTCCGCCGTGATCCATGCGGTTACCGCCCGCGTGTCGAACCCAG
GTGTGCGACGTCAGACAACGGGGGAGTGCTCCTTTTGGCTTCCTTCCAGGCGCGG
CGGCTGCTGCGCTAGCTTTTTTGGCCACTGGCCGCGCGCAGCGTAAGCGGTTAGG
CTGGAAAGCGAAAGCATTAAGTGGCTCGCTCCCTGTAGCCGGAGGGTTATTTTCC
AAGGGTTGAGTCGCGGGACCCCCGGTTCGAGTCTCGGACCGGCCGGACTGCGGC
GAACGGGGGTTTGCCTCCCCGTCATGCAAGACCCCGCTTGCAAATTCCTCCGGAA
ACAGGGACGAGCCCCTTTTTTGCTTTTCCCAGATGCATCCGGTGCTGCGGCAGAT
GCGCCCCCCTCCTCAGCAGCGGCAAGAGCAAGAGCAGCGGCAGACATGCAGGGC
ACCCTCCCCTCCTCCTACCGCGTCAGGAGGGGCGACATCCGCGGTTGACGCGGCA
GCAGATGGTGATTACGAACCCCCGCGGCGCCGGGCCCGGCACTACCTGGACTTG
GAGGAGGGCGAGGGCCTGGCGCGGCTAGGAGCGCCCTCTCCTGAGCGGCACCCA
AGGGTGCAGCTGAAGCGTGATACGCGTGAGGCGTACGTGCCGCGGCAGAACCTG
TTTCGCGACCGCGAGGGAGAGGAGCCCGAGGAGATGCGGGATCGAAAGTTCCAC
GCAGGGCGCGAGCTGCGGCATGGCCTGAATCGCGAGCGGTTGCTGCGCGAGGAG
GACTTTGAGCCCGACGCGCGAACCGGGATTAGTCCCGCGCGCGCACACGTGGCG
GCCGCCGACCTGGTAACCGCATACGAGCAGACGGTGAACCAGGAGATTAACTTT
CAAAAAAGCTTTAACAACCACGTGCGTACGCTTGTGGCGCGCGAGGAGGTGGCT
ATAGGACTGATGCATCTGTGGGACTTTGTAAGCGCGCTGGAGCAAAACCCAAAT
AGCAAGCCGCTCATGGCGCAGCTGTTCCTTATAGTGCAGCACAGCAGGGACAAC
GAGGCATTCAGGGATGCGCTGCTAAACATAGTAGAGCCCGAGGGCCGCTGGCTG
CTCGATTTGATAAACATCCTGCAGAGCATAGTGGTGCAGGAGCGCAGCTTGAGC
CTGGCTGACAAGGTGGCCGCCATCAACTATTCCATGCTTAGCCTGGGCAAGTTTT
ACGCCCGCAAGATATACCATACCCCTTACGTTCCCATAGACAAGGAGGTAAAGA
TCGAGGGGTTCTACATGCGCATGGCGCTGAAGGTGCTTACCTTGAGCGACGACCT
GGGCGTTTATCGCAACGAGCGCATCCACAAGGCCGTGAGCGTGAGCCGGCGGCG
CGAGCTCAGCGACCGCGAGCTGATGCACAGCCTGCAAAGGGCCCTGGCTGGCAC
GGGCAGCGGCGATAGAGAGGCCGAGTCCTACTTTGACGCGGGCGCTGACCTGCG
CTGGGCCCCAAGCCGACGCGCCCTGGAGGCAGCTGGGGCCGGACCTGGGCTGGC
GGTGGCACCCGCGCGCGCTGGCAACGTCGGCGGCGTGGAGGAATATGACGAGGA
CGATGAGTACGAGCCAGAGGACGGCGAGTACTAAGCGGTGATGTTTCTGATCAG
ATGATGCAAGACGCAACGGACCCGGCGGTGCGGGCGGCGCTGCAGAGCCAGCC
GTCCGGCCTTAACTCCACGGACGACTGGCGCCAGGTCATGGACCGCATCATGTCG
CTGACTGCGCGCAATCCTGACGCGTTCCGGCAGCAGCCGCAGGCCAACCGGCTC
TCCGCAATTCTGGAAGCGGTGGTCCCGGCGCGCGCAAACCCCACGCACGAGAAG
GTGCTGGCGATCGTAAACGCGCTGGCCGAAAACAGGGCCATCCGGCCCGACGAG
GCCGGCCTGGTCTACGACGCGCTGCTTCAGCGCGTGGCTCGTTACAACAGCGGCA
ACGTGCAGACCAACCTGGACCGGCTGGTGGGGGATGTGCGCGAGGCCGTGGCGC
AGCGTGAGCGCGCGCAGCAGCAGGGCAACCTGGGCTCCATGGTTGCACTAAACG
CCTTCCTGAGTACACAGCCCGCCAACGTGCCGCGGGGACAGGAGGACTACACCA
ACTTTGTGAGCGCACTGCGGCTAATGGTGACTGAGACACCGCAAAGTGAGGTGT
ACCAGTCTGGGCCAGACTATTTTTTCCAGACCAGTAGACAAGGCCTGCAGACCGT
AAACCTGAGCCAGGCTTTCAAAAACTTGCAGGGGCTGTGGGGGGTGCGGGCTCC
CACAGGCGACCGCGCGACCGTGTCTAGCTTGCTGACGCCCAACTCGCGCCTGTTG
CTGCTGCTAATAGCGCCCTTCACGGACAGTGGCAGCGTGTCCCGGGACACATACC
TAGGTCACTTGCTGACACTGTACCGCGAGGCCATAGGTCAGGCGCATGTGGACG
AGCATACTTTCCAGGAGATTACAAGTGTCAGCCGCGCGCTGGGGCAGGAGGACA
CGGGCAGCCTGGAGGCAACCCTAAACTACCTGCTGACCAACCGGCGGCAGAAGA
TCCCCTCGTTGCACAGTTTAAACAGCGAGGAGGAGCGCATTTTGCGCTACGTGCA
GCAGAGCGTGAGCCTTAACCTGATGCGCGACGGGGTAACGCCCAGCGTGGCGCT
GGACATGACCGCGCGCAACATGGAACCGGGCATGTATGCCTCAAACCGGCCGTT
TATCAACCGCCTAATGGACTACTTGCATCGCGCGGCCGCCGTGAACCCCGAGTAT
TTCACCAATGCCATCTTGAACCCGCACTGGCTACCGCCCCCTGGTTTCTACACCG
GGGGATTCGAGGTGCCCGAGGGTAACGATGGATTCCTCTGGGACGACATAGACG
ACAGCGTGTTTTCCCCGCAACCGCAGACCCTGCTAGAGTTGCAACAGCGCGAGC
AGGCAGAGGCGGCGCTGCGAAAGGAAAGCTTCCGCAGGCCAAGCAGCTTGTCCG
ATCTAGGCGCTGCGGCCCCGCGGTCAGATGCTAGTAGCCCATTTCCAAGCTTGAT
AGGGTCTCTTACCAGCACTCGCACCACCCGCCCGCGCCTGCTGGGCGAGGAGGA
GTACCTAAACAACTCGCTGCTGCAGCCGCAGCGCGAAAAAAACCTGCCTCCGGC
ATTTCCCAACAACGGGATAGAGAGCCTAGTGGACAAGATGAGTAGATGGAAGAC
GTACGCGCAGGAGCACAGGGACGTGCCAGGCCCGCGCCCGCCCACCCGTCGTCA
AAGGCACGACCGTCAGCGGGGTCTGGTGTGGGAGGACGATGACTCGGCAGACGA
CAGCAGCGTCCTGGATTTGGGAGGGAGTGGCAACCCGTTTGCGCACCTTCGCCCC
AGGCTGGGGAGAATGTTTTAAAAAAAAAAAAGCATGATGCAAAATAAAAAACTC
ACCAAGGCCATGGCACCGAGCGTTGGTTTTCTTGTATTCCCCTTAGTATGCGGCG
CGCGGCGATGTATGAGGAAGGTCCTCCTCCCTCCTACGAGAGTGTGGTGAGCGC
GGCGCCAGTGGCGGCGGCGCTGGGTTCTCCCTTCGATGCTCCCCTGGACCCGCCG
TTTGTGCCTCCGCGGTACCTGCGGCCTACCGGGGGGAGAAACAGCATCCGTTACT
CTGAGTTGGCACCCCTATTCGACACCACCCGTGTGTACCTGGTGGACAACAAGTC
AACGGATGTGGCATCCCTGAACTACCAGAACGACCACAGCAACTTTCTGACCAC
GGTCATTCAAAACAATGACTACAGCCCGGGGGAGGCAAGCACACAGACCATCAA
TCTTGACGACCGGTCGCACTGGGGCGGCGACCTGAAAACCATCCTGCATACCAA
CATGCCAAATGTGAACGAGTTCATGTTTACCAATAAGTTTAAGGCGCGGGTGATG
GTGTCGCGCTTGCCTACTAAGGACAATCAGGTGGAGCTGAAATACGAGTGGGTG
GAGTTCACGCTGCCCGAGGGCAACTACTCCGAGACCATGACCATAGACCTTATG
AACAACGCGATCGTGGAGCACTACTTGAAAGTGGGCAGACAGAACGGGGTTCTG
GAAAGCGACATCGGGGTAAAGTTTGACACCCGCAACTTCAGACTGGGGTTTGAC
CCCGTCACTGGTCTTGTCATGCCTGGGGTATATACAAACGAAGCCTTCCATCCAG
ACATCATTTTGCTGCCAGGATGCGGGGTGGACTTCACCCACAGCCGCCTGAGCAA
CTTGTTGGGCATCCGCAAGCGGCAACCCTTCCAGGAGGGCTTTAGGATCACCTAC
GATGATCTGGAGGGTGGTAACATTCCCGCACTGTTGGATGTGGACGCCTACCAG
GCGAGCTTGAAAGATGACACCGAACAGGGCGGGGGTGGCGCAGGCGGCAGCAA
CAGCAGTGGCAGCGGCGCGGAAGAGAACTCCAACGCGGCAGCCGCGGCAATGC
AGCCGGTGGAGGACATGAACGATCATGCCATTCGCGGCGACACCTTTGCCACAC
GGGCTGAGGAGAAGCGCGCTGAGGCCGAAGCAGCGGCCGAAGCTGCCGCCCCC
GCTGCGCAACCCGAGGTCGAGAAGCCTCAGAAGAAACCGGTGATCAAACCCCTG
ACAGAGGACAGCAAGAAACGCAGTTACAACCTAATAAGCAATGACAGCACCTTC
ACCCAGTACCGCAGCTGGTACCTTGCATACAACTACGGCGACCCTCAGACCGGA
ATCCGCTCATGGACCCTGCTTTGCACTCCTGACGTAACCTGCGGCTCGGAGCAGG
TCTACTGGTCGTTGCCAGACATGATGCAAGACCCCGTGACCTTCCGCTCCACGCG
CCAGATCAGCAACTTTCCGGTGGTGGGCGCCGAGCTGTTGCCCGTGCACTCCAAG
AGCTTCTACAACGACCAGGCCGTCTACTCCCAACTCATCCGCCAGTTTACCTCTC
TGACCCACGTGTTCAATCGCTTTCCCGAGAACCAGATTTTGGCGCGCCCGCCAGC
CCCCACCATCACCACCGTCAGTGAAAACGTTCCTGCTCTCACAGATCACGGGACG
CTACCGCTGCGCAACAGCATCGGAGGAGTCCAGCGAGTGACCATTACTGACGCC
AGACGCCGCACCTGCCCCTACGTTTACAAGGCCCTGGGCATAGTCTCGCCGCGCG
TCCTATCGAGCCGCACTTTTTGAGCAAGCATGTCCATCCTTATATCGCCCAGCAA
TAACACAGGCTGGGGCCTGCGCTTCCCAAGCAAGATGTTTGGCGGGGCCAAGAA
GCGCTCCGACCAACACCCAGTGCGCGTGCGCGGGCACTACCGCGCGCCCTGGGG
CGCGCACAAACGCGGCCGCACTGGGCGCACCACCGTCGATGACGCCATCGACGC
GGTGGTGGAGGAGGCGCGCAACTACACGCCCACGCCGCCACCAGTGTCCACAGT
GGACGCGGCCATTCAGACCGTGGTGCGCGGAGCCCGGCGCTATGCTAAAATGAA
GAGACGGCGGAGGCGCGTAGCACGTCGCCACCGCCGCCGACCCGGCACTGCCGC
CCAACGCGCGGCGGCGGCCCTGCTTAACCGCGCACGTCGCACCGGCCGACGGGC
GGCCATGCGGGCCGCTCGAAGGCTGGCCGCGGGTATTGTCACTGTGCCCCCCAG
GTCCAGGCGACGAGCGGCCGCCGCAGCAGCCGCGGCCATTAGTGCTATGACTCA
GGGTCGCAGGGGCAACGTGTATTGGGTGCGCGACTCGGTTAGCGGCCTGCGCGT
GCCCGTGCGCACCCGCCCCCCGCGCAACTAGATTGCAAGAAAAAACTACTTAGA
CTCGTACTGTTGTATGTATCCAGCGGCGGCGGCGCGCAACGAAGCTATGTCCAAG
CGCAAAATCAAAGAAGAGATGCTCCAGGTCATCGCGCCGGAGATCTATGGCCCC
CCGAAGAAGGAAGAGCAGGATTACAAGCCCCGAAAGCTAAAGCGGGTCAAAAA
GAAAAAGAAAGATGATGATGATGAACTTGACGACGAGGTGGAACTGCTGCACGC
TACCGCGCCCAGGCGACGGGTACAGTGGAAAGGTCGACGCGTAAAACGTGTTTT
GCGACCCGGCACCACCGTAGTCTTTACGCCCGGTGAGCGCTCCACCCGCACCTAC
AAGCGCGTGTATGATGAGGTGTACGGCGACGAGGACCTGCTTGAGCAGGCCAAC
GAGCGCCTCGGGGAGTTTGCCTACGGAAAGCGGCATAAGGACATGCTGGCGTTG
CCGCTGGACGAGGGCAACCCAACACCTAGCCTAAAGCCCGTAACACTGCAGCAG
GTGCTGCCCGCGCTTGCACCGTCCGAAGAAAAGCGCGGCCTAAAGCGCGAGTCT
GGTGACTTGGCACCCACCGTGCAGCTGATGGTACCCAAGCCCCAGCGACTGGAA
GATGTCTTGGAAAAAATGACCGTGGAACCTGGGCTGGAGCCCGAGGTCCGCGTG
CGGCCAATCAAGCAGGTGGCGCCGGGACTGGGCGTGCAGACCGTGGACGTTCAG
ATACCCACTACCAGTAGCACCAGTATTGCCACCGCCACAGAGGGCATGGAGACA
CAAACGTCCCCGGTTGCCTCAGCGGTGGCGGATGCCGCGGTGCAGGCGGTCGCT
GCGGCCGCGTCCAAGACCTCTACGGAGGTGCAAACGGACCCGTGGATGTTTCGC
GTTTCAGCCCCCCGGCGCCCGCGCCGTTCGAGGAAGTACGGCGCCGCCAGCGCG
CTACTGCCCGAATATGCCCTACATCCTTCCATTGCGCCTACCCCCGGCTATCGTG
GCTACACCTACCGCCCCAGAAGACGAGCAACTACCCGACGCCGAACCACCACTG
GAACCCGCCGCCGCCGTCGCCGTCGCCAGCCCGTGCTGGCCCCGATTTCCGTGCG
CAGGGTGGCTCGCGAAGGAGGCAGGACCCTGGTGCTGCCAACAGCGCGCTACCA
CCCCAGCATCGTTTAAAAGCCGGTCTTTGTGGTTCTTGCAGATATGGCCCTCACC
TGCCGCCTCCGTTTCCCGGTGCCGGGATTCCGAGGAAGAATGCACCGTAGGAGG
GGCATGGCCGGCCACGGCCTGACGGGCGGCATGCGTCGTGCGCACCACCGGCGG
CGGCGCGCGTCGCACCGTCGCATGCGCGGCGGTATCCTGCCCCTCCTTATTCCAC
TGATCGCCGCGGCGATTGGCGCCGTGCCCGGAATTGCATCCGTGGCCTTGCAGGC
GCAGAGACACTGATTAAAAACAAGTTGCATGTGGAAAAATCAAAATAAAAAGTC
TGGACTCTCACGCTCGCTTGGTCCTGTAACTATTTTGTAGAATGGAAGACATCAA
CTTTGCGTCTCTGGCCCCGCGACACGGCTCGCGCCCGTTCATGGGAAACTGGCAA
GATATCGGCACCAGCAATATGAGCGGTGGCGCCTTCAGCTGGGGCTCGCTGTGG
AGCGGCATTAAAAATTTCGGTTCCACCGTTAAGAACTATGGCAGCAAGGCCTGG
AACAGCAGCACAGGCCAGATGCTGAGGGATAAGTTGAAAGAGCAAAATTTCCAA
CAAAAGGTGGTAGATGGCCTGGCCTCTGGCATTAGCGGGGTGGTGGACCTGGCC
AACCAGGCAGTGCAAAATAAGATTAACAGTAAGCTTGATCCCCGCCCTCCCGTA
GAGGAGCCTCCACCGGCCGTGGAGACAGTGTCTCCAGAGGGGCGTGGCGAAAAG
CGTCCGCGCCCCGACAGGGAAGAAACTCTGGTGACGCAAATAGACGAGCCTCCC
TCGTACGAGGAGGCACTAAAGCAAGGCCTGCCCACCACCCGTCCCATCGCGCCC
ATGGCTACCGGAGTGCTGGGCCAGCACACACCCGTAACGCTGGACCTGCCTCCC
CCCGCCGACACCCAGCAGAAACCTGTGCTGCCAGGCCCGACCGCCGTTGTTGTA
ACCCGTCCTAGCCGCGCGTCCCTGCGCCGCGCCGCCAGCGGTCCGCGATCGTTGC
GGCCCGTAGCCAGTGGCAACTGGCAAAGCACACTGAACAGCATCGTGGGTCTGG
GGGTGCAATCCCTGAAGCGCCGACGATGCTTCTGATAGCTAACGTGTCGTATGTG
TGTCATGTATGCGTCCATGTCGCCGCCAGAGGAGCTGCTGAGCCGCCGCGCGCCC
GCTTTCCAAGATGGCTACCCCTTCGATGATGCCGCAGTGGTCTTACATGCACATC
TCGGGCCAGGACGCCTCGGAGTACCTGAGCCCCGGGCTGGTGCAGTTTGCCCGC
GCCACCGAGACGTACTTCAGCCTGAATAACAAGTTTAGAAACCCCACGGTGGCG
CCTACGCACGACGTGACCACAGACCGGTCCCAGCGTTTGACGCTGCGGTTCATCC
CTGTGGACCGTGAGGATACTGCGTACTCGTACAAGGCGCGGTTCACCCTAGCTGT
GGGTGATAACCGTGTGCTGGACATGGCTTCCACGTACTTTGACATCCGCGGCGTG
CTGGACAGGGGCCCTACTTTTAAGCCCTACTCTGGCACTGCCTACAACGCCCTGG
CTCCCAAGGGTGCCCCAAATCCTTGCGAATGGGATGAAGCTGCTACTGCTCTTGA
AATAAACCTAGAAGAAGAGGACGATGACAACGAAGACGAAGTAGACGAGCAAG
CTGAGCAGCAAAAAACTCACGTATTTGGGCAGGCGCCTTATTCTGGTATAAATAT
TACAAAGGAGGGTATTCAAATAGGTGTCGAAGGTCAAACACCTAAATATGCCGA
TAAAACATTTCAACCTGAACCTCAAATAGGAGAATCTCAGTGGTACGAAACAGA
AATTAATCATGCAGCTGGGAGAGTCCTAAAAAAGACTACCCCAATGAAACCATG
TTACGGTTCATATGCAAAACCCACAAATGAAAATGGAGGGCAAGGCATTCTTGT
AAAGCAACAAAATGGAAAGCTAGAAAGTCAAGTGGAAATGCAATTTTTCTCAAC
TACTGAGGCAGCCGCAGGCAATGGTGATAACTTGACTCCTAAAGTGGTATTGTAC
AGTGAAGATGTAGATATAGAAACCCCAGACACTCATATTTCTTACATGCCCACTA
TTAAGGAAGGTAACTCACGAGAACTAATGGGCCAACAATCTATGCCCAACAGGC
CTAATTACATTGCTTTTAGGGACAATTTTATTGGTCTAATGTATTACAACAGCACG
GGTAATATGGGTGTTCTGGCGGGCCAAGCATCGCAGTTGAATGCTGTTGTAGATT
TGCAAGACAGAAACACAGAGCTTTCATACCAGCTTTTGCTTGATTCCATTGGTGA
TAGAACCAGGTACTTTTCTATGTGGAATCAGGCTGTTGACAGCTATGATCCAGAT
GTTAGAATTATTGAAAATCATGGAACTGAAGATGAACTTCCAAATTACTGCTTTC
CACTGGGAGGTGTGATTAATACAGAGACTCTTACCAAGGTAAAACCTAAAACAG
GTCAGGAAAATGGATGGGAAAAAGATGCTACAGAATTTTCAGATAAAAATGAAA
TAAGAGTTGGAAATAATTTTGCCATGGAAATCAATCTAAATGCCAACCTGTGGAG
AAATTTCCTGTACTCCAACATAGCGCTGTATTTGCCCGACAAGCTAAAGTACAGT
CCTTCCAACGTAAAAATTTCTGATAACCCAAACACCTACGACTACATGAACAAGC
GAGTGGTGGCTCCCGGGCTAGTGGACTGCTACATTAACCTTGGAGCACGCTGGTC
CCTTGACTATATGGACAACGTCAACCCATTTAACCACCACCGCAATGCTGGCCTG
CGCTACCGCTCAATGTTGCTGGGCAATGGTCGCTATGTGCCCTTCCACATCCAGG
TGCCTCAGAAGTTCTTTGCCATTAAAAACCTCCTTCTCCTGCCGGGCTCATACACC
TACGAGTGGAACTTCAGGAAGGATGTTAACATGGTTCTGCAGAGCTCCCTAGGA
AATGACCTAAGGGTTGACGGAGCCAGCATTAAGTTTGATAGCATTTGCCTTTACG
CCACCTTCTTCCCCATGGCCCACAACACCGCCTCCACGCTTGAGGCCATGCTTAG
AAACGACACCAACGACCAGTCCTTTAACGACTATCTCTCCGCCGCCAACATGCTC
TACCCTATACCCGCCAACGCTACCAACGTGCCCATATCCATCCCCTCCCGCAACT
GGGCGGCTTTCCGCGGCTGGGCCTTCACGCGCCTTAAGACTAAGGAAACCCCATC
ACTGGGCTCGGGCTACGACCCTTATTACACCTACTCTGGCTCTATACCCTACCTA
GATGGAACCTTTTACCTCAACCACACCTTTAAGAAGGTGGCCATTACCTTTGACT
CTTCTGTCAGCTGGCCTGGCAATGACCGCCTGCTTACCCCCAACGAGTTTGAAAT
TAAGCGCTCAGTTGACGGGGAGGGTTACAACGTTGCCCAGTGTAACATGACCAA
AGACTGGTTCCTGGTACAAATGCTAGCTAACTATAACATTGGCTACCAGGGCTTC
TATATCCCAGAGAGCTACAAGGACCGCATGTACTCCTTCTTTAGAAACTTCCAGC
CCATGAGCCGTCAGGTGGTGGATGATACTAAATACAAGGACTACCAACAGGTGG
GCATCCTACACCAACACAACAACTCTGGATTTGTTGGCTACCTTGCCCCCACCAT
GCGCGAAGGACAGGCCTACCCTGCTAACTTCCCCTATCCGCTTATAGGCAAGACC
GCAGTTGACAGCATTACCCAGAAAAAGTTTCTTTGCGATCGCACCCTTTGGCGCA
TCCCATTCTCCAGTAACTTTATGTCCATGGGCGCACTCACAGACCTGGGCCAAAA
CCTTCTCTACGCCAACTCCGCCCACGCGCTAGACATGACTTTTGAGGTGGATCCC
ATGGACGAGCCCACCCTTCTTTATGTTTTGTTTGAAGTCTTTGACGTGGTCCGTGT
GCACCAGCCGCACCGCGGCGTCATCGAAACCGTGTACCTGCGCACGCCCTTCTCG
GCCGGCAACGCCACAACATAAAGAAGCAAGCAACATCAACAACAGCTGCCGCC
ATGGGCTCCAGTGAGCAGGAACTGAAAGCCATTGTCAAAGATCTTGGTTGTGGG
CCATATTTTTTGGGCACCTATGACAAGCGCTTTCCAGGCTTTGTTTCTCCACACAA
GCTCGCCTGCGCCATAGTCAATACGGCCGGTCGCGAGACTGGGGGCGTACACTG
GATGGCCTTTGCCTGGAACCCGCACTCAAAAACATGCTACCTCTTTGAGCCCTTT
GGCTTTTCTGACCAGCGACTCAAGCAGGTTTACCAGTTTGAGTACGAGTCACTCC
TGCGCCGTAGCGCCATTGCTTCTTCCCCCGACCGCTGTATAACGCTGGAAAAGTC
CACCCAAAGCGTACAGGGGCCCAACTCGGCCGCCTGTGGACTATTCTGCTGCATG
TTTCTCCACGCCTTTGCCAACTGGCCCCAAACTCCCATGGATCACAACCCCACCA
TGAACCTTATTACCGGGGTACCCAACTCCATGCTCAACAGTCCCCAGGTACAGCC
CACCCTGCGTCGCAACCAGGAACAGCTCTACAGCTTCCTGGAGCGCCACTCGCCC
TACTTCCGCAGCCACAGTGCGCAGATTAGGAGCGCCACTTCTTTTTGTCACTTGA
AAAACATGTAAAAATAATGTACTAGAGACACTTTCAATAAAGGCAAATGCTTTT
ATTTGTACACTCTCGGGTGATTATTTACCCCCACCCTTGCCGTCTGCGCCGTTTAA
AAATCAAAGGGGTTCTGCCGCGCATCGCTATGCGCCACTGGCAGGGACACGTTG
CGATACTGGTGTTTAGTGCTCCACTTAAACTCAGGCACAACCATCCGCGGCAGCT
CGGTGAAGTTTTCACTCCACAGGCTGCGCACCATCACCAACGCGTTTAGCAGGTC
GGGCGCCGATATCTTGAAGTCGCAGTTGGGGCCTCCGCCCTGCGCGCGCGAGTTG
CGATACACAGGGTTGCAGCACTGGAACACTATCAGCGCCGGGTGGTGCACGCTG
GCCAGCACGCTCTTGTCGGAGATCAGATCCGCGTCCAGGTCCTCCGCGTTGCTCA
GGGCGAACGGAGTCAACTTTGGTAGCTGCCTTCCCAAAAAGGGCGCGTGCCCAG
GCTTTGAGTTGCACTCGCACCGTAGTGGCATCAAAAGGTGACCGTGCCCGGTCTG
GGCGTTAGGATACAGCGCCTGCATAAAAGCCTTGATCTGCTTAAAAGCCACCTG
AGCCTTTGCGCCTTCAGAGAAGAACATGCCGCAAGACTTGCCGGAAAACTGATT
GGCCGGACAGGCCGCGTCGTGCACGCAGCACCTTGCGTCGGTGTTGGAGATCTG
CACCACATTTCGGCCCCACCGGTTCTTCACGATCTTGGCCTTGCTAGACTGCTCCT
TCAGCGCGCGCTGCCCGTTTTCGCTCGTCACATCCATTTCAATCACGTGCTCCTTA
TTTATCATAATGCTTCCGTGTAGACACTTAAGCTCGCCTTCGATCTCAGCGCAGC
GGTGCAGCCACAACGCGCAGCCCGTGGGCTCGTGATGCTTGTAGGTCACCTCTGC
AAACGACTGCAGGTACGCCTGCAGGAATCGCCCCATCATCGTCACAAAGGTCTT
GTTGCTGGTGAAGGTCAGCTGCAACCCGCGGTGCTCCTCGTTCAGCCAGGTCTTG
CATACGGCCGCCAGAGCTTCCACTTGGTCAGGCAGTAGTTTGAAGTTCGCCTTTA
GATCGTTATCCACGTGGTACTTGTCCATCAGCGCGCGCGCAGCCTCCATGCCCTT
CTCCCACGCAGACACGATCGGCACACTCAGCGGGTTCATCACCGTAATTTCACTT
TCCGCTTCGCTGGGCTCTTCCTCTTCCTCTTGCGTCCGCATACCACGCGCCACTGG
GTCGTCTTCATTCAGCCGCCGCACTGTGCGCTTACCTCCTTTGCCATGCTTGATTA
GCACCGGTGGGTTGCTGAAACCCACCATTTGTAGCGCCACATCTTCTCTTTCTTCC
TCGCTGTCCACGATTACCTCTGGTGATGGCGGGCGCTCGGGCTTGGGAGAAGGG
CGCTTCTTTTTCTTCTTGGGCGCAATGGCCAAATCCGCCGCCGAGGTCGATGGCC
GCGGGCTGGGTGTGCGCGGCACCAGCGCGTCTTGTGATGAGTCTTCCTCGTCCTC
GGACTCGATACGCCGCCTCATCCGCTTTTTTGGGGGCGCCCGGGGAGGCGGCGG
CGACGGGGACGGGGACGACACGTCCTCCATGGTTGGGGGACGTCGCGCCGCACC
GCGTCCGCGCTCGGGGGTGGTTTCGCGCTGCTCCTCTTCCCGACTGGCCATTTCCT
TCTCCTATAGGCAGAAAAAGATCATGGAGTCAGTCGAGAAGAAGGACAGCCTAA
CCGCCCCCTCTGAGTTCGCCACCACCGCCTCCACCGATGCCGCCAACGCGCCTAC
CACCTTCCCCGTCGAGGCACCCCCGCTTGAGGAGGAGGAAGTGATTATCGAGCA
GGACCCAGGTTTTGTAAGCGAAGACGACGAGGACCGCTCAGTACCAACAGAGGA
TAAAAAGCAAGACCAGGACAACGCAGAGGCAAACGAGGAACAAGTCGGGCGGG
GGGACGAAAGGCATGGCGACTACCTAGATGTGGGAGACGACGTGCTGTTGAAGC
ATCTGCAGCGCCAGTGCGCCATTATCTGCGACGCGTTGCAAGAGCGCAGCGATG
TGCCCCTCGCCATAGCGGATGTCAGCCTTGCCTACGAACGCCACCTATTCTCACC
GCGCGTACCCCCCAAACGCCAAGAAAACGGCACATGCGAGCCCAACCCGCGCCT
CAACTTCTACCCCGTATTTGCCGTGCCAGAGGTGCTTGCCACCTATCACATCTTTT
TCCAAAACTGCAAGATACCCCTATCCTGCCGTGCCAACCGCAGCCGAGCGGACA
AGCAGCTGGCCTTGCGGCAGGGCGCTGTCATACCTGATATCGCCTCGCTCAACGA
AGTGCCAAAAATCTTTGAGGGTCTTGGACGCGACGAGAAGCGCGCGGCAAACGC
TCTGCAACAGGAAAACAGCGAAAATGAAAGTCACTCTGGAGTGTTGGTGGAACT
CGAGGGTGACAACGCGCGCCTAGCCGTACTAAAACGCAGCATCGAGGTCACCCA
CTTTGCCTACCCGGCACTTAACCTACCCCCCAAGGTCATGAGCACAGTCATGAGT
GAGCTGATCGTGCGCCGTGCGCAGCCCCTGGAGAGGGATGCAAATTTGCAAGAA
CAAACAGAGGAGGGCCTACCCGCAGTTGGCGACGAGCAGCTAGCGCGCTGGCTT
CAAACGCGCGAGCCTGCCGACTTGGAGGAGCGACGCAAACTAATGATGGCCGCA
GTGCTCGTTACCGTGGAGCTTGAGTGCATGCAGCGGTTCTTTGCTGACCCGGAGA
TGCAGCGCAAGCTAGAGGAAACATTGCACTACACCTTTCGACAGGGCTACGTAC
GCCAGGCCTGCAAGATCTCCAACGTGGAGCTCTGCAACCTGGTCTCCTACCTTGG
AATTTTGCACGAAAACCGCCTTGGGCAAAACGTGCTTCATTCCACGCTCAAGGGC
GAGGCGCGCCGCGACTACGTCCGCGACTGCGTTTACTTATTTCTATGCTACACCT
GGCAGACGGCCATGGGCGTTTGGCAGCAGTGCTTGGAGGAGTGCAACCTCAAGG
AGCTGCAGAAACTGCTAAAGCAAAACTTGAAGGACCTATGGACGGCCTTCAACG
AGCGCTCCGTGGCCGCGCACCTGGCGGACATCATTTTCCCCGAACGCCTGCTTAA
AACCCTGCAACAGGGTCTGCCAGACTTCACCAGTCAAAGCATGTTGCAGAACTTT
AGGAACTTTATCCTAGAGCGCTCAGGAATCTTGCCCGCCACCTGCTGTGCACTTC
CTAGCGACTTTGTGCCCATTAAGTACCGCGAATGCCCTCCGCCGCTTTGGGGCCA
CTGCTACCTTCTGCAGCTAGCCAACTACCTTGCCTACCACTCTGACATAATGGAA
GACGTGAGCGGTGACGGTCTACTGGAGTGTCACTGTCGCTGCAACCTATGCACCC
CGCACCGCTCCCTGGTTTGCAATTCGCAGCTGCTTAACGAAAGTCAAATTATCGG
TACCTTTGAGCTGCAGGGTCCCTCGCCTGACGAAAAGTCCGCGGCTCCGGGGTTG
AAACTCACTCCGGGGCTGTGGACGTCGGCTTACCTTCGCAAATTTGTACCTGAGG
ACTACCACGCCCACGAGATTAGGTTCTACGAAGACCAATCCCGCCCGCCTAATGC
GGAGCTTACCGCCTGCGTCATTACCCAGGGCCACATTCTTGGCCAATTGCAAGCC
ATCAACAAAGCCCGCCAAGAGTTTCTGCTACGAAAGGGACGGGGGGTTTACTTG
GACCCCCAGTCCGGCGAGGAGCTCAACCCAATCCCCCCGCCGCCGCAGCCCTAT
CAGCAGCAGCCGCGGGCCCTTGCTTCCCAGGATGGCACCCAAAAAGAAGCTGCA
GCTGCCGCCGCCACCCACGGACGAGGAGGAATACTGGGACAGTCAGGCAGAGG
ACGTTTTGGACGAGGAGGAGGAGGACATGATGGAAGACTGGGAGAGCCTAGAC
GAGGAAGCTTCCGAGGTCGAAGAGGTGTCAGACGAAACACCGTCACCCTCGGTC
GCATTCCCCTCGCCGGCGCCCCAGAAATCGGCAACCGGTTCCAGCATGGCTACA
ACCTCCGCTCCTCAGGCGCCGCCGGCACTGCCCGTTCGCCGACCCAACCGTAGAT
GGGACACCACTGGAACCAGGGCCGGTAAGTCCAAGCAGCCGCCGCCGTTAGCCC
AAGAGCAACAACAGCGCCAAGGCTACCGCTCATGGCGCGGGCACAAGAACGCC
ATAGTTGCTTGCTTGCAAGACTGTGGGGGCAACATCTCCTTCGCCCGCCGCTTTC
TTCTCTACCATCACGGCGTGGCCTTCCCCCGTAACATCCTGCATTACTACCGTCAT
CTCTACAGCCCATACTGCACCGGCGGCAGCGGCAGCAACAGCAGCGGCCACACA
GAAGCAAAGGCGACCGGATAGCAAGACTCTGACAAAGCCCAAGAAATCCACAG
CGGCGGCAGCAGCAGGAGGAGGAGCGCTGCGTCTGGCGCCCAACGAACCCGTAT
CGACCCGCGAGCTTAGAAACAGGATTTTTCCCACTCTGTATGCTATATTTCAACA
GAGCAGGGGCCAAGAACAAGAGCTGAAAATAAAAAACAGGTCTCTGCGATCCCT
CACCCGCAGCTGCCTGTATCACAAAAGCGAAGATCAGCTTCGGCGCACGCTGGA
AGACGCGGAGGCTCTCTTCAGTAAATACTGCGCGCTGACTCTTAAGGACTAGTTT
CGCGCCCTTTCTCAAATTTAAGCGCGAAAACTACGTCATCTCCAGCGGCCACACC
CGGCGCCAGCACCTGTTGTCAGCGCCATTATGAGCAAGGAAATTCCCACGCCCTA
CATGTGGAGTTACCAGCCACAAATGGGACTTGCGGCTGGAGCTGCCCAAGACTA
CTCAACCCGAATAAACTACATGAGCGCGGGACCCCACATGATATCCCGGGTCAA
CGGAATACGCGCCCACCGAAACCGAATTCTCCTGGAACAGGCGGCTATTACCAC
CACACCTCGTAATAACCTTAATCCCCGTAGTTGGCCCGCTGCCCTGGTGTACCAG
GAAAGTCCCGCTCCCACCACTGTGGTACTTCCCAGAGACGCCCAGGCCGAAGTTC
AGATGACTAACTCAGGGGCGCAGCTTGCGGGCGGCTTTCGTCACAGGGTGCGGT
CGCCCGGGCAGGGTATAACTCACCTGACAATCAGAGGGCGAGGTATTCAGCTCA
ACGACGAGTCGGTGAGCTCCTCGCTTGGTCTCCGTCCGGACGGGACATTTCAGAT
CGGCGGCGCCGGCCGCTCTTCATTCACGCCTCGTCAGGCAATCCTAACTCTGCAG
ACCTCGTCCTCTGAGCCGCGCTCTGGAGGCATTGGAACTCTGCAATTTATTGAGG
AGTTTGTGCCATCGGTCTACTTTAACCCCTTCTCGGGACCTCCCGGCCACTATCCG
GATCAATTTATTCCTAACTTTGACGCGGTAAAGGACTCGGCGGACGGCTACGACT
GAATGTTAAGTGGAGAGGCAGAGCAACTGCGCCTGAAACACCTGGTCCACTGTC
GCCGCCACAAGTGCTTTGCCCGCGACTCCGGTGAGTTTTGCTACTTTGAATTGCC
CGAGGATCATATCGAGGGCCCGGCGCACGGCGTCCGGCTTACCGCCCAGGGAGA
GCTTGCCCGTAGCCTGATTCGGGAGTTTACCCAGCGCCCCCTGCTAGTTGAGCGG
GACAGGGGACCCTGTGTTCTCACTGTGATTTGCAACTGTCCTAACCCTGGATTAC
ATCAAGATCCTCTAGTTAATGTCAGGTCGCCTAAGTCGATTAACTAGAGTACCCG
GGGATCTTATTCCCTTTAACTAATAAAAAAAAATAATAAAGCATCACTTACTTAA
AATCAGTTAGCAAATTTCTGTCCAGTTTATTCAGCAGCACCTCCTTGCCCTCCTCC
CAGCTCTGGTATTGCAGCTTCCTCCTGGCTGCAAACTTTCTCCACAATCTAAATG
GAATGTCAGTTTCCTCCTGTTCCTGTCCATCCGCACCCACTATCTTCATGTTGTTG
CAGATGAAGCGCGCAAGACCGTCTGAAGATACCTTCAACCCCGTGTATCCATAT
GACACGGAAACCGGTCCTCCAACTGTGCCTTTTCTTACTCCTCCCTTTGTATCCCC
CAATGGGTTTCAAGAGAGTCCCCCTGGGGTACTCTCTTTGCGCCTATCCGAACCT
CTAGTTACCTCCAATGGCATGCTTGCGCTCAAAATGGGCAACGGCCTCTCTCTGG
ACGAGGCCGGCAACCTTACCTCCCAAAATGTAACCACTGTGAGCCCACCTCTCAA
AAAAACCAAGTCAAACATAAACCTGGAAATATCTGCACCCCTCACAGTTACCTC
AGAAGCCCTAACTGTGGCTGCCGCCGCACCTCTAATGGTCGCGGGCAACACACT
CACCATGCAATCACAGGCCCCGCTAACCGTGCACGACTCCAAACTTAGCATTGCC
ACCCAAGGACCCCTCACAGTGTCAGAAGGAAAGCTAGCCCTGCAAACATCAGGC
CCCCTCACCACCACCGATAGCAGTACCCTTACTATCACTGCCTCACCCCCTCTAA
CTACTGCCACTGGTAGCTTGGGCATTGACTTGAAAGAGCCCATTTATACACAAAA
TGGAAAACTAGGACTAAAGTACGGGGCTCCTTTGCATGTAACAGACGACCTAAA
CACTTTGACCGTAGCAACTGGTCCAGGTGTGACTATTAATAATACTTCCTTGCAA
ACTAAAGTTACTGGAGCCTTGGGTTTTGATTCACAAGGCAATATGCAACTTAATG
TAGCAGGAGGACTAAGGATTGATTCTCAAAACAGACGCCTTATACTTGATGTTAG
TTATCCGTTTGATGCTCAAAACCAACTAAATCTAAGACTAGGACAGGGCCCTCTT
TTTATAAACTCAGCCCACAACTTGGATATTAACTACAACAAAGGCCTTTACTTGT
TTACAGCTTCAAACAATTCCAAAAAGCTTGAGGTTAACCTAAGCACTGCCAAGG
GGTTGATGTTTGACGCTACAGCCATAGCCATTAATGCAGGAGATGGGCTTGAATT
TGGTTCACCTAATGCACCAAACACAAATCCCCTCAAAACAAAAATTGGCCATGG
CCTAGAATTTGATTCAAACAAGGCTATGGTTCCTAAACTAGGAACTGGCCTTAGT
TTTGACAGCACAGGTGCCATTACAGTAGGAAACAAAAATAATGATAAGCTAACT
TTGTGGACCACACCAGCTCCATCTCCTAACTGTAGACTAAATGCAGAGAAAGAT
GCTAAACTCACTTTGGTCTTAACAAAATGTGGCAGTCAAATACTTGCTACAGTTT
CAGTTTTGGCTGTTAAAGGCAGTTTGGCTCCAATATCTGGAACAGTTCAAAGTGC
TCATCTTATTATAAGATTTGACGAAAATGGAGTGCTACTAAACAATTCCTTCCTG
GACCCAGAATATTGGAACTTTAGAAATGGAGATCTTACTGAAGGCACAGCCTAT
ACAAACGCTGTTGGATTTATGCCTAACCTATCAGCTTATCCAAAATCTCACGGTA
AAACTGCCAAAAGTAACATTGTCAGTCAAGTTTACTTAAACGGAGACAAAACTA
AACCTGTAACACTAACCATTACACTAAACGGTACACAGGAAACAGGAGACACAA
CTCCAAGTGCATACTCTATGTCATTTTCATGGGACTGGTCTGGCCACAACTACAT
TAATGAAATATTTGCCACATCCTCTTACACTTTTTCATACATTGCCCAAGAATAA
AGAATCGTTTGTGTTATGTTTCAACGTGTTTATTTTTCAATTGCAGAAAATTTCAA
GTCATTTTTCATTCAGTAGTATAGCCCCACCACCACATAGCTTATACAGATCACC
GTACCTTAATCAAACTCACAGAACCCTAGTATTCAACCTGCCACCTCCCTCCCAA
CACACAGAGTACACAGTCCTTTCTCCCCGGCTGGCCTTAAAAAGCATCATATCAT
GGGTAACAGACATATTCTTAGGTGTTATATTCCACACGGTTTCCTGTCGAGCCAA
ACGCTCATCAGTGATATTAATAAACTCCCCGGGCAGCTCACTTAAGTTCATGTCG
CTGTCCAGCTGCTGAGCCACAGGCTGCTGTCCAACTTGCGGTTGCTTAACGGGCG
GCGAAGGAGAAGTCCACGCCTACATGGGGGTAGAGTCATAATCGTGCATCAGGA
TAGGGCGGTGGTGCTGCAGCAGCGCGCGAATAAACTGCTGCCGCCGCCGCTCCG
TCCTGCAGGAATACAACATGGCAGTGGTCTCCTCAGCGATGATTCGCACCGCCCG
CAGCATAAGGCGCCTTGTCCTCCGGGCACAGCAGCGCACCCTGATCTCACTTAAA
TCAGCACAGTAACTGCAGCACAGCACCACAATATTGTTCAAAATCCCACAGTGC
AAGGCGCTGTATCCAAAGCTCATGGCGGGGACCACAGAACCCACGTGGCCATCA
TACCACAAGCGCAGGTAGATTAAGTGGCGACCCCTCATAAACACGCTGGACATA
AACATTACCTCTTTTGGCATGTTGTAATTCACCACCTCCCGGTACCATATAAACCT
CTGATTAAACATGGCGCCATCCACCACCATCCTAAACCAGCTGGCCAAAACCTGC
CCGCCGGCTATACACTGCAGGGAACCGGGACTGGAACAATGACAGTGGAGAGCC
CAGGACTCGTAACCATGGATCATCATGCTCGTCATGATATCAATGTTGGCACAAC
ACAGGCACACGTGCATACACTTCCTCAGGATTACAAGCTCCTCCCGCGTTAGAAC
CATATCCCAGGGAACAACCCATTCCTGAATCAGCGTAAATCCCACACTGCAGGG
AAGACCTCGCACGTAACTCACGTTGTGCATTGTCAAAGTGTTACATTCGGGCAGC
AGCGGATGATCCTCCAGTATGGTAGCGCGGGTTTCTGTCTCAAAAGGAGGTAGA
CGATCCCTACTGTACGGAGTGCGCCGAGACAACCGAGATCGTGTTGGTCGTAGT
GTCATGCCAAATGGAACGCCGGACGTAGTCATATTTCCTGAAGCAAAACCAGGT
GCGGGCGTGACAAACAGATCTGCGTCTCCGGTCTCGCCGCTTAGATCGCTCTGTG
TAGTAGTTGTAGTATATCCACTCTCTCAAAGCATCCAGGCGCCCCCTGGCTTCGG
GTTCTATGTAAACTCCTTCATGCGCCGCTGCCCTGATAACATCCACCACCGCAGA
ATAAGCCACACCCAGCCAACCTACACATTCGTTCTGCGAGTCACACACGGGAGG
AGCGGGAAGAGCTGGAAGAACCATGTTTTTTTTTTTATTCCAAAAGATTATCCAA
AACCTCAAAATGAAGATCTATTAAGTGAACGCGCTCCCCTCCGGTGGCGTGGTCA
AACTCTACAGCCAAAGAACAGATAATGGCATTTGTAAGATGTTGCACAATGGCT
TCCAAAAGGCAAACGGCCCTCACGTCCAAGTGGACGTAAAGGCTAAACCCTTCA
GGGTGAATCTCCTCTATAAACATTCCAGCACCTTCAACCATGCCCAAATAATTCT
CATCTCGCCACCTTCTCAATATATCTCTAAGCAAATCCCGAATATTAAGTCCGGC
CATTGTAAAAATCTGCTCCAGAGCGCCCTCCACCTTCAGCCTCAAGCAGCGAATC
ATGATTGCAAAAATTCAGGTTCCTCACAGACCTGTATAAGATTCAAAAGCGGAA
CATTAACAAAAATACCGCGATCCCGTAGGTCCCTTCGCAGGGCCAGCTGAACAT
AATCGTGCAGGTCTGCACGGACCAGCGCGGCCACTTCCCCGCCAGGAACCATGA
CAAAAGAACCCACACTGATTATGACACGCATACTCGGAGCTATGCTAACCAGCG
TAGCCCCGATGTAAGCTTGTTGCATGGGCGGCGATATAAAATGCAAGGTGCTGCT
CAAAAAATCAGGCAAAGCCTCGCGCAAAAAAGAAAGCACATCGTAGTCATGCTC
ATGCAGATAAAGGCAGGTAAGCTCCGGAACCACCACAGAAAAAGACACCATTTT
TCTCTCAAACATGTCTGCGGGTTTCTGCATAAACACAAAATAAAATAACAAAAA
AACATTTAAACATTAGAAGCCTGTCTTACAACAGGAAAAACAACCCTTATAAGC
ATAAGACGGACTACGGCCATGCCGGCGTGACCGTAAAAAAACTGGTCACCGTGA
TTAAAAAGCACCACCGACAGCTCCTCGGTCATGTCCGGAGTCATAATGTAAGACT
CGGTAAACACATCAGGTTGATTCACATCGGTCAGTGCTAAAAAGCGACCGAAAT
AGCCCGGGGGAATACATACCCGCAGGCGTAGAGACAACATTACAGCCCCCATAG
GAGGTATAACAAAATTAATAGGAGAGAAAAACACATAAACACCTGAAAAACCC
TCCTGCCTAGGCAAAATAGCACCCTCCCGCTCCAGAACAACATACAGCGCTTCCA
CAGCGGCAGCCATAACAGTCAGCCTTACCAGTAAAAAAGAAAACCTATTAAAAA
AACACCACTCGACACGGCACCAGCTCAATCAGTCACAGTGTAAAAAAGGGCCAA
GTGCAGAGCGAGTATATATAGGACTAAAAAATGACGTAACGGTTAAAGTCCACA
AAAAACACCCAGAAAACCGCACGCGAACCTACGCCCAGAAACGAAAGCCAAAA
AACCCACAACTTCCTCAAATCGTCACTTCCGTTTTCCCACGTTACGTCACTTCCCA
TTTTAAGAAAACTACAATTCCCAACACATACAAGTTACTCCGCCCTAAAACCTAC
GTCACCCGCCCCGTTCCCACGCCCCGCGCCACGTCACAAACTCCACCCCCTCATT
ATCATATTGGCTTCAATCCAAAATAAGGTATATT
SEQ ID NO: 12: Amino Sequence of S1-N
MFVFLVLLPLVSSQCVNLTTRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRDLPQGFSALEPLVDLP
IGINITRFQTLLALHRSYLTPGDSSSGWTAGAAAYYVGYLQPRTFLLKYNENGTITDA
VDCALDPLSETKCTLKSFTVEKGIYQTSNFRVQPTESIVRFPNITNLCPFGEVFNATRF
ASVYAWNRKRISNCVADYSVLYNSASFSTFKCYGVSPTKLNDLCFTNVYADSFVIRG
DEVRQIAPGQTGKIADYNYKLPDDFTGCVIAWNSNNLDSKVGGNYNYLYRLFRKSN
LKPFERDISTEIYQAGSTPCNGVEGFNCYFPLQSYGFQPTNGVGYQPYRVVVLSFELL
HAPATVCGPKKSTNLVKNKCVNFNFNGLTGTGVLTESNKKFLPFQQFGRDIADTTD
AVRDPQTLEILDITPCSFGGVSVITPGTNTSNQVAVLYQDVNCTEVPVAIHADQLTPT
WRVYSTGSNVFQTRAGCLIGAEHVNNSYECDIPIGAGICASYQTQTNSPRRARSVAS
QSIIAYTMMSDNGPQNQRNAPRITFGGPSDSTGSNQNGERSGARSKQRRPQGLPNNT
ASWFTALTQHGKEDLKFPRGQGVPINTNSSPDDQIGYYRRATRRIRGGDGKMKDLSP
RWYFYYLGTGPEAGLPYGANKDGIIWVATEGALNTPKDHIGTRNPANNAAIVLQLP
QGTTLPKGFYAEGSRGGSQASSRSSSRSRNSSRNSTPGSSRGTSPARMAGNGGDAAL
ALLLLDRLNQLESKMSGKGQQQQGQTVTKKSAAEASKKPRQKRTATKAYNVTQAF
GRRGPEQTQGNFGDQELIRQGTDYKHWPQIAQFAPSASAFFGMSRIGMEVTPSGTWL
TYTGAIKLDDKDPNFKDQVILLNKHIDAYKTFPPTEPKKDKKKKADETQALPQRQKK
QQTVTLLPAADLDDFSKQLQQSMSSADSTQA
SEQ ID NO: 13: TLR-3 agonist sequence
GAAACGATATGGGCTGAATACTTAAGTATTCAGCCCATATCGTTTC
SEQ ID NO: 14: TLR-3 agonist sequence
CGGGCCCCCCCTCGAGGTCGACGGTATCGATAAGCTTGATATCGAATTCGCCCTT
AGATATCGTCGACGCCCAGCACCCCAAGGCGGCCAACGCCAAAACTCTCCCTCC
TCCTCTTCCTCAATCTCGCTCTCGCTCTTTTTTTTTTTCGCAAAAGGAGGGGAGAG
GGGGTAAAAAAATGCTGCACTGTGCGGCGAAGCCGGTGAGTGAGCGGCGCGGG
GCCAATCAGCGTGCGCCGTTCCGAAAGTTGCCTTTTATGGCTCGAGCGGCCGCGG
CGGCGCCCTATAAAACCCAGCGGCGCGACGCGCCACCACCGCCGAGACATCGAT
GATATCTAAAGGGCGAATTCCTGCAGCCCGGGGGATCCACTAGTCTAGATGCAT
GCTCGAGCGGCCGCCAGTGTGATGGATATCTGCAGAATTCGCCCTTCAGCTGCGG
ATCCATTCGCCATTCAGGCTGCGCAACTGTTGGGAAGGGCGATCGGTGCGGGCCT
CTTCGCTATTACGCCAGCTGGCGAAAGGGGGATGTGCTGCAAGGCGATTAAGTT
GGGTAACGCCAGGGTTTTCCCAGTCACGACGTTGTAAAACGACGGCCAGTGAAT
TGTAATACGACTCACTATAGGGCGAATTGGGTACCGGGCCCCCCCTCGAGGTCG
ACGGTATCGATAAGCTTGATATCGAATTCCTGCAGCCCGGGGGATCCACTAGTTT
CTAGAAATAAAATATCTTTATTTTCATTACATCTGTGTGTTGGTTTTTTGTGTGGC
GGCCGCCACCGCGGTGGAGCTATCGAATTCAAGCTTGTCGACTCGAAGATCCTA
GACTAGTGGATCCCCCGGGCTGCAGGAATTCGCCCTTTAGATATCATCGATGTCT
CGGCGGTGGTGGCGCGTCGCGCCGCTGGGTTTTATAGGGCGCCGCCGCGGCCGC
TCGAGCCATAAAAGGCAACTTTCGGAACGGCGCACGCTGATTGGCCCCGCGCCG
CTCACTCACCGGCTTCGCCGCACAGTGCAGCATTTTTTTACCCCCTCTCCCCTCCT
TTTGCGAAAAAAAAAAAGAGCGAGAGCGAGATTGAGGAAGAGGAGGAGGGAGA
GTTTTGGCGTTGGCCGCCTTGGGGTGCTGGGCGTCGACGATATCTAAGGGCGAAT
TCGATATCAAGCTTATCGATACCGTCGACCTCGAGGGGGGGCCCG
SEQ ID NO: 15: TLR-3 agonist sequence
CGGGCCCCCCCTCGAGGTCGACGGTATCGATAAGCTTGATATCGAATTCGCCCTT
AGATATCGTCGACGCCCAGCACCCCAAGGCGGCCAACGCCAAAACTCTCCCTCC
TCCTCTTCCTCAATCTCGCTCTCGCTCTTTTTTTTTTTCGCAAAAGGAGGGGAGAG
GGGGTAAAAAAATGCTGCACTGTGCGGCGAAGCCGGTGAGTGAGCGGCGCGGG
GCCAATCAGCGTGCGCCGTTCCGAAAGTTGCCTTTTATGGCTCGAGCGGCCGCGG
CGGCGCCCTATAAAACCCAGCGGCGCGACGCGCCACCACCGCCGAGACATCGAT
GATATCTAAAGGGCGAATTCCTGCAGCCCGGGGGATCCACTAGTCTAGATGCAT
GCTCGAGCGGCCGCCAGTGTGATGGATATCTGCAGAATTCGCCCTTCAGCTGCGG
ATCCATTCGCCATTCAGGCTGCGCAACTGTTGGGAAGGGCGATCGGTGCGGGCCT
CTTCGCTATTACGCCAGCTGGCGAAAGGGGGATGTGCTGCAAGGCGATTAAGTT
GGGTAACGCCAGGGTTTTCCCAGTCACGACGTTGTAAAACGACGGCCAGTGAAT
TGTAATACGACTCACTATAGGGCGAATTGGGTACCGGGCCCCCCCTCGAGGTCG
ACGGTATCGATAAGCTTGATATCGAATTCCTGCAGCCCGGGGGATCCACTAGTTT
CTAGAAATAAAATATCTTTATTTTCATTACATCTGTGTGTTGGTTTTTTGTGTGGC
GGCCGCCACCGCGGTGGAGCTATCGAATTCAAGCTTGTCGACTCGAAGATCGTA
CACAGGAAGTGACAATTTTCGCGCGGTTTTAGGCGGATGTTGTAGTAAATTTGGG
CGTAACCGAGTAAGATTTGGCCATTTTCGCGGGAAAACTGAATAAGAGGAAGTG
AAATCTGAATAATTTTGTGTTACTCATAGCGCGTAATACTGGTACCGGGCCCCCC
CTCGAGGTCGACGGTATCGATAAGCTTGATATCGAATTCGCCCTTAGATATCGTC
GACGCCCAGCACCCCAAGGCGGCCAACGCCAAAACTCTCCCTCCTCCTCTTCCTC
AATCTCGCTCTCGCTCTTTTTTTTTTTCGCAAAAGGAGGGGAGAGGGGGTAAAAA
AATGCTGCACTGTGCGGCGAAGCCGGTGAGTGAGCGGCGCGGGGCCAATCAGCG
TGCGCCGTTCCGAAAGTTGCCTTTTATGGCTCGAGCGGCCGCGGCGGCGCCCTAT
AAAACCCAGCGGCGCGACGCGCCACCACCGCCGAGACATCGATGATATCTAAAG
GGCGAATTCCTGCAGCCCGGGGGATCCACTAGTCTAGAACTAGTGGATCCCCCG
GGCTGCAGGAATTCGATATCAAGCTTATCGATACCGTCGACCTCGAGGGGGGGC
CCGGTACCCAATTCGCCCTATAGTGAGTCGTATTACAATTCACTGGCCGTCGTTTT
ACAACGTCGTGACTGGGAAAACCCTGGCGTTACCCAACTTAATCGCCTTGCAGCA
CATCCCCCTTTCGCCAGCTGGCGTAATAGCGAAGAGGCCCGCACCGATCGCCCTT
CCCAACAGTTGCGCAGCCTGAATGGCGAATGGATCCGCAGCTGAAGGGCGAATT
CTGCAGATATCCATCACACTGGCGGCCGCTCGAGCATGCATCTAGAAATAAAAT
ATCTTTATTTTCATTACATCTGTGTGTTGGTTTTTTGTGTGGCGGCCGCCACCGCG
GTGGAGCTA
SEQ ID NO: 16: TLR-3 agonist sequence
GATGGTGCTTCAAGCTAGTACTTAAGTACTAGCTTGAAGCACCATC
SEQ ID NO: 17: TLR-3 agonist sequence
GATGGTGCTTCAAGCTAGTACGGATCCGTACTAGCTTGAAGCACCATC
SEQ ID NO: 18: TLR-3 agonist sequence
GAAACGATATGGGCTGAATACGGATCCGTATTCAGCCCATATCGTTTC
SEQ ID NO: 19: TLR-3 agonist sequence
CCTAATAATTATCAAAATGTGGATCCACATTTTGATAATTATTAGG
SEQ ID NO: 20: TLR-3 agonist sequence
CCTAATAATTATCAAAATGTAATTACATTTTGATAATTATTAGG
SEQ ID NO: 21
UK B.1.1.7 S Protein Variant
GISAID Accession #EPI_ISL_601443
MFVFLVLLPLVSSQCVNLTTRTQLPPAYTNSFTRGVYYPDKVFRSSVLHSTQDLFLPF
FSNVTWFHAISGTNGTKRFDNPVLPFNDGVYFASTEKSNIIRGWIFGTTLDSKTQSLLI
VNNATNVVIKVCEFQFCNDPFLGVYHKNNKSWMESEFRVYSSANNCTFEYVSQPFL
MDLEGKQGNFKNLREFVFKNIDGYFKIYSKHTPINLVRDLPQGFSALEPLVDLPIGINI
TRFQTLLALHRSYLTPGDSSSGWTAGAAAYYVGYLQPRTFLLKYNENGTITDAVDC
ALDPLSETKCTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSFVIRGDEV
RQIAPGQTGKIADYNYKLPDDFTGCVIAWNSNNLDSKVGGNYNYLYRLFRKSNLKP
FERDISTEIYQAGSTPCNGVEGFNCYFPLQSYGFQPTYGVGYQPYRVVVLSFELLHAP
ATVCGPKKSTNLVKNKCVNFNFNGLTGTGVLTESNKKFLPFQQFGRDIDDTTDAVR
DPQTLEILDITPCSFGGVSVITPGTNTSNQVAVLYQGVNCTEVPVAIHADQLTPTWRV
YSTGSNVFQTRAGCLIGAEHVNNSYECDIPIGAGICASYQTQTNSHRRARSVASQSIIA
YTMSLGAENSVAYSNNSIAIPINFTISVTTEILPVSMTKTSVDCTMYICGDSTECSNLLL
QYGSFCTQLNRALTGIAVEQDKNTQEVFAQVKQIYKTPPIKDFGGFNFSQILPDPSKP
SKRSFIEDLLFNKVTLADAGFIKQYGDCLGDIAARDLICAQKFNGLTVLTPLLTDEMI
AQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILARL
DKVEAEVQIDRLITGRLQSLQTYVTQQLIRAAEIRASANLAATKMSECVLGQSKRVD
FCGKGYHLMSFPQSAPHGVVFLHVTYVPAQEKNFTTAPAICHDGKAHFPREGVFVS
NGTHWFVTQRNFYEPQIITTHNTFVSGNCDVVIGIVNNTVYDPLQPELDSFKEELDKY
FKNHTSPDVDLGDISGINASVVNIQKEIDRLNEVAKNLNESLIDLQELGKYEQYIKWP
WYIWLGFIAGLIAIVMVTIMLCCMTSCCSCLKGCCSCGSCCKFDEDDSEPVLKGVKL
HYT*
SEQ ID NO: 22
S. African B.1.351 501Y.V2 S Protein Variant
GISAID Accession #EPI_ISL_678597
MFVFLVLLPLVSSQCVNLTTRTQLPPAYTNSFTRGVYYPDKVFRSSVLHSTQDLFLPF
FSNVTWFHAIHVSGTNGTKRFANPVLPFNDGVYFASTEKSNIIRGWIFGTTLDSKTQS
LLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRGLPQGFSALEPLVDLP
IGINITRFQTLHRSYLTPGDSSSGWTAGAAAYYVGYLQPRTFLLKYNENGTITDAVDC
ALDPLSETKCTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSFVIRGDEV
RQIAPGQTGNIADYNYKLPDDFTGCVIAWNSNNLDSKVGGNYNYLYRIFRKSNLKP
FERDISTEIYQAGSTPCNGVKGFNCYFPLQSYGFQPTYGVGYQPYRVVVLSFELLHAP
ATVCGPKKSTNLVKNKCVNFNFNGLTGTGVLTESNKKFLPFQQFGRDIADTTDAVR
DPQTLEILDITPCSFGGVSVITPGTNTSNQVAVLYQGVNCTEVPVAIHADQLTPTWRV
YSTGSNVFQTRAGCLIGAEHVNNSYECDIPIGAGICASYQTQTNSPRRARSVASQSIIA
YTMSLGVENSVAYSNNSIAIPTNFTISVTTEILPVSMTKTSVDCTMYICGDSTECSNLL
LQYGSFCTQLNRALTGIAVEQDKNTQEVFAQVKQIYKTPPIKDFGGFNFSQILPDPSK
PSKRSFIEDLLFNKVTLADAGFIKQYGDCLGDIAARDLICAQKFNGLTVLPPLLTDEMI
AQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRL
DKVEAEVQIDRLITGRLQSLQTYVTQQLIRAAEIRASANLAATKMSECVLGQSKRVD
FCGKGYHLMSFPQSAPHGVVFLHVTYVPAQEKNFTTAPAICHDGKAHFPREGVFVS
NGTHWFVTQRNFYEPQIITTDNTFVSGNCDVVIGIVNNTVYDPLQPELDSFKEELDKY
FKNHTSPDVDLGDISGINASVVNIQKEIDRLNEVAKNLNESLIDLQELGKYEQYIKWP
WYIWLGFIAGLIAIVMVTIMLCCMTSCCSCLKGCCSCGSCCKFDEDDSEPVLKGVKL
HYT*
SEQ ID NO: 23
UK B.1.1.7 S protein variant GISAID accession #epi_isl_601443, human codon-optimized
nucleic acid sequence:
ATGTTTGTTTTCTTGGTGCTCCTTCCACTGGTGTCCTCCCAGTGTGTGAACCTCAC
CACTCGCACGCAACTGCCCCCAGCCTACACGAACTCTTTCACCCGAGGGGTATAT
TACCCTGATAAAGTCTTCAGGTCCTCTGTTCTCCATAGCACTCAGGATCTTTTCCT
GCCTTTCTTTAGCAACGTAACTTGGTTTCACGCAATTTCAGGCACGAACGGCACG
AAGAGATTTGATAACCCAGTGCTCCCTTTCAATGACGGCGTTTACTTCGCTTCTA
CCGAGAAAAGCAACATCATCAGGGGATGGATTTTCGGGACAACATTGGATTCAA
AGACCCAGAGCCTTCTCATTGTGAATAATGCCACGAACGTGGTAATCAAGGTGT
GCGAGTTCCAGTTCTGCAACGACCCATTCCTTGGAGTTTACCACAAAAACAATAA
GAGCTGGATGGAGAGCGAGTTTAGAGTGTATAGCAGCGCCAACAACTGCACCTT
TGAGTACGTTAGCCAACCCTTTCTGATGGACCTGGAGGGCAAACAGGGAAACTT
TAAGAACCTCCGCGAATTCGTTTTCAAGAACATTGATGGGTATTTTAAGATATAC
TCTAAGCATACCCCTATTAACTTGGTGCGAGATCTTCCTCAGGGGTTCAGCGCGC
TCGAACCTCTCGTGGACTTGCCTATTGGTATCAACATCACCCGATTCCAGACCCT
GCTGGCTCTGCACAGGTCATACTTGACTCCCGGCGATTCATCCAGCGGATGGACT
GCGGGTGCCGCCGCATATTATGTGGGCTACTTGCAGCCACGAACCTTTCTTTTGA
AATATAACGAGAATGGCACAATCACCGACGCCGTTGATTGCGCCCTGGATCCCCT
TTCCGAGACGAAGTGTACGCTGAAGTCTTTCACAGTGGAAAAGGGAATCTACCA
GACATCAAACTTCCGCGTCCAACCTACCGAGTCAATAGTGCGCTTCCCAAATATC
ACCAATCTCTGCCCCTTTGGGGAAGTGTTTAATGCCACCCGGTTCGCTTCTGTCTA
TGCCTGGAACAGGAAGCGCATTTCAAACTGCGTTGCTGACTATTCCGTGCTGTAT
AACTCTGCAAGCTTTTCTACCTTTAAGTGCTATGGTGTTAGTCCGACAAAACTGA
ATGATCTGTGCTTTACCAACGTTTACGCCGACTCATTCGTGATTCGAGGAGATGA
GGTCAGACAAATTGCTCCTGGGCAGACCGGCAAAATCGCCGACTACAACTATAA
GTTGCCTGATGACTTCACCGGCTGCGTGATTGCCTGGAACTCTAACAACCTTGAT
TCTAAAGTCGGAGGGAACTATAATTACCTCTATCGCCTCTTTAGAAAGTCTAATC
TGAAGCCGTTTGAGAGAGATATCTCTACGGAAATATACCAGGCCGGATCAACTC
CTTGTAACGGCGTAGAGGGCTTCAACTGCTATTTTCCACTGCAATCCTACGGGTT
CCAACCTACTTACGGAGTGGGCTATCAACCCTACAGGGTTGTGGTGCTGTCATTT
GAGCTGCTCCACGCACCTGCTACCGTGTGCGGTCCCAAGAAGTCAACCAACCTG
GTCAAGAACAAGTGCGTGAATTTCAATTTTAACGGTCTGACCGGAACAGGGGTG
CTCACAGAGTCAAACAAGAAGTTTCTGCCCTTCCAGCAGTTCGGGCGCGATATTG
ATGACACTACCGACGCAGTTCGCGACCCTCAGACTCTTGAGATTCTTGATATCAC
TCCCTGCAGCTTTGGGGGGGTTTCAGTCATCACCCCAGGCACTAACACATCCAAT
CAAGTGGCCGTGCTGTATCAGGGAGTCAATTGCACCGAGGTCCCAGTGGCAATC
CACGCGGATCAACTCACACCAACATGGAGAGTGTACAGCACCGGGTCTAATGTG
TTCCAAACTAGAGCCGGTTGTCTCATTGGCGCAGAACACGTGAACAACAGCTAC
GAGTGCGACATTCCAATTGGAGCCGGAATCTGTGCCTCTTACCAAACACAGACC
AACTCCCACAGACGGGCTCGATCTGTGGCCTCCCAAAGCATTATCGCTTACACGA
TGAGTCTGGGTGCAGAGAATAGCGTTGCCTACAGTAATAATTCTATCGCTATACC
TATCAATTTCACCATCTCTGTAACCACTGAGATCCTTCCAGTCAGTATGACGAAG
ACTTCTGTTGACTGCACAATGTATATATGCGGAGATTCCACAGAGTGCAGTAACC
TGCTGCTGCAATATGGCTCCTTTTGCACTCAGCTCAATAGGGCCCTTACAGGAAT
CGCCGTGGAGCAAGACAAGAATACTCAAGAAGTCTTCGCGCAGGTGAAGCAGAT
CTACAAAACGCCTCCTATAAAAGACTTTGGCGGGTTCAATTTTTCCCAGATCTTG
CCTGATCCCTCAAAACCCTCTAAGAGATCCTTCATCGAGGATCTTCTGTTTAATA
AGGTCACCCTGGCAGACGCAGGCTTCATTAAGCAGTACGGAGACTGCCTCGGGG
ACATCGCTGCAAGAGACCTTATTTGTGCCCAGAAGTTTAATGGACTCACCGTACT
TCCACCACTGCTCACAGATGAGATGATTGCACAGTACACCTCTGCCCTGCTTGCC
GGCACTATCACCAGCGGCTGGACTTTCGGAGCCGGAGCTGCCCTTCAGATCCCTT
TCGCCATGCAGATGGCATATAGATTCAACGGGATCGGAGTCACCCAGAACGTGC
TTTACGAAAATCAGAAACTGATCGCGAACCAATTCAACAGTGCCATCGGCAAGA
TCCAGGATAGCCTCTCATCCACTGCGAGTGCGTTGGGGAAACTGCAAGATGTGGT
CAATCAGAATGCGCAGGCCCTCAACACGCTGGTGAAGCAGCTCTCCTCTAATTTC
GGGGCTATCAGCTCTGTTCTGAACGATATCTTGGCTAGACTGGATAAGGTGGAGG
CTGAAGTTCAGATTGATAGATTGATTACTGGCCGGCTGCAGTCACTCCAGACTTA
TGTTACGCAGCAGTTGATTCGCGCTGCGGAGATACGGGCCTCAGCTAATCTTGCC
GCTACTAAGATGTCCGAGTGTGTGTTGGGGCAATCCAAACGCGTGGATTTCTGCG
GCAAAGGTTACCATCTTATGTCATTCCCCCAGAGCGCCCCTCACGGAGTGGTTTT
TCTCCATGTGACATATGTCCCAGCCCAGGAAAAAAATTTTACCACAGCCCCAGCT
ATATGCCACGACGGCAAAGCTCACTTTCCTCGCGAGGGGGTCTTCGTATCCAACG
GCACACACTGGTTTGTAACCCAGAGGAATTTCTACGAACCGCAGATCATCACAA
CTCATAACACGTTTGTTTCCGGTAATTGTGATGTAGTAATCGGCATCGTTAATAAT
ACAGTGTATGATCCTCTTCAACCCGAACTGGATAGCTTCAAGGAGGAACTCGATA
AGTACTTCAAGAATCACACTTCTCCTGACGTGGACCTTGGTGATATATCCGGCAT
AAATGCTAGTGTGGTGAACATCCAAAAAGAGATAGACAGGCTCAATGAGGTTGC
TAAGAATTTGAACGAATCTCTTATCGACCTCCAGGAGCTCGGCAAGTACGAGCA
GTATATTAAGTGGCCTTGGTACATCTGGCTGGGTTTCATCGCTGGCTTGATAGCA
ATCGTAATGGTCACCATTATGTTGTGCTGCATGACTTCCTGTTGTAGCTGTCTCAA
AGGGTGTTGCAGCTGTGGCTCATGCTGCAAATTTGACGAAGATGACTCTGAACCA
GTCCTCAAGGGCGTCAAGCTTCACTACACGTGA

### ADDITIONAL REFERENCES

1. Aylward B, Liang W. 2020. Report of the WHO-China Joint Mission on Coronavirus Disease 2019 (COVID-19). WHO Report.
2. Yeo C, Kaushal S, Yeo D. 2020. Enteric involvement of coronaviruses: is faecal-oral transmission of SARS-CoV-2 possible? Lancet Gastroenterol Hepatol doi:10.1016/S2468-1253(20)30048-0.
3. Han C, Duan C, Zhang S, Spiegel B, Shi H, Wang W, Zhang L, Lin R, Liu J, Ding Z, Hou X. 2020. Digestive Symptoms in COVID-19 Patients with Mild Disease Severity: Clinical Presentation, Stool Viral RNA Testing, and Outcomes. American Journal of Gastroenterology.
4. Kam Y W, Kien F, Roberts A, Cheung Y C, Lamirande E W, Vogel L, Chu S L, Tse J, Guarner J, Zaki S R, Subbarao K, Peiris M, Nal B, Altmeyer R. 2007. Antibodies against trimeric S glycoprotein protect hamsters against SARS-CoV challenge despite their capacity to mediate FcgammaRII-dependent entry into B cells in vitro. Vaccine 25:729-40.
5. Wang Q, Zhang L, Kuwahara K, Li L, Liu Z, Li T, Zhu H, Liu J, Xu Y, Xie J, Morioka H, Sakaguchi N, Qin C, Liu G. 2016. Immunodominant SARS Coronavirus Epitopes in Humans Elicited both Enhancing and Neutralizing Effects on Infection in Non-human Primates. ACS Infect Dis 2:361-76.
6. Ji W, Wang W, Zhao X, Zai J, Li X. 2020. Cross-species transmission of the newly identified coronavirus 2019-nCoV. J Med Virol 92:433-440.
7. Kumar S, Maurya V K, Prasad A K, Bhatt M L B, Saxena S K. 2020. Structural, glycosylation and antigenic variation between 2019 novel coronavirus (2019-nCoV) and SARS coronavirus (SARS-CoV). Virusdisease 31:13-21.
8. Wan Y, Shang J, Graham R, Baric R S, Li F. 2020. Receptor Recognition by the Novel Coronavirus from Wuhan: an Analysis Based on Decade-Long Structural Studies of SARS Coronavirus. J Virol 94.
9. Xu H, Zhong L, Deng J, Peng J, Dan H, Zeng X, Li T, Chen Q. 2020. High expression of ACE2 receptor of 2019-nCoV on the epithelial cells of oral mucosa. Int J Oral Sci 12:8.
10. Okba N M, Raj V S, Haagmans B L. 2017. Middle East respiratory syndrome coronavirus vaccines: current status and novel approaches. Curr Opin Virol 23:49-58.
11. Wang L, Shi W, Joyce M G, Modjarrad K, Zhang Y, Leung K, Lees C R, Zhou T, Yassine H M, Kanekiyo M, Yang Z Y, Chen X, Becker M M, Freeman M, Vogel L, Johnson J C, Olinger G, Todd J P, Bagci U, Solomon J, Mollura D J, Hensley L, Jahrling P, Denison M R, Rao S S, Subbarao K, Kwong P D, Mascola J R, Kong W P, Graham B S. 2015. Evaluation of candidate vaccine approaches for MERS-CoV. Nat Commun 6:7712.
12. Adney D R, Wang L, van Doremalen N, Shi W, Zhang Y, Kong W P, Miller M R, Bushmaker T, Scott D, de Wit E, Modjarrad K, Petrovsky N, Graham B S, Bowen R A, Munster V J. 2019. Efficacy of an Adjuvanted Middle East Respiratory Syndrome Coronavirus Spike Protein Vaccine in Dromedary Camels and Alpacas. Viruses 11.
13. Haagmans B L, van den Brand J M, Raj V S, Volz A, Wohlsein P, Smits S L, Schipper D, Bestebroer T M, Okba N, Fux R, Bensaid A, Solanes Foz D, Kuiken T, Baumgartner W, Segales J, Sutter G, Osterhaus A D. 2016. An orthopoxvirus-based vaccine reduces virus excretion after MERS-CoV infection in dromedary camels. Science 351:77-81.
14. Liebowitz D, Gottlieb K, Kolhatkar N S, Garg S J, Asher J M, Nazareno J, Kim K, McIIwain DR, Tucker S N. 2020. Efficacy and immune correlates of protection induced by an oral influenza vaccine evaluated in a phase 2, placebo-controlled human experimental infection study. Lancet Infect Dis 20:435-444.
15. Kim L, Liebowitz D, Lin K, Kasparek K, Pasetti M F, Garg S J, Gottlieb K, Trager G, Tucker S N. 2018. Safety and immunogenicity of an oral tablet norovirus vaccine, a phase I randomized, placebo-controlled trial. JCI Insight 3:e121077.
16. Kim L, Martinez C J, Hodgson K A, Trager G R, Brandl J R, Sandefer E P, Doll W J, Liebowitz D, Tucker S N. 2016. Systemic and mucosal immune responses following oral adenoviral delivery of influenza vaccine to the human intestine by radio controlled capsule. Scientific Reports 6:37295.
17. He T C, Zhou S, da Costa L T, Yu J, Kinzler K W, Vogelstein B. 1998. A simplified system for generating recombinant adenoviruses. Proc Natl Acad Sci USA 95:2509-14.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A chimeric adenoviral expression vector, comprising an expression cassette comprising the following elements:
(a) a first promoter operably linked to a nucleic acid encoding a first severe acute respiratory syndrome coronavirus 2 (SARS-CoV-2) protein;
(b) a second promoter operably linked to a nucleic acid encoding a toll-like receptor-3 (TLR-3) agonist; and
(c) a third promoter operably linked to a nucleic acid encoding a SARS-CoV-2 N protein.

2. The chimeric adenoviral expression vector of claim 1, wherein:
the SARS-CoV-2 N protein comprises an amino acid sequence having at least 95%, 96%, 97%, 98%, 99%, or 100% identity to the amino acid sequence of SEQ ID NO:2; and/or
the SARS-CoV-2 protein of (a) comprises a SARS-CoV-2 S protein having a sequence with at least 95%, 96%, 97%, 98%, 99%, or 100% identity to the sequence of SEO ID NO:1 or SEQ ID NO: 21 or SEQ ID NO:22; and/or
the nucleic acid encoding the TLR-3 agonist comprises a nucleic acid encoding a dsRNA, optionally wherein the nucleic acid encoding the TLR-3 agonist comprises a sequence selected from the group consisting of: SEQ ID NOS:13-20.

3. The chimeric adenoviral expression vector of claim 1, wherein element (c) is situated between elements (a) and (b) in the expression cassette.

4-6. (canceled)

7. The chimeric adenoviral expression vector of claim 1, wherein the nucleic acid encoding the first SARS-CoV-2 protein in element (a) comprises a sequence having at least 85%, 90%, 95%, 97%, 99%, or 100% identity to the sequence of SEQ ID NO:3.

8. The chimeric adenoviral expression vector of claim 1, wherein the nucleic acid encoding the SARS-CoV-2 N protein comprises a sequence having at least 85%, 90%, 95%, 97%, 99%, or 100% identity to the sequence of SEQ ID NO:4.

9. The chimeric adenoviral expression vector claim 1, wherein the first promoter and the second promoter are identical.

10. The chimeric adenoviral expression vector of claim 9, wherein the first promoter and the second promoter are each a CMV promoter.

11. The chimeric adenoviral expression vector claim 10, wherein the first promoter is a CMV promoter, the second promoter is a CMV promoter, and the third promoter is a beta-actin promoter.

12. The chimeric adenoviral expression vector of claim 1, wherein element (c) is situated between elements (a) and (b), and elements (a), (c), and (b) together are encoded by a sequence at least 85%, at least 90%, or at least 95% identical to SEQ ID NO:7 or is encoded by the sequence of SEQ ID NO:7.

13. The chimeric adenoviral expression vector of claim 1, wherein the chimeric adenoviral expression vector comprises a sequence at least 85%, at least 90%, or at least 95% identical to SEQ ID NO:10 or comprises the sequence of SEQ ID NO:10.

14. A chimeric adenoviral expression vector, comprising an expression cassette comprising the following elements:
(a) a first promoter operably linked to a nucleic acid encoding a fusion protein comprising an S1 region of a SARS-CoV-2 S protein, a furin site, and a SARS-CoV-2 N protein; and
(b) a second promoter operably linked to a nucleic acid encoding a toll-like receptor-3 (TLR-3) agonist.

15. The chimeric adenoviral expression vector of claim 14, wherein the fusion protein comprises a sequence having at least 95%, 96%, 97%, 98%, 99%, or 100% identity to the sequence of SEQ ID NO:12.

16. The chimeric adenoviral expression vector of claim 14, wherein the nucleic acid encoding the SARS-CoV-2 fusion protein is at least 85%, at least 90%, or at least 95% identical to SEQ ID NO:5 or comprises SEQ ID NO:5.

17-18. (canceled)

19. The chimeric adenoviral expression vector claim 14, wherein elements (a) and (b) together are encoded by the sequence of SEQ ID NO:8 or a sequence at least 85%, at least 90%, or at least 95% identical to SEQ ID NO:8; optionally wherein the chimeric adenoviral expression vector is encoded by the sequence of SEQ ID NO:11 or a sequence at least 85%, at least 90%, or at least 95% identical to SEQ ID NO:11.

20. (canceled)

21. A chimeric adenoviral expression vector, comprising an expression cassette comprising the following elements:
(a) a first promoter operably linked to a nucleic acid encoding a SARS-CoV-2 S protein; and
(b) a second promoter operably linked to a nucleic acid encoding a toll-like receptor-3 (TLR-3) agonist.

22. The chimeric adenoviral expression vector of claim 21, wherein:
the SARS-CoV-2 S protein has at least 95%, 96%, 97%, 98%, or 99% identity to any one of SEQ ID NOS: 1, 21, or 22, or comprises the SARS-CoV-2 S protein sequence of SEO ID NO:1 or SEQ ID NO:21 or SEQ ID NO:22; optionally wherein the nucleic acid encoding the SARS-CoV-2 S protein is at least 85%, 90%, or 95% identical to the polynucleotide sequence of SEQ ID NO:3 or comprises the sequence of SEQ ID NO:3.

23-26. (canceled)

27. The chimeric adenoviral expression vector claim 21, wherein elements (a) and (b) together are encoded by the sequence of SEQ ID NO:6 or a sequence having at least 85%, at least 90%, or at least 95% identity to SEQ ID NO:6, optionally wherein the chimeric adenoviral expression vector is encoded by a sequence having at least 85%, at least 90%, or at least 95% identity to SEQ ID NO:9 or is encoded by the sequence of SEQ ID NO:9.

28. (canceled)

29. An immunogenic composition comprising the chimeric adenoviral expression vector of claim 1 and a pharmaceutically acceptable carrier.

30. A method for eliciting an immune response towards a SARS-CoV-2 protein in a subject, comprising administering to the subject an immunogenically effective amount of the chimeric adenoviral expression vector of claim 1 to a mammalian subject optionally wherein the route of administration is oral, intranasal, or mucosal.

31-33. (canceled)

34. The method of claim 30, wherein the subject is a human.

35. A chimeric polynucleotide, comprising an expression cassette comprising the following elements:
(a) a first promoter operably linked to a nucleic acid encoding an antigenic protein;
(b) a second promoter operably linked to a nucleic acid encoding a toll-like receptor-3 (TLR-3) agonist; and
(c) a third promoter operably linked to a nucleic acid encoding a SARS-CoV-2 N-protein.

36. The chimeric polynucleotide of claim 35, optionally wherein:
the SARS-CoV-2 N protein has at least 95%, 96%, 97%, 98%, 99%, or 100% identity to SEQ ID NO:2; and/or
the nucleic acid encoding the TLR-3 agonist comprises a nucleic acid encoding a dsRNA, optionally wherein the nucleic acid encoding the TLR-3 agonist comprises a sequence selected from the group consisting of: SEQ ID NOS:13-20.

37-39. (canceled)

40. The chimeric polynucleotide of claim 35, wherein element (c) is situated between elements (a) and (b) in the expression cassette.

41. The method of claim 35, wherein the antigenic protein is from a bacteria, fungus, virus, or parasite: or the antigenic protein is a cancer antigen.

42. (canceled)

43. A method of inducing an immune response in a subject, the method comprising administering a chimeric polynucleotide of claim 35 to the subject.

44-61. (canceled)


SEQUENCE LISTING (24 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 419 aa, Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 3 - DNA, 3840 bp, Severe acute respiratory syndrome coronavirus 2
ggtaccgccaccatgtttgtttttctcgtactcctgcccctggtttcctcccaatgtgtcaatctgactacccggacccaacttcctcccgcctacaccaattcctttacccgaggtgtttactacccagacaaagtgttcaggtcatccgtcctccatagtacccaagacctcttcctcccttttttttctaacgttacctggtttcacgctattcacgttagcggcaccaacggcaccaaaagattcgataaccccgtactgccgttcaacgacggggtatattttgcctctactgaaaaatcaaacatcatacgcggatggatctttgggactaccctggactcaaaaactcagtccctgctgattgtgaataacgctaccaacgtggtgatcaaagtctgtgaattccagttttgcaacgatccttttctcggcgtttattatcacaaaaataacaaatcctggatggagagcgagttccgggtgtactcctccgcgaataattgcaccttcgaatatgtgtctcagccattcctcatggacctcgaggggaagcagggcaattttaagaatctgcgagaattcgtgttcaagaatatagacggttacttcaagatttactccaaacacaccccgattaacctggttagggacttgcctcagggcttttctgcattggagcccctcgtggacctcccaatcggcataaacattacaagatttcagactttgcttgcattgcacaggagctatttgacacccggcgattcttcttccggatggaccgctggagcagctgcttattacgtgggctatctgcagcctcgaacctttcttttgaagtacaacgaaaatggaactatcaccgatgcagttgactgcgccctggaccccctgtccgaaactaagtgcacgctcaaaagtttcacagtagagaaggggatataccagactagcaatttccgcgttcagccaaccgaaagtatagtgcgctttcctaatataactaacctgtgtcctttcggggaagtgtttaacgccactagattcgcttccgtctacgcctggaatagaaagaggatctcaaattgcgttgctgactatagtgttttgtacaattccgcctctttctcaaccttcaaatgttacggggtgagccctaccaaactgaacgacctgtgctttacaaacgtatacgccgacagctttgttatcagaggagacgaggttcgccagattgctccgggtcagacaggcaagattgctgattataattacaaactgcccgacgactttacaggatgtgtgatcgcgtggaacagtaacaatcttgactcaaaggttgggggtaattataattatctttaccggctgttcagaaaaagcaatttgaaacccttcgaaagggacatatccaccgagatctatcaggccgggtccactccatgcaatggtgtggaaggttttaattgctacttcccattgcagtcttatggattccaaccaaccaatggcgtaggctaccagccgtatcgcgttgtcgtgctcagcttcgagctgctccacgcccccgcgaccgtatgcggtcctaagaagtccaccaatcttgttaagaacaagtgtgtaaactttaactttaacgggctgaccgggaccggcgttctgactgaatctaacaaaaaattcctgcctttccagcagttcggccgcgatattgctgacaccactgacgctgtaagagaccctcagacccttgaaattctcgatatcacaccttgcagctttgggggcgtgtccgtcatcactccaggaactaacacaagcaaccaggtggcagtgttgtaccaggatgttaattgtaccgaggtgccagtggccatccacgccgatcaattgacacctacctggagggtttacagcacagggtccaatgtttttcagacaagagccggatgtctgatcggtgccgagcatgtcaacaattcctacgagtgtgatatccccattggtgcgggaatttgtgcatcatatcagacccagactaatagcccaagaagagctagatccgtcgctagtcaatccatcattgcatatacaatgtccctgggagctgagaattcagtcgcgtattcaaacaattccattgctattcctactaatttcactatctccgtcacgaccgagatcctgccagtttccatgactaagacttctgttgactgcaccatgtatatctgtggcgatagcaccgagtgcagtaatctgcttctgcagtacggctccttctgcacacaactcaatcgagcactgaccggtattgcagttgagcaggacaagaacacacaggaggtctttgcacaggtcaaacaaatttacaaaaccccccccataaaagactttggtgggttcaacttcagccaaatcctcccagatcccagcaagccctccaaaagatccttcatcgaagaccttttgttcaataaggtaaccctggccgacgcaggcttcatcaaacaatatggcgattgccttggagacattgctgcgcgcgatttgatctgtgctcagaaatttaacggtttgaccgtgctgcccccacttctgactgatgagatgatagcacagtatacttctgctcttctggcaggaacaatcacttccgggtggacctttggcgctggtgcagcactgcaaatccccttcgcaatgcaaatggcctaccgattcaatggtattggtgttacccagaacgtgctctatgagaatcagaaactcatcgccaatcagttcaatagcgctattggcaagattcaggattccctcagctctaccgccagcgctctggggaagctccaggacgtggtgaaccaaaatgctcaagcgctcaatacccttgtgaaacagctcagctccaattttggcgcaattagcagcgttctgaatgatattctgtcccggctggacaaggtagaagcagaagtccagatcgacaggctgatcaccgggcggttgcagagtctccagacctatgtcacacaacagctgatccgcgccgccgagatcagggcttccgctaacctggccgccactaagatgtccgaatgcgtgttggggcagagtaagcgggtcgacttttgcgggaagggataccatctgatgagcttccctcagtctgcaccccacggagtagtgttcctccacgtcacatatgtgcccgctcaggaaaagaatttcacaaccgcacctgctatctgtcacgacggcaaggcccactttcctagagaaggagttttcgtatctaacggcacccactggttcgtgacacagcggaacttttacgagcctcagattataactacggacaacactttcgtgtcaggcaactgtgacgtggtgattgggatcgtgaacaacacagtctacgacccattgcagcccgagttggactccttcaaagaggagcttgataagtatttcaagaaccatacctctcccgacgtggacctgggggacattagcggcatcaatgcatccgttgtgaatatccagaaagaaatcgataggctgaatgaggtcgcaaaaaatcttaatgagtcactgattgatctgcaggaactcggcaaatatgagcagtatattaagtggccgtggtacatatggctcggctttatcgccggtctgattgccatcgtgatggtgaccattatgctgtgttgtatgacaagctgctgttcatgtctcaaaggatgctgctcctgcggtagctgctgtaagttcgatgaagacgacagtgagcccgtgctcaaaggagtgaaactccactacacataacgatcg

SEQ ID NO: 4 - DNA, 1278 bp, Severe acute respiratory syndrome coronavirus 2
ggtaccgccaccatgtccgataacggcccccagaatcagagaaacgctccccgcatcacgttcggcggaccaagtgacagcacaggcagtaaccagaacggagaacgctccggtgctcgctccaagcagcgacggccgcaagggcttcccaacaataccgccagctggtttacggctctgacccaacacgggaaagaagatcttaaattccccaggggccagggcgtccctatcaatactaactccagcccggatgatcagataggctactatagacgcgctacccgacggatacgagggggggacggcaaaatgaaggacctttccccccggtggtatttctattacttgggcaccggaccagaagccggactgccttacggcgctaacaaagacggaataatctgggttgcgacggagggcgccctgaatacacctaaagaccatatcggcacaagaaatcctgctaacaatgccgcgattgtgctccagctgcctcagggaaccacgctgcctaaagggttttacgctgaggggtcaaggggggggagtcaagcgtctagtaggtcatcctctcgctctcgcaatagttcccggaactcaaccccaggcagcagcagaggaacctctcccgcacggatggctggcaatgggggagatgctgcccttgctctccttctgctggatcgccttaaccagctcgaatcaaagatgtctggaaaaggtcagcagcagcaaggccagaccgtgacaaagaagagtgcagctgaagctagtaaaaagccacgccaaaaacggaccgcaactaaggcatataacgtaacacaggccttcggcagaagaggtccagaacaaacacagggaaactttggcgatcaagagctgattagacagggcacagattacaaacactggccacagatcgcgcagtttgcaccaagcgcctctgcattcttcgggatgagtcggattgggatggaagtcactccatccgggacctggcttacctacacaggggcaataaaactcgacgacaaagacccaaactttaaagatcaggtcatcctgctgaataaacacatcgatgcctacaaaactttccccccaaccgaaccaaagaaagacaagaaaaaaaaggcagacgaaacgcaagcgctccctcagcgccagaagaagcagcagaccgttacactgttgccagcagcagatctggatgatttttccaagcagcttcaacagagtatgtcaagcgctgacagcactcaggcttgacgatcg

SEQ ID NO: 5 - DNA, 3369 bp, Artificial Sequence
  Description of Artificial Sequence Synthetic polynucleotide
ggtaccgccaccatgtttgtttttctcgtactcctgcccctggtttcctcccaatgtgtcaatctgactacccggacccaacttcctcccgcctacaccaattcctttacccgaggtgtttactacccagacaaagtgttcaggtcatccgtcctccatagtacccaagacctcttcctcccttttttttctaacgttacctggtttcacgctattcacgttagcggcaccaacggcaccaaaagattcgataaccccgtactgccgttcaacgacggggtatattttgcctctactgaaaaatcaaacatcatacgcggatggatctttgggactaccctggactcaaaaactcagtccctgctgattgtgaataacgctaccaacgtggtgatcaaagtctgtgaattccagttttgcaacgatccttttctcggcgtttattatcacaaaaataacaaatcctggatggagagcgagttccgggtgtactcctccgcgaataattgcaccttcgaatatgtgtctcagccattcctcatggacctcgaggggaagcagggcaattttaagaatctgcgagaattcgtgttcaagaatatagacggttacttcaagatttactccaaacacaccccgattaacctggttagggacttgcctcagggcttttctgcattggagcccctcgtggacctcccaatcggcataaacattacaagatttcagactttgcttgcattgcacaggagctatttgacacccggcgattcttcttccggatggaccgctggagcagctgcttattacgtgggctatctgcagcctcgaacctttcttttgaagtacaacgaaaatggaactatcaccgatgcagttgactgcgccctggaccccctgtccgaaactaagtgcacgctcaaaagtttcacagtagagaaggggatataccagactagcaatttccgcgttcagccaaccgaaagtatagtgcgctttcctaatataactaacctgtgtcctttcggggaagtgtttaacgccactagattcgcttccgtctacgcctggaatagaaagaggatctcaaattgcgttgctgactatagtgttttgtacaattccgcctctttctcaaccttcaaatgttacggggtgagccctaccaaactgaacgacctgtgctttacaaacgtatacgccgacagctttgttatcagaggagacgaggttcgccagattgctccgggtcagacaggcaagattgctgattataattacaaactgcccgacgactttacaggatgtgtgatcgcgtggaacagtaacaatcttgactcaaaggttgggggtaattataattatctttaccggctgttcagaaaaagcaatttgaaacccttcgaaagggacatatccaccgagatctatcaggccgggtccactccatgcaatggtgtggaaggttttaattgctacttcccattgcagtcttatggattccaaccaaccaatggcgtaggctaccagccgtatcgcgttgtcgtgctcagcttcgagctgctccacgcccccgcgaccgtatgcggtcctaagaagtccaccaatcttgttaagaacaagtgtgtaaactttaactttaacgggctgaccgggaccggcgttctgactgaatctaacaaaaaattcctgcctttccagcagttcggccgcgatattgctgacaccactgacgctgtaagagaccctcagacccttgaaattctcgatatcacaccttgcagctttgggggcgtgtccgtcatcactccaggaactaacacaagcaaccaggtggcagtgttgtaccaggatgttaattgtaccgaggtgccagtggccatccacgccgatcaattgacacctacctggagggtttacagcacagggtccaatgtttttcagacaagagccggatgtctgatcggtgccgagcatgtcaacaattcctacgagtgtgatatccccattggtgcgggaatttgtgcatcatatcagacccagactaatagcccaagaagagctagatccgtcgctagtcaatccatcattgcatatacaatgatgtccgataacggcccccagaatcagagaaacgctccccgcatcacgttcggcggaccaagtgacagcacaggcagtaaccagaacggagaacgctccggtgctcgctccaagcagcgacggccgcaagggcttcccaacaataccgccagctggtttacggctctgacccaacacgggaaagaagatcttaaattccccaggggccagggcgtccctatcaatactaactccagcccggatgatcagataggctactatagacgcgctacccgacggatacgagggggggacggcaaaatgaaggacctttccccccggtggtatttctattacttgggcaccggaccagaagccggactgccttacggcgctaacaaagacggaataatctgggttgcgacggagggcgccctgaatacacctaaagaccatatcggcacaagaaatcctgctaacaatgccgcgattgtgctccagctgcctcagggaaccacgctgcctaaagggttttacgctgaggggtcaaggggggggagtcaagcgtctagtaggtcatcctctcgctctcgcaatagttcccggaactcaaccccaggcagcagcagaggaacctctcccgcacggatggctggcaatgggggagatgctgcccttgctctccttctgctggatcgccttaaccagctcgaatcaaagatgtctggaaaaggtcagcagcagcaaggccagaccgtgacaaagaagagtgcagctgaagctagtaaaaagccacgccaaaaacggaccgcaactaaggcatataacgtaacacaggccttcggcagaagaggtccagaacaaacacagggaaactttggcgatcaagagctgattagacagggcacagattacaaacactggccacagatcgcgcagtttgcaccaagcgcctctgcattcttcgggatgagtcggattgggatggaagtcactccatccgggacctggcttacctacacaggggcaataaaactcgacgacaaagacccaaactttaaagatcaggtcatcctgctgaataaacacatcgatgcctacaaaactttccccccaaccgaaccaaagaaagacaagaaaaaaaaggcagacgaaacgcaagcgctccctcagcgccagaagaagcagcagaccgttacactgttgccagcagcagatctggatgatttttccaagcagcttcaacagagtatgtcaagcgctgacagcactcaggcttgacgatcg

SEQ ID NO: 6 - DNA, 5752 bp, Artificial Sequence
  Description of Artificial Sequence Synthetic polynucleotide
tagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctgactctagcctagctctgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtctgagcctaggagatctctcgaggtcgacggtatcgatgggtaccgccaccatgtttgtttttctcgtactcctgcccctggtttcctcccaatgtgtcaatctgactacccggacccaacttcctcccgcctacaccaattcctttacccgaggtgtttactacccagacaaagtgttcaggtcatccgtcctccatagtacccaagacctcttcctcccttttttttctaacgttacctggtttcacgctattcacgttagcggcaccaacggcaccaaaagattcgataaccccgtactgccgttcaacgacggggtatattttgcctctactgaaaaatcaaacatcatacgcggatggatctttgggactaccctggactcaaaaactcagtccctgctgattgtgaataacgctaccaacgtggtgatcaaagtctgtgaattccagttttgcaacgatccttttctcggcgtttattatcacaaaaataacaaatcctggatggagagcgagttccgggtgtactcctccgcgaataattgcaccttcgaatatgtgtctcagccattcctcatggacctcgaggggaagcagggcaattttaagaatctgcgagaattcgtgttcaagaatatagacggttacttcaagatttactccaaacacaccccgattaacctggttagggacttgcctcagggcttttctgcattggagcccctcgtggacctcccaatcggcataaacattacaagatttcagactttgcttgcattgcacaggagctatttgacacccggcgattcttcttccggatggaccgctggagcagctgcttattacgtgggctatctgcagcctcgaacctttcttttgaagtacaacgaaaatggaactatcaccgatgcagttgactgcgccctggaccccctgtccgaaactaagtgcacgctcaaaagtttcacagtagagaaggggatataccagactagcaatttccgcgttcagccaaccgaaagtatagtgcgctttcctaatataactaacctgtgtcctttcggggaagtgtttaacgccactagattcgcttccgtctacgcctggaatagaaagaggatctcaaattgcgttgctgactatagtgttttgtacaattccgcctctttctcaaccttcaaatgttacggggtgagccctaccaaactgaacgacctgtgctttacaaacgtatacgccgacagctttgttatcagaggagacgaggttcgccagattgctccgggtcagacaggcaagattgctgattataattacaaactgcccgacgactttacaggatgtgtgatcgcgtggaacagtaacaatcttgactcaaaggttgggggtaattataattatctttaccggctgttcagaaaaagcaatttgaaacccttcgaaagggacatatccaccgagatctatcaggccgggtccactccatgcaatggtgtggaaggttttaattgctacttcccattgcagtcttatggattccaaccaaccaatggcgtaggctaccagccgtatcgcgttgtcgtgctcagcttcgagctgctccacgcccccgcgaccgtatgcggtcctaagaagtccaccaatcttgttaagaacaagtgtgtaaactttaactttaacgggctgaccgggaccggcgttctgactgaatctaacaaaaaattcctgcctttccagcagttcggccgcgatattgctgacaccactgacgctgtaagagaccctcagacccttgaaattctcgatatcacaccttgcagctttgggggcgtgtccgtcatcactccaggaactaacacaagcaaccaggtggcagtgttgtaccaggatgttaattgtaccgaggtgccagtggccatccacgccgatcaattgacacctacctggagggtttacagcacagggtccaatgtttttcagacaagagccggatgtctgatcggtgccgagcatgtcaacaattcctacgagtgtgatatccccattggtgcgggaatttgtgcatcatatcagacccagactaatagcccaagaagagctagatccgtcgctagtcaatccatcattgcatatacaatgtccctgggagctgagaattcagtcgcgtattcaaacaattccattgctattcctactaatttcactatctccgtcacgaccgagatcctgccagtttccatgactaagacttctgttgactgcaccatgtatatctgtggcgatagcaccgagtgcagtaatctgcttctgcagtacggctccttctgcacacaactcaatcgagcactgaccggtattgcagttgagcaggacaagaacacacaggaggtctttgcacaggtcaaacaaatttacaaaaccccccccataaaagactttggtgggttcaacttcagccaaatcctcccagatcccagcaagccctccaaaagatccttcatcgaagaccttttgttcaataaggtaaccctggccgacgcaggcttcatcaaacaatatggcgattgccttggagacattgctgcgcgcgatttgatctgtgctcagaaatttaacggtttgaccgtgctgcccccacttctgactgatgagatgatagcacagtatacttctgctcttctggcaggaacaatcacttccgggtggacctttggcgctggtgcagcactgcaaatccccttcgcaatgcaaatggcctaccgattcaatggtattggtgttacccagaacgtgctctatgagaatcagaaactcatcgccaatcagttcaatagcgctattggcaagattcaggattccctcagctctaccgccagcgctctggggaagctccaggacgtggtgaaccaaaatgctcaagcgctcaatacccttgtgaaacagctcagctccaattttggcgcaattagcagcgttctgaatgatattctgtcccggctggacaaggtagaagcagaagtccagatcgacaggctgatcaccgggcggttgcagagtctccagacctatgtcacacaacagctgatccgcgccgccgagatcagggcttccgctaacctggccgccactaagatgtccgaatgcgtgttggggcagagtaagcgggtcgacttttgcgggaagggataccatctgatgagcttccctcagtctgcaccccacggagtagtgttcctccacgtcacatatgtgcccgctcaggaaaagaatttcacaaccgcacctgctatctgtcacgacggcaaggcccactttcctagagaaggagttttcgtatctaacggcacccactggttcgtgacacagcggaacttttacgagcctcagattataactacggacaacactttcgtgtcaggcaactgtgacgtggtgattgggatcgtgaacaacacagtctacgacccattgcagcccgagttggactccttcaaagaggagcttgataagtatttcaagaaccatacctctcccgacgtggacctgggggacattagcggcatcaatgcatccgttgtgaatatccagaaagaaatcgataggctgaatgaggtcgcaaaaaatcttaatgagtcactgattgatctgcaggaactcggcaaatatgagcagtatattaagtggccgtggtacatatggctcggctttatcgccggtctgattgccatcgtgatggtgaccattatgctgtgttgtatgacaagctgctgttcatgtctcaaaggatgctgctcctgcggtagctgctgtaagttcgatgaagacgacagtgagcccgtgctcaaaggagtgaaactccactacacataacgatcggatatcgctagcgtaccggcggccgccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccaaagcttacgcgttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatatcgggccactgcaggaaacgatatgggctgaatacggatccgtattcagcccatatcgtttctctagaaataaaatatctttattttcattacatctgtgtgttggttttttgtgtg

SEQ ID NO: 7 - DNA, 7310 bp, Artificial Sequence
  Description of Artificial Sequence Synthetic polynucleotide
tagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctgactctagcctagctctgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtctgagcctaggagatctctcgaggtcgacggtatcgatgggtaccgccaccatgtttgtttttctcgtactcctgcccctggtttcctcccaatgtgtcaatctgactacccggacccaacttcctcccgcctacaccaattcctttacccgaggtgtttactacccagacaaagtgttcaggtcatccgtcctccatagtacccaagacctcttcctcccttttttttctaacgttacctggtttcacgctattcacgttagcggcaccaacggcaccaaaagattcgataaccccgtactgccgttcaacgacggggtatattttgcctctactgaaaaatcaaacatcatacgcggatggatctttgggactaccctggactcaaaaactcagtccctgctgattgtgaataacgctaccaacgtggtgatcaaagtctgtgaattccagttttgcaacgatccttttctcggcgtttattatcacaaaaataacaaatcctggatggagagcgagttccgggtgtactcctccgcgaataattgcaccttcgaatatgtgtctcagccattcctcatggacctcgaggggaagcagggcaattttaagaatctgcgagaattcgtgttcaagaatatagacggttacttcaagatttactccaaacacaccccgattaacctggttagggacttgcctcagggcttttctgcattggagcccctcgtggacctcccaatcggcataaacattacaagatttcagactttgcttgcattgcacaggagctatttgacacccggcgattcttcttccggatggaccgctggagcagctgcttattacgtgggctatctgcagcctcgaacctttcttttgaagtacaacgaaaatggaactatcaccgatgcagttgactgcgccctggaccccctgtccgaaactaagtgcacgctcaaaagtttcacagtagagaaggggatataccagactagcaatttccgcgttcagccaaccgaaagtatagtgcgctttcctaatataactaacctgtgtcctttcggggaagtgtttaacgccactagattcgcttccgtctacgcctggaatagaaagaggatctcaaattgcgttgctgactatagtgttttgtacaattccgcctctttctcaaccttcaaatgttacggggtgagccctaccaaactgaacgacctgtgctttacaaacgtatacgccgacagctttgttatcagaggagacgaggttcgccagattgctccgggtcagacaggcaagattgctgattataattacaaactgcccgacgactttacaggatgtgtgatcgcgtggaacagtaacaatcttgactcaaaggttgggggtaattataattatctttaccggctgttcagaaaaagcaatttgaaacccttcgaaagggacatatccaccgagatctatcaggccgggtccactccatgcaatggtgtggaaggttttaattgctacttcccattgcagtcttatggattccaaccaaccaatggcgtaggctaccagccgtatcgcgttgtcgtgctcagcttcgagctgctccacgcccccgcgaccgtatgcggtcctaagaagtccaccaatcttgttaagaacaagtgtgtaaactttaactttaacgggctgaccgggaccggcgttctgactgaatctaacaaaaaattcctgcctttccagcagttcggccgcgatattgctgacaccactgacgctgtaagagaccctcagacccttgaaattctcgatatcacaccttgcagctttgggggcgtgtccgtcatcactccaggaactaacacaagcaaccaggtggcagtgttgtaccaggatgttaattgtaccgaggtgccagtggccatccacgccgatcaattgacacctacctggagggtttacagcacagggtccaatgtttttcagacaagagccggatgtctgatcggtgccgagcatgtcaacaattcctacgagtgtgatatccccattggtgcgggaatttgtgcatcatatcagacccagactaatagcccaagaagagctagatccgtcgctagtcaatccatcattgcatatacaatgtccctgggagctgagaattcagtcgcgtattcaaacaattccattgctattcctactaatttcactatctccgtcacgaccgagatcctgccagtttccatgactaagacttctgttgactgcaccatgtatatctgtggcgatagcaccgagtgcagtaatctgcttctgcagtacggctccttctgcacacaactcaatcgagcactgaccggtattgcagttgagcaggacaagaacacacaggaggtctttgcacaggtcaaacaaatttacaaaaccccccccataaaagactttggtgggttcaacttcagccaaatcctcccagatcccagcaagccctccaaaagatccttcatcgaagaccttttgttcaataaggtaaccctggccgacgcaggcttcatcaaacaatatggcgattgccttggagacattgctgcgcgcgatttgatctgtgctcagaaatttaacggtttgaccgtgctgcccccacttctgactgatgagatgatagcacagtatacttctgctcttctggcaggaacaatcacttccgggtggacctttggcgctggtgcagcactgcaaatccccttcgcaatgcaaatggcctaccgattcaatggtattggtgttacccagaacgtgctctatgagaatcagaaactcatcgccaatcagttcaatagcgctattggcaagattcaggattccctcagctctaccgccagcgctctggggaagctccaggacgtggtgaaccaaaatgctcaagcgctcaatacccttgtgaaacagctcagctccaattttggcgcaattagcagcgttctgaatgatattctgtcccggctggacaaggtagaagcagaagtccagatcgacaggctgatcaccgggcggttgcagagtctccagacctatgtcacacaacagctgatccgcgccgccgagatcagggcttccgctaacctggccgccactaagatgtccgaatgcgtgttggggcagagtaagcgggtcgacttttgcgggaagggataccatctgatgagcttccctcagtctgcaccccacggagtagtgttcctccacgtcacatatgtgcccgctcaggaaaagaatttcacaaccgcacctgctatctgtcacgacggcaaggcccactttcctagagaaggagttttcgtatctaacggcacccactggttcgtgacacagcggaacttttacgagcctcagattataactacggacaacactttcgtgtcaggcaactgtgacgtggtgattgggatcgtgaacaacacagtctacgacccattgcagcccgagttggactccttcaaagaggagcttgataagtatttcaagaaccatacctctcccgacgtggacctgggggacattagcggcatcaatgcatccgttgtgaatatccagaaagaaatcgataggctgaatgaggtcgcaaaaaatcttaatgagtcactgattgatctgcaggaactcggcaaatatgagcagtatattaagtggccgtggtacatatggctcggctttatcgccggtctgattgccatcgtgatggtgaccattatgctgtgttgtatgacaagctgctgttcatgtctcaaaggatgctgctcctgcggtagctgctgtaagttcgatgaagacgacagtgagcccgtgctcaaaggagtgaaactccactacacataacgatcgacgcgtagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccaaagcttgcggccgcgcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagaccctgcaggccgccaccatgtccgataacggcccccagaatcagagaaacgctccccgcatcacgttcggcggaccaagtgacagcacaggcagtaaccagaacggagaacgctccggtgctcgctccaagcagcgacggccgcaagggcttcccaacaataccgccagctggtttacggctctgacccaacacgggaaagaagatcttaaattccccaggggccagggcgtccctatcaatactaactccagcccggatgatcagataggctactatagacgcgctacccgacggatacgagggggggacggcaaaatgaaggacctttccccccggtggtatttctattacttgggcaccggaccagaagccggactgccttacggcgctaacaaagacggaataatctgggttgcgacggagggcgccctgaatacacctaaagaccatatcggcacaagaaatcctgctaacaatgccgcgattgtgctccagctgcctcagggaaccacgctgcctaaagggttttacgctgaggggtcaaggggggggagtcaagcgtctagtaggtcatcctctcgctctcgcaatagttcccggaactcaaccccaggcagcagcagaggaacctctcccgcacggatggctggcaatgggggagatgctgcccttgctctccttctgctggatcgccttaaccagctcgaatcaaagatgtctggaaaaggtcagcagcagcaaggccagaccgtgacaaagaagagtgcagctgaagctagtaaaaagccacgccaaaaacggaccgcaactaaggcatataacgtaacacaggccttcggcagaagaggtccagaacaaacacagggaaactttggcgatcaagagctgattagacagggcacagattacaaacactggccacagatcgcgcagtttgcaccaagcgcctctgcattcttcgggatgagtcggattgggatggaagtcactccatccgggacctggcttacctacacaggggcaataaaactcgacgacaaagacccaaactttaaagatcaggtcatcctgctgaataaacacatcgatgcctacaaaactttccccccaaccgaaccaaagaaagacaagaaaaaaaaggcagacgaaacgcaagcgctccctcagcgccagaagaagcagcagaccgttacactgttgccagcagcagatctggatgatttttccaagcagcttcaacagagtatgtcaagcgctgacagcactcaggcttgaggcgcgccgctgaccgataaataaaatatctttattttcattacatctgtgtgttggttttttgtgtgacgcgttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatatcgggccactgcaggaaacgatatgggctgaatacggatccgtattcagcccatatcgtttctctagaaataaaatatctttattttcattacatctgtgtgttggttttttgtgtg

SEQ ID NO: 8 - DNA, 5281 bp, Artificial Sequence
  Description of Artificial Sequence Synthetic polynucleotide
tagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctgactctagcctagctctgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtctgagcctaggagatctctcgaggtcgacggtatcgatgggtaccgccaccatgtttgtttttctcgtactcctgcccctggtttcctcccaatgtgtcaatctgactacccggacccaacttcctcccgcctacaccaattcctttacccgaggtgtttactacccagacaaagtgttcaggtcatccgtcctccatagtacccaagacctcttcctcccttttttttctaacgttacctggtttcacgctattcacgttagcggcaccaacggcaccaaaagattcgataaccccgtactgccgttcaacgacggggtatattttgcctctactgaaaaatcaaacatcatacgcggatggatctttgggactaccctggactcaaaaactcagtccctgctgattgtgaataacgctaccaacgtggtgatcaaagtctgtgaattccagttttgcaacgatccttttctcggcgtttattatcacaaaaataacaaatcctggatggagagcgagttccgggtgtactcctccgcgaataattgcaccttcgaatatgtgtctcagccattcctcatggacctcgaggggaagcagggcaattttaagaatctgcgagaattcgtgttcaagaatatagacggttacttcaagatttactccaaacacaccccgattaacctggttagggacttgcctcagggcttttctgcattggagcccctcgtggacctcccaatcggcataaacattacaagatttcagactttgcttgcattgcacaggagctatttgacacccggcgattcttcttccggatggaccgctggagcagctgcttattacgtgggctatctgcagcctcgaacctttcttttgaagtacaacgaaaatggaactatcaccgatgcagttgactgcgccctggaccccctgtccgaaactaagtgcacgctcaaaagtttcacagtagagaaggggatataccagactagcaatttccgcgttcagccaaccgaaagtatagtgcgctttcctaatataactaacctgtgtcctttcggggaagtgtttaacgccactagattcgcttccgtctacgcctggaatagaaagaggatctcaaattgcgttgctgactatagtgttttgtacaattccgcctctttctcaaccttcaaatgttacggggtgagccctaccaaactgaacgacctgtgctttacaaacgtatacgccgacagctttgttatcagaggagacgaggttcgccagattgctccgggtcagacaggcaagattgctgattataattacaaactgcccgacgactttacaggatgtgtgatcgcgtggaacagtaacaatcttgactcaaaggttgggggtaattataattatctttaccggctgttcagaaaaagcaatttgaaacccttcgaaagggacatatccaccgagatctatcaggccgggtccactccatgcaatggtgtggaaggttttaattgctacttcccattgcagtcttatggattccaaccaaccaatggcgtaggctaccagccgtatcgcgttgtcgtgctcagcttcgagctgctccacgcccccgcgaccgtatgcggtcctaagaagtccaccaatcttgttaagaacaagtgtgtaaactttaactttaacgggctgaccgggaccggcgttctgactgaatctaacaaaaaattcctgcctttccagcagttcggccgcgatattgctgacaccactgacgctgtaagagaccctcagacccttgaaattctcgatatcacaccttgcagctttgggggcgtgtccgtcatcactccaggaactaacacaagcaaccaggtggcagtgttgtaccaggatgttaattgtaccgaggtgccagtggccatccacgccgatcaattgacacctacctggagggtttacagcacagggtccaatgtttttcagacaagagccggatgtctgatcggtgccgagcatgtcaacaattcctacgagtgtgatatccccattggtgcgggaatttgtgcatcatatcagacccagactaatagcccaagaagagctagatccgtcgctagtcaatccatcattgcatatacaatgatgtccgataacggcccccagaatcagagaaacgctccccgcatcacgttcggcggaccaagtgacagcacaggcagtaaccagaacggagaacgctccggtgctcgctccaagcagcgacggccgcaagggcttcccaacaataccgccagctggtttacggctctgacccaacacgggaaagaagatcttaaattccccaggggccagggcgtccctatcaatactaactccagcccggatgatcagataggctactatagacgcgctacccgacggatacgagggggggacggcaaaatgaaggacctttccccccggtggtatttctattacttgggcaccggaccagaagccggactgccttacggcgctaacaaagacggaataatctgggttgcgacggagggcgccctgaatacacctaaagaccatatcggcacaagaaatcctgctaacaatgccgcgattgtgctccagctgcctcagggaaccacgctgcctaaagggttttacgctgaggggtcaaggggggggagtcaagcgtctagtaggtcatcctctcgctctcgcaatagttcccggaactcaaccccaggcagcagcagaggaacctctcccgcacggatggctggcaatgggggagatgctgcccttgctctccttctgctggatcgccttaaccagctcgaatcaaagatgtctggaaaaggtcagcagcagcaaggccagaccgtgacaaagaagagtgcagctgaagctagtaaaaagccacgccaaaaacggaccgcaactaaggcatataacgtaacacaggccttcggcagaagaggtccagaacaaacacagggaaactttggcgatcaagagctgattagacagggcacagattacaaacactggccacagatcgcgcagtttgcaccaagcgcctctgcattcttcgggatgagtcggattgggatggaagtcactccatccgggacctggcttacctacacaggggcaataaaactcgacgacaaagacccaaactttaaagatcaggtcatcctgctgaataaacacatcgatgcctacaaaactttccccccaaccgaaccaaagaaagacaagaaaaaaaaggcagacgaaacgcaagcgctccctcagcgccagaagaagcagcagaccgttacactgttgccagcagcagatctggatgatttttccaagcagcttcaacagagtatgtcaagcgctgacagcactcaggcttgacgatcggatatcgctagcgtaccggcggccgccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccaaagcttacgcgttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatatcgggccactgcaggaaacgatatgggctgaatacggatccgtattcagcccatatcgtttctctagaaataaaatatctttattttcattacatctgtgtgttggttttttgtgtg

SEQ ID NO: 9 - DNA, 36177 bp, Artificial Sequence
  Description of Artificial Sequence Synthetic polynucleotide
taaggatcccatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactgctagagatctggcgaaagggggatgtgctgcaaggcgattaagttgggtaacgccagggttttcccagtcacgacgttgtaaaacgacggccagtgaattgtaatacgactcactatagggcgaattgggtactggccacaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctgactctagcctagctctgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtctgagcctaggagatctctcgaggtcgacggtatcgatgggtaccgccaccatgtttgtttttctcgtactcctgcccctggtttcctcccaatgtgtcaatctgactacccggacccaacttcctcccgcctacaccaattcctttacccgaggtgtttactacccagacaaagtgttcaggtcatccgtcctccatagtacccaagacctcttcctcccttttttttctaacgttacctggtttcacgctattcacgttagcggcaccaacggcaccaaaagattcgataaccccgtactgccgttcaacgacggggtatattttgcctctactgaaaaatcaaacatcatacgcggatggatctttgggactaccctggactcaaaaactcagtccctgctgattgtgaataacgctaccaacgtggtgatcaaagtctgtgaattccagttttgcaacgatccttttctcggcgtttattatcacaaaaataacaaatcctggatggagagcgagttccgggtgtactcctccgcgaataattgcaccttcgaatatgtgtctcagccattcctcatggacctcgaggggaagcagggcaattttaagaatctgcgagaattcgtgttcaagaatatagacggttacttcaagatttactccaaacacaccccgattaacctggttagggacttgcctcagggcttttctgcattggagcccctcgtggacctcccaatcggcataaacattacaagatttcagactttgcttgcattgcacaggagctatttgacacccggcgattcttcttccggatggaccgctggagcagctgcttattacgtgggctatctgcagcctcgaacctttcttttgaagtacaacgaaaatggaactatcaccgatgcagttgactgcgccctggaccccctgtccgaaactaagtgcacgctcaaaagtttcacagtagagaaggggatataccagactagcaatttccgcgttcagccaaccgaaagtatagtgcgctttcctaatataactaacctgtgtcctttcggggaagtgtttaacgccactagattcgcttccgtctacgcctggaatagaaagaggatctcaaattgcgttgctgactatagtgttttgtacaattccgcctctttctcaaccttcaaatgttacggggtgagccctaccaaactgaacgacctgtgctttacaaacgtatacgccgacagctttgttatcagaggagacgaggttcgccagattgctccgggtcagacaggcaagattgctgattataattacaaactgcccgacgactttacaggatgtgtgatcgcgtggaacagtaacaatcttgactcaaaggttgggggtaattataattatctttaccggctgttcagaaaaagcaatttgaaacccttcgaaagggacatatccaccgagatctatcaggccgggtccactccatgcaatggtgtggaaggttttaattgctacttcccattgcagtcttatggattccaaccaaccaatggcgtaggctaccagccgtatcgcgttgtcgtgctcagcttcgagctgctccacgcccccgcgaccgtatgcggtcctaagaagtccaccaatcttgttaagaacaagtgtgtaaactttaactttaacgggctgaccgggaccggcgttctgactgaatctaacaaaaaattcctgcctttccagcagttcggccgcgatattgctgacaccactgacgctgtaagagaccctcagacccttgaaattctcgatatcacaccttgcagctttgggggcgtgtccgtcatcactccaggaactaacacaagcaaccaggtggcagtgttgtaccaggatgttaattgtaccgaggtgccagtggccatccacgccgatcaattgacacctacctggagggtttacagcacagggtccaatgtttttcagacaagagccggatgtctgatcggtgccgagcatgtcaacaattcctacgagtgtgatatccccattggtgcgggaatttgtgcatcatatcagacccagactaatagcccaagaagagctagatccgtcgctagtcaatccatcattgcatatacaatgtccctgggagctgagaattcagtcgcgtattcaaacaattccattgctattcctactaatttcactatctccgtcacgaccgagatcctgccagtttccatgactaagacttctgttgactgcaccatgtatatctgtggcgatagcaccgagtgcagtaatctgcttctgcagtacggctccttctgcacacaactcaatcgagcactgaccggtattgcagttgagcaggacaagaacacacaggaggtctttgcacaggtcaaacaaatttacaaaaccccccccataaaagactttggtgggttcaacttcagccaaatcctcccagatcccagcaagccctccaaaagatccttcatcgaagaccttttgttcaataaggtaaccctggccgacgcaggcttcatcaaacaatatggcgattgccttggagacattgctgcgcgcgatttgatctgtgctcagaaatttaacggtttgaccgtgctgcccccacttctgactgatgagatgatagcacagtatacttctgctcttctggcaggaacaatcacttccgggtggacctttggcgctggtgcagcactgcaaatccccttcgcaatgcaaatggcctaccgattcaatggtattggtgttacccagaacgtgctctatgagaatcagaaactcatcgccaatcagttcaatagcgctattggcaagattcaggattccctcagctctaccgccagcgctctggggaagctccaggacgtggtgaaccaaaatgctcaagcgctcaatacccttgtgaaacagctcagctccaattttggcgcaattagcagcgttctgaatgatattctgtcccggctggacaaggtagaagcagaagtccagatcgacaggctgatcaccgggcggttgcagagtctccagacctatgtcacacaacagctgatccgcgccgccgagatcagggcttccgctaacctggccgccactaagatgtccgaatgcgtgttggggcagagtaagcgggtcgacttttgcgggaagggataccatctgatgagcttccctcagtctgcaccccacggagtagtgttcctccacgtcacatatgtgcccgctcaggaaaagaatttcacaaccgcacctgctatctgtcacgacggcaaggcccactttcctagagaaggagttttcgtatctaacggcacccactggttcgtgacacagcggaacttttacgagcctcagattataactacggacaacactttcgtgtcaggcaactgtgacgtggtgattgggatcgtgaacaacacagtctacgacccattgcagcccgagttggactccttcaaagaggagcttgataagtatttcaagaaccatacctctcccgacgtggacctgggggacattagcggcatcaatgcatccgttgtgaatatccagaaagaaatcgataggctgaatgaggtcgcaaaaaatcttaatgagtcactgattgatctgcaggaactcggcaaatatgagcagtatattaagtggccgtggtacatatggctcggctttatcgccggtctgattgccatcgtgatggtgaccattatgctgtgttgtatgacaagctgctgttcatgtctcaaaggatgctgctcctgcggtagctgctgtaagttcgatgaagacgacagtgagcccgtgctcaaaggagtgaaactccactacacataacgatcggatatcgctagcgtaccggcggccgccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccaaagcttacgcgttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatatcgggccactgcaggaaacgatatgggctgaatacggatccgtattcagcccatatcgtttctctagaaataaaatatctttattttcattacatctgtgtgttggttttttgtgtgaatcgatagtactaacatacgctctccatctcgagcctaagcttgtcgactcgaagatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaatgtcaggtcgcctaagtcgattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatatt

SEQ ID NO: 10 - DNA, 37735 bp, Artificial Sequence
  Description of Artificial Sequence Synthetic polynucleotide
taaggatcccatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactgctagagatctggcgaaagggggatgtgctgcaaggcgattaagttgggtaacgccagggttttcccagtcacgacgttgtaaaacgacggccagtgaattgtaatacgactcactatagggcgaattgggtactggccacaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctgactctagcctagctctgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtctgagcctaggagatctctcgaggtcgacggtatcgatgggtaccgccaccatgtttgtttttctcgtactcctgcccctggtttcctcccaatgtgtcaatctgactacccggacccaacttcctcccgcctacaccaattcctttacccgaggtgtttactacccagacaaagtgttcaggtcatccgtcctccatagtacccaagacctcttcctcccttttttttctaacgttacctggtttcacgctattcacgttagcggcaccaacggcaccaaaagattcgataaccccgtactgccgttcaacgacggggtatattttgcctctactgaaaaatcaaacatcatacgcggatggatctttgggactaccctggactcaaaaactcagtccctgctgattgtgaataacgctaccaacgtggtgatcaaagtctgtgaattccagttttgcaacgatccttttctcggcgtttattatcacaaaaataacaaatcctggatggagagcgagttccgggtgtactcctccgcgaataattgcaccttcgaatatgtgtctcagccattcctcatggacctcgaggggaagcagggcaattttaagaatctgcgagaattcgtgttcaagaatatagacggttacttcaagatttactccaaacacaccccgattaacctggttagggacttgcctcagggcttttctgcattggagcccctcgtggacctcccaatcggcataaacattacaagatttcagactttgcttgcattgcacaggagctatttgacacccggcgattcttcttccggatggaccgctggagcagctgcttattacgtgggctatctgcagcctcgaacctttcttttgaagtacaacgaaaatggaactatcaccgatgcagttgactgcgccctggaccccctgtccgaaactaagtgcacgctcaaaagtttcacagtagagaaggggatataccagactagcaatttccgcgttcagccaaccgaaagtatagtgcgctttcctaatataactaacctgtgtcctttcggggaagtgtttaacgccactagattcgcttccgtctacgcctggaatagaaagaggatctcaaattgcgttgctgactatagtgttttgtacaattccgcctctttctcaaccttcaaatgttacggggtgagccctaccaaactgaacgacctgtgctttacaaacgtatacgccgacagctttgttatcagaggagacgaggttcgccagattgctccgggtcagacaggcaagattgctgattataattacaaactgcccgacgactttacaggatgtgtgatcgcgtggaacagtaacaatcttgactcaaaggttgggggtaattataattatctttaccggctgttcagaaaaagcaatttgaaacccttcgaaagggacatatccaccgagatctatcaggccgggtccactccatgcaatggtgtggaaggttttaattgctacttcccattgcagtcttatggattccaaccaaccaatggcgtaggctaccagccgtatcgcgttgtcgtgctcagcttcgagctgctccacgcccccgcgaccgtatgcggtcctaagaagtccaccaatcttgttaagaacaagtgtgtaaactttaactttaacgggctgaccgggaccggcgttctgactgaatctaacaaaaaattcctgcctttccagcagttcggccgcgatattgctgacaccactgacgctgtaagagaccctcagacccttgaaattctcgatatcacaccttgcagctttgggggcgtgtccgtcatcactccaggaactaacacaagcaaccaggtggcagtgttgtaccaggatgttaattgtaccgaggtgccagtggccatccacgccgatcaattgacacctacctggagggtttacagcacagggtccaatgtttttcagacaagagccggatgtctgatcggtgccgagcatgtcaacaattcctacgagtgtgatatccccattggtgcgggaatttgtgcatcatatcagacccagactaatagcccaagaagagctagatccgtcgctagtcaatccatcattgcatatacaatgtccctgggagctgagaattcagtcgcgtattcaaacaattccattgctattcctactaatttcactatctccgtcacgaccgagatcctgccagtttccatgactaagacttctgttgactgcaccatgtatatctgtggcgatagcaccgagtgcagtaatctgcttctgcagtacggctccttctgcacacaactcaatcgagcactgaccggtattgcagttgagcaggacaagaacacacaggaggtctttgcacaggtcaaacaaatttacaaaaccccccccataaaagactttggtgggttcaacttcagccaaatcctcccagatcccagcaagccctccaaaagatccttcatcgaagaccttttgttcaataaggtaaccctggccgacgcaggcttcatcaaacaatatggcgattgccttggagacattgctgcgcgcgatttgatctgtgctcagaaatttaacggtttgaccgtgctgcccccacttctgactgatgagatgatagcacagtatacttctgctcttctggcaggaacaatcacttccgggtggacctttggcgctggtgcagcactgcaaatccccttcgcaatgcaaatggcctaccgattcaatggtattggtgttacccagaacgtgctctatgagaatcagaaactcatcgccaatcagttcaatagcgctattggcaagattcaggattccctcagctctaccgccagcgctctggggaagctccaggacgtggtgaaccaaaatgctcaagcgctcaatacccttgtgaaacagctcagctccaattttggcgcaattagcagcgttctgaatgatattctgtcccggctggacaaggtagaagcagaagtccagatcgacaggctgatcaccgggcggttgcagagtctccagacctatgtcacacaacagctgatccgcgccgccgagatcagggcttccgctaacctggccgccactaagatgtccgaatgcgtgttggggcagagtaagcgggtcgacttttgcgggaagggataccatctgatgagcttccctcagtctgcaccccacggagtagtgttcctccacgtcacatatgtgcccgctcaggaaaagaatttcacaaccgcacctgctatctgtcacgacggcaaggcccactttcctagagaaggagttttcgtatctaacggcacccactggttcgtgacacagcggaacttttacgagcctcagattataactacggacaacactttcgtgtcaggcaactgtgacgtggtgattgggatcgtgaacaacacagtctacgacccattgcagcccgagttggactccttcaaagaggagcttgataagtatttcaagaaccatacctctcccgacgtggacctgggggacattagcggcatcaatgcatccgttgtgaatatccagaaagaaatcgataggctgaatgaggtcgcaaaaaatcttaatgagtcactgattgatctgcaggaactcggcaaatatgagcagtatattaagtggccgtggtacatatggctcggctttatcgccggtctgattgccatcgtgatggtgaccattatgctgtgttgtatgacaagctgctgttcatgtctcaaaggatgctgctcctgcggtagctgctgtaagttcgatgaagacgacagtgagcccgtgctcaaaggagtgaaactccactacacataacgatcgacgcgtagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccaaagcttgcggccgcgcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagaccctgcaggccgccaccatgtccgataacggcccccagaatcagagaaacgctccccgcatcacgttcggcggaccaagtgacagcacaggcagtaaccagaacggagaacgctccggtgctcgctccaagcagcgacggccgcaagggcttcccaacaataccgccagctggtttacggctctgacccaacacgggaaagaagatcttaaattccccaggggccagggcgtccctatcaatactaactccagcccggatgatcagataggctactatagacgcgctacccgacggatacgagggggggacggcaaaatgaaggacctttccccccggtggtatttctattacttgggcaccggaccagaagccggactgccttacggcgctaacaaagacggaataatctgggttgcgacggagggcgccctgaatacacctaaagaccatatcggcacaagaaatcctgctaacaatgccgcgattgtgctccagctgcctcagggaaccacgctgcctaaagggttttacgctgaggggtcaaggggggggagtcaagcgtctagtaggtcatcctctcgctctcgcaatagttcccggaactcaaccccaggcagcagcagaggaacctctcccgcacggatggctggcaatgggggagatgctgcccttgctctccttctgctggatcgccttaaccagctcgaatcaaagatgtctggaaaaggtcagcagcagcaaggccagaccgtgacaaagaagagtgcagctgaagctagtaaaaagccacgccaaaaacggaccgcaactaaggcatataacgtaacacaggccttcggcagaagaggtccagaacaaacacagggaaactttggcgatcaagagctgattagacagggcacagattacaaacactggccacagatcgcgcagtttgcaccaagcgcctctgcattcttcgggatgagtcggattgggatggaagtcactccatccgggacctggcttacctacacaggggcaataaaactcgacgacaaagacccaaactttaaagatcaggtcatcctgctgaataaacacatcgatgcctacaaaactttccccccaaccgaaccaaagaaagacaagaaaaaaaaggcagacgaaacgcaagcgctccctcagcgccagaagaagcagcagaccgttacactgttgccagcagcagatctggatgatttttccaagcagcttcaacagagtatgtcaagcgctgacagcactcaggcttgaggcgcgccgctgaccgataaataaaatatctttattttcattacatctgtgtgttggttttttgtgtgacgcgttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatatcgggccactgcaggaaacgatatgggctgaatacggatccgtattcagcccatatcgtttctctagaaataaaatatctttattttcattacatctgtgtgttggttttttgtgtgaatcgatagtactaacatacgctctccatctcgagcctaagcttgtcgactcgaagatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaatgtcaggtcgcctaagtcgattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatatt

SEQ ID NO: 11 - DNA, 35706 bp, Artificial Sequence
  Description of Artificial Sequence Synthetic polynucleotide
taaggatcccatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactgctagagatctggcgaaagggggatgtgctgcaaggcgattaagttgggtaacgccagggttttcccagtcacgacgttgtaaaacgacggccagtgaattgtaatacgactcactatagggcgaattgggtactggccacaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctgactctagcctagctctgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtctgagcctaggagatctctcgaggtcgacggtatcgatgggtaccgccaccatgtttgtttttctcgtactcctgcccctggtttcctcccaatgtgtcaatctgactacccggacccaacttcctcccgcctacaccaattcctttacccgaggtgtttactacccagacaaagtgttcaggtcatccgtcctccatagtacccaagacctcttcctcccttttttttctaacgttacctggtttcacgctattcacgttagcggcaccaacggcaccaaaagattcgataaccccgtactgccgttcaacgacggggtatattttgcctctactgaaaaatcaaacatcatacgcggatggatctttgggactaccctggactcaaaaactcagtccctgctgattgtgaataacgctaccaacgtggtgatcaaagtctgtgaattccagttttgcaacgatccttttctcggcgtttattatcacaaaaataacaaatcctggatggagagcgagttccgggtgtactcctccgcgaataattgcaccttcgaatatgtgtctcagccattcctcatggacctcgaggggaagcagggcaattttaagaatctgcgagaattcgtgttcaagaatatagacggttacttcaagatttactccaaacacaccccgattaacctggttagggacttgcctcagggcttttctgcattggagcccctcgtggacctcccaatcggcataaacattacaagatttcagactttgcttgcattgcacaggagctatttgacacccggcgattcttcttccggatggaccgctggagcagctgcttattacgtgggctatctgcagcctcgaacctttcttttgaagtacaacgaaaatggaactatcaccgatgcagttgactgcgccctggaccccctgtccgaaactaagtgcacgctcaaaagtttcacagtagagaaggggatataccagactagcaatttccgcgttcagccaaccgaaagtatagtgcgctttcctaatataactaacctgtgtcctttcggggaagtgtttaacgccactagattcgcttccgtctacgcctggaatagaaagaggatctcaaattgcgttgctgactatagtgttttgtacaattccgcctctttctcaaccttcaaatgttacggggtgagccctaccaaactgaacgacctgtgctttacaaacgtatacgccgacagctttgttatcagaggagacgaggttcgccagattgctccgggtcagacaggcaagattgctgattataattacaaactgcccgacgactttacaggatgtgtgatcgcgtggaacagtaacaatcttgactcaaaggttgggggtaattataattatctttaccggctgttcagaaaaagcaatttgaaacccttcgaaagggacatatccaccgagatctatcaggccgggtccactccatgcaatggtgtggaaggttttaattgctacttcccattgcagtcttatggattccaaccaaccaatggcgtaggctaccagccgtatcgcgttgtcgtgctcagcttcgagctgctccacgcccccgcgaccgtatgcggtcctaagaagtccaccaatcttgttaagaacaagtgtgtaaactttaactttaacgggctgaccgggaccggcgttctgactgaatctaacaaaaaattcctgcctttccagcagttcggccgcgatattgctgacaccactgacgctgtaagagaccctcagacccttgaaattctcgatatcacaccttgcagctttgggggcgtgtccgtcatcactccaggaactaacacaagcaaccaggtggcagtgttgtaccaggatgttaattgtaccgaggtgccagtggccatccacgccgatcaattgacacctacctggagggtttacagcacagggtccaatgtttttcagacaagagccggatgtctgatcggtgccgagcatgtcaacaattcctacgagtgtgatatccccattggtgcgggaatttgtgcatcatatcagacccagactaatagcccaagaagagctagatccgtcgctagtcaatccatcattgcatatacaatgatgtccgataacggcccccagaatcagagaaacgctccccgcatcacgttcggcggaccaagtgacagcacaggcagtaaccagaacggagaacgctccggtgctcgctccaagcagcgacggccgcaagggcttcccaacaataccgccagctggtttacggctctgacccaacacgggaaagaagatcttaaattccccaggggccagggcgtccctatcaatactaactccagcccggatgatcagataggctactatagacgcgctacccgacggatacgagggggggacggcaaaatgaaggacctttccccccggtggtatttctattacttgggcaccggaccagaagccggactgccttacggcgctaacaaagacggaataatctgggttgcgacggagggcgccctgaatacacctaaagaccatatcggcacaagaaatcctgctaacaatgccgcgattgtgctccagctgcctcagggaaccacgctgcctaaagggttttacgctgaggggtcaaggggggggagtcaagcgtctagtaggtcatcctctcgctctcgcaatagttcccggaactcaaccccaggcagcagcagaggaacctctcccgcacggatggctggcaatgggggagatgctgcccttgctctccttctgctggatcgccttaaccagctcgaatcaaagatgtctggaaaaggtcagcagcagcaaggccagaccgtgacaaagaagagtgcagctgaagctagtaaaaagccacgccaaaaacggaccgcaactaaggcatataacgtaacacaggccttcggcagaagaggtccagaacaaacacagggaaactttggcgatcaagagctgattagacagggcacagattacaaacactggccacagatcgcgcagtttgcaccaagcgcctctgcattcttcgggatgagtcggattgggatggaagtcactccatccgggacctggcttacctacacaggggcaataaaactcgacgacaaagacccaaactttaaagatcaggtcatcctgctgaataaacacatcgatgcctacaaaactttccccccaaccgaaccaaagaaagacaagaaaaaaaaggcagacgaaacgcaagcgctccctcagcgccagaagaagcagcagaccgttacactgttgccagcagcagatctggatgatttttccaagcagcttcaacagagtatgtcaagcgctgacagcactcaggcttgacgatcggatatcgctagcgtaccggcggccgccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccaaagcttacgcgttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatatcgggccactgcaggaaacgatatgggctgaatacggatccgtattcagcccatatcgtttctctagaaataaaatatctttattttcattacatctgtgtgttggttttttgtgtgaatcgatagtactaacatacgctctccatctcgagcctaagcttgtcgactcgaagatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaatgtcaggtcgcctaagtcgattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatatt

SEQ ID NO: 12 - protein, 1116 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 13 - DNA, 46 bp, Artificial Sequence
  Description of Artificial Sequence Synthetic oligonucleotide
gaaacgatatgggctgaatacttaagtattcagcccatatcgtttc

SEQ ID NO: 14 - DNA, 1190 bp, Artificial Sequence
  Description of Artificial Sequence Synthetic polynucleotide
cgggccccccctcgaggtcgacggtatcgataagcttgatatcgaattcgcccttagatatcgtcgacgcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagacatcgatgatatctaaagggcgaattcctgcagcccgggggatccactagtctagatgcatgctcgagcggccgccagtgtgatggatatctgcagaattcgcccttcagctgcggatccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgacggtatcgataagcttgatatcgaattcctgcagcccgggggatccactagtttctagaaataaaatatctttattttcattacatctgtgtgttggttttttgtgtggcggccgccaccgcggtggagctatcgaattcaagcttgtcgactcgaagatcctagactagtggatcccccgggctgcaggaattcgccctttagatatcatcgatgtctcggcggtggtggcgcgtcgcgccgctgggttttatagggcgccgccgcggccgctcgagccataaaaggcaactttcggaacggcgcacgctgattggccccgcgccgctcactcaccggcttcgccgcacagtgcagcatttttttaccccctctcccctccttttgcgaaaaaaaaaaagagcgagagcgagattgaggaagaggaggagggagagttttggcgttggccgccttggggtgctgggcgtcgacgatatctaagggcgaattcgatatcaagcttatcgataccgtcgacctcgagggggggcccg

SEQ ID NO: 15 - DNA, 1757 bp, Artificial Sequence
  Description of Artificial Sequence Synthetic polynucleotide
cgggccccccctcgaggtcgacggtatcgataagcttgatatcgaattcgcccttagatatcgtcgacgcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagacatcgatgatatctaaagggcgaattcctgcagcccgggggatccactagtctagatgcatgctcgagcggccgccagtgtgatggatatctgcagaattcgcccttcagctgcggatccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgacggtatcgataagcttgatatcgaattcctgcagcccgggggatccactagtttctagaaataaaatatctttattttcattacatctgtgtgttggttttttgtgtggcggccgccaccgcggtggagctatcgaattcaagcttgtcgactcgaagatcgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactggtaccgggccccccctcgaggtcgacggtatcgataagcttgatatcgaattcgcccttagatatcgtcgacgcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagacatcgatgatatctaaagggcgaattcctgcagcccgggggatccactagtctagaactagtggatcccccgggctgcaggaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggatccgcagctgaagggcgaattctgcagatatccatcacactggcggccgctcgagcatgcatctagaaataaaatatctttattttcattacatctgtgtgttggttttttgtgtggcggccgccaccgcggtggagcta

SEQ ID NO: 16 - DNA, 46 bp, Artificial Sequence
  Description of Artificial Sequence Synthetic oligonucleotide
gatggtgcttcaagctagtacttaagtactagcttgaagcaccatc

SEQ ID NO: 17 - DNA, 48 bp, Artificial Sequence
  Description of Artificial Sequence Synthetic oligonucleotide
gatggtgcttcaagctagtacggatccgtactagcttgaagcaccatc

SEQ ID NO: 18 - DNA, 48 bp, Artificial Sequence
  Description of Artificial Sequence Synthetic oligonucleotide
gaaacgatatgggctgaatacggatccgtattcagcccatatcgtttc

SEQ ID NO: 19 - DNA, 46 bp, Artificial Sequence
  Description of Artificial Sequence Synthetic oligonucleotide
cctaataattatcaaaatgtggatccacattttgataattattagg

SEQ ID NO: 20 - DNA, 44 bp, Artificial Sequence
  Description of Artificial Sequence Synthetic oligonucleotide
cctaataattatcaaaatgtaattacattttgataattattagg

SEQ ID NO: 21 - protein, 1270 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 22 - protein, 1270 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 23 - protein, 3813 aa, Artificial Sequence
  Description of Artificial Sequence Synthetic polypeptide
ATGTTTGTTTTCTTGGTGCTCCTTCCACTGGTGTCCTCCCAGTGTGTGAACCTCACCACTCGCACGCAACTGCCCCCAGCCTACACGAACTCTTTCACCCGAGGGGTATATTACCCTGATAAAGTCTTCAGGTCCTCTGTTCTCCATAGCACTCAGGATCTTTTCCTGCCTTTCTTTAGCAACGTAACTTGGTTTCACGCAATTTCAGGCACGAACGGCACGAAGAGATTTGATAACCCAGTGCTCCCTTTCAATGACGGCGTTTACTTCGCTTCTACCGAGAAAAGCAACATCATCAGGGGATGGATTTTCGGGACAACATTGGATTCAAAGACCCAGAGCCTTCTCATTGTGAATAATGCCACGAACGTGGTAATCAAGGTGTGCGAGTTCCAGTTCTGCAACGACCCATTCCTTGGAGTTTACCACAAAAACAATAAGAGCTGGATGGAGAGCGAGTTTAGAGTGTATAGCAGCGCCAACAACTGCACCTTTGAGTACGTTAGCCAACCCTTTCTGATGGACCTGGAGGGCAAACAGGGAAACTTTAAGAACCTCCGCGAATTCGTTTTCAAGAACATTGATGGGTATTTTAAGATATACTCTAAGCATACCCCTATTAACTTGGTGCGAGATCTTCCTCAGGGGTTCAGCGCGCTCGAACCTCTCGTGGACTTGCCTATTGGTATCAACATCACCCGATTCCAGACCCTGCTGGCTCTGCACAGGTCATACTTGACTCCCGGCGATTCATCCAGCGGATGGACTGCGGGTGCCGCCGCATATTATGTGGGCTACTTGCAGCCACGAACCTTTCTTTTGAAATATAACGAGAATGGCACAATCACCGACGCCGTTGATTGCGCCCTGGATCCCCTTTCCGAGACGAAGTGTACGCTGAAGTCTTTCACAGTGGAAAAGGGAATCTACCAGACATCAAACTTCCGCGTCCAACCTACCGAGTCAATAGTGCGCTTCCCAAATATCACCAATCTCTGCCCCTTTGGGGAAGTGTTTAATGCCACCCGGTTCGCTTCTGTCTATGCCTGGAACAGGAAGCGCATTTCAAACTGCGTTGCTGACTATTCCGTGCTGTATAACTCTGCAAGCTTTTCTACCTTTAAGTGCTATGGTGTTAGTCCGACAAAACTGAATGATCTGTGCTTTACCAACGTTTACGCCGACTCATTCGTGATTCGAGGAGATGAGGTCAGACAAATTGCTCCTGGGCAGACCGGCAAAATCGCCGACTACAACTATAAGTTGCCTGATGACTTCACCGGCTGCGTGATTGCCTGGAACTCTAACAACCTTGATTCTAAAGTCGGAGGGAACTATAATTACCTCTATCGCCTCTTTAGAAAGTCTAATCTGAAGCCGTTTGAGAGAGATATCTCTACGGAAATATACCAGGCCGGATCAACTCCTTGTAACGGCGTAGAGGGCTTCAACTGCTATTTTCCACTGCAATCCTACGGGTTCCAACCTACTTACGGAGTGGGCTATCAACCCTACAGGGTTGTGGTGCTGTCATTTGAGCTGCTCCACGCACCTGCTACCGTGTGCGGTCCCAAGAAGTCAACCAACCTGGTCAAGAACAAGTGCGTGAATTTCAATTTTAACGGTCTGACCGGAACAGGGGTGCTCACAGAGTCAAACAAGAAGTTTCTGCCCTTCCAGCAGTTCGGGCGCGATATTGATGACACTACCGACGCAGTTCGCGACCCTCAGACTCTTGAGATTCTTGATATCACTCCCTGCAGCTTTGGGGGGGTTTCAGTCATCACCCCAGGCACTAACACATCCAATCAAGTGGCCGTGCTGTATCAGGGAGTCAATTGCACCGAGGTCCCAGTGGCAATCCACGCGGATCAACTCACACCAACATGGAGAGTGTACAGCACCGGGTCTAATGTGTTCCAAACTAGAGCCGGTTGTCTCATTGGCGCAGAACACGTGAACAACAGCTACGAGTGCGACATTCCAATTGGAGCCGGAATCTGTGCCTCTTACCAAACACAGACCAACTCCCACAGACGGGCTCGATCTGTGGCCTCCCAAAGCATTATCGCTTACACGATGAGTCTGGGTGCAGAGAATAGCGTTGCCTACAGTAATAATTCTATCGCTATACCTATCAATTTCACCATCTCTGTAACCACTGAGATCCTTCCAGTCAGTATGACGAAGACTTCTGTTGACTGCACAATGTATATATGCGGAGATTCCACAGAGTGCAGTAACCTGCTGCTGCAATATGGCTCCTTTTGCACTCAGCTCAATAGGGCCCTTACAGGAATCGCCGTGGAGCAAGACAAGAATACTCAAGAAGTCTTCGCGCAGGTGAAGCAGATCTACAAAACGCCTCCTATAAAAGACTTTGGCGGGTTCAATTTTTCCCAGATCTTGCCTGATCCCTCAAAACCCTCTAAGAGATCCTTCATCGAGGATCTTCTGTTTAATAAGGTCACCCTGGCAGACGCAGGCTTCATTAAGCAGTACGGAGACTGCCTCGGGGACATCGCTGCAAGAGACCTTATTTGTGCCCAGAAGTTTAATGGACTCACCGTACTTCCACCACTGCTCACAGATGAGATGATTGCACAGTACACCTCTGCCCTGCTTGCCGGCACTATCACCAGCGGCTGGACTTTCGGAGCCGGAGCTGCCCTTCAGATCCCTTTCGCCATGCAGATGGCATATAGATTCAACGGGATCGGAGTCACCCAGAACGTGCTTTACGAAAATCAGAAACTGATCGCGAACCAATTCAACAGTGCCATCGGCAAGATCCAGGATAGCCTCTCATCCACTGCGAGTGCGTTGGGGAAACTGCAAGATGTGGTCAATCAGAATGCGCAGGCCCTCAACACGCTGGTGAAGCAGCTCTCCTCTAATTTCGGGGCTATCAGCTCTGTTCTGAACGATATCTTGGCTAGACTGGATAAGGTGGAGGCTGAAGTTCAGATTGATAGATTGATTACTGGCCGGCTGCAGTCACTCCAGACTTATGTTACGCAGCAGTTGATTCGCGCTGCGGAGATACGGGCCTCAGCTAATCTTGCCGCTACTAAGATGTCCGAGTGTGTGTTGGGGCAATCCAAACGCGTGGATTTCTGCGGCAAAGGTTACCATCTTATGTCATTCCCCCAGAGCGCCCCTCACGGAGTGGTTTTTCTCCATGTGACATATGTCCCAGCCCAGGAAAAAAATTTTACCACAGCCCCAGCTATATGCCACGACGGCAAAGCTCACTTTCCTCGCGAGGGGGTCTTCGTATCCAACGGCACACACTGGTTTGTAACCCAGAGGAATTTCTACGAACCGCAGATCATCACAACTCATAACACGTTTGTTTCCGGTAATTGTGATGTAGTAATCGGCATCGTTAATAATACAGTGTATGATCCTCTTCAACCCGAACTGGATAGCTTCAAGGAGGAACTCGATAAGTACTTCAAGAATCACACTTCTCCTGACGTGGACCTTGGTGATATATCCGGCATAAATGCTAGTGTGGTGAACATCCAAAAAGAGATAGACAGGCTCAATGAGGTTGCTAAGAATTTGAACGAATCTCTTATCGACCTCCAGGAGCTCGGCAAGTACGAGCAGTATATTAAGTGGCCTTGGTACATCTGGCTGGGTTTCATCGCTGGCTTGATAGCAATCGTAATGGTCACCATTATGTTGTGCTGCATGACTTCCTGTTGTAGCTGTCTCAAAGGGTGTTGCAGCTGTGGCTCATGCTGCAAATTTGACGAAGATGACTCTGAACCAGTCCTCAAGGGCGTCAAGCTTCACTACACGTGA

SEQ ID NO: 24 - protein, 27 aa, Escherichia virus T4
GYIPEAPRDGQAYVRKDGEWVLLSTFL